(19) United States
(12) Patent Application Publication
(10) Pub. No.: US 2009/0305321 A1
(43) Pub. Date: Dec. 10, 2009

(54) Assays for Allosteric Modulators of G-Protein Coupled Receptors (GPCRs)

(75) Inventors: Caroline Grahames, Leicestershire (GB); Philip Mallinder, Leicestershire (GB); Fraser McIntosh, Leicestershire (GB); Nicholas Tomkinson, Leicestershire (GB); Tracey Wright, Leicestershire (GB)

Correspondence Address: FISH & RICHARDSON P.C., P.O BOX 1022, MINNEAPOLIS, MN, 55440-1022, US

(73) Assignee: ASTRAZENECA AB, Sodertalje (SE)

(21) Appl. No.: 11/909,451

(22) PCT Filed: Mar. 22, 2006
(86) PCT No.: PCT/SE06/00355

§ 371 Date: Jul. 27, 2009

(30) Foreign Application Priority Data
Mar. 23, 2005 (SE) 0500668-9

(51) Int. Cl. C12Q 1/02; G01N 33/68; G01N 33/53

(52) U.S. Cl. 435/29; 436/89; 436/501; 436/86

(57) ABSTRACT

The present invention relates to G protein-coupled receptors (GPCRs) and allosteric modulators thereof. More specifically, the invention relates to allosteric modulators of GPCRs that interact at an intracellular binding site. It also relates to methods for designing or identifying small molecule allosteric modulators, including assays (such as competitive binding assays) and methods employing a homology model for the GPCR intracellular site.

The present invention relates to G protein-coupled receptors (GPCRs) and allosteric modulators thereof. More specifically, the invention relates to allosteric modulators of GPCRs that interact at an intracellular binding site, and methods for designing or identifying small molecule allosteric modulators.

### BACKGROUND TO THE INVENTION

G protein-coupled receptors (GPCRs) from all species have been characterised based on sequence homologies (Kolakowski, L. F., 1994, Gcrdb-a G-protein-coupled receptor database, Recept. Channels 2, 1-7). These are defined as class A—Rhodopsin-like, Class B—Secretin-like, Class C—metabotropic glutamate/pheromone type receptors, Class D—is fungal pheromone receptors, Class E—cAMP receptors (Dictyostelium), and finally the Frizzled/smoothened family members. Further information can be obtained from the G Protein-Coupled Receptor Data Base (http://www.gpcr.org/7tm/htmls/consortium.html).

Class A GPCRs are classed as rhodopsin-like GPCRs. They contain the following family members: Amine receptors (eg Muscarinic acetylcholine, Adrenoceptors, Dopamine receptors, Histamine and Serotonin receptors); Peptide receptors (eg angiotensin receptors, Chemokine receptors, melanocortin receptors); Hormone protein receptors; Rhodopsin receptors; Olfactory receptors; Prostanoid receptors; Nucleotide like receptors; Cannabinoid receptors; Platelet activating factor receptor; Gonadotrophin-releasing hormone receptors; Thyrotropin receptors; Melatonin receptors; Viral receptor; Lysosphingolipid receptors; Leukotriene B4 receptor; Class A orphan GPCRs where no ligand has been identified.

The Chemokine receptors (and their ligands) are discussed in more detail below, as an example of GPCRs (in particular of Class A GPCRs).

Chemokines play an important role in immune and inflammatory responses in various diseases and disorders, including asthma and allergic diseases, as well as autoimmune pathologies such as rheumatoid arthritis and atherosclerosis. These small secreted molecules are a growing superfamily of 8-14 kDa proteins characterised by a conserved cysteine motif. At the present time, the chemokine superfamily comprises three groups exhibiting characteristic structural motifs, the C—X—C, C—C and C—X₃—C families. The C—X—C and C—C families have sequence similarity and are distinguished from one another on the basis of a single amino acid insertion between the NH-proximal pair of cysteine residues. The C—X₃—C family is distinguished from the other two families on the basis of having a triple amino acid insertion between the NH-proximal pair of cysteine residues.

The C—X—C chemokines include several potent chemoattractants and activators of neutrophils such as interleukin-8 (CXCL8 or IL-8) or CXCL1 (Growth related oncogene-alpha or GROα) and neutrophil-activating peptide 2 (CXCL7 or NAP-2).

The C—C chemokines include potent chemoattractants of monocytes and lymphocytes but not neutrophils. Examples include human monocyte chemotactic proteins 1-3 (MCP-1, MCP-2 and MCP-3), RANTES (Regulated on Activation, Normal T Expressed and Secreted), eotaxin and the macrophage inflammatory proteins 1α and 1β (MIP-1α and MIP-1β).

The C—X₃—C chemokine (also known as fractalkine) is a potent chemoattractant and activator of microglia in the central nervous system (CNS) as well as of monocytes, T cells, NK cells and mast cells.

Studies have demonstrated that the actions of the chemokines are mediated by subfamilies of G protein-coupled receptors (GPCRs), among which are the receptors designated CCR1, CCR2 (including the two splice variants CCR2A and CCR2B), CCR3, CCR4, CCR5, CCR6, CCR7, CCR8, CCR9, CCR10 and CCR11 (for the C—C family); CXCR1, CXCR2, CXCR3, CXCR4 and CXCR5 (for the C—X—C family) and CX₃CR1 for the C—X₃—C family. These receptors represent good targets for drug development since agents which modulate these receptors would be useful in the treatment of disorders and diseases such as those mentioned above.

The C—X—C chemokine IL-8 (CXCL8) is one of the most potent chemoattractants for neutrophils and is produced by many cell types in response to inflammatory stimuli. IL-8 induces angiogenesis, mediates cytokine induced trans-endothelial neutrophil migration and triggers a variety of other effects associated with the inflammatory response. In vivo data indicates that IL-8 induces neutrophil infiltration to the site of inflammation thus causing tissue injury.

The effects of IL-8 are mediated through two receptors CXCR1 and CXCR2. These receptors display distinct ligand specificities. CXCR1 only binds IL-8 with high affinity; however, CXCR2 binds to other chemokines with high affinity such as NAP2 and GROα as well as IL-8.

In animal models of disease such as ARDS and acid induced lung injury anti-IL-8 antibodies have proved beneficial in reducing inflammatory tissue damage and improving survival. In light of this strong animal model in vivo data it has been proposed that inhibition of the action of CXC chemokine neutrophil attractants, such as IL-8, GROalpha and NAP2 by inhibiting binding to their respective receptors, could prove to be beneficial in diseases such as rheumatoid arthritis, COPD, severe asthma, oncology, IBD (inflammatory bowel disease) and psoriasis.

A number of compounds have been described in the literature that can bind to GPCRs including CXCR1 and CXCR2 (see, for example, WO2000/035442, WO2001/025242 and WO2004/052830). In general GPCR-inhibitory compounds are thought to interfere with binding of endogenous ligands at the extracellular receptor domain of the receptor. In vivo, the interaction with GPCR receptors by chemokines is a complex interaction and endogenous ligands are often produced in situ on demand. They might also be subject to rapid and extensive break down, often already at the site of action. Both processes of production and degradation effectively cause transient receptor stimulation. By contrast, a synthetic compound intended to bind to a receptor is often designed to be metabolically stable and may, therefore, lead to a more continuous stimulation or blockade of the receptor.

Recently, it has been recognized that a more controlled and selective “tuning” action on a receptor may be feasible through allosteric modulation and this is reviewed for example by Soujin et al. (Drug Discovery Today, 9, 17, September 2004, p 752-758). “Allosteric” refers to binding sites that are different from the primary substrate or ligand binding sites. Binding of modulators to the allosteric site results in conformational changes which influence receptor function. In such an interaction, the endogenous ligand remains such that the overall pharmacology resembles normal physiology more closely than with the use of synthetic ligands.

However, to date there has been no significant progress in determining the structural features required for allosteric modulation of GPCR activity. Therefore, there remains a need for such information in order to derive models for identifying compounds which can act as allosteric modulators.

### STATEMENT OF INVENTION

This present invention relates to the identification of a binding site for small molecular weight compounds on the intracellular side of CXCR2, a G-protein coupled receptor. Compounds binding CXCR2 at this cytoplasmic site are able to allosterically modify the activity of agonists acting at an extracellular site. By alignment and homology modelling, the intracellular binding site is predicted to be present in GPCRs and, in particular, in all class A GPCRs.

The elucidation of this novel binding site facilitates the identification of specific and potent inhibitory small molecule compounds for therapeutic purposes.

Accordingly, the present invention relates to methods for identifying small molecule allosteric modulators of GPCRs.

For example, the present invention relates to assays for a candidate compound capable of allosterically modulating a GPCR, and to methods employing a homology model for the GPCR intracellular site to identify lead compounds.

Aspects of the invention are presented in the accompanying claims and are further described in the following paragraphs.

Any GPCR may be used in an assay or method according to the invention. The term “GPCR” thus includes any GPCR. In particular, the term “GPCR” includes Class A receptors (or rhodopsin-like receptors) as well as Class B and Class C receptors. Class A receptors include adenosine receptors and muscarinic receptors and peptide receptors (such as Chemokine receptors); Class B receptors include corticotropin-releasing factor I receptors and Class C receptors include metabotropic glutamate receptors and calcium-sensing receptors.

In particular, the term “GPCR” includes Chemokine receptors including the receptors designated CCR1 (also referred to as CMKBR1, CMKR1, CKR-1, HM145, MIP1aR, SCYAR1, CMKR-1), CCR2 (also referred to as CMBR2, CKR2, CCR2A, CCR2B, CKR2A, CKR2B, MCP-1-R, CC-CKR-2, ccr2), CCR2A, CCR2B, CCR3 (also referred to as CKR3, CMKBR3, CC-CKR-3), CCR4 (also referred to as CKR4, KS-5, CMKBR4, ChemR13, CC-CKR-4, MGC88293, HGCN:14099, c-c ckr-4), CCR5 (also referred to as CMKBR5, CKR5, CD195, CKR-5, CCCKR5, CC-CKR-5, ccr5), CCR6 (also referred to as BN-1, CKR6, DCR2, CKRL3, DRY-6, GPR29, CKR-L3, CMKBR6, GPRCY4, STRL22, GPR-CY4, CCR6, DRY6), CCR7 (also referred to as CMKBR7, EBI1, BLR2, CDw197, EVI1, EBI 1), CCR8 (also referred to as CKRL1, CMKBR8, CY6, TER1, CKR-L1, CMKBRL2, GPR-CY6, ChemR1, CKR8), CCR9 (also referred to as GPR28, GPR-9-6, CMKBR9), CCR10 (also referred to as GPR2) and CCR11 (for the C—C family); CXCR1 (also referred to as IL8RA, CMKAR1, CD128, IL8R1, IL8RBA, CDw128a, C—C CKR-1, C—C-CKR-1), CXCR2 (also referred to as IL8RB, IL8R2, CMKAR2, CDw128b), CXCR3 (also referred to as GPR9, IP10, MigR, CD183, Mig-R, CKR-L2, CMKAR3, IP10-R), CXCR4 (also referred to as FB22, HM89, LAP3, LCR1, NPYR, WHIM, LESTR, NPY3R, NPYRL, HSY3RR, NPYY3R, D2S201E), CXCR5 (also referred to as BLR1, CXCR5, MDR15) and CXCR6 (also referred to as BONZO, STRL33, TYMSTR, Bonzo) (for the C—X—C family) and CX₃CR1 (also referred to as CX3CR1, GPR13, V28, CCRL1, CMKDR1, GPRV28, CMKBRL1, C3X1) for the C—X₃—C family and XCR1 (also referred to as GPR5, CCXCR1, CXC1).

Preferred embodiments of any aspect of the invention include each of the following:
i. an assay or method wherein the GPCR is a Class A receptor;
ii. an assay or method wherein the GPCR is a Chemokine receptor
iii. an assay or method wherein the GPCR is selected from the group consisting of CCR1, CCR2, CCR2A, CCR2B, CCR3, CCR4, CCR5, CCR6, CCR7, CCR8, CCR9, CCR10, CCR11, CXCR1, CXCR2, CXCR3, CXCR4, CXCR5 and CX₃CR1;
iv. an assay or method wherein the GPCR is selected from the group consisting of CCR1, CCR2, CX₃CR1, CCR4, CCR5 and CCR7 and a C—X—C family receptor;
v. an assay or method wherein the GPCR is a C—X—C family receptor;
vi. an assay or method wherein the GPCR is selected from the group consisting of CXCR1 and CXCR2;
vii. an assay or method wherein the GPCR is CXCR2.

In a particularly preferred embodiment, the GPCR referred to in any aspect of the present invention is a Chemokine receptor and, preferably is selected from CCR1, CCR2, CX₃CR1, CCR4, CCR5, CCR7 and a C—X—C family receptor. Most preferably, the C—X—C family receptor is the CXCR2 receptor.

### DETAILED DESCRIPTION

### Identification of the Intracellular Binding Site and Generation of the CXCR2Homology Structure

Two approaches have been utilised in obtaining structural information and to generate models for an allosteric intracellular binding site for GPCRs. These include (1) an analysis of structures derived from sequence homology with CXCR2 and bovine rhodopsin and (2) domain swap experiments, where the residues differing in the intracellular region of CXCR2 have been replaced with the corresponding residues of the CXCR1 intracellular region as well as site-directed mutagenesis studies.

With reference to CXCR2, based on alignments with bovine rhodopsin, the intracellular region comprises four intracellular domains: domain 1 residues S67 to D94, domain 2 residues G133 to S173, domain 3 residues I221 to F260 and domain 4 amino acids S307 to L360 (FIG. 1A). Because CXCR2 is a GPCR which has seven transmembrane spanning helices it has four regions which can be defined as intracellular. This can be seen schematically in FIGS. 1B and 1C.

The present invention employs domain swap experiments and site-directed mutagenesis methods in conjunction with the homology modelling approach to identify amino acids within the intracellular region of GPCRs. Based on analysis of the sequence and residues in the intracellular region, it was possible to determine the specific residues involved in an allosteric intracellular binding site and to identify the interactions that could be exploited in the design of compounds which specifically bind the intracellular binding site for each GPCR and inhibit signalling from each receptor.

The residues involved in compound binding in a variety of CXCR and CCR molecules are compared in FIGS. 18, 19 and 20. As can be seen, one key amino acid is residue Lysine320 (K320) of CXCR2. This residue is in the last intracellular domain in the C-terminal portion. Lysine320 (K320) is involved in mediating binding of CXCR2 inhibitors from different series of small molecule antagonists. Compounds that interact with this binding site inhibit the binding of IL-8 to CXCR2 via an allosteric mechanism.

Other important amino acids are found in the other intracellular loops or the intracellular part of helices 2, 3, 6, 7 and 8. Such other residues include:

On the intracellular part of helix 2 residues S81, V82, T83, D84, Y86, L87, L90

On the intracellular part of helix 3 residues G133, L136, L137, I140, D143, R144, A147 (particularly residues G133, L136, I140, D143, R144, A147)

On intracellular loop 2 residue Q157

On intracellular loop 3 residues Q245, K246

On the intracellular part of helix 6 residues A249, V252, I253, V256, I259

On the intracellular part of helix 7 residues L309, N310, P311, I313, Y314, I317, G318,

On helix 8 residues Q319, K320, F321

An alignment of CXCR2 with other receptors (FIG. 19, and FIGS. 18 and 20) shows that corresponding residues, including those corresponding to K320 of CXCR2, are present in the other Chemokine receptors (for example CCR2b, CX₃CR1, CCR4, CCR5 and CCR7).

The intracellular allosteric compound-binding site may enable compounds of a similar series to have significant activity at more than one Chemokine receptor. Furthermore, this intracellular allosteric binding site may be critical for inhibition of GPCRs including Class A GPCRs by small molecule compounds for the treatment of human diseases such as inflammatory disorders, in particular, rheumatoid arthritis, COPD, severe asthma, oncology, IBD (inflammatory bowel disease) and psoriasis.

### Assays for Intracellular Allosteric Modulators

In a first aspect, the invention relates to an assay for a candidate compound capable of allosterically modulating a GPCR, said assay comprising the steps of:
a) contacting said candidate compound with a GPCR or a mutant, variant, homologue, derivative or fragment thereof; and
b) detecting whether said candidate compound forms associations with one or more amino acid residues corresponding to any one of amino acid residues S81, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 or to any one of amino acid residues 301 to 360 of CXCR2.

In a preferred aspect, the invention relates to an assay for a candidate compound capable of allosterically modulating a GPCR, said assay comprising the steps of:
a) contacting said candidate compound with a GPCR or a mutant, variant, homologue, derivative or fragment thereof; and
b) detecting whether said candidate compound forms associations with one or more amino acid residues corresponding to any one of amino acid residues S81, V82, T83, D84, Y86, L87, L90, G133, L136, I140, D143, R144, A147, Q157, Q245, K246, A249, V252, I253, V256, I259, L309, N310, P311, I313, Y314, I317, G318, Q319, K320, F321 or to any one of amino acid residues 301 to 360 of CXCR2.

In another preferred aspect, the invention relates to an assay for a candidate compound capable of allosterically modulating a GPCR, said assay comprising the steps of:
(a) contacting said candidate compound with a GPCR or a mutant, variant, homologue, derivative or fragment thereof; and
(b) detecting whether said candidate compound forms associations with one or more amino acid residues corresponding to any one of amino acid residues S81, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 or to any one of amino acid residues 304 to 326 of CXCR2.

In a particularly preferred aspect, the invention relates to an assay for a candidate compound capable of allosterically modulating a GPCR, said assay comprising the steps of:
(a) contacting said candidate compound with a GPCR or a mutant, variant, homologue, derivative or fragment thereof; and
(b) detecting whether said candidate compound forms associations with one or more amino acid residues corresponding to any one of amino acid residues S81, V82, T83, D84, Y86, L87, L90, G133, L136, I140, D143, R144, A147, Q157, Q245, K246, A249, V252, I253, V256, I259, L309, N310, P311, I313, Y314, I317, G318, Q319, K320, F321 or to any one of amino acid residues 304 to 326 of CXCR2.

In a preferred embodiment, the GPCR in step a) is a fragment comprising amino acid residues corresponding to all or part of residues 304 to 326 and, preferably, residues 301 to 360 of CXCR2. Suitably said fragment may be linked to a molecule to facilitate expression and structural conformation or for detection of a binding reaction. For example, the fragment may be linked to form a GST-fusion protein. Other suitable detection molecules will be familiar to those skilled in the art.

Thus preferred embodiments of the first aspect of the invention include each of the following:
i. an assay wherein the GPCR in step a) is a polypeptide comprising amino acid residues corresponding to all or part of residues 301 to 360 of CXCR2;
ii. an assay wherein the GPCR in step a) is a polypeptide comprising amino acid residues corresponding to residues 301 to 360 of CXCR2;
iii. an assay wherein the GPCR in step a) is a polypeptide comprising amino acid residues corresponding to all or part of residues 304 to 326 of CXCR2;
iv. an assay wherein the GPCR in step a) is a polypeptide comprising amino acid residues corresponding to residues 304 to 326 of CXCR2.

In a preferred embodiment, step b) comprises detecting whether said candidate compound forms associations with amino acids selected from those corresponding to amino acids S81, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 of CXCR2 and, in particular, K320 of CXCR2.

In a particularly preferred embodiment, step b) comprises detecting whether said candidate compound forms associations with amino acids selected from those corresponding to amino acids S81, V82, T83, D84, Y86, L87, L90, G133, L136, I140, D143, R144, A147, Q157, Q245, K246, A249, V252, I253, V256, I259, L309, N310, P311, I313, Y314, I317, G318, Q319, K320, F321 of CXCR2 and, in particular, K320 of CXCR2.

In other particularly preferred embodiments, step b) comprises detecting whether the candidate compound forms associations with one or more amino acid residues corresponding to any one of amino acid residues 301 to 360 of CXCR2 (and most particularly with one or more amino acid residues corresponding to any one of amino acid residues 304 to 326 of CXCR2).

“Amino acid residues corresponding to residues of CXCR2” may be determined by performing alignments of sequences from other GPCRs and, in particular, other Chemokine receptors. Methods for performing such alignments are described herein and are known in the art. For example, an alignment of sequences from Class A GPCRs is available at http://www.gpcr.org/7tm/seq/001/001.html (GPCRDB, the G Protein-Coupled Receptor Data Base).

Suitably, “amino acid residues corresponding to residues of CXCR2” are defined by alignment with bovine rhodopsin as shown in FIG. 19.

For example, the amino acid residues corresponding to amino acid residues S81, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 of CXCR2 or to amino acid residues 301 to 360 of CXCR2 are listed below for specified GPCRs:

Bovine rhodopsin: T70, P71, L72, N73, I75, L76, L79, A124, S127, L128, L131, E134, R135 V138, F148, A246, E247, V250, M253, V254, M257, A260, Y301, N302, P303, I305, Y306, M309, N310, K311, Q312, F313 or amino acid residues 293 to 348.
CKR1: N67, M68, T69, S70, Y72, L73, L76, E120, F123, I124, L127, D130, R131, A134, A144, K233, K234, A237, L240, I241, I244, I247, V296, N297, P298, I300, Y301, V304, G305, E306, R307, F308 or amino acid residues 288 to 355.
CKR2: C75, L76, T77, D78, Y80, L81, L84, G127, F130, I131, L134, D137, R138, A141, A151, K237, R238, A241, V244, I245, I248, V251, I300, N301, P302, I304, Y305, V308, G309, E310, K311, F312 or amino acid residues 292 to 374.
CKR3: 167, M68, T69, N70, Y72, L73, L76, E120, F123, I124, L127, D130, R131, A134, A144, K233, K234, A237, L240, I241, I244, V247, M296, N297, P298, I300, Y301, V304, G305, E306, R307, F308 or amino acid residues 288 to 355.
CKR4: S72, M73, T74, D75, Y77, L78, L81, G124, F127, V128, M131, D134, R135, A138, A148, K236, K237, A240, M243, I244, V247, L250, L299, N300, P301, I303, Y304, L307, G308, E309, K310, F311 or amino acid residues 291 to 360.
CKR5: S63, M64, T65, D66, Y68, L69, L72, G115, F118, I119, L122, D125, R126, A129, A139, K229, R230, A233, L236, I237, I240, V243, I292, N293, P294, I296, Y297, V300, G301, E302, K303, F304 or amino acid residues 284 to 352.
CCR6: S79, M80, T81, D82, Y84, L85, M88, G132, L135, L136, I139, D142, R143, A146, L156, K248, R249, A252, V255, I256, V259, V262, L311, N312, P313, L315, Y316, I319, G320, Q321, K322, F323 or amino acid residues 303 to 374.
CCR7: T91, M92, T93, D94, Y96, L97, L100, G143, L146, L147, I150, D153, R154, A157, H167, E257, R258, A261, V264, I265, V268, V271, V321, N322, P323, L325, Y326, I329, G330, V331, K332, F333 or amino acid residues 313 to 378.
CKR8: S68, I69, T70, D71, Y73, L74, L77, S120, F123, I124, M127, D130, R131, A134, V144, N232, K233, A236, L239, V240, V243, A246, V295, N296, P297, I299, Y300, V303, G304, E305, K306, F307 or amino acid residues 287 to 355.
CCR9: T69, M70, T71, D72, F74, L75, L78, C121, L124, I125, I128, D131, R132, A135, W145, S236, K237, A240, V243, T244, V247, V250, L300, N301, P302, L304, Y305, V308, G309, E310, R311, F312 or amino acid residues 292 to 357.
CCR10: S75, P76, T77, S78, H80, L81, L84, G127, F130, L131, I134, D137, R138, A141, R151, E231, R232, A235, V238, V239, L242, A245, L305, N306, P307, L309, Y310, is L313, G314, L315, R316, F317 or amino acid residues 297 to 362.
CXCR1: S72, V73, T74, D75, Y77, L78, L81, G124, L127, L128, I131, D134, R135, A138, Q148, Q236, K237, A240, V243, I244, V247, I250, L300, N301, P302, I304, Y305, I308, G309, Q310, N311, F312 or amino acid residues 292 to 350.
CXCR3: S86, S87, T88, D89, F91, L92, L95, G138, L141, L142, I145, D148, R149, N152, R162, R249, R250, A253, L256, V257, V260, A263, L313, N314, P315, L317, Y318, V321, G322, V323, K324, F325, or amino acid residues 305 to 368.
CXCR4: S71, M72, T73, D74, Y76, R77, L80, S123, I126, L127, I130, D133, R134, A137, P147, Q233, K234, A237, T240, T241, L244, A247, L297, N298, P299, L301, Y302, L305, G306, A307, K308, F309 or amino acid residues 289 to 352.
CXCR5: S84, S85, T86, E87, F89, L90, L93, S136, L139, L140, I143, D146, R147, A150, H160, Q253, R254, A257, V260, A261, V264, I267, L317, N318, P319, L321, Y322, A325, G326, V327, K328, F329 or amino acid residues 309 to 372.
CXCR6: S64, L65, T66, D67, F69, L70, L73, S16, I119, L120, I123, D126, R127, V130, Q140, Q225, K226, S229, I232, I233, V236, V239, L283, N284, P285, L287, Y288, V291, S292, L293, K294, F295 and residues 275 to 342.
CX₃CR1: S64, V65, T66, D67, Y69, L70, L73, S116, F119, I120, I123, D126, R127, A130, N140, K225, K226, A229, L232, I233, V236, V239, L288, N289, P290, I292, Y293, A296, G297, E298, K299, F300 or amino acid residues 280 to 355.
XCR1: S64, L65, T66, N67, F69, I70, L73, S116, F119, L120, M123, H126, R127, S130, V140, R219, R220, T223, L226, I227, I230, A233, F282, N283, P284, L286, Y287, V290, G291, V292, K293, F294, or amino acid residues 274 to 333.

In an assay according to the first aspect of the invention, detecting whether the candidate compound forms associations with one or more particular amino acid residues may be achieved by suitable methods known in the art. Such methods include, for example: disulfide trapping (for example, as described by Buck E and Wells J A, 2005, PNAS USA 102(8):2719-24; or in Example 10); or photoaffinity labelling with proteomic characterisation (for example, as described by Murray et al, Nature Chemical Biology 2005, 1:371; or in Example 11 which describes a possible photoaffinity labelling assay involving cells expressing a whole GPCR).

In an assay according to the first aspect of the invention wherein the GPCR in step a) is a GPCR fragment consisting of a polypeptide comprising amino acid residues corresponding to all or part of residues 301 to 360 of CXCR2 (such as a polypeptide comprising amino acid residues corresponding to all or part of residues 304 to 326 of CXCR2), detection of any association between the fragment and the candidate compound can be achieved by a competitive binding assay. The methods of producing such an assay system with a polypeptide and probe compound which are suitable for testing in such an assay are well known to those skilled in the art. To form an assay, generally both the polypeptide and probe compound assay components would be tagged or labelled in such a way to enable the detection of binding of one assay component to the other. Such methods may include systems such as SPA, FRET, etc. The ability of unlabelled candidate compounds to inhibit the interaction between polypeptide and probe can then be measured.

A second aspect of the invention relates to a competitive binding assay for a Candidate Compound X capable of allosterically modulating a GPCR which comprises the steps of:

i) providing a GPCR polypeptide comprising amino acid residues corresponding to all or part of residues 301 to 360 of CXCR2;
ii) contacting said polypeptide with a binding agent;
iii) contacting said polypeptide with a Candidate Compound X; and
iv) detecting displacement of the binding agent as an indication of the Candidate Compound X being capable of modulating said GPCR.

Preferred embodiments of the second aspect of the invention include each of the following:
i. a competitive binding assay wherein the polypeptide comprises residues corresponding to residues 301 to 360 of CXCR2;
ii. a competitive binding assay wherein the polypeptide comprises residues corresponding to residues 304 to 326 of CXCR2;
iii. a competitive binding assay wherein the polypeptide comprises residues corresponding to residues 318 to 360 of CXCR2;
iv. a competitive binding assay wherein the binding agent is a Candidate Compound Y identified by an assay according to the first aspect of the invention, or a pharmaceutically acceptable salt thereof;
v. a competitive binding assay wherein the binding agent is selected from the group consisting of Compound A or a pharmaceutically acceptable salt thereof, Compound B or a pharmaceutically acceptable salt thereof, Compound C or a pharmaceutically acceptable salt thereof, and Compound F or a pharmaceutically acceptable salt thereof, wherein Compounds A, B, C and F are as defined herein (we have shown that Compounds A, B, C and F bind at an intracellular allosteric site);
vi. a competitive binding assay wherein the binding agent is selected from the group consisting of Compound A or a pharmaceutically acceptable salt thereof, Compound B or a pharmaceutically acceptable salt thereof, and Compound C or a pharmaceutically acceptable salt thereof, wherein Compounds A, B and C are as defined herein;
vii. a competitive binding assay wherein the binding agent is Compound C or a pharmaceutically acceptable salt thereof, wherein Compound C is as defined herein.

The polypeptide may be provided in any suitable way, as known in the art. Non-limiting examples are given below.

The polypeptide may be provided as an isolated or purified polypeptide in a suitable format for contacting with the binding agent and Candidate Compound X (for example: in a suitable solution; in a suitable plate; on a resin support; etc).

The polypeptide may be provided as an isolated or purified fusion protein, wherein the polypeptide is fused to a suitable carrier protein (for example, GST). The fusion protein is provided in a suitable format for contacting with the binding agent and Candidate Compound X (for example: in a suitable solution; in a suitable plate; on a resin support; etc). Suitably, a fusion protein (such as a GST-fusion protein) includes a polypeptide comprising residues corresponding to residues 301 to 360 of CXCR2, or to residues 304 to 326 of CXCR2, or to residues 318 to 360 of CXCR2. An example of a GST-fusion protein is described in Example 12 (GST fused to the last 43 amino acid residues of human CXCR2, that is residues G318 to L360).

The polypeptide (including a polypeptide provided as a fusion protein) may be expressed in a cell or cell membrane system. This involves providing a cell or cell membrane that is capable of expressing the polypeptide, contacting said cell or cell membrane with the binding agent, and incubating said cell or cell membrane with the Candidate Compound X. In a particular embodiment, the polypeptide (including a polypeptide provided as a fusion protein) may be fused to a target peptide, expressed in a cell and targeted to the cell membrane.

A preferred embodiment of the second aspect of the invention relates to the use of a compound selected from the compound series exemplified by Compounds A, B or C, as described herein, or a pharmaceutically acceptable salt thereof, in an assay for identifying candidate compounds capable of selectively modulating a GPCR. Suitably said selective modulation is through binding at an intracellular allosteric site as identified herein. Preferably, the competitive binding assay comprises the steps of:

i) providing a cell or cell membrane that is capable of expressing a GPCR polypeptide comprising amino acid residues corresponding to all or part of residues 301 to 360 of CXCR2;
ii) contacting said cell or cell membrane with Compound C;
iii) incubating said cell or cell membrane with a Candidate Compound X; and
iv) detecting displacement of Compound C as an indication of a Candidate Compound X being capable of selectively modulating said GPCR.

Suitably said Compound C is detectably labelled and, preferably, radiolabelled (for example with tritium or ¹⁴C).

In a preferred embodiment of the first or second aspects of the invention, the assay is a membrane assay. Suitable membrane assays are described herein.

In an alternative embodiment of any aspect of the invention, the assay is a whole cell assay. Suitable whole cell assays are described herein.

Advantageously, the compound binds to an intracellular binding site. By definition if a compound binds to an intracellular binding site of a GPCR it can not be competing at the same site with the endogenous ligand. This could be an advantage in that the degree of inhibition observed with the compound may not be influenced by the quantity of endogenous ligand on the extracellular surface.

Furthermore, CXCR2 and some other Chemokine receptors are “promiscuous” that is they have more than one ligand. In the case of CXCR2, for example, the ligands include IL8, NAP2 and GROalpha. Each ligand has a slightly different binding site on the extracellular part of the receptor. By binding to a single intracellular allosteric site the compound is able to prevent the signalling by all ligands on that receptor.

As can be seen in the alignment with other GPCRs (FIG. 19), the residues in the intracellular binding pocket tend to be more conserved than the residues on extracellular portions of the receptor which are involved in direct ligand binding. This could give the advantage that a small molecule inhibitor binding at a conserved intracellular site would be able to inhibit more than one Chemokine receptor such as one of CXCR1, CCR1, CCR2, CCR4, CCR5, CX₃CR1, CCR7 as well as CXCR2. Advantageously, binding to an intracellular site gives an alternative binding site to target in all GPCRs where targeting the retinal binding pocket has proved unsuccessful, for example where there is either lack of active chemical hits or where active compounds have unwanted activities against other proteins.

### Methods Employing a Homology Model for the GPCR Intracellular Site

As used herein, the term “model” refers to a structural model such as a three dimensional (3D) structural model (or representation thereof) comprising a GPCR such as CXCR2. Preferably, the model comprising a GPCR such as CXCR2 is built from the co-ordinates of the bovine rhodopsin crystal structure. An example of the model for CXCR2 thus generated has the structure co-ordinates presented in Table 3. The homology model of the invention enables candidate compounds to be identified that bind spatially and preferentially to a GPCR such as CXCR2, particularly to the intracellular binding site of a GPCR such as CXCR2.

As described herein, a homology model of a GPCR can be derived from comparison and modelling the crystal structure of a related GPCR such as rhodopsin. It will be recognised by those skilled in the art that a number of suitable homology models may be generated from any suitable starting structure. The present application describes one such homology model although the present invention also applies to other models sharing the same common defining features. Thus, as used herein, the term “model” is not limited to the structural model having the structure co-ordinates presented in Table 3. The homology model to be used in methods of the invention may be any suitable structural model comprising a GPCR. The skilled person will be able to generate a range of suitable structural models.

Accordingly, in a third aspect of the invention, there is provided a method of screening for an allosteric modulator of a GPCR, wherein the method comprises using a structure having co-ordinates corresponding to those set out in Table 3 or to those co-ordinates of a similar model derived in a similar way.

Preferably said method comprises using the structure co-ordinates of Table 3.

Suitably, said method comprises the steps of:
(a) providing at least a portion of the structure co-ordinates of Table 3;
(b) employing at least a portion of the structure co-ordinates of Table 3 to design or select or synthesise a putative allosteric modulator of a GPCR;
(c) contacting the putative allosteric modulator of a GPCR with a GPCR or a mutant, variant, homologue, derivative or fragment thereof; and
(d) determining whether said putative allosteric modulator of a GPCR modulates said GPCR.

In a preferred embodiment, at least a portion of the structure co-ordinates of Table 3 and/or the putative allosteric modulator of a GPCR and/or the substrate are provided on a machine-readable data storage medium comprising a data storage material encoded with machine readable data.

In another embodiment, the putative allosteric modulator of a GPCR is from a library of compounds. Preferably, the library is an in silico library. Suitable in silico libraries will be familiar to those skilled in the art, and include the Available Chemical Directory (MDL Inc), the Derwent World Drug Index (WDI), BioByteMasterFile, the National Cancer Institute database (NCI), and the Maybridge catalogue.

In another embodiment, the putative allosteric modulator of a GPCR is selected from a database, designed de novo or designed from a known GPCR modulator.

Suitably, the design or selection of the putative allosteric modulator of a GPCR is performed in conjunction with computer modelling.

In a preferred embodiment, the allosteric modulator of a GPCR inhibits GPCR activity.

Methods for monitoring GPCR activity will be familiar to those skilled in the art. “GPCR activity” includes GPCR signalling. In particular, and with reference to CXCR2, methods for determining activity include measuring cell calcium flux. Furthermore, the allosteric modulation of a GPCR can be determined by measuring the binding of the receptor ligand in the presence or absence of the candidate compound. In particular, the ability of a compound to allosterically modulate CXCR2 can be detected by measuring the binding of is a CXCR2 ligand in the presence or absence of the candidate compound. Suitable CXCR2 ligands include IL-8.

Suitably, the method in accordance with any embodiment of the third aspect of the invention provides an allosteric modulator of a GPCR which is useful in the prevention and/or treatment of a GPCR-related disorder, condition or disease in human and non-human animals.

In a preferred embodiment, where the GPCR is a Chemokine receptor the GPCR related disorder is an inflammatory disorder such as rheumatoid arthritis, COPD, severe asthma, oncology, IBD (inflammatory bowel disease) or psoriasis.

Another aspect of the invention relates to a computer for producing a three-dimensional representation of a GPCR wherein said computer comprises:
a) a computer-readable data storage medium comprising a data storage material encoded with computer-readable data, wherein said data comprises the structure co-ordinates of Table 3 or to those co-ordinates of a similar model derived in a similar way;
b) a working memory for storing instructions for processing said computer-readable data;
c) a central-processing unit coupled to said working memory and to said computer-readable data storage medium for processing said computer-machine readable data into said three-dimensional representation; and
d) a display coupled to said central-processing unit for displaying said three-dimensional representation.

Another aspect of the invention relates to a machine-readable data storage medium comprising a data storage material encoded with machine-readable data, wherein the data is defined by at least a portion of the structure co-ordinates of Table 3 or to those co-ordinates of a similar model derived in a similar way.

A further aspect of the invention relates to the use of the above-described computer or machine-readable data storage medium to predict the structure and/or function of potential allosteric modulators of a GPCR.

Another aspect relates to the use of at least a portion of the structure co-ordinates of Table 3 or a similar GPCR-specific model based on the structure of bovine rhodopsin or any other suitable starting point to screen for allosteric modulators of a GPCR. Suitably, in a preferred embodiment of any aspect of the invention, said portion corresponds to the amino acids which define the intracellular region of said GPCR. With reference to CXCR2, based on alignments with bovine rhodopsin, the intracellular region suitably comprises four intracellular domains; domain 1 residues S67 to D94, domain 2 residues G133 to S173, domain 3 residues I221 to F260 and domain 4 amino acids S307 to L360 (FIG. 1A).

In particular, the portion employed to design or select a putative allosteric modulator corresponds to the amino acids which define the intracellular region of the GPCR.

According to the third aspect of the invention, there is provided a method of designing or screening for an intracellular allosteric modulator of a GPCR comprising the steps of:
a) providing at least a portion of the structure co-ordinates of the GPCR corresponding to those set out in Table 3;
b) employing at least a portion of the structure co-ordinates corresponding to those set out in Table 3 to design or select a putative allosteric modulator of the GPCR, wherein the portion employed corresponds to the amino acids which define the intracellular region of the GPCR;
c) obtaining or synthesising the putative allosteric modulator of the GPCR;
d) contacting the putative allosteric modulator of the GPCR with the GPCR or a mutant, variant, homologue, derivative or fragment thereof; and
e) determining whether said putative allosteric modulator of the GPCR modulates said GPCR.

Preferably, the putative allosteric modulator interacts with any one or more amino acid residues corresponding to any one of amino acid residues S81, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 or to any one of amino acid residues 301 to 360 of CXCR2.

More preferably, the putative allosteric modulator interacts with any one or more amino acid residues corresponding to any one of amino acid residues S81, V82, T83, D84, Y86, L87, L90, G133, L136, I140, D143, R144, A147, Q157, Q245, K246, A249, V252, I253, V256, I259, L309, N310, P311, I313, Y314, I317, G318, Q319, K320, F321 or to any one of amino acid residues 301 to 360 of CXCR2.

As a preferred embodiment, the putative allosteric modulator interacts with any one or more amino acid residues corresponding to any one of amino acid residues S8 1, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, or F321 of CXCR2, and most preferably with an amino acid residue corresponding to K320 of CXCR2.

As another preferred embodiment, the putative allosteric modulator interacts with any one or more amino acid residues corresponding to any one of amino acid residues S81, V82, T83, D84, Y86, L87, L90, G133, L136, I140, D143, R144, A147, Q157, Q245, K246, A249, V252, I253, V256, I259, L309, N310, P311, I313, Y314, I317, G318, Q319, K320, or F321 of CXCR2, and most preferably with an amino acid residue corresponding to K320 of CXCR2.

As another preferred embodiment, the putative allosteric modulator interacts with any one or more amino acid residues corresponding to any one of amino acid residues 301 to 360 of CXCR2, more preferably with any one or more amino acid residues corresponding to any one of amino acid residues 304 to 326 of CXCR2, and most preferably with an amino acid residue corresponding to K320 of CXCR2.

A further aspect relates to the use of at least a portion of the structure co-ordinates of Table 3 to solve the structure of the crystalline form of any other protein with significant amino acid sequence homology to an intracellular allosteric domain of a GPCR.

Preferably, the structure of the crystalline form of any other protein with significant amino acid sequence homology to an intracellular allosteric domain of a GPCR is solved using molecular replacement.

Yet another aspect of the invention relates to the use of at least a portion of the structure co-ordinates of Table 3 in molecular design techniques to design, select and synthesise modulators of a GPCR which bind to an intracellular allosteric domain.

Another aspect of the invention relates to the use of at least a portion of the structure co-ordinates of Table 3 to screen small molecule databases for chemical entities or compounds that modulate a GPCR.

Preferably, the modulator of a GPCR, chemical entity, substrate or compound selectively inhibits a GPCR.

### GPCR Modulators

A further aspect of the invention relates to a GPCR modulator identified by the above-described methods, or a candidate compound identified by the above-described assays.

Preferably, the GPCR modulator or candidate compound of the invention inhibits GPCR activity.

More preferably, the GPCR modulator or candidate compound of the invention selectively inhibits a GPCR through an allosteric interaction in the intracellular domain of said GPCR. In particular, the GPCR modulator specifically binds the intracellular binding site identified herein and thus inhibits signalling from the receptor.

In another preferred embodiment, the GPCR modulator or candidate compound of the invention comprises a functional group capable of forming an interaction such as a charged electrostatic interaction or hydrogen bond with the amino acid residue corresponding to K320 of CXCR2.

In one preferred embodiment, the GPCR modulator or candidate compound of the invention is capable of forming associations with one or more amino acid residues corresponding to: on the intracellular part of helix 2 residues S81, V82, T83, D84, Y86, L87, L90; on the intracellular part of helix 3 residues G133, L136, L137, I140, D143, R144, A147 (particularly residues G133, L136, I140, D143, R144, A147); on Intracellular loop 2 residue Q157; on Intracellular loop 3 residues Q245, K246; on the intracellular part of helix 6 residues A249, V252, I253, V256, I259; on the intracellular part of helix 7 residues L309, N310, P311, I313, Y314, I317, G318, or on helix 8 residues Q319, K320, F321 of CXCR2 or the equivalent residues in all Chemokine receptors or Class A GPCRs, as determined by their alignment with the bovine rhodopsin structure (see FIG. 19).

In a preferred embodiment, said GPCR modulator is capable of forming associations with one or more amino acid residues corresponding to amino acids 304 to 326 of CXCR2.

In another preferred embodiment, said GPCR modulator is capable of forming associations with one or more amino acid residues corresponding to amino acids 301 to 366 of CXCR2.

Suitably said GPCR modulator is capable of sterically interacting with said residues to induce an allosteric modulation of said GPCR.

In a preferred embodiment, the GPCR modulator or candidate compound of the invention is an allosteric modulator.

The present invention permits the use of molecular design techniques to design, select and synthesise chemical entities and compounds, including GPCR modulating compounds, capable of binding to an intracellular binding site of a GPCR, in whole or in part.

Suitably, molecular design can exploit the sequence and structural information of the active site by fragment based screening. The sequence information in conjunction with structural knowledge can be used either manually or computationally (using docking programs such as LUDI, GLIDE, DOCK, GOLD or FRED) to suggest small molecular is weight fragments for NMR or high concentration screening. The same approach can also be used for reagent selection by a reagent-based or product-based approach for library synthesis and screening.

Other suitable approaches include Charlie (Tripos), which may be used to connect fragments together. De Novo approaches may include Sprout (Peter Johnston, Sheffield) or Rachel (Tripos).

Small molecule databases or candidate compounds may be screened for chemical entities or compounds that can bind in whole, or in part, to an intracellular binding site of a GPCR. Thus, in a preferred embodiment, the putative GPCR modulator is from a library of compounds or a database. In this screening, the quality of fit of such entities or compounds to the binding site may be judged by various methods—such as shape complementarity or estimated interaction energy (Meng, E. C. et al., J. Comp. Chem., 13, pp. 505-524 (1992)).

The structure co-ordinates of Table 3, or portions thereof, may also be useful in solving the structure of crystal forms of the intracellular binding site of homologous GPCRs. They may also be used to solve the structure of GPCR mutants, GPCR variants, GPCR homologues, GPCR derivatives, GPCR fragments and GPCR complexes. Suitable GPCR homologues are described herein and include, in particular, the Chemokine receptors, for example, molecular replacement may be used.

In a preferred embodiment of the present invention, the GPCR crystal of unknown structure further comprises an entity bound to the GPCR protein or a portion thereof, for example, an entity that is an allosteric inhibitor of the GPCR.

The crystal structures of such complexes may be solved by molecular replacement or in combination with MAD (Multiwavelength Anomalous Dispersion) and/or MIRAS (Multiple Isomorphous Replacement with Anomalous Scattering) procedures—and compared with that of the wild-type GPCR. Potential sites for modification within the intracellular binding site of the enzyme may thus be identified. This information provides is an additional tool for determining the most efficient binding interactions, for example, increased hydrophobic interactions, between a GPCR and a chemical entity or compound.

The structures and complexes of the GPCR may be refined using computer software—such as X-PLOR (Meth. Enzymol., vol. 114 & 115, H. W. Wyckoff et al., eds., Academic Press (1985)), MLPHARE (Collaborative computational project Number 4. The CCP4 Suite: Programs for Protein Crystallography (1994) Acta Crystallogr. D 50, 760-763) and SHARP [De La Fortelle, E. & Bricogne, G. Maximum-likelihood heavy-atom parameters refinement in the MIR and MAD methods (1997) Methods Enzymol. 276, 472-494). Preferably, the complexes are refined using the program CNS (Brünger et al. (1998) Acta Crystallogr. D 54, 905-921). During the final stages of refinement water molecules, ions and inhibitor molecules may be inserted in the structure. This information may thus be used to optimise known classes of GPCR modulators, e.g. GPCR inhibitors, and more importantly, to design and synthesise novel classes of GPCR allosteric modulators.

The overall figure of merit may be improved by iterative solvent flattening, phase combination and phase extension with the program SOLOMON [Abrahams, J. P. & Leslie, A. G. W. Methods used in structure determination of bovine mitochondrial F1 ATPase. (1996) Acta Crystallogr. D 52, 110-119].

The structure co-ordinates of the homology model of the present invention may also facilitate the identification of related proteins or enzymes analogous to GPCR in function, structure or both, thereby further leading to novel therapeutic modes for treating or preventing GPCR related diseases.

The design of compounds that bind to or modulate a GPCR according to the present invention generally involves consideration of two factors. Firstly, the compound must be capable of physically and structurally associating with a GPCR. Non-covalent molecular interactions important in the association of a GPCR with its substrate may include electrostatic interactions, hydrogen bonding, van der Waals and hydrophobic interactions. Secondly, the compound must be able to assume a conformation that allows it to associate with a GPCR. Although certain portions of the compound may not directly participate in the association with a GPCR, those portions may still influence the overall conformation of the molecule. This may have a significant impact on potency. Such conformational requirements include the overall three-dimensional structure and orientation of the chemical entity or compound in relation to all or a portion of a binding site of a GPCR, or the spacing between functional groups of a compound comprising several chemical entities that directly interact with a GPCR.

The potential modulating or binding effect of a chemical compound on a GPCR may be analysed prior to its actual synthesis and testing by the use of computer modelling techniques. If the theoretical structure of the given compound suggests insufficient interaction and association with a GPCR, then synthesis and testing of the compound may be obviated. However, if computer modelling indicates a strong interaction, the molecule may be synthesised and tested for its ability to bind to a GPCR and modulate (eg. inhibit) using the fluorescent substrate assay of Thornberry et al. (2000)Methods Enzymol. 322, pp 100-110. In this manner, synthesis of inactive compounds may be avoided.

A modulating or other binding compound of a GPCR may be computationally evaluated and designed by means of a series of steps in which chemical entities or candidate compounds are screened and selected for their ability to associate with a GPCR.

A person skilled in the art may use one of several methods to screen chemical entities or candidate compounds for their ability to associate with a GPCR and more particularly with the intracellular binding sites of a GPCR. This process may begin by visual inspection of, for example, the active site on the computer screen based on the GPCR co-ordinates of the present invention. Selected chemical entities or candidate compounds may then be positioned in a variety of orientations, or docked, with the GPCR. Docking may be accomplished using software such as Quanta and Sybyl, followed by energy minimisation and molecular dynamics with standard molecular mechanics force fields—such as CHARMM and AMBER. Other suitable docking programs include GOLD, DOCK, GLIDE and FRED.

Specialised computer programs may also assist in the process of selecting chemical entities or candidate compounds. These include but are not limited to MCSS (Miranker and Karplus (1991) Proteins: Structure, Function and Genetics, 11, pp. 29-34); GRID (Goodford (1985) J. Med. Chem., 28, pp. 849-857) and AUTODOCK (Goodsell and Olsen (1990), Proteins: Structure. Function, and Genetics, 8, pp. 195-202.

Once suitable chemical entities or candidate compounds have been selected, they may be assembled into a single compound, such as a GPCR modulator. Assembly may proceed by visual inspection of the relationship of the chemical entities or candidate compounds in relation to the structure co-ordinates of a GPCR. This may be followed by manual model building using software—such as Quanta, Sybyl, 0, HOOK or CAVEAT [Jones, T. A., Zou, J. Y., Cowan, S. W. & Kjeldgaard, M. Improved methods for building protein models in electron density maps and the location of errors in these models (1991) Acta Crystallogr. A47, 110-119].

Refinement of the model may be carried out using the program CNS [Brünger, A. T. et al. Crystallography & NMR System: A new software suite for macromolecular structure determination. (1998) Acta Crystallogr. D 54, 905-921].

Various programs may be used by a skilled person to connect the individual chemical entities or candidate compounds, such as 3D Database systems (Martin (1992) J. Med. Chem., 35, pp. 2145-2154) and CAVEAT (Bartlett et al. (1989) Royal Chem. Soc. 78, pp. 182-196).

Rather than build a GPCR inhibitor one chemical entity at a time, modulating or other GPCR binding compounds may be designed as a whole or de novo using either an empty binding site or optionally including some portion(s) of a known inhibitor(s). Such compounds may be designed using programs that may include but are not limited to LEGEND (Nishibata and Itai (1991) Tetrahedron, 47, p. 8985) and LUDI (Bohm (1992) J. Comp. Aid. Molec. Design, 6, pp. 61-78).

Other molecular modelling techniques may also be employed in accordance with this invention—such as those described by Cohen et al., J. Med. Chem., 33, pp. 883-894 (1990); Navia and Murcko (1992) Current Opinions in Structural Biology, 2, pp. 202-210 (1992).

Once a compound has been designed or selected by the above methods, the efficiency with which that compound may bind to a GPCR may be computationally evaluated. Specific computer software may be used to evaluate the efficiency of binding (eg. to evaluate compound deformation energy and electrostatic interaction), such as QUANTA/CHARMM (Accelrys Inc., USA) and Insight II/Discover (Biosym Technologies Inc., San Diego, Calif., USA). These programs may be implemented, for instance, using a suitable workstation. Other hardware systems and software packages will be known to those persons skilled in the art.

Once a GPCR-modulating compound has been selected or designed, as described above, substitutions may be made (eg. in atoms or side groups) to improve or modify the binding properties. The substitutions may be conservative i.e. the replacement group may have approximately the same size, shape, hydrophobicity and charge as the original group. Such substituted chemical compounds may then be analysed for efficiency of binding to a GPCR by the same computer methods described above.

Candidate compounds and modulators of a GPCR etc., which are identified using the methods of the present invention, may be screened in assays. Screening can be, for example in vitro, in cell culture, and/or in vivo. Biological screening assays preferably centre on activity-based response models, binding assays (which measure how well a compound binds), and bacterial, yeast and animal cell lines (which measure the biological effect of a compound in a cell). Suitable assays are described herein. The assays can be automated for high capacity-high throughput screening (HTS) in which large numbers of compounds can be tested to identify compounds with the desired activity.

Current screening technologies are described in Handbook of Drug Screening, edited by Ramakrishna Seethala, Prabhavathi B. Fernandes. New York, N.Y., Marcel Dekker, (2001).

The conformation can be used to define a starting point for pharmacophore derivation, shape based database searching, CoMFA, Fieldscreen (Cresset, J. G. Vinter et al.) etc. The sequence information can be used in conjunction with the structure of the protein or a homologous protein and a homology model and the knowledge of the binding site residue locations to define pharmacophores for use for searching databases or for predicting activity using programs such as Catalyst (Accelrys), Unity (Tripos), Phase (Schrodinger).

### DEFINITIONS

As herein, the term “modulating” or “modulates” refers to preventing, suppressing, inhibiting, alleviating, restorating, elevating, increasing or otherwise affecting GPCR activity. Suitably GPCR activity is GPCR signalling activity.

The term “allosteric modulator” may refer to a single entity or a combination of entities.

The allosteric modulator of a GPCR may be an antagonist or an agonist of said GPCR.

As used herein, the term “agonist” means any entity, which is capable of interacting (eg. binding) with a GPCR resulting in an increased or modified biological response. Suitably an agonist can be a protein ligand, peptide, chemokine, chemoattractant, lipid derivative or cytokine.

As used herein, the term “antagonist” means any entity, which is capable of interacting (eg. binding) with a GPCR resulting in a decreased biological response to the agonist.

Preferably, the allosteric GPCR modulators of the present invention are antagonists of GPCR and modulate the GPCR to reduce ligand binding and activation of the GPCR. In another embodiment, the allosteric GPCR modulators are activators and modulate the GPCR to increase activation of the GPCR.

The allosteric modulator of a GPCR may be an organic compound or other chemical. The allosteric modulator of a GPCR may be a compound, which is obtainable from or produced by any suitable source, whether natural or artificial. The allosteric modulator of a GPCR may be an amino acid molecule, a polypeptide, or a chemical derivative thereof, or a combination thereof. The allosteric modulator of a GPCR may even be a polynucleotide molecule, which may be a sense or an anti-sense molecule. The allosteric modulator of a GPCR may even be an antibody.

The allosteric modulator of a GPCR may be designed or obtained from a library of compounds, which may comprise peptides, as well as other compounds, such as small organic molecules.

By way of example, the allosteric modulator of a GPCR may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetic, a derivatised agent, a peptide cleaved from a whole protein, or a peptide synthesised synthetically (such as, by way of example, either using a peptide synthesiser or by recombinant techniques or combinations thereof, a recombinant agent, an antibody, a natural or a non-natural agent, a fusion protein or equivalent thereof and mutants, derivatives or combinations thereof).

Typically, the allosteric modulator of a GPCR will be an organic compound. Typically, the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the allosteric modulator of a GPCR comprises at least one cyclic group. The cyclic group may be a polycyclic group, such as a non-fused polycyclic group. For some applications, the allosteric modulator of a GPCR comprises at least the one of said cyclic groups linked to another hydrocarbyl group.

The allosteric modulator of a GPCR may contain halo groups, for example, fluoro, chloro, bromo or iodo groups, or one or more of alkyl, alkoxy, alkenyl, alkylene and alkenylene groups, each of which may be branched or unbranched.

The allosteric modulator of a GPCR may be a structurally novel allosteric modulator of a GPCR, or may be an analogue of a known allosteric modulator of a GPCR.

Preferably, the allosteric modulators of a GPCR have improved properties over those GPCR modulators previously available, for example, fewer side effects.

The allosteric modulator of a GPCR may be a mimetic, or may be chemically modified.

The allosteric modulator of a GPCR may be capable of displaying other therapeutic properties.

The allosteric modulator of a GPCR may be used in combination with one or more other pharmaceutically active agents. If combinations of active agents are administered, then they may be administered simultaneously, separately or sequentially.

### Candidate Compounds

As used herein, the term “candidate compound” includes, but is not limited to, a compound which may be obtainable from or produced by any suitable source, whether natural or not.

The candidate compound may be designed or obtained from a library of compounds, which may comprise peptides, as well as other compounds, such as small organic molecules and particularly new lead compounds. By way of example, the candidate compound may be a natural substance, a biological macromolecule, or an extract made from biological materials—such as bacteria, fungi, or animal (particularly mammalian) cells or tissues, an organic or an inorganic molecule, a synthetic candidate compound, a semi-synthetic candidate compound, a structural or functional mimetic, a peptide, a peptidomimetic, a derivatised candidate compound, a peptide cleaved from a whole protein, or a peptide synthesised synthetically, for example, either using a peptide synthesiser or by recombinant techniques or combinations thereof, a recombinant candidate compound, a natural or a non-natural candidate compound, a fusion protein or equivalent thereof and mutants, derivatives or combinations thereof. The candidate compound may even be a compound that is a modulator of a GPCR, such as a known inhibitor of a GPCR, that has been modified in some way eg. by recombinant DNA techniques or chemical synthesis techniques.

Typically, the candidate compound will be prepared by recombinant DNA techniques and/or chemical synthesis techniques.

Once a candidate compound capable of allosteric interaction with a GPCR has been identified, further steps may be carried out to select and/or to modify the candidate compounds and/or to modify existing compounds, such that they are able to modulate the GPCR.

In one aspect, the modulator of a GPCR may act as a model (for example, a template) for the development of other compounds.

A further aspect relates to the use of candidate compounds or allosteric GPCR modulators identified by the assays and methods of the invention in one or more model systems, for example, in a biological model, a disease model, or a model for GPCR inhibition. Such is models may be used for research purposes and for elucidating further details of the biological, physicochemical, pharmacological and/or pharmacokinetic activity of a particular candidate compound. By way of example, the candidate compounds or GPCR modulators of the present invention may be used in biological models or systems in which chemokine signalling is known to be of particular significance.

### Mimetic

As used herein, the term “mimetic” relates to any chemical which includes, but is not limited to, a peptide, polypeptide, antibody or other organic chemical which has the same qualitative activity or effect as a known compound. That is, the mimetic is a functional equivalent of a known compound.

### Chemical Synthesis Methods

Preferably, the modulator of GPCR of the present invention may be prepared by chemical synthesis techniques.

It will be apparent to those skilled in the art that sensitive functional groups may need to be protected and deprotected during synthesis of a compound of the invention. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be racemised, for example if a base is used in a reaction with a substrate having an optical centre comprising a base-sensitive group. This is possible during e.g. a guanylation step. It should be possible to circumvent potential problems such as this by choice of reaction sequence, conditions, reagents, protection/deprotection regimes, etc. as is well-known in the art.

The compounds and salts may be separated and purified by conventional methods.

Separation of diastereomers may be achieved by conventional techniques, e.g. by fractional crystallisation, chromatography or H.P.L.C. of a stereoisomeric mixture of a compounds or suitable salts or derivatives thereof. An individual enantiomer of a compound may also be prepared from a corresponding optically pure intermediate or by resolution, such as by H.P.L.C. of the corresponding racemate using a suitable chiral support or by fractional crystallisation of the diastereomeric salts formed by reaction of the corresponding racemate with a suitably optically active acid or base.

GPCRs, allosteric modulators of a GPCR or variants, homologues, derivatives, fragments or mimetics thereof may be produced using chemical methods to synthesise the GPCR or the modulator of a GPCR in whole or in part. For example, a GPCR peptide or a modulator of a GPCR that is a peptide can be synthesised by solid phase techniques, cleaved from the resin, and purified by preparative high performance liquid chromatography (e.g., Creighton (1983) Proteins Structures And Molecular Principles, WH Freeman and Co, New York N.Y.). The composition of the synthetic peptides may be confirmed by amino acid analysis or sequencing (e.g., the Edman degradation procedure; Creighton, supra).

Synthesis of peptides (or variants, homologues, derivatives, fragments or mimetics thereof) may be performed using various solid-phase techniques (Roberge J Y et al (1995) Science 269: 202-204) and automated synthesis may be achieved, for example, using the ABI 431 A Peptide Synthesizer (Perkin Elmer) in accordance with the instructions provided by the manufacturer. Additionally, the amino acid sequences comprising the modulator of a GPCR, may be altered during direct synthesis and/or combined using chemical methods with a sequence from other subunits, or any part thereof, to produce a variant modulator of a GPCR.

### Chemical Modification

In one embodiment, the modulator of a GPCR may be a chemically modified modulator of a GPCR. The chemical modification of a modulator of a GPCR may either enhance or reduce interactions between the modulator of a GPCR and the target, such as hydrogen bonding interactions, charge interactions, hydrophobic interactions, Van der Waals interactions or dipole interactions.

### Process

Another aspect of the invention relates to a process comprising the steps of:
(a) performing the method according to the invention, or an assay according to the invention;
(b) identifying one or more modulators of a GPCR; and
(c) preparing a quantity of said one or more GPCR modulators.

A further aspect of the invention relates to a process comprising the steps of:
(a) performing the method according to the invention, or an assay according to the invention;
(b) identifying one or more GPCR modulators; and
(c) preparing a pharmaceutical composition comprising said one or more identified GPCR modulators.

A further aspect relates to a process comprising the steps of:
(a) performing the method according to the invention, or an assay according to the invention;
(b) identifying one or more GPCR modulators;
(c) modifying said one or more GPCR modulators; and
(d) optionally preparing a pharmaceutical composition comprising said one or more GPCR modulators.

### Pharmaceutical Compositions

Another aspect of the invention relates to a pharmaceutical composition comprising a GPCR modulator or candidate compound of the invention and a pharmaceutically acceptable carrier, diluent, excipient or adjuvant or any combination thereof. Even though the GPCR modulators or candidate compounds (including their pharmaceutically acceptable salts, esters and pharmaceutically acceptable solvates) can be administered alone, they will generally be administered in admixture with a pharmaceutical carrier, excipient or diluent, particularly for human therapy. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

### Salts/Esters

The GPCR modulators or candidate compounds of the present invention can be present as salts or esters, in particular pharmaceutically acceptable salts or esters.

Pharmaceutically acceptable salts of the GPCR modulators or candidate compounds of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium is hydroxide, calcium hydroxide, aluminium hydroxide. Alcohols include alkanealcohols of 1-12 carbon atoms which may be unsubstituted or substituted, e.g. by a halogen).

In a preferred embodiment, the allosteric modulators identified in accordance with the present invention are rendered cell permeable. For example, modulators may be designed to be cell permeable as a result of their combined physicochemical properties including number of hydrogen bond donors, logD, logP molecular weight etc. In addition, modulators may be carried in by another agent such as a virus capsule or administered in lipid micelles.

### Enantiomers/Tautomers

In all aspects of the present invention previously discussed, the invention includes, where appropriate all enantiomers and tautomers of the GPCR modulators or candidate compounds of the invention. The man skilled in the art will recognise compounds that possess optical properties (one or more chiral carbon atoms) or tautomeric characteristics. The corresponding enantiomers and/or tautomers may be isolated/prepared by methods known in the art.

### Stereo and Geometric Isomers

Some of the GPCR modulators or candidate compounds of the invention may exist as stereoisomers and/or geometric isomers, e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

The present invention also includes all suitable isotopic variations of the GPCR modulators or candidate compounds, or pharmaceutically acceptable salts thereof. An isotopic variation of a GPCR modulator or candidate compound of the present invention or a pharmaceutically acceptable salt thereof is defined as one in which at least one atom is is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the GPCR modulators or candidate compounds of the present invention can generally be prepared by conventional procedures using appropriate isotopic variations of suitable reagents.

### Solvates

The present invention also includes solvate forms of the GPCR modulators or candidate compounds. The terms used in the claims encompass these forms.

### Polymorphs

The invention furthermore relates to GPCR modulators or candidate compounds of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Prodrugs

The invention further includes GPCR modulators or candidate compounds of the present invention in prodrug form. Such prodrugs are generally compounds of the invention wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include ester (for example, any of those described above), wherein the reversion may be carried out be an esterase etc. Other such systems will be well known to those skilled in the art.

Examples of ester compounds which act as prodrugs are described herein. Suitable modifications render the compound cell permeable.

### Therapeutic Use

Allosteric modulators of GPCRs including Chemokine receptors identified in accordance with the invention have activity as pharmaceuticals, in particular as modulators of Chemokine receptors, and may be used in the treatment (therapeutic or prophylactic) of conditions/diseases in human and non-human animals which are exacerbated or caused by excessive or unregulated production of chemokines. Examples of such conditions/diseases include:

(1) (the respiratory tract) obstructive airways diseases including chronic obstructive pulmonary disease (COPD); asthma, such as bronchial, allergic, intrinsic, extrinsic and dust asthma, particularly chronic or inveterate asthma (e.g. late asthma and airways hyper-responsiveness); bronchitis; acute, allergic, atrophic rhinitis and chronic rhinitis including rhinitis caseosa, hypertrophic rhinitis, rhinitis purulenta, rhinitis sicca and rhinitis medicamentosa; membranous rhinitis including croupous, fibrinous and pseudomembranous rhinitis and scrofoulous rhinitis; seasonal rhinitis including rhinitis nervosa (hay fever) and vasomotor rhinitis, idiopathic pulmonary fibrosis (IPF); sarcoidosis, farmer's lung and related diseases, fibroid lung and idiopathic interstitial pneumonia;
(2) (bone and joints) rheumatoid arthritis, seronegative spondyloarthropathies (including ankylosing spondylitis, psoriatic arthritis and Reiter's disease), Behcet's disease, Siogren's syndrome and systemic sclerosis, gout, osteoporosis and osteoarthritis.
(3) (skin) psoriasis, atopical dermatitis, contact dermatitis and other eczmatous dermitides, seborrhoetic dermatitis, Lichen planus, Pemphigus, bullous Pemphigus, Epidermolysis bullosa, urticaria, angiodermas, vasculitides, erythemas, cutaneous eosinophilias, uveitis, Alopecia greata and vemal conjunctivitis;
(4) (gastrointestinal tract) Coeliac disease, proctitis, eosinopilic gastro-enteritis, mastocytosis, Crohn's disease, ulcerative colitis, food-related allergies which have effects remote from the gut, e.g., migraine, rhinitis and eczema;
(5) (other tissues and systemic disease) multiple sclerosis, atherosclerosis, Acquired Immunodeficiency Syndrome (AIDS), lupus erythematosus, systemic lupus, erythematosus, Hashimoto's thyroiditis, myasthenia gravis, type I diabetes, nephrotic syndrome, eosinophilia fascitis, hyper IgE syndrome, lepromatous leprosy, sezary syndrome and idiopathic thrombocytopenia pupura; post-operative adhesions, and sepsis.
(6) (allograft rejection) acute and chronic following, for example, transplantation of kidney, heart, liver, lung, bone marrow, skin and cornea; and chronic graft versus host disease;
(7) Cancers, especially non-small cell lung cancer (NSCLC), malignant melanoma, prostate cancer and squamous sarcoma, and tumour metastasis;
(8) Diseases in which angiogenesis is associated with raised CXCR2 chemokine levels (e.g. NSCLC, diabetic retinopathy).
(9) Cystic fibrosis, stroke, re-perfusion injury in the heart, brain, peripheral limbs and other organs.
(10) Burn wounds & chronic skin ulcers
(11) Reproductive Diseases (e.g. Disorders of ovulation, menstruation and implantation, Pre-term labour, Endometriosis)

Thus, compounds identified in accordance with the invention, or pharmaceutically-acceptable salts or solvates thereof, are for use in therapy.

Preferably the compounds identified in accordance with the invention are used to treat diseases in which the Chemokine receptor belongs to the CXC Chemokine receptor subfamily, more preferably the target Chemokine receptor is the CXCR2 receptor.

Particular conditions which can be treated with the compounds of the invention are rheumatoid arthritis, psoriasis, diseases in which angiogenesis is associated with raised CXCR2 chemokine levels, IBD and COPD. It is preferred that the compounds of the invention are used to treat rheumatoid arthritis and COPD.

In the context of the present specification, the term “therapy” also includes “prophylaxis” unless there are specific indications to the contrary. The terms “therapeutic” and “therapeutically” should be construed accordingly.

A further aspect of the invention therefore relates to a method of treating a GPCR related disorder, said method comprising administering to a subject in need thereof a compound identified in accordance with the invention.

A further aspect of the invention relates to the use of a GPCR modulator or candidate compound according to the invention in the preparation of a medicament for treating a GPCR-related disorder.

As used herein the phrase “preparation of a medicament” includes the use of the compound directly as the medicament in addition to its use in a screening programme for further therapeutic agents or in any stage of the manufacture of such a medicament.

In one preferred embodiment, the compound of the invention is administered orally.

Yet another aspect relates to a method of selectively inhibiting a GPCR in a cell comprising contacting said cell with an amount of a compound identified in accordance with the invention, such that a GPCR is selectively inhibited in said cell.

### Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 250 mg and more preferably from 10-100 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-250 mg, of active ingredient per dose.

Compositions may be formulated in unit dosage form, i.e., in the form of discrete portions containing a unit dose, or a multiple or sub-unit of a unit dose.

### Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.01 to 30 mg/kg body weight, such as from 0.1 to 10 mg/kg, more preferably from 0.1 to 1 mg/kg body weight.

In an exemplary embodiment, one or more doses of 10 to 150 mg/day will be administered to the patient for the treatment of malignancy.

### GPCR Fragment

Another aspect of the invention relates to a fragment of GPCR, or a homologue, mutant, or derivative thereof, comprising a ligand binding domain, said ligand binding domain being defined by the amino acid residue structural coordinates corresponding to the following amino acids in CXCR2: S81, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 (particularly S81, V82, T83, D84, Y86, L87, L90, G133, L136, I140, D143, R144, A147, Q157, Q245, K246, A249, V252, I253, V256, I259, L309, N310, P311, I313, Y314, I317, G318, Q319, K320, F321), or by amino acid residues 301 to 360 of CXCR2 (particularly residues 304 to 326 of CXCR2).

As used herein, the term “allosteric intracellular binding site” means the intracellular region of a GPCR which is responsible for an allosteric modification when a compound is bound. The term “allosteric intracellular binding site” also includes a homologue of the allosteric intracellular binding site, or a portion thereof.

As used herein, the term “portion thereof” means the structural co-ordinates corresponding to a sufficient number of amino acid residues of the intracellular binding site of the GPCR sequence (or homologue thereof) that are capable of interacting with a candidate compound capable of binding to the allosteric intracellular binding site and eliciting an allosteric modulation of the GPCR.

In one preferred embodiment, the fragment of a GPCR corresponding to the allosteric intracellular binding site, or a homologue, mutant or derivative thereof, corresponds to a portion of the structure co-ordinates of Table 3. In particular, said site corresponds to amino acids 304 to 326 of CXCR2.

Another aspect of the invention relates to the use of the above-described fragment of a GPCR, or a homologue, mutant, or derivative thereof, in an assay for identifying candidate compounds capable of modulating a GPCR.

For use in the assays of the present invention, GPCR proteins or fragments thereof may be produced by a host recombinant cell may be secreted or may be contained intracellularly depending on the nucleotide sequence and/or the vector used.

As will be understood by those skilled in the art, expression vectors containing a GPCR encoding nucleotide sequence or a mutant, variant, homologue, derivative or fragment thereof, may be designed with signal sequences which direct secretion of the GPCR coding sequences through a particular prokaryotic or eukaryotic cell membrane.

The GPCR encoding sequence may be fused (eg. ligated) to nucleotide sequences encoding a polypeptide domain which will facilitate purification of soluble proteins (Kroll D J et al (1993) DNA Cell Biol 12:441-53). Preferably, the polypeptide domain, which facilitates purification of soluble proteins, is fused in frame with the GPCR encoding sequence. Such purification facilitating domains include, but are not limited to, metal chelating peptides—such as histidine-tryptophan modules that allow purification on immobilised metals (Porath J (1992) Protein Expr Purif 3, 263-281), protein A domains that allow purification on immobilised immunoglobulin, and the domain utilised in the FLAGS extension/affinity purification system (Immunex Corp, Seattle, Wash.). The inclusion of a cleavable linker sequence such as Factor XA or enterokinase (Invitrogen, San Diego, Calif.) between the purification domain and GPCR is useful to facilitate purification.

### Nucleotide Sequences

As used herein, the term “nucleotide sequence” refers to nucleotide sequences, oligonucleotide sequences, polynucleotide sequences and variants, homologues, fragments and derivatives thereof (such as portions thereof) which comprise the nucleotide sequences encoding GPCR.

The nucleotide sequence may be DNA or RNA of genomic or synthetic or recombinant origin, which may be double-stranded or single-stranded whether representing the sense or antisense strand or combinations thereof.

Preferably, the term nucleotide sequence is prepared by use of recombinant DNA techniques (e.g. recombinant DNA). The nucleotide sequences may include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the nucleotide sequences described herein may be modified by any method available in the art.

It will be understood by a skilled person that numerous different nucleotide sequences can encode the same protein as a result of the degeneracy of the genetic code. In addition, it is to be understood that skilled persons may, using routine techniques, make nucleotide substitutions that do not substantially affect the activity encoded by the nucleotide sequence of the present invention to reflect the codon usage of any particular host organism in which the target is to be expressed. Thus, the terms “variant”, “homologue” or “derivative” in relation to nucleotide sequences include any substitution of, variation of, modification of, replacement of, deletion of or addition of one (or more) nucleic acids from or to the sequence providing the resultant nucleotide sequence encodes a functional protein according to the present invention (or even a modulator of a GPCR according to the present invention if said modulator comprises a nucleotide sequence or an amino acid sequence).

### Amino Acid Sequences

As used herein, the term “amino acid sequence” is synonymous with the term “polypeptide” and/or the term “protein”. In some instances, the term “amino acid sequence” is synonymous with the term “peptide”.

The amino acid sequence may be isolated from a suitable source, or it may be made synthetically or it may be prepared by use of recombinant DNA techniques.

### Variants/Homologues/Derivatives/Fragments

The GPCR described herein is intended to include any polypeptide, which has the activity of the naturally occurring GPCR and includes all vertebrate and mammalian forms. Such terms also include polypeptides that differ from naturally occurring forms of the GPCR by having amino acid deletions, substitutions, and additions, but which retain the activity of the GPCR.

The term “variant” is used to mean a naturally occurring polypeptide or nucleotide sequences which differs from a wild-type or a native sequence.

The term “fragment” indicates that a polypeptide or nucleotide sequence comprises a fraction of a wild-type or a native sequence. It may comprise one or more large contiguous sections of sequence or a plurality of small sections. The sequence may also comprise other elements of sequence, for example, it may be a fusion protein with another protein. Preferably the sequence comprises at least 50%, more preferably at least 65%, more preferably at least 80%, most preferably at least 90% of the wild-type sequence.

The present invention also encompasses the use of variants, homologues and derivatives of nucleotide and amino acid sequences. Here, the term “homologue” means an entity having a certain homology with amino acid sequences or nucleotide sequences. Here, the term “homology” can be equated with “identity”.

In the present context, an homologous sequence is taken to include an amino acid sequence which may be at least 75, 85 or 90% identical, preferably at least 95 or 98% identical to the subject sequence. Although homology can also be considered in terms of similarity (i.e. amino acid residues having similar chemical properties/functions), it is preferred to express homology in terms of sequence identity.

An homologous sequence is taken to include a nucleotide sequence which may be at least 75, 85 or 90% identical, preferably at least 95 or 98% identical to the subject sequence.

Homology comparisons can be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8). Another alternative is to align manually, using known alignment motifs.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues, which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

Homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as 0), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-I-phenylalanine*, L-allyl-glycine*, β-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#}*, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

The term “derivative” or “derivatised” as used herein includes chemical modification of an entity, such as candidate compound or a GPCR modulator. Illustrative of such chemical modifications would be replacement of hydrogen by a halo group, an alkyl group, an acyl group or an amino group.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

### Mutant

As used herein, the term “mutant” refers to a GPCR comprising one or more changes in the wild-type GPCR sequence.

The term “mutant” is not limited to amino acid substitutions of the amino acid residues in a GPCR, but also includes deletions or insertions of nucleotides which may result in changes in the amino acid residues in the amino acid sequence of a GPCR.

The present invention also enables the solving of the crystal structure of GPCR mutants. More particularly, by virtue of the present invention, the location of the active site of the intracellular binding site of a GPCR based on the structural coordinates of Table 3 permits the identification of desirable sites for mutation. For example, one or more mutations may be directed to a particular site—such as the active site—or combination of sites of a GPCR. Similarly, only a location on, at or near the enzyme surface may be replaced, resulting in an altered surface charge of one or more charge units, as compared to the wild-type enzyme. Alternatively, an amino acid residue in a GPCR may be chosen for replacement based on its hydrophilic or hydrophobic characteristics.

Such mutants may be characterised by any one of several different properties as compared with the wild-type GPCR. For example, such mutants may have altered surface charge of one or more charge units, or have an increased stability to subunit dissociation, or an altered substrate specificity in comparison with, or a higher specific activity than, the wild-type GPCR.

The mutants may be prepared in a number of ways that are known by a person skilled in the art. For example, mutations may be introduced by means of oligonucleotide-directed mutagenesis or other conventional methods. Alternatively, mutants of a GPCR may be generated by site-specific replacement of a particular amino acid with an unnaturally occurring amino acid. This may be achieved by growing a host organism capable of expressing either the wild-type or mutant polypeptide on a growth medium depleted of one or more natural amino acids but enriched in one or more corresponding unnaturally occurring amino acids.

### Host Cells

As used herein, the term “host cell” refers to any cell that comprises nucleotide sequences that are of use in the present invention, for example, nucleotide sequences encoding GPCR.

Host cells may be transformed or transfected with a nucleotide sequence contained in a vector e.g. a cloning vector. Preferably, said nucleotide sequence is carried in a vector for the replication and/or expression of the nucleotide sequence. The cells will be chosen to be compatible with the said vector and may for example be prokaryotic (for example bacterial), fungal, yeast or plant cells.

The gram-negative bacterium E. coli is widely used as a host for cloning nucleotide sequences. This organism is also widely used for heterologous nucleotide sequence expression. However, large amounts of heterologous protein tend to accumulate inside the cell. Subsequent purification of the desired protein from the bulk of E. coli intracellular proteins can sometimes be difficult.

In contrast to E. coli, bacteria from the genus Bacillus are very suitable as heterologous hosts because of their capability to secrete proteins into the culture medium. Other bacteria suitable as hosts are those from the genera Streptomyces and Pseudomonas.

Depending on the nature of the polynucleotide and/or the desirability for further processing of the expressed protein, eukaryotic hosts including yeasts or other fungi may be preferred. In general, yeast cells are preferred over fungal cells because yeast cells are easier to manipulate. However, some proteins are either poorly secreted from the yeast cell, or in some cases are not processed properly (e.g. hyperglycosylation in yeast). In these instances, a different fungal host organism should be selected. Examples of expression hosts are fungi—such as Aspergillus species (such as those described in EP-A-0184438 and EP-A-0284603) and Trichoderma species; bacteria—such as Bacillus species (such as those described in EP-A-0134048 and EP-A-0253455), Streptomyces species and Pseudomonas species; yeasts—such as Kluyveromyces species (such as those described in EP-A-0096430 and EP-A-0301670) and Saccharomyces species; and mammalian cells—such as CHO-K1 cells.

The use of host cells may provide for post-translational modifications as may be needed to confer optimal biological activity on recombinant expression products of the present invention.

Aspects of the present invention also relate to host cells comprising the GPCR constructs of the present invention. The GPCR constructs may comprise a nucleotide sequence for as replication and expression of the sequence. The cells will be chosen to be compatible with the vector and may for example be prokaryotic (for example bacterial), fungal, yeast or plant cells.

In a preferred embodiment, the host cells are mammalian cells, such as CHO-K1 cells or HEK293 cells.

### Vector

Aspects of the present invention relate to a vector comprising a nucleotide sequence, such as a nucleotide sequence encoding a GPCR or a modulator of a GPCR, administered to a subject.

Preferably, the GPCR or the modulator of a GPCR is prepared and/or delivered using a genetic vector.

As it is well known in the art, a vector is a tool that allows or facilitates the transfer of an entity from one environment to another. In accordance with the present invention, and by way of example, some vectors used in recombinant DNA techniques allow entities, such as a segment of DNA (such as a heterologous DNA segment, such as a heterologous cDNA segment), to be transferred into a host and/or a target cell for the purpose of replicating the vectors comprising nucleotide sequences and/or expressing the proteins encoded by the nucleotide sequences. Examples of vectors used in recombinant DNA techniques include, but are not limited to, plasmids, chromosomes, artificial chromosomes or viruses.

The term “vector” includes expression vectors and/or transformation vectors.

The term “expression vector” means a construct capable of in vivo or in vitro/ex vivo expression.

The term “transformation vector” means a construct capable of being transferred from one species to another.

### Regulatory Sequences

In some applications, nucleotide sequences are operably linked to a regulatory sequence which is capable of providing for the expression of the nucleotide sequence, such as by a chosen host cell. By way of example, a vector comprising the GPCR nucleotide sequence is operably linked to such a regulatory sequence i.e. the vector is an expression vector.

The term “operably linked” refers to a juxtaposition wherein the components described are in a relationship permitting them to function in their intended manner. A regulatory sequence “operably linked” to a coding sequence is ligated in such a way that expression of the coding sequence is achieved under conditions compatible with the control sequences.

The term “regulatory sequences” includes promoters and enhancers and other expression regulation signals.

The term “promoter” is used in the normal sense of the art, e.g. an RNA polymerase binding site.

Enhanced expression of a nucleotide sequence, for example, a nucleotide sequence encoding a GPCR, may also be achieved by the selection of heterologous regulatory regions, e.g. promoter, secretion leader and terminator regions, which serve to increase expression and, if desired, secretion levels of the protein of interest from the chosen expression host and/or to provide for the inducible control of the expression of a GPCR. In eukaryotes, polyadenylation sequences may be operably connected to the GPCR nucleotide sequence.

Preferably, the GPCR nucleotide sequence is operably linked to at least a promoter.

Aside from the promoter native to the gene encoding the GPCR nucleotide sequence, other promoters may be used to direct expression of the GPCR polypeptide. The promoter may be selected for its efficiency in directing the expression of the GPCR nucleotide sequence in the desired expression host.

In another embodiment, a constitutive promoter may be selected to direct the expression of the GPCR nucleotide sequence. Such an expression construct may provide additional advantages since it circumvents the need to culture the expression hosts on a medium containing an inducing substrate.

Hybrid promoters may also be used to improve inducible regulation of the expression construct.

The promoter can additionally include features to ensure or to increase expression in a suitable host. For example, the features can be conserved regions such as a Pribnow Box or a TATA box. The promoter may even contain other sequences to affect (such as to maintain, enhance, decrease) the levels of expression of the GPCR nucleotide sequence. For example, suitable other sequences include the Sh1-intron or an ADH intron. Other sequences include inducible elements—such as temperature, chemical, light or stress inducible elements. Also, suitable elements to enhance transcription or translation may be present.

### Expression Vector

Preferably, nucleotide sequences, such as nucleotide sequences encoding a GPCR or modulators of a GPCR, are inserted into a vector that is operably linked to a control sequence that is capable of providing for the expression of the coding sequence by the host cell.

Nucleotide sequences produced by a host recombinant cell may be secreted or may be contained intracellularly depending on the sequence and/or the vector used. As will be understood by those of skill in the art, expression vectors can be designed with signal sequences, which direct secretion of the nucleotide sequence through a particular prokaryotic or eukaryotic cell membrane.

Preferably, the expression vectors are stably expressed in wild type HEK293 cells expressing promiscuous Gqi5 on a stably integrated expression vector. Suitable expression vectors include pIRESneo2 (BD Biosciences Clontech) and/or pGENiresneo.

### Fusion Proteins

A GPCR or a modulator of a GPCR may be expressed as a fusion protein to aid extraction and purification and/or delivery of the modulator of a GPCR or the GPCR protein to an individual and/or to facilitate the development of a screen for modulators of a GPCR. Examples of fusion protein partners include glutathione-S-transferase (GST), 6×His, GAL4 (DNA binding and/or transcriptional activation domains) and β-galactosidase.

It may also be convenient to include a proteolytic cleavage site between the fusion protein partner and the protein sequence of interest to allow removal of fusion protein sequences. Preferably, the fusion protein will not hinder the activity of the protein of interest.

The fusion protein may comprise an antigen or an antigenic determinant fused to the substance of the present invention. In this embodiment, the fusion protein may be a non-naturally occurring fusion protein comprising a substance, which may act as an adjuvant in the sense of providing a generalised stimulation of the immune system. The antigen or antigenic determinant may be attached to either the amino or carboxy terminus of the substance.

### Organism

The term “organism” in relation to the present invention includes any organism that could comprise GPCR and/or modulators of a GPCR. Examples of organisms may include mammals, fungi, yeast or plants.

Preferably, the organism is a mammal. More preferably, the organism is a human.

### Transformation

As indicated earlier, the host organism can be a prokaryotic or a eukaryotic organism. Examples of suitable prokaryotic hosts include E. coli and Bacillus subtilis. Teachings on the transformation of prokaryotic hosts are well documented in the art, for example see Sambrook et al (Molecular Cloning: A Laboratory Manual, 2nd edition, 1989, Cold Spring Harbor Laboratory Press) and Ausubel et al., Current Protocols in Molecular Biology (1995), John Wiley & Sons, Inc. Examples of suitable eukaryotic hosts include mammalian cells.

If a prokaryotic host is used then the nucleotide sequence, such as the GPCR nucleotide sequence, may need to be suitably modified before transformation—such as by removal of introns.

Thus, the present invention also relates to the transformation of a host cell with a nucleotide sequence, such as GPCR or a modulator of a GPCR. Host cells transformed with the nucleotide sequence may be cultured under conditions suitable for the expression and recovery of the encoded protein from cell culture. The protein produced by a recombinant cell may be secreted or may be contained intracellularly depending on the sequence and/or the vector used. As will be understood by those of skill in the art, expression vectors containing coding sequences can be designed with signal sequences which direct secretion of the coding sequences through a particular prokaryotic or eukaryotic cell membrane. Other recombinant constructions may join the coding sequence to nucleotide sequence encoding a polypeptide domain, which will facilitate purification of soluble proteins (Kroll D J et al (1993) DNA Cell Biol 12:441-53) e.g. 6-His or Glutathione-S-transferase.

### Transfection

Vectors comprising for example, the GPCR nucleotide sequence, may be introduced into host cells, for example, mammalian cells, using a variety of methods.

Typical transfection methods include electroporation, DNA biolistics, lipid-mediated transfection, compacted DNA-mediated transfection, liposomes, immunoliposomes, lipofectin, cationic agent-mediated, cationic facial amphiphiles (CFAs) (Nature Biotech. (1996) 14, 556), multivalent cations such as spermine, cationic lipids or polylysine, 1, 2,-bis(oleoyloxy)-3-(trimethylammonio) propane (DOTAP)-cholesterol complexes (Wolff and Trubetskoy 1998 Nature Biotechnology 16: 421) and combinations thereof.

Uptake of nucleic acid constructs by mammalian cells is enhanced by several known transfection techniques for example those including the use of transfection agents. Examples of these agents include cationic agents (for example calcium phosphate and DEAE-dextran) and lipofectants (for example Lipofectam™ and Transfectam™). Typically, nucleic acid constructs are mixed with the transfection agent to produce a composition.

Such methods are described in many standard laboratory manuals—such as Sambrook et al., Molecular Cloning: A Laboratory Manual, 2d ed. (1989) Cold Spring Harbor Laboratory Press, Cold Spring Harbor, N.Y.

The present invention is further described by way of example, and with reference to the following Figures wherein:

### FIGURES

FIG. 1A shows an alignment of human CXCR1 and CXCR2. 1111 etc represent equivalent amino acids in bovine rhodopsin in alpha helix 1 etc. Boxed areas represent binding portions. Dashed lines represent amino acids in membrane spanning regions.

FIG. 1B shows a schematic representation of the structure of CXCR2.

FIG. 1C shows a schematic view of the intracellular face of a GPCR. The intracellular binding site is represented by an ellipse.

FIG. 2 shows inhibition of [¹²⁵I] IL8 binding in a CXCR2 membrane binding assay.

FIG. 3 shows Potency Correlation for a range of compounds displacing [¹²⁵I] IL-8 and [³H] Compound C.

FIG. 4 shows compound activity in a membrane binding assay versus compound activity in a whole cell calcium flux assay.

FIG. 5 shows a schematic diagram of constructs CXCR1(1-290)-CXCR2(301-360) (CXCR1/2), and CXCR2(1-300)-CXCR1(291-350) (CXCR2/1).

FIG. 6 shows GROalpha- and IL-8-induced calcium release for wild type CXCR1 and CXCR1(1-290)-CXCR2 (301 to 360) tail swap measured in FLIPR assay.

FIG. 7 shows GROalpha- and IL-8-induced calcium release for wild type CXCR2 and CXCR2(1-300)-CXCR1(291-350) tail swap measured in FLIPR assay.

FIG. 8 shows IL-8-induced calcium release for wild type CXCR1 and CXCR1(1-290)-CXCR2(301-360) tail swap in the presence and absence of 30 nM Compound A.

FIG. 9 shows IL-8-induced calcium release for wild type CXCR2 and CXCR2(1-300)-CXCR1(291-350) tail swap in the presence and absence of 30 nM Compound A.

FIG. 10 shows a schematic diagram of the constructs CXCR1(1-316)/CXCR2(327-360) (CXCR1/2short), and CXCR2(1-326)/CXCR1 (317-350) (CXCR2/1short).

FIG. 11 shows IL-8-induced calcium release for CXCR1/2short and CXCR2/1short in the presence and absence of Compound A and Compound B.

FIG. 12 shows a schematic diagram of CXCR1 and CXCR2 mutants: CXCR1 N311K/F316L, CXCR2 K320N/L325F, CXCR1 F316L, CXCR2 K320N and CXCR1 N311K.

FIG. 13 shows IL-8-induced calcium release for CXCR1N311K/F316L and CXCR2 K320N/L325 in the presence of Compound A and Compound B.

FIG. 14 shows IL-8-induced calcium release for CXCR1 F316L in the presence of Compound A and Compound B.

FIG. 15 shows IL-8-induced calcium release for CXCR1 N311K and CXCR2 K320N in the presence of Compound A and Compound B.

FIG. 16 shows an alignment of amino acids in first shell of intracellular binding site from a variety of GPCRs.

FIG. 17 shows principal components analysis of the intracellular binding site residues based upon e-state key descriptors (1^{st} and 2^{nd} components). The targets currently known to have intracellular binding sites are highlighted as stars.

FIG. 18 shows an alignment of the C-terminal amino acids in several Chemokine receptors (7777777 indicates the helix which the equivalent amino acid in bovine rhodopsin resides). Lys 320 and its equivalents are circled.

FIG. 19 shows an alignment of Chemokine receptors indicating residues which form the intracellular binding site.

FIG. 20 shows CXCR1, CXCR2, CCR4 and CCR2b alignment generated using CLUSTALW. Residues highlighted by shading indicate amino acids which form part of the compound binding pocket.

FIG. 21 shows results of a radioligand binding assay using [³H]Compound A with GST-CXCR2 fusion protein and wild type GST.

### EXAMPLES

### General Methods

The methods described here may employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref: A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### Methods and Materials

The following compounds are used in the following Examples. Compounds A and C are exemplified in WO2001025242 and Compound B in WO2000035442.

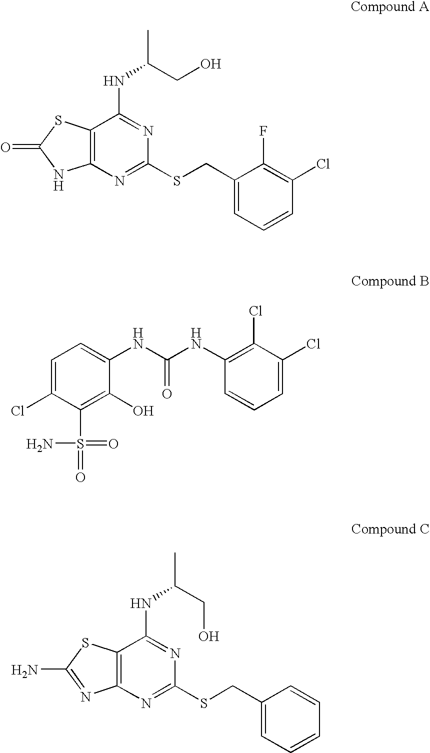

### Example 1

### CXCR2 Allosteric Antagonist Compounds Such as Compound a Displace IL-8 in a Membrane Binding Assay

### Method for Binding Assay

### Preparation of Membranes

The cDNA encoding the human Chemokine receptor CXCR2 was cloned into pIRESneo2 using standard methods as described in Sambrook et al., (1989) and confirmed by sequencing.

Adherent HEK293 cells were maintained as monolayer cultures in Dulbecco's modified Eagle's medium (DMEM) supplemented with 10% FBS and 2 mM L-glutamine (All from Sigma). HEK 293 cells were transfected with CXCR2 receptor using the lipofection reagent Fugene 6 (Roche). HEK 293 cells were seeded at 4×10⁵ cells per well in a 6 well plate (Costar) and grown to reach 70% confluency for transfection. Fugene 6 lipofection reagent and plasmid DNA (1 μg) was mixed at a ratio of 6:1 in a final volume of 500 μl PBS and incubated for 15 minutes at room temperature before addition to HEK 293 cells in culture media (drop wise). Cells were then incubated over night at 37° C. with an atmosphere of 5% CO₂/95% air. Stable transfectants expressing CXCR2 were selected for and maintained by addition of Geneticin G418 at 1 mg/ml (Invitrogen). Stable HEK-CXCR2 transfectants were grown to approximately 80% confluence in 10-layer cell factories in DMEM medium containing 10% (v/v) foetal calf serum and glutamine (2 mM) in a humidified incubator at 37° C., 5% CO₂. Cells were harvested from the flask using Accutase at 37° C. for 3 to 5 minutes.

Cells were resuspended on ice in 60 mL hypotonic buffer at a density of 2×10⁷ cells/mL. Membranes were prepared on ice by homogenisaton using a polytron tissue homogenizer set at 22000 rpm. The membrane preparation was purified by sucrose gradient centrifugation. In each of 4 centrifuge tubes 15 mL of cell membranes was layered onto 10 mL 41% (w/v) sucrose solution and the tubes centrifuged at 140000 g for 1 hour at 4° C. The membrane fraction was harvested at the interface, diluted 4-fold with HEPES-buffered Tyrode's solution and centrifuged at 100000 g for 20 minutes at 4° C. The membrane pellet is was re-suspended at 1×10⁸ cell equivalents/mL in HEPES-buffered Tyrode's solution and subsequently stored in aliquots at −80° C.

All buffers used for membrane preparation and storage were made in the presence of protease inhibitors.

### Assay Protocol Radioligand Binding Assay

Prior to use in an experiment, aliquots of membranes at 1×10⁸ cell equivalents/mL were defrosted at room temperature and diluted in HEPES-buffered salt solution.

To each well in a 96-well polypropylene plate was added [¹²⁵I]IL-8 (10 μL, to a final concentration of 0.06 nM, pre-diluted to 0.6 nM in HEPES-buffered salt solution from a 9.6 nM stock) and either vehicle (10 μL, 10% (v/v) DMSO in HEPES-buffered salt solution: for determination of total binding (B0)), Compound A (10 μL, 10 μM solution in 10% (v/v) DMSO HEPES-buffered salt solution: for determination of non-specific binding (NSB)) or the appropriate solution of the different compounds or IL-8 to be tested (10 μL, 10% (v/v) DMSO in HEPES-buffered salt solution) were added to the wells of a 96-well multiscreen-filter plate (pre-wet with HEPES-buffered salt solution). Membranes (80 μL of 1×10⁶ cell equivalents/mL) were then added, giving a total incubation volume of 100 μL per well. The plates were incubated for 2 hours at room temperature.

Following incubation, the plate containing the assay mixture was filter-washed with 200 μL cold HEPES-buffered salt solution using a Millipore vacuum manifold. The filtration plate was allowed to air dry then either the individual filters were punched out into polypropylene test tubes and the radioactivity measured by direct gamma counting using a Cobra II Gamma counter (Packard BioScience) for 1 minute per sample or alternatively, the whole filtration plate was placed in a carrier plate and 50 μL of MicroScint-O added to each well. 96-well plate scintillation counting was performed using a TopCount instrument (Packard BioScience) for 1 minute per sample well.

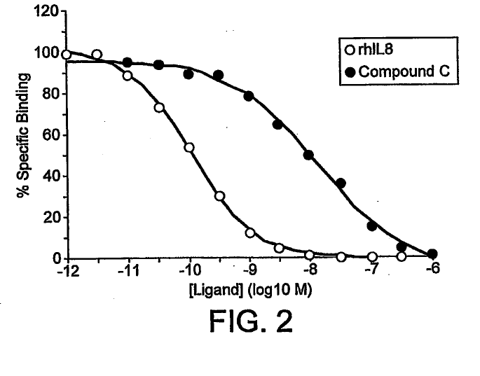

FIG. 2 shows that the presence of Compound C is able to inhibit the binding of [¹²⁵I]-IL-8 in a CXCR2 membrane binding assay as described in example 1 and also non-radioactive IL-8 is also able to inhibit the binding of [¹²⁵I]-IL-8.

### Method for [³H]-Compound C Binding Assay

To each well in a 96-well polypropylene plate was added [³H]Compound C (10 μL, to a final concentration of 20 nM, from a working solution of 200 nM in HEPES-buffered salt solution) and either vehicle (10 μL, 10% (v/v) DMSO in HEPES-buffered salt solution: for determination of total binding (B0)), Compound A (10 μL, 10 μM solution in 10% (v/v) DMSO HEPES-buffered salt solution: for determination of non-specific binding (NSB)) or the appropriate solution of the different compounds or IL-8 to be tested (10 μL, 10% (v/v) DMSO in HEPES-buffered salt solution) were added to the wells of a 96-well polypropylene plate. Membranes (80 μL of 3×10⁶ cell equivalents/mL) were then added, giving a total incubation volume of 100 μL per well. The plates were incubated for 2 hours at room temperature

Following incubation, the plate containing the assay mixture was transferred to GF-B plates and filter-washed with cold HEPES-buffered salt solution using a Tomtec harvester. The filtration plate was allowed to air dry then 20 μl of MicroScint-0 added to each well. 96-well plate scintillation counting was performed using a TopCount instrument (Packard BioScience) for 1 minute per sample well.

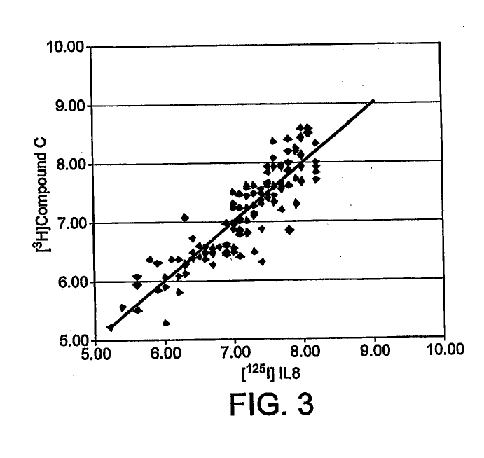

As Compound C can bind to CXCR2, the ability of other compounds to bind in the same site can be described by displacement of a radio-labelled Compound C. For many compounds which bind in the same site as Compound C, this displacement correlates with inhibition of IL-8 agonist binding (FIG. 3).

### Example 2

In this example, the method for membrane binding assay was used as in Example 1.

### Method for HEK FLIPR Assay Intracellular Calcium Measurements

HEK cells, transfected with the human recombinant CXCR2 receptor, were grown to approximately 80% confluence in 225 cm² flasks in DMEM-Glutamax medium containing, non-essential amino acids, 10% (v/v) FCS in a humidified incubator at 37° C., 5% CO₂. Cells were harvested from the flask using 10× trypsin at 37° C. for 1 to 2 minutes.

HEK 293 transfectants were seeded at 5×10⁴ cells per well in 96 well poly-D lysine coated black with clear bottom plates (Becton Dickinson) and cultured for 16 h at 37° C. with an atmosphere of 5% CO₂/95% air to form confluent monolayers. The following day cells were loaded with 5 μM Flura 3 AM diluted in tyrodes solution (137 mM NaCl, 10 mM HEPES, 0.441 mM KH₂PO₄, 2.67 mM KCl, 1.8 mM CaCL₂, 1 mM MgCl₂) with 6.25 mM probenecid at pH7.4, 100 μl per well and incubated at 37° C. with an atmosphere of 5% CO₂/95% air for 1 h. After which the loading media was removed and the cells washed twice with PBS/HEPES solution (100 μl per well) then overlayed with 50 μl assay buffer (tyrodes buffer without probenecid). 50 μl inhibitor compound or 50 μl 0.2% (v/v) DMSO carrier diluted in assay buffer was then added per well and incubated for 30 minutes at room temperature before the addition of 50 μl 3 mM Carbachol per well. Calcium flux responses were measured using a Fluorometric Imaging Plate Reader (Molecular Devices) for 3 minutes before the addition of either IL-8 (50 μl, 1×10⁻⁷-3×10⁻¹¹ M final concentration) or GROalpha. All points were reproduced in triplicate on each plate. Fluorescence readings were taken every 2 seconds for a total run time of 6 minutes.

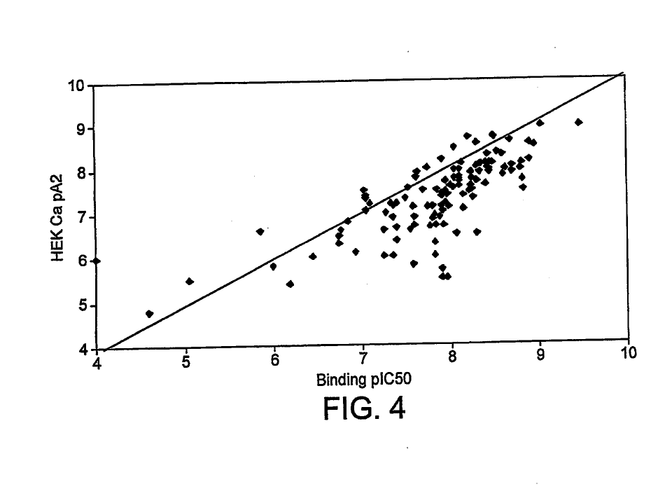

FIG. 4 shows that across a series of compounds similar to Compounds A and C there is a tendency to show reduced potency in a whole cell functional assay as measured by the increase in intracellular calcium concentration using a FLIPR assay compared to a CXCR2 membrane binding assay. One interpretation of these data are that to be active in the whole cell calcium flux assay the compounds need to penetrate the membrane and therefore have a drop in potency when compared to the binding assay. If this is so then the compounds may be acting on an intracellular site.

### Example 3

The activity of Compounds A (30 nM) and B (100 nM) can be switched from being active against CXCR2 and inactive against CXCR1 to inactive against CXCR2 and active against CXCR1, by exchanging the last 60 amino acid residues in the C-terminal tail of CXCR2 (residues 301 to 360) with the last 60 amino acids of CXCR1 (residues 291-350). These last sixty residues are part of transmembrane domain 7 and the cytoplasmic C-terminal tail which is known to be involved in downstream signaling (Ben-Baruch et al. Journal of Biological Chemistry (1995) 9121).

### Methods

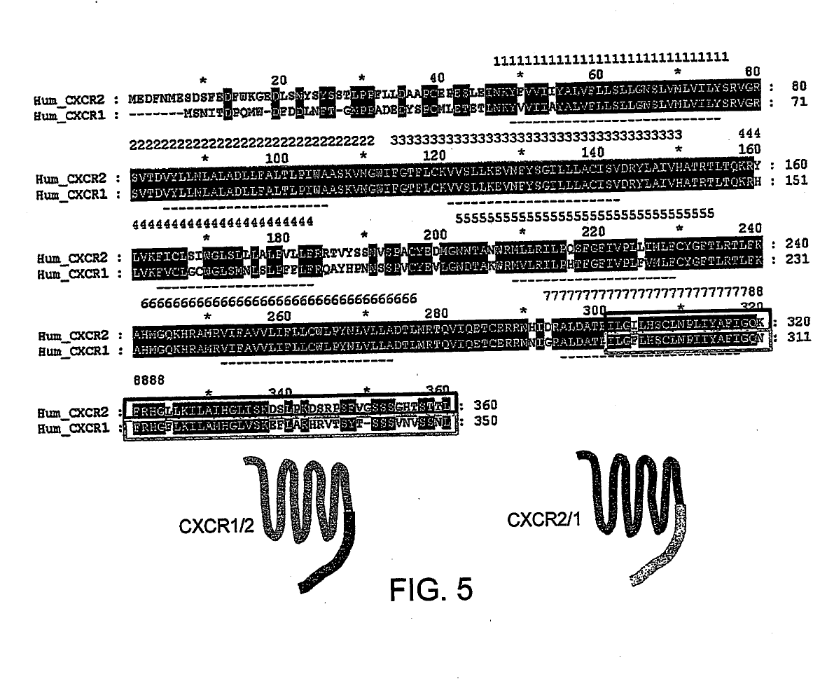

The cDNAs encoding the human Chemokine receptors CXCR1 and CXCR2 were cloned into pIRESneo2 using standard methods as described in Sambrook et al., (1989) and confirmed by sequencing. Using these plasmids as a template CXCR1 and CXCR2 Chimeras, CXCR1 (amino acids 1-290)/CXCR2 (amino acids 301 to 360) and CXCR2 (amino acids 1-300)/CXCR1 (amino acids 291-350) were generated by ligating Xcm I restriction fragments of CXCR1 and CXCR2 (where the last 60 amino acids of the carboxy terminus from each receptor are swapped). Briefly 3 μg CXCR1/pIRESneo2 and 3 μg CXCR2/pIRESneo2 were digested with the restriction enzymes Xcm I and Not I then separated on a 1% agarose gel. Restriction enzyme digests resulted in the presence of two DNA fragments per plasmid. The larger fragment encoding the plasmid pIRESneo2 and either CXCR1 (amino acids 1-290) or CXCR2 (amino acids 1-300) and the smaller DNA fragment encoding the last 60 amino acids of each receptor CXCR1 (amino acids 291-350) or CXCR2 (amino acids 301 to 360). DNA fractions were excised from the gel and purified using QIAquick Gel Extraction kit (Qiagen) following manufacturer's instructions. The DNA fractions encoding the plasmid DNA and either CXCR1 (amino acids 1-290) or CXCR2 (amino acids 1-300) were dephosphorylated using calf intestinal alkaline phosphatase (Invitrogen). CXCR1 and CXCR2 chimeras were generated by ligating pIRESneo2 CXCR1 (amino acids 1-290) DNA with the DNA encoding CXCR2 (amino acids 300-360) and pIRESneo2 CXCR2 (amino acids 1-300) DNA with the DNA encoding CXCR1 (amino acids 291-350) using T4 DNA ligase (New England Biolabs) at 16° C. overnight. DNA ligations were transformed into competent TOP 10 E. coli (Invitrogen) and positive colonies were screened and confirmed by DNA sequencing. A schematic diagram of the constructs generated is shown in FIG. 5.

Adherent HEK 293 cells were maintained as monolayer cultures in Dulbecco's modified Eagle's medium (DMEM) supplemented with 10% FBS and 2 mM L-glutamine (All from Sigma). HEK 293 cells were transfected with CXCR1 and CXCR2 receptor chimeras and mutant plasmids using the lipofection reagent Fugene 6 (Roche). HEK 293 cells were seeded at 4×10⁵ cells per well in a 6 well plate (Costar) and grown to reach 70% confluency for transfection. Fugene 6 lipofection reagent and plasmid DNA (1 μg) was mixed at a ratio of 6:1 in a final volume of 5001 PBS and incubated for 15 minutes at room temperature before addition to HEK 293 cells in culture media (drop wise). Cells were then incubated over night at 37° C. with an atmosphere of 5% CO₂/95% air. Stable transfectants expressing CXCR1 and CXCR2 mutants and chimeras were selected for and maintained by addition of Geneticin G418 at 1 mg/ml (Invitrogen). Transfected cell populations were then screened for responses to IL-8 and GRO-α by measuring intracellular calcium flux using the method as described in Example 2.

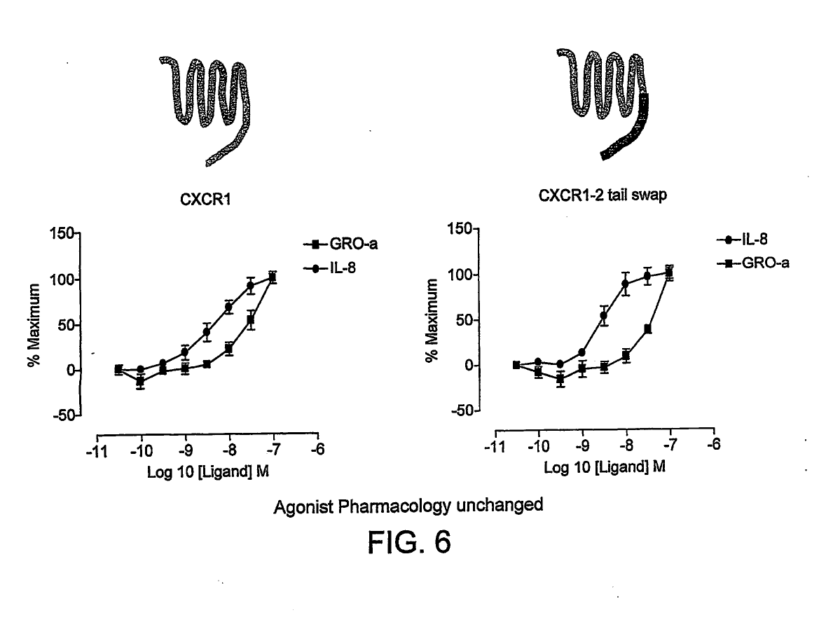

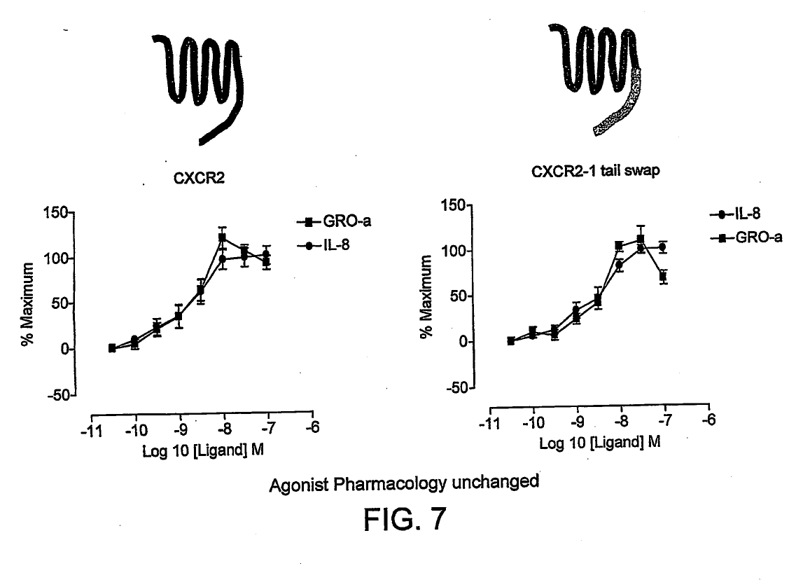

Initially, experiments were performed to check that the chimeras still had correct functional responses to their ligands. FIGS. 6 and 7 show that both CXCR1/2 and CXCR2/1 chimeras responded correctly to their respective ligand despite having the C-terminal residues of the opposing receptor. These data confirm that of Denise Merz et al. presented at ACR, (“The Phosphate Transporter Pit-1 is Upregulated in Osteoarthritic Cartilage and Promotes Chondrocyte Hypertrophy” Category: 06 Cartilage biology and pathogenesis of osteoarthritis Presentation Number: 1076 and that of Ahjuja et al. (Journal of Biological Chemistry 271:225-232 (1996)).

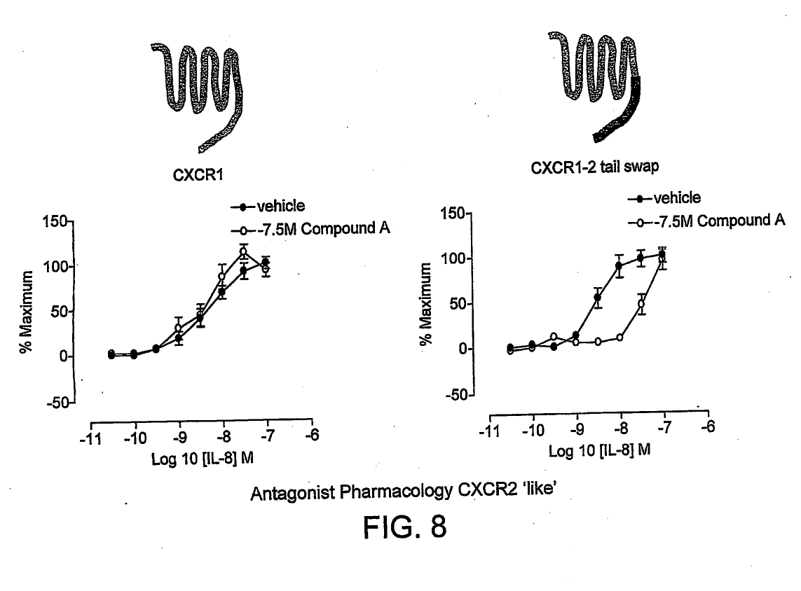

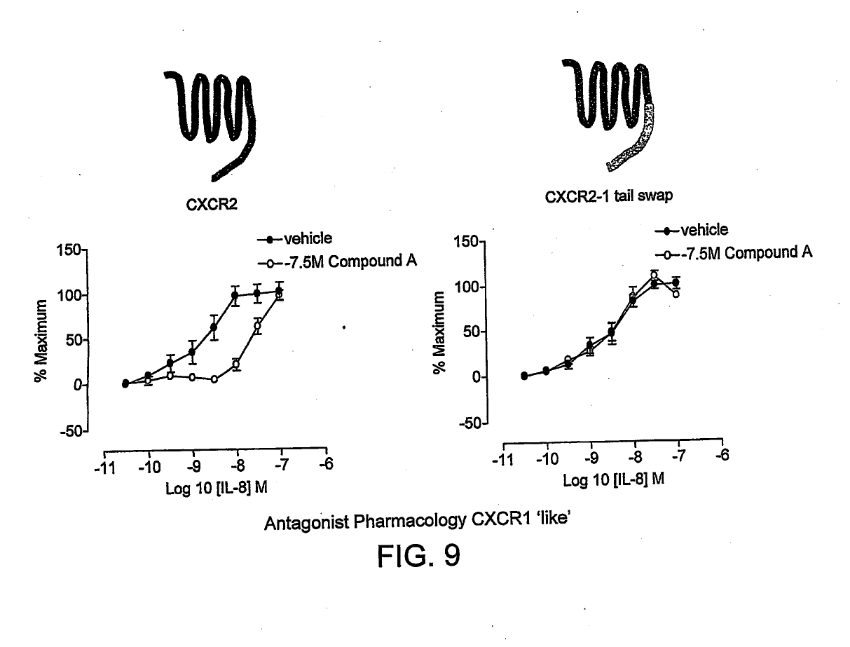

Secondly, the ability of Compound A (at 30 nM) to inhibit the calcium flux induced by the chimeric proteins in HEK cells was tested. The results are shown in FIGS. 8 and 9.

Very similar data to that obtained with 30 nM Compound A were obtained using Compound B at 100 nM (not shown). These data suggest that a particular amino acid or amino acids found in the last 56 (not 60 as the first 4 of these are identical in CXCR2 and CXCR1) amino acids of CXCR2 are at least in part responsible for compound binding as the capability of the chimeric receptors to be inhibited by compounds is changed but their ability to signal in the presence of IL-8 ligand remains unaltered.

### Example 4

Using similar methods to Example 3 it was shown that compound activity is not affected by exchanging the last 35 amino acid residues in the C-terminal tail of CXCR1 and CXCR2.

### Methods

To generate CXCR1 and CXCR2 chimeras CXCR1 (amino acids 1-316)/CXCR2 (amino acids 327-360) (CXCR1/2short) and CXCR2 (amino acids 1-326)/CXCR1 (amino acids 317-350) (CXCR2/1 short), AflII restriction enzymes sites were introduced into the DNA sequences of both receptors by mutagenesis using the QuikChange XL Site Directed Mutagenesis Kit (Stratagene) following manufacturer's directions. Mutagenesis primers are detailed in Table 1 (CXCR1 AflF, CXCR1AFlR, CXCR2 AflF and CXCR2 AFlR).

TABLE 1
CXCR1/CXCR2 MUTAGENESIS PRIMERS

| Mutagenesis | |
|---|---|
| Primer | Sequence 5′-3′ |
| CXCR1 AflF | CCATGGATTCCTTAAGATCCTGGCTATG |
| CXCR1 AflR | CATAGCCAGGATCTTAAGGAATCCATGG |
| CXCR2 AflF | GCCATGGACTCCTTAAGATTCTAGCTATAC |
| CXCR2 AflR | GTATAGCTAGAATCTTAAGGAGTCCATGGC |
| CXCR1 N311K F | CTTCATCGGCCAAAAGTTTCGCCATGGATTC |
| CXCR1 N311K R | GAATCCATGGCGAAACTTTTGGCCGATGAAG |
| CXCR1 F316L F | CAAAATTTTCGCCATGGACTCCTCAAGATCCTGG |
| CXCR1 F316L R | CCAGGATCTTGAGGAGTCCATGGCGAAAATTTTG |
| CXCR2 K320N F | CCTTCATTGGCCAGAATTTTCGCCATGGACTC |
| CXCR2 K320N R | GAGTCCATGGCGAAAATTCTGGCCAATGAAGG |
| CXCR1 N311K/ | CATCGGCCAAAAGTTTCGCCATGGACTCCTCAAGATC |
| F316L F | |
| CXCR1 N311K/ | GATCTTGAGGAGTCCATGGCGAAACTTTTGGCCGATG |
| F316L R | |
| CXCR2 K320N/ | CTTCATTGGCCAGAATTTTCGCCATGGATTCCTCAA |
| L325F F | GATTC |
| CXCR2 K320N/ | GAATCTTGAGGAATCCATGGCGAAAATTCTGGCCA |
| L325F R | ATGAAG |

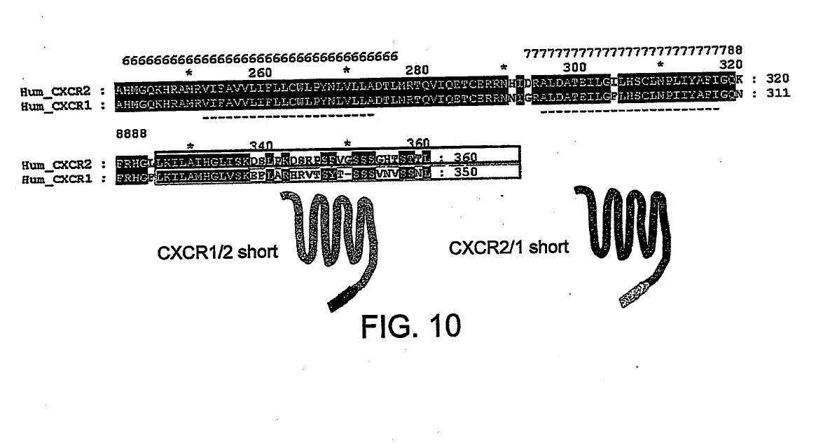

The human Chemokine receptors CXCR1 and CXCR2 cloned into pIRESneo2 were used as a template for these reactions. Positive colonies were screened and confirmed by DNA sequencing. Using the AflII mutated plasmids as a template CXCR1 and CXCR2 Chimera's CXCR1 (amino acids 1-316)/CXCR2 (amino acids 327-360) and CXCR2 (amino acids 1-326)/CXCR1 (amino acids 317-350) were generated by ligating AflII restriction fragments of CXCR1 and CXCR2 (where the last 34 amino acids of the carboxy terminus from each receptor are swapped). Briefly 1 μg CXCR1/pIRESneo2 Afl II mutant DNA and 1 μg CXCR2/pIRESneo2 AflII mutant DNA were digested with the restriction enzymes AflII and NotI then separated on a 1% agarose gel. Restriction enzyme digests resulted in the presence of two DNA fragments per plasmid. The larger fragment encoding the plasmid pIRESneo2 and either CXCR1 (amino acids 1-316) or CXCR2 (amino acids 1-326) and the smaller DNA fragment encoding the last 34 amino acids of each receptor CXCR1 (amino acids 317-350) or CXCR2 (amino acids 327-360). DNA fractions were excised from the gel and purified using QIAquick Gel Extraction kit (Qiagen) following manufacturer's instructions. The DNA fractions encoding the plasmid DNA and either CXCR1 (amino acids 1-316) or CXCR2 (amino acids 1-326) were dephosphorylated using calf intestinal alkaline phosphatase (Invitrogen). The CXCR1 and CXCR2 chimeras were therefore generated by ligating pIRESneo2 CXCR1 large fragment (encoding amino acids 1-316) with CXCR2 small fragment (encoding amino acids 327-360) and pIRESneo2 CXCR2 large fragment (amino acids 1-326) with CXCR1 small fragment (amino acids 317-350) using T4 DNA ligase (New England Biolabs) at 16° C. overnight. DNA ligations were transformed into competent TOP 10 E. coli (Invitrogen) and positive colonies were screened and confirmed by DNA sequencing. A schematic diagram of the constructs generated is shown in FIG. 10.

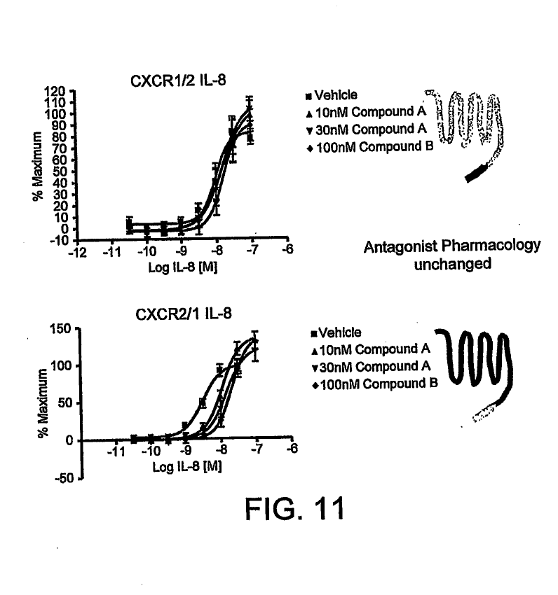

These two new constructs were transfected into HEK 293 cells as described in Example 3. The ability of the chimeric constructs to signal to IL-8 and GROalpha (data not shown) was unchanged and, when tested in the presence of Compound A and B at 30 nM and 100 nM respectively, the compounds did not inhibit the CXCR1/2short but did inhibit the CXCR2/1short (FIG. 11).

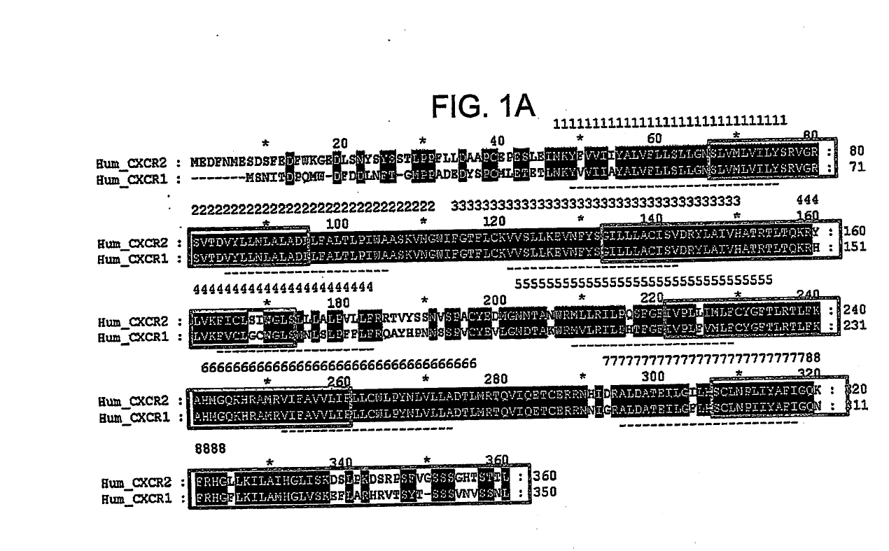

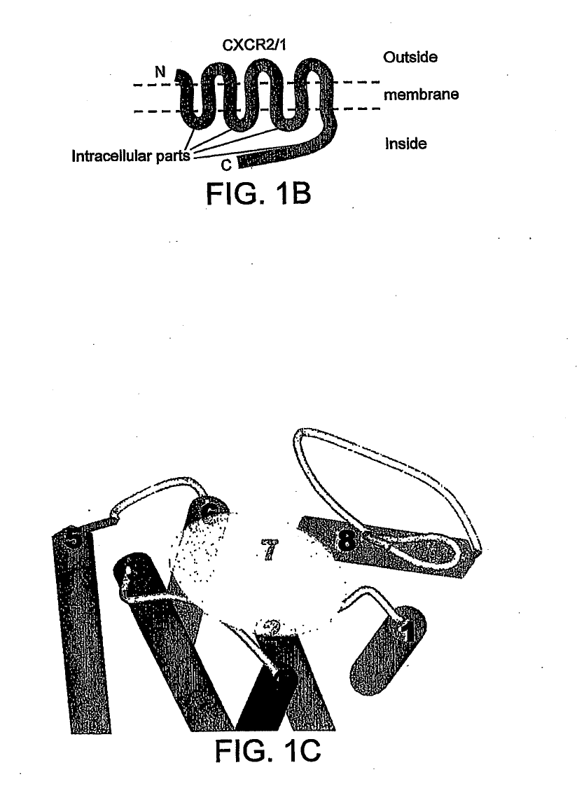

The data in FIG. 11 coupled with information in Example 3 indicates that residues required for Compound A and B binding, and compounds in similar series, are located between amino acids 304 to 326 in CXCR2. The majority of this region lies within the intracellular part of the receptor (FIG. 1).

### Example 5

The activity of Compounds A (10 nM and 30 nM) and B (100 nM) can be switched from being active against CXCR2 and inactive against CXCR1 to inactive against CXCR2 and active against CXCR1 by exchanging the single amino acid residue lysine320 in CXCR2 and the equivalent asparagine residue in CXCR1 (asn310).

### Cloning CXCR1 and CXCR2 Receptor Mutants CXCR1N311K, CXCR1F316L, CXCR2 K320N. CXCR1N311K/F316L and CXCR2 K320N/L325F

Using CXCR1 pIRESneo2 and CXCR2 pIRESneo2 plasmids as a templates, DNA primers with single or double base mismatches (Table 1) were designed to generate the following receptor mutants CXCR1 N311K, CXCR1 F316L, CXCR2 K320N, CXCR1 N311K/F316L and CXCR2 K320N/L325F using the QuikChange XL Site-Directed Mutagenesis Kit (Stratagene) following manufacturer's directions. Positive colonies were screened and confirmed by DNA sequencing.

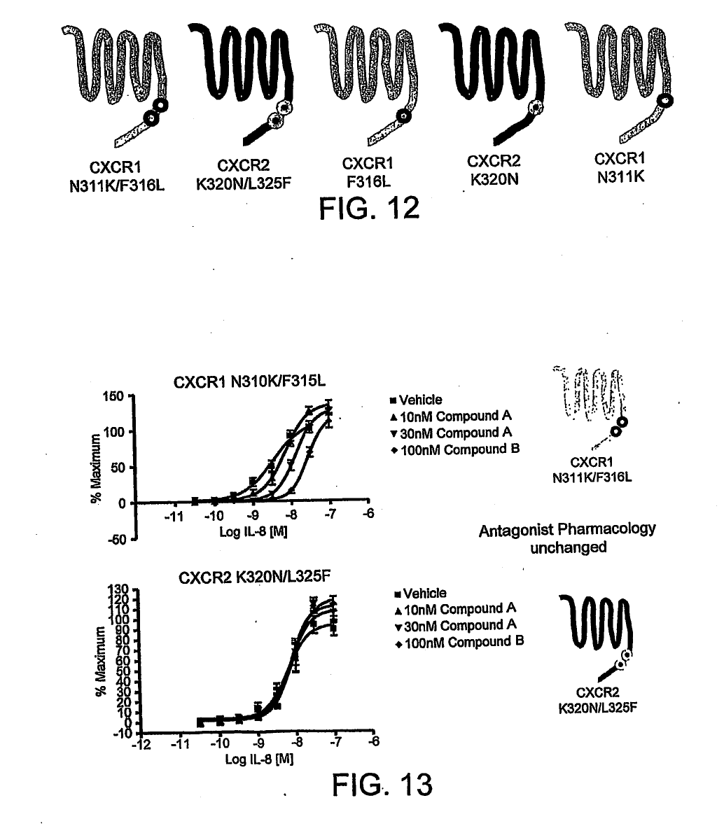

A schematic diagram of these new constructs is shown in FIG. 12.

The constructs were transfected into HEK cells as above (Example 5) and selected in media containing G418. Their response to the ligand IL-8 was tested using the whole cell calcium flux assays in the presence and absence of Compounds A and B. FIGS. 13, 14 and 15 show the effect of the compounds on the ability of the ligand to cause a change in intracellular calcium concentrations.

FIG. 13 shows that substituting both the asn at position 311 and the phe at position 316 in CXCR1 with a lysine and leucine respectively causes an increase in the ability of the compounds to inhibit the mutant receptor. The equivalent substitution of amino acids in CXCR2 causes a decrease in the ability of the compounds to inhibit the mutant CXCR2 receptor.

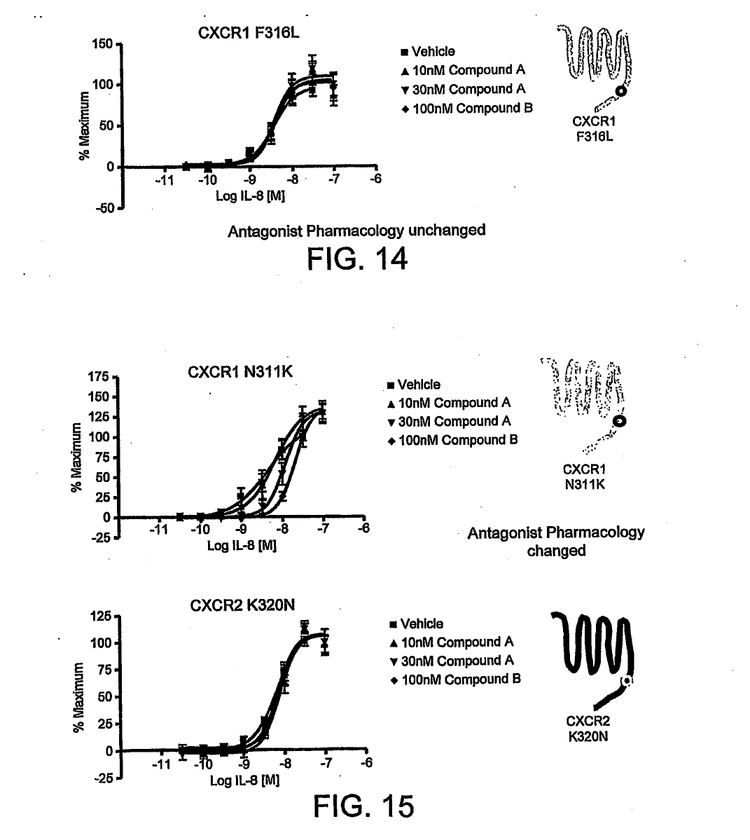

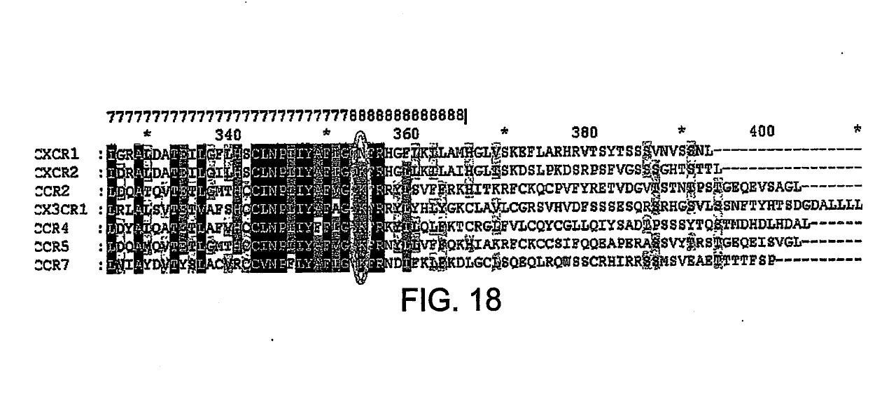

FIG. 14 shows that when only phe316 in CXCR1 is mutated to leucine there is no effect on agonist potency or antagonist pharmacology. However when only the asn at position 311 in CXCR1 is mutated to a lysine (FIG. 15) there is a dramatic increase in antagonist potency in the mutant receptor and a similar decrease in antagonist potency on the equivalent CXCR2 K320N mutant. These data suggest that the antagonists exemplified by Compound A and Compound B require lysine 320 in order to functionally antagonise CXCR2. Because wild type CXCR1 does not have this residue the compounds are inactive at the concentration tested against CXCR1. A sequence alignment of the CXCR2 coding sequence with other GPCRs and bovine rhodopsin was generated using ClustalW and it was shown that the equivalent residue to K320 in CXCR2 is also a lysine in several Chemokine receptors including, CCR2, CX₃CR1, CCR4, CCR5 and CCR7 (FIG. 18).

### Example 6

Using assays similar to those in Example 1 and Example 2 it was shown that Compound A and Compound F are active at the concentration tested against a number of Chemokine receptors. These include CCR1, CXCR2, CXCR1, CCR2, CX₃CR1, CCR4, CCR5 and CCR7 (Table 2). As Compound A binds to an intracellular allosteric binding site of CXCR2, and Compound F is a very closely related compound from the same series, it indicates that a similar novel intracellular binding site as described for CXCR2 exists in a variety of related Chemokine receptors and can be used to inhibit them via an allosteric interaction. Changes in activity against different receptors is likely to be caused by small differences in receptor sequence or to changes in a second shell of residues around the binding site which affects the precise position and orientation of the amino acids in the first shell.

TABLE 2

| | |
|---|---|
| | Compound F |
| 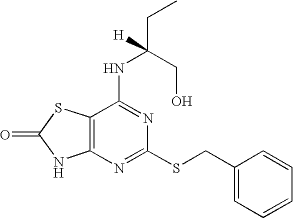 | |

| | Compound A (pIC50) | Compound F (pIC50) |
|---|---|---|
| CXCR2 | 8.9 | 7.7 |
| CXCR1 | 6.6 | ND |
| CCR1 | 62% Inhibition at 10 uM | 46% Inhibition at 10 uM |
| CCR2b | 6.8 | 6.4 |
| CCR4 | 5.1 | 5.4 |
| CCR5 | 72% Inhibition at 10 uM | 100% Inhibition at 10 uM |
| CCR7 | ND | 5.7 |
| CX₃CR1 | 6.6 | 6.0 |

ND = Not determined

### Example 7

### 1) CXCR2 Model Generation

A sequence alignment of the CXCR2 coding sequence with other GPCRs and bovine rhodopsin was generated using ClustalW with default settings and then modified to reflect known trans-membrane defining motifs. This alignment along with the structure of bovine rhodopsin (119h.pdb) were used as input to Modeller version 5, run through the insightII 2000 interface with default settings. A total of 20 models were produced. Of these, for the initial analysis, the structure with the lowest penalty function was used. Hydrogens and charges were subsequently added in Sybyl version 6.9 and siteID was used to visualise the binding site cavities. The resulting model was then used as the structure for subsequent dockings using GOLD version 2.12 and for selecting residues for mutagenesis.

TABLE 3 lists the PDB coordinate file of CXCR2 homology model

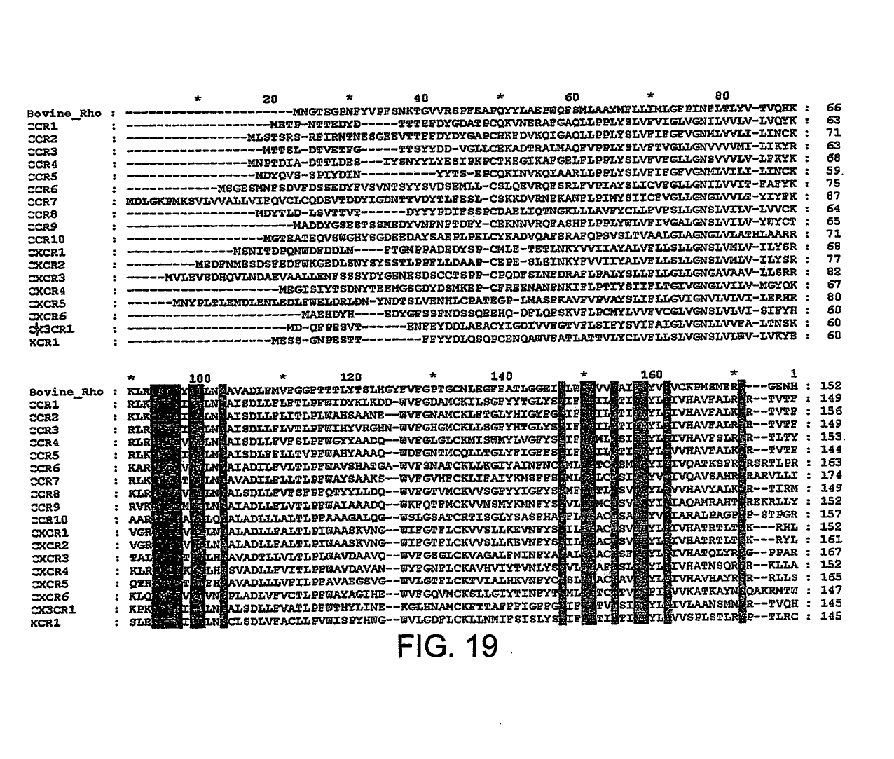

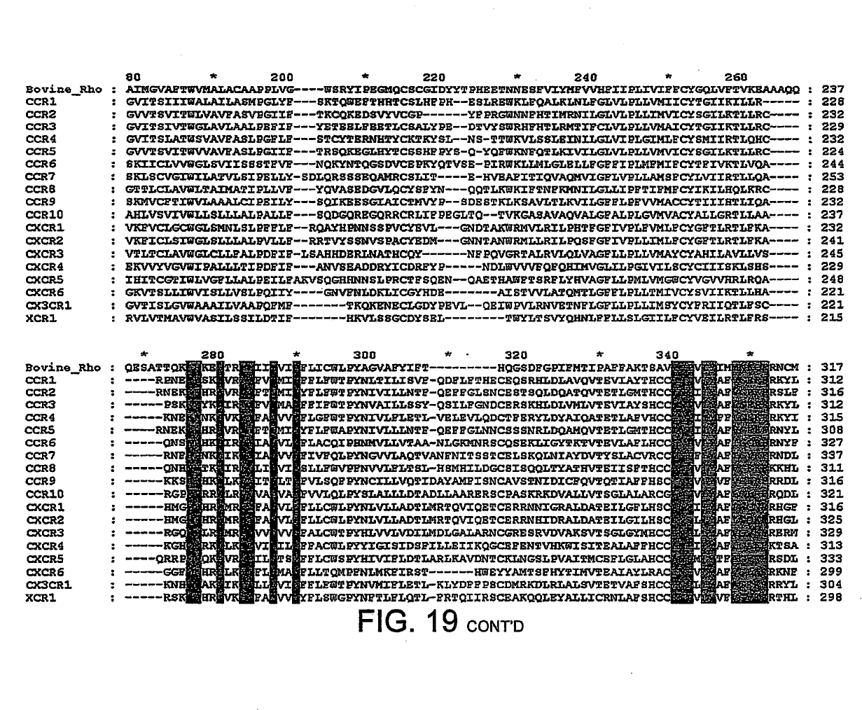

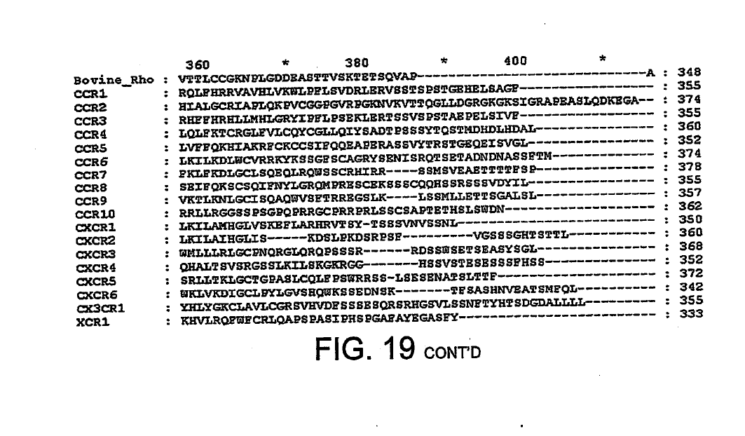

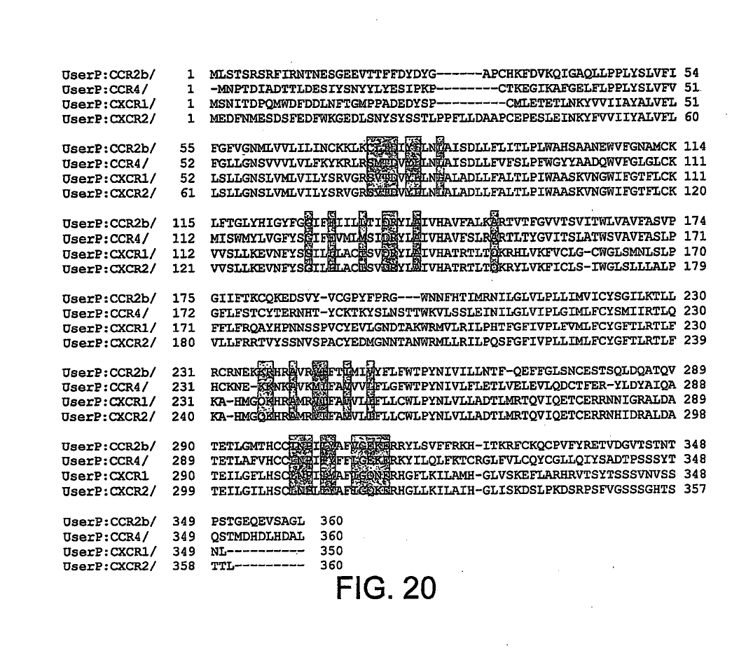

The intracellular domain portion of CXCR2, based on alignments with bovine rhodopsin comprises amino acid residues S67 to D94, residues G133 to S173, residues I221 to F260 and residues S307 to L360. The intracellular allosteric binding site contains one or more of amino acids S81, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 of CXCR2 and their equivalent residues based on alignment with bovine rhodopsin as seen in FIG. 19.

### Example 8

Docking of ligands into the intracellular binding site using GOLD. The native protein homology model structure may be further refined by first manually docking an active compound into the site and rearranging a minimal number of side chains to accommodate the ligand and to match known SAR and mutagenesis. The protein co-ordinates may then be refined, such as by minimisation, if necessary. Once prepared, a set of ligands—built using CORINA, or another suitable 3D coordinate generation program—would be docked in using the program GOLD. This is an automated procedure, but several parameters may be optimised to reproduce known SAR in the dockings. Once the docking protocol is established and is demonstrated to successfully dock in known ligands in a manner consistent with known SAR, novel compounds with unknown affinity for the target protein can then be docked in. Top ranking compounds can then be selected for purchase, synthesis, used as reagents in library synthesis and/or testing. The top ranking compounds could also be scored using an external scoring routine—e.g. CSCORE to further refine the selection. The above protocol would be very similar for other docking routines, such as GLIDE.

### Example 9

### Design of compounds using sequence analysis and 1D and 2D information

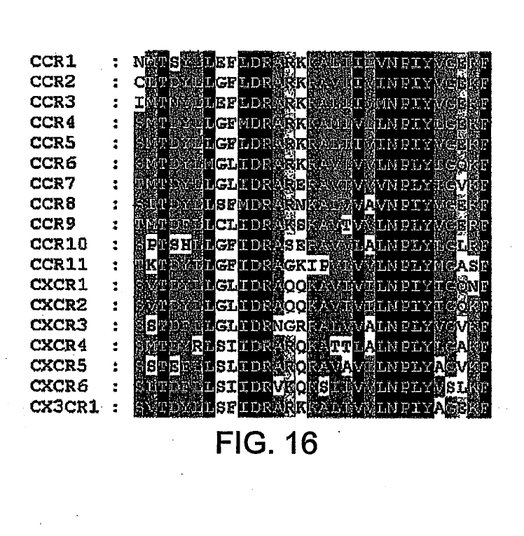

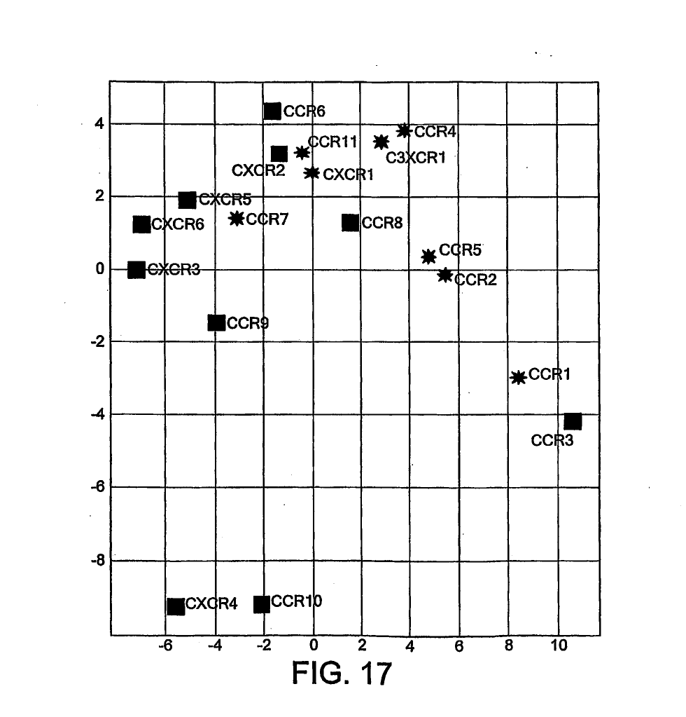

FIG. 16 shows the sequence alignment of the first shell of residues surrounding the proposed binding site identified in CXCR2 and applied to other GPCRs. There is considerable conservation of sequence. A PCA plot of the properties of the residues is shown in FIG. 17. The targets with known intracellular binding sites as described in Example 6 are highlighted as stars. The position of the targets with known intracellular binding sites relative to other Chemokine receptors, and the sequence similarity suggests that it is likely that other Chemokine receptors and GPCRs will also have similar sites. Differences in SAR between Chemokine receptors may be due either to small differences in sequence or to changes in a second shell of residues around the binding site which affects the precise position and orientation of the amino acids in the first shell. Descriptors of the amino acids and the type of functional groups that individual amino acid types associate with, can be used in the absence of structural information to suggest chemical scaffolds, reagents or chemistries for library synthesis and/or to suggest compounds for purchase/synthesis.

### Example 10

### Disulfide Trapping: a Method to Detect Whether a Candidate Compound Forms Associations with One or More Amino Particular Acid Residues

Disulfide trapping is a method that was used by Buck E and Wells J A (2005, PNAS USA 102(8):2719-24) to localize small-molecule agonists and antagonists for the C5a receptor, a GPCR. The method may be used in an assay according to the first aspect of the present invention.

Disulfide trapping may be used to identify candidate compounds that bind to one or more particular amino acid residues of the intracellular allosteric site of a GPCR, wherein the particular amino acids are those corresponding to any one of amino acid residues S8 1, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 of CXCR2, or to any one of amino acid residues 301 to 360 of CXCR2 (including to any one of amino acid residues 304 to 326 of CXCR2).

Firstly GPCR mutants (or variants, homologues, derivatives or fragments thereof) are specifically engineered, where one or more of the particular amino acid residues is converted to a cysteine. Binding studies are then performed on cells or membranes isolated from cells transfected with the mutant, using a library of thiol-containing small molecules or cysteine-containing peptide receptors under reducing conditions, allowing the formation of disulfide bonds. This methodology allows for the identification of weak-binding ligands (candidate compounds) that are associated specifically with the particular amino acid residue(s) of interest.

### Example 11

### Photoaffinity Labelling: a Method to Detect Whether a Candidate Compound Forms Associations with One or More Amino Particular Acid Residues

The method of photoaffinity labelling and proteomic characterisation was described in Murray et al (Nature Chemical Biology 2005, 1:371). The method may be used in an assay according to the first aspect of the present invention, by identifying the residues directly involved in candidate compound binding.

Photoaffinity labelling and proteomic characterisation may be used to identify candidate compounds that bind to one or more particular amino acid residues of the intracellular allosteric site of a GPCR, wherein the particular amino acids are those corresponding to any one of amino acid residues S81, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 of CXCR2, or to any one of amino acid residues 301 to 360 of CXCR2 (including to any one of amino acid residues 304 to 326 of CXCR2).

One example uses Compound A (which we have shown to interact with the intracellular site of CXCR2) or compounds from the same chemical series. The compound is labelled with a radio- and photoaffinity label (probe compound). The precise position of the radiolabel and photoaffinity label in the new compound is determined by the SAR of the compound series, the physico-chemical properties of the compound series and the route of synthesis of the compound as determined by one skilled in the art. This new photoaffinity compound is then used to probe membranes of cells expressing the GPCR of interest and should be able to bind covalently upon photoaffinity labelling to amino acid residues which make up the compound binding site. The specificity of photoaffinity compound binding is determined by carrying out the experiment in increasing concentrations of cold compound (eg Compound A) which binds to the same site and so competes off the probe compound before affinity labelling at increasing concentrations of cold compound.

Once the specific binding of the probe compound has been identified, cell membranes containing the target GPCR are photoaffinity labelled with the probe compound. The membranes are then treated with a suitable detergent (eg. digitonin) to extract the proteins and the extracted protein is resolved by polyacrylamide gel electrophoresis to check that the protein is labelled.

To identify the position where the protein is labelled, the solubilised, photoaffinity labelled protein and control-untreated protein are proteolytically cleaved with agents well known in the art such as trypsin and cyanogen bromide to release peptides with known cleavage sites. Due to their cleavage site sequence, the mass and properties of these peptides can be predicted from the primary amino-acid sequence of the target GPCR protein.

The peptides are fractionated on the basis of size, charge and hydrophobicity using techniques well known in the art in order to separate them. The fractions containing radiolabel are likely to contain a peptide which has the photoaffinity compound covalently attached to it.

Any individual peptide isolated with the photoaffinity label covalently attached will be larger in molecular mass then the equivalent peptide from membranes which have not been photoaffinity labelled.

As the sequence of the target GPCR is known, any peptide which is found to be larger molecular mass in photo-affinity membranes compared to peptides released from untreated membranes contains specific amino acid residues which are adjacent to, or form part of the compound binding site in the nascent molecule.

### Example 12

### Radio-Ligand Binding to GST-CXCR2 C Terminal Tail

An expression construct which encodes for S. Japonicum glutathione-S-transferase (GST) fused to the last 43 amino acids of human CXCR2 (residues G318 to L360) was generated by standard molecular biological techniques.

The predicted sequence of the GST-CXCR2-C-tail fusion protein is shown below (CXCR2 sequence is underlined, GST sequence is not underlined).

MSPILGYWKI KGLVQPTRLL LEYLEEKYEE
HLYERDEGDK WRNKKFELGL EFPNLPYYID
GDVKLTQSMA IIRYIADKHN MLGGCPKERA
EISMLEGAVL DIRYGVSRIA YSKDFETLKV
DFLSKLPEML KMFEDRLCHK TYLNGDHVTH
PDFMLYDALD VVLYMDPMCL DAFPKLVCFK
KRIEAIPQID KYLKSSKYIA WPLQGWQATF
GGGDHPPKGS LVPRGSGQKF RHGLLKILAI
HGLISKDSLP KDSRPSFVGS SSGHTSTTL

The protein was expressed in E. coli and the resulting fusion protein was purified from the cell extract via chromatography using a 5 ml GSTrap column (Amersham) which binds GST containing proteins, and eluted using 10 mM reduced glutathione in the elution buffer. At the same time GST without a CXCR2 C-terminal tail was also expressed and purified as a control. Both proteins had the excess 10 mM glutathione removed using a XK26/10 desalting column (Amersham).

The purified proteins were used in a radioligand binding assay.

### Radioligand Binding Assay

To each well in a 96-well polypropylene plate was added [³H]Compound A (20 μL, to a final concentration of 20 nM, from a working solution of 50 mM Phosphate-buffered salt solution) and either vehicle (20 μL, 10% (v/v) DMSO in Phosphate-buffered salt solution: for determination of total binding (B0)), or Compound A (20 μL, 1 mM solution in 10% (v/v) DMSO phosphate-buffered salt solution: for determination of non-specific binding (NSB)) were added to the wells of a 96-well polypropylene plate. Protein solutions (50 μL dilutions starting at 1.28 mg/ml) of either GST-CXCR2 fusion or wild type GST were then added, giving a total incubation volume of 200 μL per well. The plates were incubated for 2 hours at room temperature.

A GF-B filter plate was set up containing 50 μl of 1 mg/ml gelatine in PBS in each well, which was then filtered through the plate and discarded. 100 μl filter mix (160 mg/ml charcoal (dextran coated) in PBS containing 1 mg/ml gelatine) was then filtered through the plate and then 150 μl assay mix was then filtered through the prepared filter plate (The charcoal catches the excess un-bound compound). After filtering, 100 μl filtrate was transferred to a lumaplate and dried down in a 50° C. oven overnight and then counted on the TopCount instrument (Packard BioScience)

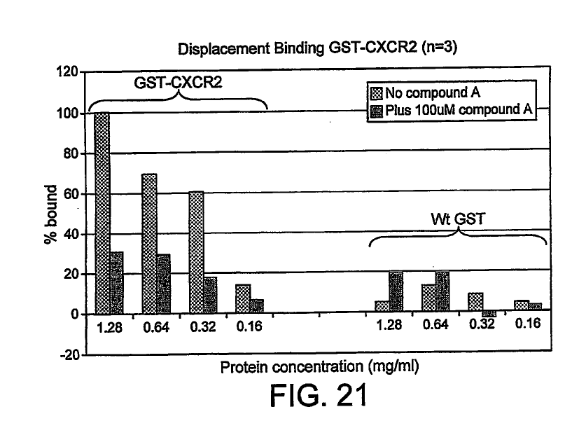

FIG. 21 shows results of the radioligand binding assay using [³H]Compound A with GST-CXCR2 fusion protein and wild type GST. The data in FIG. 21 is the average of three separate experiments. As can be seen as expected on diluting the GST-CXCR2 protein less radio-labelled compound can bind. Using the wild type GST control protein there is minimal compound binding. On the addition of excess cold compound there is a decrease in radiolabelled binding to the GST-CXCR2 fusion protein but no effect with the wild type GST.

### Example 13

### Alternative Method to Express the C-Terminal Portion of a G-Protein Coupled Receptor to Generate Protein that is Membrane Bound or Extracted from a Cell Lysate Using a Detergent

The C-terminal domain and transmembrane helix 7 domain of GPCRs is expressed by a method described in Carillo et al. Molecular Pharmacology 66 1123-1137 (2004). This paper describes expression of various mutant transmembrane domains of the α1b-adrenoceptor in HEK293 cells. The paper exemplifies a TM7 construct that contains the N-terminal 44 amino acids of the α1b-adrenoceptor fused to the TM7 helix and the C-terminal tail of the same receptor. The N-terminal 44 amino acids are necessary for targeting expression of the protein to the membrane. In the paper, this mutant construct was shown to express in HEK293 cells after transient transfection and the mutant protein was extracted from the cell using a detergent-containing buffer.

Similar mutant proteins derived from the sequence of other GPCRs could be suitable proteins to test in compound binding. Alternatively the membrane fraction from these cells expressing the mutant proteins could be used to measure compound binding to the C-terminal portion of the protein expressed in the membranes.

TABLE 3

| HEADER | CXCR2 | | | | | | | | | | | | | | 1MOD |
|---|---|---|---|---|---|---|---|---|---|---|---|---|---|---|---|
| COMPND | CXCR2 | | | | | | | | | | | | | | 1MOD |
| REMARK | GENERATED BY SYBYL (TRIPOS, INC.) | | | | | | | | | | | | 15-DEC-04 | | 1MOD |
| SEQRES | 1 | 356 | MET | GLU | ASP | PHE | ASN | MET | GLU | SER | ASP | SER | PHE | GLU | 1MOD |
| SEQRES | 2 | 356 | PHE | TRP | LYS | GLY | GLU | ASP | LEU | SER | ASN | TYR | SER | TYR | 1MOD |
| SEQRES | 3 | 356 | SER | THR | LEU | PRO | PRO | PHE | LEU | LEU | ASP | ALA | ALA | PRO | 1MOD |
| SEQRES | 4 | 356 | GLU | PRO | GLU | SER | LEU | GLU | ILE | ASN | LYS | TYR | PHE | VAL | 1MOD |
| SEQRES | 5 | 356 | ILE | ILE | TYR | ALA | LEU | VAL | PHE | LEU | LEU | SER | LEU | LEU | 1MOD |
| SEQRES | 6 | 356 | ASN | SER | LEU | VAL | MET | LEU | VAL | ILE | LEU | TYR | SER | ARG | 1MOD |
| SEQRES | 7 | 356 | GLY | ARG | SER | VAL | THR | ASP | VAL | TYR | LEU | LEU | ASN | LEU | 1MOD |
| SEQRES | 8 | 356 | LEU | ALA | ASP | LEU | LEU | PHE | ALA | LEU | THR | LEU | PRO | ILE | 1MOD |
| SEQRES | 9 | 356 | ALA | ALA | SER | LYS | VAL | ASN | GLY | TRP | ILE | PHE | GLY | THR | 1MOD |
| SEQRES | 10 | 356 | LEU | CYS | LYS | VAL | VAL | SER | LEU | LEU | LYS | GLU | VAL | ASN | 1MOD |
| SEQRES | 11 | 356 | TYR | SER | GLY | ILE | LEU | LEU | LEU | ALA | CYS | ILE | SER | VAL | 1MOD |
| SEQRES | 12 | 356 | ARG | TYR | LEU | ALA | ILE | VAL | HIS | ALA | THR | ARG | THR | LEU | 1MOD |
| SEQRES | 13 | 356 | GLN | LYS | ARG | TYR | LEU | VAL | LYS | PHE | ILE | CYS | LEU | SER | 1MOD |
| SEQRES | 14 | 356 | TRP | GLY | LEU | SER | LEU | LEU | LEU | ALA | LEU | PRO | VAL | LEU | 1MOD |
| SEQRES | 15 | 356 | PHE | ARG | ARG | THR | VAL | TYR | SER | SER | ASN | VAL | SER | PRO | 1MOD |
| SEQRES | 16 | 356 | CYS | TYR | GLU | ASP | MET | GLY | ASN | ASN | THR | ALA | ASN | TRP | 1MOD |
| SEQRES | 17 | 356 | MET | LEU | LEU | ARG | ILE | LEU | PRO | GLN | SER | PHE | GLY | PHE | 1MOD |
| SEQRES | 18 | 356 | VAL | PRO | LEU | LEU | ILE | MET | LEU | PHE | CYS | TYR | GLY | PHE | 1MOD |
| SEQRES | 19 | 356 | LEU | ARG | THR | LEU | PHE | LYS | ALA | HIS | MET | GLY | GLN | LYS | 1MOD |
| SEQRES | 20 | 356 | ARG | ALA | MET | ARG | VAL | ILE | PHE | ALA | VAL | VAL | LEU | ILE | 1MOD |
| SEQRES | 21 | 356 | LEU | LEU | CYS | TRP | LEU | PRO | TYR | ASN | LEU | VAL | LEU | LEU | 1MOD |
| SEQRES | 22 | 356 | ASP | THR | LEU | MET | ARG | THR | GLN | VAL | ILE | GLN | GLU | THR | 1MOD |
| SEQRES | 23 | 356 | GLU | ARG | ARG | ASN | HIS | ILE | ASP | ARG | ALA | LEU | ASP | ALA | 1MOD |
| SEQRES | 24 | 356 | GLU | ILE | LEU | GLY | ILE | LEU | HIS | SER | CYS | LEU | ASN | PRO | 1MOD |
| SEQRES | 25 | 356 | ILE | TYR | ALA | PHE | ILE | GLY | GLN | LYS | PHE | ARG | HIS | GLY | 1MOD |
| SEQRES | 26 | 356 | LEU | LYS | ILE | LEU | ALA | ILE | HIS | GLY | LEU | ILE | SER | LYS | 1MOD |
| SEQRES | 27 | 356 | SER | LEU | PRO | LYS | ASP | SER | ARG | PRO | SER | PHE | VAL | GLY | 1MOD |
| SEQRES | 28 | 356 | SER | SER | GLY | HIS | THR | | | | | | | | 1MOD |

| | | | | | | | | | | |
|---|---|---|---|---|---|---|---|---|---|---|
| ATOM | 1 | N | MET | 1 | −9.842 | −43.057 | −21.943 | 1.00 | 0.00 | 1MOD |
| ATOM | 2 | CA | MET | 1 | −10.643 | −43.934 | −22.828 | 1.00 | 0.00 | 1MOD |
| ATOM | 3 | C | MET | 1 | −10.288 | −43.657 | −24.246 | 1.00 | 0.00 | 1MOD |
| ATOM | 4 | O | MET | 1 | −9.142 | −43.337 | −24.558 | 1.00 | 0.00 | 1MOD |
| ATOM | 5 | CB | MET | 1 | −12.146 | −43.668 | −22.639 | 1.00 | 0.00 | 1MOD |
| ATOM | 6 | CG | MET | 1 | −12.645 | −44.019 | −21.235 | 1.00 | 0.00 | 1MOD |
| ATOM | 7 | SD | MET | 1 | −14.389 | −43.621 | −20.905 | 1.00 | 0.00 | 1MOD |
| ATOM | 8 | CE | MET | 1 | −14.273 | −43.898 | −19.113 | 1.00 | 0.00 | 1MOD |
| ATOM | 9 | N | GLU | 2 | −11.271 | −43.782 | −25.155 | 1.00 | 0.00 | 1MOD |
| ATOM | 10 | CA | GLU | 2 | −10.962 | −43.520 | −26.526 | 1.00 | 0.00 | 1MOD |
| ATOM | 11 | C | GLU | 2 | −10.605 | −42.075 | −26.611 | 1.00 | 0.00 | 1MOD |
| ATOM | 12 | O | GLU | 2 | −11.382 | −41.212 | −26.211 | 1.00 | 0.00 | 1MOD |
| ATOM | 13 | CB | GLU | 2 | −12.155 | −43.762 | −27.468 | 1.00 | 0.00 | 1MOD |
| ATOM | 14 | CG | GLU | 2 | −12.552 | −45.235 | −27.579 | 1.00 | 0.00 | 1MOD |
| ATOM | 15 | CD | GLU | 2 | −13.868 | −45.305 | −28.337 | 1.00 | 0.00 | 1MOD |
| ATOM | 16 | OE1 | GLU | 2 | −14.597 | −44.278 | −28.348 | 1.00 | 0.00 | 1MOD |
| ATOM | 17 | OE2 | GLU | 2 | −14.164 | −46.387 | −28.912 | 1.00 | 0.00 | 1MOD |
| ATOM | 18 | N | ASP | 3 | −9.398 | −41.776 | −27.123 | 1.00 | 0.00 | 1MOD |
| ATOM | 19 | CA | ASP | 3 | −9.002 | −40.406 | −27.252 | 1.00 | 0.00 | 1MOD |
| ATOM | 20 | C | ASP | 3 | −9.849 | −39.767 | −28.299 | 1.00 | 0.00 | 1MOD |
| ATOM | 21 | O | ASP | 3 | −10.264 | −38.619 | −28.156 | 1.00 | 0.00 | 1MOD |
| ATOM | 22 | CB | ASP | 3 | −7.528 | −40.221 | −27.660 | 1.00 | 0.00 | 1MOD |
| ATOM | 23 | CG | ASP | 3 | −6.674 | −40.544 | −26.445 | 1.00 | 0.00 | 1MOD |
| ATOM | 24 | OD1 | ASP | 3 | −7.247 | −40.598 | −25.324 | 1.00 | 0.00 | 1MOD |
| ATOM | 25 | OD2 | ASP | 3 | −5.441 | −40.735 | −26.613 | 1.00 | 0.00 | 1MOD |
| ATOM | 26 | N | PHE | 4 | −10.153 | −40.506 | −29.382 | 1.00 | 0.00 | 1MOD |
| ATOM | 27 | CA | PHE | 4 | −10.887 | −39.884 | −30.442 | 1.00 | 0.00 | 1MOD |
| ATOM | 28 | C | PHE | 4 | −12.309 | −40.326 | −30.368 | 1.00 | 0.00 | 1MOD |
| ATOM | 29 | O | PHE | 4 | −12.608 | −41.469 | −30.027 | 1.00 | 0.00 | 1MOD |
| ATOM | 30 | CB | PHE | 4 | −10.392 | −40.262 | −31.848 | 1.00 | 0.00 | 1MOD |
| ATOM | 31 | CG | PHE | 4 | −8.961 | −39.868 | −31.958 | 1.00 | 0.00 | 1MOD |
| ATOM | 32 | CD1 | PHE | 4 | −8.608 | −38.602 | −32.365 | 1.00 | 0.00 | 1MOD |
| ATOM | 33 | CD2 | PHE | 4 | −7.973 | −40.772 | −31.641 | 1.00 | 0.00 | 1MOD |
| ATOM | 34 | CE1 | PHE | 4 | −7.283 | −38.251 | −32.463 | 1.00 | 0.00 | 1MOD |
| ATOM | 35 | CE2 | PHE | 4 | −6.647 | −40.423 | −31.741 | 1.00 | 0.00 | 1MOD |
| ATOM | 36 | CZ | PHE | 4 | −6.301 | −39.160 | −32.152 | 1.00 | 0.00 | 1MOD |
| ATOM | 37 | N | ASN | 5 | −13.231 | −39.396 | −30.678 | 1.00 | 0.00 | 1MOD |
| ATOM | 38 | CA | ASN | 5 | −14.620 | −39.728 | −30.705 | 1.00 | 0.00 | 1MOD |
| ATOM | 39 | C | ASN | 5 | −15.072 | −39.472 | −32.104 | 1.00 | 0.00 | 1MOD |
| ATOM | 40 | O | ASN | 5 | −14.488 | −38.654 | −32.814 | 1.00 | 0.00 | 1MOD |
| ATOM | 41 | CB | ASN | 5 | −15.489 | −38.889 | −29.749 | 1.00 | 0.00 | 1MOD |
| ATOM | 42 | CG | ASN | 5 | −15.381 | −37.431 | −30.165 | 1.00 | 0.00 | 1MOD |
| ATOM | 43 | OD1 | ASN | 5 | −14.289 | −36.869 | −30.232 | 1.00 | 0.00 | 1MOD |
| ATOM | 44 | ND2 | ASN | 5 | −16.548 | −36.798 | −30.461 | 1.00 | 0.00 | 1MOD |
| ATOM | 45 | N | MET | 6 | −16.117 | −40.194 | −32.547 | 1.00 | 0.00 | 1MOD |
| ATOM | 46 | CA | MET | 6 | −16.578 | −40.036 | −33.893 | 1.00 | 0.00 | 1MOD |
| ATOM | 47 | C | MET | 6 | −17.253 | −38.706 | −33.983 | 1.00 | 0.00 | 1MOD |
| ATOM | 48 | O | MET | 6 | −17.801 | −38.215 | −32.998 | 1.00 | 0.00 | 1MOD |
| ATOM | 49 | CB | MET | 6 | −17.574 | −41.124 | −34.322 | 1.00 | 0.00 | 1MOD |
| ATOM | 50 | CG | MET | 6 | −17.720 | −41.251 | −35.838 | 1.00 | 0.00 | 1MOD |
| ATOM | 51 | SD | MET | 6 | −18.787 | −42.625 | −36.355 | 1.00 | 0.00 | 1MOD |
| ATOM | 52 | CE | MET | 6 | −17.805 | −43.885 | −35.489 | 1.00 | 0.00 | 1MOD |
| ATOM | 53 | N | GLU | 7 | −17.204 | −38.072 | −35.173 | 1.00 | 0.00 | 1MOD |
| ATOM | 54 | CA | GLU | 7 | −17.796 | −36.775 | −35.317 | 1.00 | 0.00 | 1MOD |
| ATOM | 55 | C | GLU | 7 | −18.499 | −36.693 | −36.628 | 1.00 | 0.00 | 1MOD |
| ATOM | 56 | O | GLU | 7 | −18.278 | −37.500 | −37.530 | 1.00 | 0.00 | 1MOD |
| ATOM | 57 | CB | GLU | 7 | −16.773 | −35.628 | −35.350 | 1.00 | 0.00 | 1MOD |
| ATOM | 58 | CG | GLU | 7 | −16.151 | −35.297 | −33.995 | 1.00 | 0.00 | 1MOD |
| ATOM | 59 | CD | GLU | 7 | −17.125 | −34.371 | −33.279 | 1.00 | 0.00 | 1MOD |
| ATOM | 60 | OE1 | GLU | 7 | −18.102 | −33.925 | −33.939 | 1.00 | 0.00 | 1MOD |
| ATOM | 61 | OE2 | GLU | 7 | −16.904 | −34.089 | −32.071 | 1.00 | 0.00 | 1MOD |
| ATOM | 62 | N | SER | 8 | −19.401 | −35.699 | −36.733 | 1.00 | 0.00 | 1MOD |
| ATOM | 63 | CA | SER | 8 | −20.056 | −35.382 | −37.963 | 1.00 | 0.00 | 1MOD |
| ATOM | 64 | C | SER | 8 | −19.536 | −34.021 | −38.278 | 1.00 | 0.00 | 1MOD |
| ATOM | 65 | O | SER | 8 | −19.465 | −33.164 | −37.397 | 1.00 | 0.00 | 1MOD |
| ATOM | 66 | CB | SER | 8 | −21.587 | −35.291 | −37.839 | 1.00 | 0.00 | 1MOD |
| ATOM | 67 | OG | SER | 8 | −22.128 | −36.568 | −37.535 | 1.00 | 0.00 | 1MOD |
| ATOM | 68 | N | ASP | 9 | −19.132 | −33.777 | −39.537 | 1.00 | 0.00 | 1MOD |
| ATOM | 69 | CA | ASP | 9 | −18.545 | −32.498 | −39.800 | 1.00 | 0.00 | 1MOD |
| ATOM | 70 | C | ASP | 9 | −19.607 | −31.455 | −39.844 | 1.00 | 0.00 | 1MOD |
| ATOM | 71 | O | ASP | 9 | −20.756 | −31.717 | −40.196 | 1.00 | 0.00 | 1MOD |
| ATOM | 72 | CB | ASP | 9 | −17.704 | −32.445 | −41.085 | 1.00 | 0.00 | 1MOD |
| ATOM | 73 | CG | ASP | 9 | −16.392 | −33.148 | −40.759 | 1.00 | 0.00 | 1MOD |
| ATOM | 74 | OD1 | ASP | 9 | −16.038 | −33.187 | −39.550 | 1.00 | 0.00 | 1MOD |
| ATOM | 75 | OD2 | ASP | 9 | −15.725 | −33.652 | −41.701 | 1.00 | 0.00 | 1MOD |
| ATOM | 76 | N | SER | 10 | −19.227 | −30.222 | −39.459 | 1.00 | 0.00 | 1MOD |
| ATOM | 77 | CA | SER | 10 | −20.168 | −29.147 | −39.434 | 1.00 | 0.00 | 1MOD |
| ATOM | 78 | C | SER | 10 | −19.571 | −28.018 | −40.203 | 1.00 | 0.00 | 1MOD |
| ATOM | 79 | O | SER | 10 | −18.386 | −28.025 | −40.529 | 1.00 | 0.00 | 1MOD |
| ATOM | 80 | CB | SER | 10 | −20.455 | −28.611 | −38.020 | 1.00 | 0.00 | 1MOD |
| ATOM | 81 | OG | SER | 10 | −21.380 | −27.537 | −38.083 | 1.00 | 0.00 | 1MOD |
| ATOM | 82 | N | PHE | 11 | −20.417 | −27.030 | −40.544 | 1.00 | 0.00 | 1MOD |
| ATOM | 83 | CA | PHE | 11 | −19.974 | −25.875 | −41.263 | 1.00 | 0.00 | 1MOD |
| ATOM | 84 | C | PHE | 11 | −19.509 | −24.884 | −40.249 | 1.00 | 0.00 | 1MOD |
| ATOM | 85 | O | PHE | 11 | −19.874 | −24.964 | −39.077 | 1.00 | 0.00 | 1MOD |
| ATOM | 86 | CB | PHE | 11 | −21.081 | −25.238 | −42.120 | 1.00 | 0.00 | 1MOD |
| ATOM | 87 | CG | PHE | 11 | −20.515 | −24.047 | −42.813 | 1.00 | 0.00 | 1MOD |
| ATOM | 88 | CD1 | PHE | 11 | −19.634 | −24.191 | −43.861 | 1.00 | 0.00 | 1MOD |
| ATOM | 89 | CD2 | PHE | 11 | −20.883 | −22.781 | −42.423 | 1.00 | 0.00 | 1MOD |
| ATOM | 90 | CE1 | PHE | 11 | −19.119 | −23.086 | −44.497 | 1.00 | 0.00 | 1MOD |
| ATOM | 91 | CE2 | PHE | 11 | −20.374 | −21.672 | −43.054 | 1.00 | 0.00 | 1MOD |
| ATOM | 92 | CZ | PHE | 11 | −19.487 | −21.823 | −44.093 | 1.00 | 0.00 | 1MOD |
| ATOM | 93 | N | GLU | 12 | −18.665 | −23.925 | −40.677 | 1.00 | 0.00 | 1MOD |
| ATOM | 94 | CA | GLU | 12 | −18.155 | −22.966 | −39.744 | 1.00 | 0.00 | 1MOD |
| ATOM | 95 | C | GLU | 12 | −17.464 | −23.726 | −38.667 | 1.00 | 0.00 | 1MOD |
| ATOM | 96 | O | GLU | 12 | −17.613 | −23.413 | −37.486 | 1.00 | 0.00 | 1MOD |
| ATOM | 97 | CB | GLU | 12 | −19.254 | −22.151 | −39.042 | 1.00 | 0.00 | 1MOD |
| ATOM | 98 | CG | GLU | 12 | −20.045 | −21.214 | −39.953 | 1.00 | 0.00 | 1MOD |
| ATOM | 99 | CD | GLU | 12 | −21.107 | −20.562 | −39.080 | 1.00 | 0.00 | 1MOD |
| ATOM | 100 | OE1 | GLU | 12 | −22.096 | −21.265 | −38.735 | 1.00 | 0.00 | 1MOD |
| ATOM | 101 | OE2 | GLU | 12 | −20.938 | −19.366 | −38.727 | 1.00 | 0.00 | 1MOD |
| ATOM | 102 | N | ASP | 13 | −16.691 | −24.757 | −39.050 | 1.00 | 0.00 | 1MOD |
| ATOM | 103 | CA | ASP | 13 | −16.015 | −25.514 | −38.044 | 1.00 | 0.00 | 1MOD |
| ATOM | 104 | C | ASP | 13 | −15.137 | −24.574 | −37.300 | 1.00 | 0.00 | 1MOD |
| ATOM | 105 | O | ASP | 13 | −14.316 | −23.870 | −37.882 | 1.00 | 0.00 | 1MOD |
| ATOM | 106 | CB | ASP | 13 | −15.119 | −26.635 | −38.592 | 1.00 | 0.00 | 1MOD |
| ATOM | 107 | CG | ASP | 13 | −16.019 | −27.787 | −38.997 | 1.00 | 0.00 | 1MOD |
| ATOM | 108 | OD1 | ASP | 13 | −17.089 | −27.944 | −38.352 | 1.00 | 0.00 | 1MOD |
| ATOM | 109 | OD2 | ASP | 13 | −15.651 | −28.526 | −39.950 | 1.00 | 0.00 | 1MOD |
| ATOM | 110 | N | PHE | 14 | −15.294 | −24.561 | −35.965 | 1.00 | 0.00 | 1MOD |
| ATOM | 111 | CA | PHE | 14 | −14.545 | −23.677 | −35.131 | 1.00 | 0.00 | 1MOD |
| ATOM | 112 | C | PHE | 14 | −13.885 | −24.437 | −34.033 | 1.00 | 0.00 | 1MOD |
| ATOM | 113 | O | PHE | 14 | −12.660 | −24.520 | −33.975 | 1.00 | 0.00 | 1MOD |
| ATOM | 114 | CB | PHE | 14 | −15.400 | −22.587 | −34.462 | 1.00 | 0.00 | 1MOD |
| ATOM | 115 | CG | PHE | 14 | −14.666 | −22.189 | −33.222 | 1.00 | 0.00 | 1MOD |
| ATOM | 116 | CD1 | PHE | 14 | −13.452 | −21.544 | −33.259 | 1.00 | 0.00 | 1MOD |
| ATOM | 117 | CD2 | PHE | 14 | −15.226 | −22.470 | −31.997 | 1.00 | 0.00 | 1MOD |
| ATOM | 118 | CE1 | PHE | 14 | −12.811 | −21.210 | −32.087 | 1.00 | 0.00 | 1MOD |
| ATOM | 119 | CE2 | PHE | 14 | −14.592 | −22.134 | −30.825 | 1.00 | 0.00 | 1MOD |
| ATOM | 120 | CZ | PHE | 14 | −13.377 | −21.501 | −30.871 | 1.00 | 0.00 | 1MOD |
| ATOM | 121 | N | TRP | 15 | −14.693 | −25.006 | −33.121 | 1.00 | 0.00 | 1MOD |
| ATOM | 122 | CA | TRP | 15 | −14.135 | −25.619 | −31.955 | 1.00 | 0.00 | 1MOD |
| ATOM | 123 | C | TRP | 15 | −14.356 | −27.090 | −32.035 | 1.00 | 0.00 | 1MOD |
| ATOM | 124 | O | TRP | 15 | −15.142 | −27.586 | −32.842 | 1.00 | 0.00 | 1MOD |
| ATOM | 125 | CB | TRP | 15 | −14.881 | −25.236 | −30.663 | 1.00 | 0.00 | 1MOD |
| ATOM | 126 | CG | TRP | 15 | −16.291 | −25.799 | −30.650 | 1.00 | 0.00 | 1MOD |
| ATOM | 127 | CD1 | TRP | 15 | −17.114 | −26.072 | −31.706 | 1.00 | 0.00 | 1MOD |
| ATOM | 128 | CD2 | TRP | 15 | −16.996 | −26.230 | −29.476 | 1.00 | 0.00 | 1MOD |
| ATOM | 129 | NE1 | TRP | 15 | −18.271 | −26.668 | −31.264 | 1.00 | 0.00 | 1MOD |
| ATOM | 130 | CE2 | TRP | 15 | −18.218 | −26.767 | −29.893 | 1.00 | 0.00 | 1MOD |
| ATOM | 131 | CE3 | TRP | 15 | −16.657 | −26.194 | −28.155 | 1.00 | 0.00 | 1MOD |
| ATOM | 132 | CZ2 | TRP | 15 | −19.113 | −27.275 | −28.997 | 1.00 | 0.00 | 1MOD |
| ATOM | 133 | CZ3 | TRP | 15 | −17.564 | −26.700 | −27.249 | 1.00 | 0.00 | 1MOD |
| ATOM | 134 | CH2 | TRP | 15 | −18.768 | −27.230 | −27.664 | 1.00 | 0.00 | 1MOD |
| ATOM | 135 | N | LYS | 16 | −13.610 | −27.846 | −31.213 | 1.00 | 0.00 | 1MOD |
| ATOM | 136 | CA | LYS | 16 | −13.939 | −29.230 | −31.100 | 1.00 | 0.00 | 1MOD |
| ATOM | 137 | C | LYS | 16 | −14.389 | −29.410 | −29.694 | 1.00 | 0.00 | 1MOD |
| ATOM | 138 | O | LYS | 16 | −13.607 | −29.289 | −28.752 | 1.00 | 0.00 | 1MOD |
| ATOM | 139 | CB | LYS | 16 | −12.807 | −30.240 | −31.353 | 1.00 | 0.00 | 1MOD |
| ATOM | 140 | CG | LYS | 16 | −13.328 | −31.674 | −31.196 | 1.00 | 0.00 | 1MOD |
| ATOM | 141 | CD | LYS | 16 | −12.463 | −32.766 | −31.825 | 1.00 | 0.00 | 1MOD |
| ATOM | 142 | CE | LYS | 16 | −13.100 | −34.155 | −31.715 | 1.00 | 0.00 | 1MOD |
| ATOM | 143 | NZ | LYS | 16 | −12.261 | −35.164 | −32.398 | 1.00 | 0.00 | 1MOD |
| ATOM | 144 | N | GLY | 17 | −15.694 | −29.675 | −29.515 | 1.00 | 0.00 | 1MOD |
| ATOM | 145 | CA | GLY | 17 | −16.183 | −29.841 | −28.185 | 1.00 | 0.00 | 1MOD |
| ATOM | 146 | C | GLY | 17 | −15.779 | −31.192 | −27.717 | 1.00 | 0.00 | 1MOD |
| ATOM | 147 | O | GLY | 17 | −15.648 | −32.126 | −28.507 | 1.00 | 0.00 | 1MOD |
| ATOM | 148 | N | GLU | 18 | −15.558 | −31.320 | −26.399 | 1.00 | 0.00 | 1MOD |
| ATOM | 149 | CA | GLU | 18 | −15.280 | −32.606 | −25.841 | 1.00 | 0.00 | 1MOD |
| ATOM | 150 | C | GLU | 18 | −15.954 | −32.617 | −24.519 | 1.00 | 0.00 | 1MOD |
| ATOM | 151 | O | GLU | 18 | −16.345 | −31.568 | −24.009 | 1.00 | 0.00 | 1MOD |
| ATOM | 152 | CB | GLU | 18 | −13.799 | −32.973 | −25.662 | 1.00 | 0.00 | 1MOD |
| ATOM | 153 | CG | GLU | 18 | −13.645 | −34.446 | −25.274 | 1.00 | 0.00 | 1MOD |
| ATOM | 154 | CD | GLU | 18 | −12.268 | −34.928 | −25.702 | 1.00 | 0.00 | 1MOD |
| ATOM | 155 | OE1 | GLU | 18 | −11.792 | −34.490 | −26.782 | 1.00 | 0.00 | 1MOD |
| ATOM | 156 | OE2 | GLU | 18 | −11.684 | −35.762 | −24.962 | 1.00 | 0.00 | 1MOD |
| ATOM | 157 | N | ASP | 19 | −16.152 | −33.809 | −23.938 | 1.00 | 0.00 | 1MOD |
| ATOM | 158 | CA | ASP | 19 | −16.838 | −33.824 | −22.686 | 1.00 | 0.00 | 1MOD |
| ATOM | 159 | C | ASP | 19 | −16.008 | −33.044 | −21.714 | 1.00 | 0.00 | 1MOD |
| ATOM | 160 | O | ASP | 19 | −16.512 | −32.166 | −21.016 | 1.00 | 0.00 | 1MOD |
| ATOM | 161 | CB | ASP | 19 | −17.001 | −35.244 | −22.118 | 1.00 | 0.00 | 1MOD |
| ATOM | 162 | CG | ASP | 19 | −18.116 | −35.197 | −21.082 | 1.00 | 0.00 | 1MOD |
| ATOM | 163 | OD1 | ASP | 19 | −19.195 | −34.629 | −21.407 | 1.00 | 0.00 | 1MOD |
| ATOM | 164 | OD2 | ASP | 19 | −17.904 | −35.708 | −19.952 | 1.00 | 0.00 | 1MOD |
| ATOM | 165 | N | LEU | 20 | −14.698 | −33.356 | −21.657 | 1.00 | 0.00 | 1MOD |
| ATOM | 166 | CA | LEU | 20 | −13.808 | −32.711 | −20.734 | 1.00 | 0.00 | 1MOD |
| ATOM | 167 | C | LEU | 20 | −13.541 | −31.277 | −21.089 | 1.00 | 0.00 | 1MOD |
| ATOM | 168 | O | LEU | 20 | −13.569 | −30.417 | −20.209 | 1.00 | 0.00 | 1MOD |
| ATOM | 169 | CB | LEU | 20 | −12.447 | −33.407 | −20.608 | 1.00 | 0.00 | 1MOD |
| ATOM | 170 | CG | LEU | 20 | −11.514 | −32.707 | −19.602 | 1.00 | 0.00 | 1MOD |
| ATOM | 171 | CD1 | LEU | 20 | −12.106 | −32.727 | −18.185 | 1.00 | 0.00 | 1MOD |
| ATOM | 172 | CD2 | LEU | 20 | −10.098 | −33.296 | −19.658 | 1.00 | 0.00 | 1MOD |
| ATOM | 173 | N | SER | 21 | −13.275 | −30.957 | −22.373 | 1.00 | 0.00 | 1MOD |
| ATOM | 174 | CA | SER | 21 | −12.924 | −29.591 | −22.656 | 1.00 | 0.00 | 1MOD |
| ATOM | 175 | C | SER | 21 | −13.378 | −29.230 | −24.032 | 1.00 | 0.00 | 1MOD |
| ATOM | 176 | O | SER | 21 | −14.122 | −29.984 | −24.655 | 1.00 | 0.00 | 1MOD |
| ATOM | 177 | CB | SER | 21 | −11.412 | −29.306 | −22.583 | 1.00 | 0.00 | 1MOD |
| ATOM | 178 | OG | SER | 21 | −10.950 | −29.447 | −21.246 | 1.00 | 0.00 | 1MOD |
| ATOM | 179 | N | ASN | 22 | −13.033 | −28.008 | −24.502 | 1.00 | 0.00 | 1MOD |
| ATOM | 180 | CA | ASN | 22 | −13.305 | −27.733 | −25.877 | 1.00 | 0.00 | 1MOD |
| ATOM | 181 | C | ASN | 22 | −12.134 | −26.974 | −26.412 | 1.00 | 0.00 | 1MOD |
| ATOM | 182 | O | ASN | 22 | −11.751 | −25.922 | −25.903 | 1.00 | 0.00 | 1MOD |
| ATOM | 183 | CB | ASN | 22 | −14.606 | −26.961 | −26.189 | 1.00 | 0.00 | 1MOD |
| ATOM | 184 | CG | ASN | 22 | −14.444 | −25.485 | −25.899 | 1.00 | 0.00 | 1MOD |
| ATOM | 185 | OD1 | ASN | 22 | −14.068 | −24.744 | −26.804 | 1.00 | 0.00 | 1MOD |
| ATOM | 186 | ND2 | ASN | 22 | −14.710 | −25.056 | −24.635 | 1.00 | 0.00 | 1MOD |
| ATOM | 187 | N | TYR | 23 | −11.516 | −27.514 | −27.472 | 1.00 | 0.00 | 1MOD |
| ATOM | 188 | CA | TYR | 23 | −10.364 | −26.866 | −28.012 | 1.00 | 0.00 | 1MOD |
| ATOM | 189 | C | TYR | 23 | −10.897 | −25.865 | −28.976 | 1.00 | 0.00 | 1MOD |
| ATOM | 190 | O | TYR | 23 | −11.878 | −26.121 | −29.668 | 1.00 | 0.00 | 1MOD |
| ATOM | 191 | CB | TYR | 23 | −9.425 | −27.787 | −28.802 | 1.00 | 0.00 | 1MOD |
| ATOM | 192 | CG | TYR | 23 | −8.126 | −27.069 | −28.938 | 1.00 | 0.00 | 1MOD |
| ATOM | 193 | CD1 | TYR | 23 | −7.878 | −26.227 | −29.996 | 1.00 | 0.00 | 1MOD |
| ATOM | 194 | CD2 | TYR | 23 | −7.152 | −27.234 | −27.981 | 1.00 | 0.00 | 1MOD |
| ATOM | 195 | CE1 | TYR | 23 | −6.675 | −25.567 | −30.110 | 1.00 | 0.00 | 1MOD |
| ATOM | 196 | CE2 | TYR | 23 | −5.947 | −26.578 | −28.085 | 1.00 | 0.00 | 1MOD |
| ATOM | 197 | CZ | TYR | 23 | −5.705 | −25.744 | −29.152 | 1.00 | 0.00 | 1MOD |
| ATOM | 198 | OH | TYR | 23 | −4.469 | −25.069 | −29.262 | 1.00 | 0.00 | 1MOD |
| ATOM | 199 | N | SER | 24 | −10.287 | −24.671 | −28.988 | 1.00 | 0.00 | 1MOD |
| ATOM | 200 | CA | SER | 24 | −10.666 | −23.597 | −29.853 | 1.00 | 0.00 | 1MOD |
| ATOM | 201 | C | SER | 24 | −10.091 | −23.834 | −31.212 | 1.00 | 0.00 | 1MOD |
| ATOM | 202 | O | SER | 24 | −9.694 | −22.878 | −31.875 | 1.00 | 0.00 | 1MOD |
| ATOM | 203 | CB | SER | 24 | −10.142 | −22.233 | −29.370 | 1.00 | 0.00 | 1MOD |
| ATOM | 204 | OG | SER | 24 | −10.711 | −21.921 | −28.108 | 1.00 | 0.00 | 1MOD |
| ATOM | 205 | N | TYR | 25 | −9.927 | −25.106 | −31.628 | 1.00 | 0.00 | 1MOD |
| ATOM | 206 | CA | TYR | 25 | −9.391 | −25.330 | −32.939 | 1.00 | 0.00 | 1MOD |
| ATOM | 207 | C | TYR | 25 | −10.291 | −26.285 | −33.651 | 1.00 | 0.00 | 1MOD |
| ATOM | 208 | O | TYR | 25 | −10.887 | −27.175 | −33.044 | 1.00 | 0.00 | 1MOD |
| ATOM | 209 | CB | TYR | 25 | −7.964 | −25.912 | −32.921 | 1.00 | 0.00 | 1MOD |
| ATOM | 210 | CG | TYR | 25 | −7.443 | −25.883 | −34.313 | 1.00 | 0.00 | 1MOD |
| ATOM | 211 | CD1 | TYR | 25 | −7.117 | −24.683 | −34.902 | 1.00 | 0.00 | 1MOD |
| ATOM | 212 | CD2 | TYR | 25 | −7.254 | −27.045 | −35.022 | 1.00 | 0.00 | 1MOD |
| ATOM | 213 | CE1 | TYR | 25 | −6.634 | −24.639 | −36.187 | 1.00 | 0.00 | 1MOD |
| ATOM | 214 | CE2 | TYR | 25 | −6.770 | −27.010 | −36.309 | 1.00 | 0.00 | 1MOD |
| ATOM | 215 | CZ | TYR | 25 | −6.461 | −25.805 | −36.893 | 1.00 | 0.00 | 1MOD |
| ATOM | 216 | OH | TYR | 25 | −5.964 | −25.759 | −38.214 | 1.00 | 0.00 | 1MOD |
| ATOM | 217 | N | SER | 26 | −10.441 | −26.105 | −34.977 | 1.00 | 0.00 | 1MOD |
| ATOM | 218 | CA | SER | 26 | −11.305 | −26.988 | −35.702 | 1.00 | 0.00 | 1MOD |
| ATOM | 219 | C | SER | 26 | −10.510 | −28.205 | −36.032 | 1.00 | 0.00 | 1MOD |
| ATOM | 220 | O | SER | 26 | −9.348 | −28.121 | −36.422 | 1.00 | 0.00 | 1MOD |
| ATOM | 221 | CB | SER | 26 | −11.770 | −26.401 | −37.038 | 1.00 | 0.00 | 1MOD |
| ATOM | 222 | OG | SER | 26 | −12.342 | −25.123 | −36.816 | 1.00 | 0.00 | 1MOD |
| ATOM | 223 | N | SER | 27 | −11.115 | −29.394 | −35.906 | 1.00 | 0.00 | 1MOD |
| ATOM | 224 | CA | SER | 27 | −10.317 | −30.534 | −36.240 | 1.00 | 0.00 | 1MOD |
| ATOM | 225 | C | SER | 27 | −10.724 | −30.965 | −37.601 | 1.00 | 0.00 | 1MOD |
| ATOM | 226 | O | SER | 27 | −11.413 | −31.973 | −37.760 | 1.00 | 0.00 | 1MOD |
| ATOM | 227 | CB | SER | 27 | −10.544 | −31.732 | −35.298 | 1.00 | 0.00 | 1MOD |
| ATOM | 228 | OG | SER | 27 | −9.729 | −32.832 | −35.679 | 1.00 | 0.00 | 1MOD |
| ATOM | 229 | N | THR | 28 | −10.294 | −30.212 | −38.634 | 1.00 | 0.00 | 1MOD |
| ATOM | 230 | CA | THR | 28 | −10.708 | −30.659 | −39.917 | 1.00 | 0.00 | 1MOD |
| ATOM | 231 | C | THR | 28 | −10.093 | −31.997 | −40.156 | 1.00 | 0.00 | 1MOD |
| ATOM | 232 | O | THR | 28 | −10.824 | −32.964 | −40.367 | 1.00 | 0.00 | 1MOD |
| ATOM | 233 | CB | THR | 28 | −10.360 | −29.717 | −41.047 | 1.00 | 0.00 | 1MOD |
| ATOM | 234 | OG1 | THR | 28 | −10.827 | −30.246 | −42.281 | 1.00 | 0.00 | 1MOD |
| ATOM | 235 | CG2 | THR | 28 | −8.841 | −29.459 | −41.099 | 1.00 | 0.00 | 1MOD |
| ATOM | 236 | N | LEU | 29 | −8.742 | −32.112 | −40.169 | 1.00 | 0.00 | 1MOD |
| ATOM | 237 | CA | LEU | 29 | −8.311 | −33.461 | −40.361 | 1.00 | 0.00 | 1MOD |
| ATOM | 238 | C | LEU | 29 | −8.527 | −34.258 | −39.084 | 1.00 | 0.00 | 1MOD |
| ATOM | 239 | O | LEU | 29 | −9.652 | −34.353 | −38.613 | 1.00 | 0.00 | 1MOD |
| ATOM | 240 | CB | LEU | 29 | −6.856 | −33.465 | −40.882 | 1.00 | 0.00 | 1MOD |
| ATOM | 241 | CG | LEU | 29 | −6.712 | −32.967 | −42.331 | 1.00 | 0.00 | 1MOD |
| ATOM | 242 | CD1 | LEU | 29 | −5.246 | −33.000 | −42.797 | 1.00 | 0.00 | 1MOD |
| ATOM | 243 | CD2 | LEU | 29 | −7.658 | −33.736 | −43.266 | 1.00 | 0.00 | 1MOD |
| ATOM | 244 | N | PRO | 30 | −7.530 | −34.826 | −38.444 | 1.00 | 0.00 | 1MOD |
| ATOM | 245 | CA | PRO | 30 | −7.739 | −35.255 | −37.084 | 1.00 | 0.00 | 1MOD |
| ATOM | 246 | C | PRO | 30 | −6.957 | −34.410 | −36.137 | 1.00 | 0.00 | 1MOD |
| ATOM | 247 | O | PRO | 30 | −6.862 | −34.865 | −35.001 | 1.00 | 0.00 | 1MOD |
| ATOM | 248 | CB | PRO | 30 | −7.316 | −36.720 | −36.983 | 1.00 | 0.00 | 1MOD |
| ATOM | 249 | CG | PRO | 30 | −6.308 | −36.885 | −38.116 | 1.00 | 0.00 | 1MOD |
| ATOM | 250 | CD | PRO | 30 | −6.819 | −35.891 | −39.161 | 1.00 | 0.00 | 1MOD |
| ATOM | 251 | N | PRO | 31 | −6.397 | −33.282 | −36.511 | 1.00 | 0.00 | 1MOD |
| ATOM | 252 | CA | PRO | 31 | −5.378 | −32.657 | −35.704 | 1.00 | 0.00 | 1MOD |
| ATOM | 253 | C | PRO | 31 | −5.704 | −32.518 | −34.252 | 1.00 | 0.00 | 1MOD |
| ATOM | 254 | O | PRO | 31 | −4.822 | −32.775 | −33.435 | 1.00 | 0.00 | 1MOD |
| ATOM | 255 | CB | PRO | 31 | −5.154 | −31.279 | −36.316 | 1.00 | 0.00 | 1MOD |
| ATOM | 256 | CG | PRO | 31 | −6.550 | −30.921 | −36.845 | 1.00 | 0.00 | 1MOD |
| ATOM | 257 | CD | PRO | 31 | −7.144 | −32.276 | −37.257 | 1.00 | 0.00 | 1MOD |
| ATOM | 258 | N | PHE | 32 | −6.949 | −32.152 | −33.908 | 1.00 | 0.00 | 1MOD |
| ATOM | 259 | CA | PHE | 32 | −7.288 | −31.905 | −32.537 | 1.00 | 0.00 | 1MOD |
| ATOM | 260 | C | PHE | 32 | −7.129 | −33.130 | −31.706 | 1.00 | 0.00 | 1MOD |
| ATOM | 261 | O | PHE | 32 | −7.411 | −34.248 | −32.133 | 1.00 | 0.00 | 1MOD |
| ATOM | 262 | CB | PHE | 32 | −8.715 | −31.363 | −32.335 | 1.00 | 0.00 | 1MOD |
| ATOM | 263 | CG | PHE | 32 | −9.002 | −31.444 | −30.876 | 1.00 | 0.00 | 1MOD |
| ATOM | 264 | CD1 | PHE | 32 | −8.454 | −30.548 | −29.988 | 1.00 | 0.00 | 1MOD |
| ATOM | 265 | CD2 | PHE | 32 | −9.834 | −32.432 | −30.405 | 1.00 | 0.00 | 1MOD |
| ATOM | 266 | CE1 | PHE | 32 | −8.734 | −30.645 | −28.645 | 1.00 | 0.00 | 1MOD |
| ATOM | 267 | CE2 | PHE | 32 | −10.117 | −32.531 | −29.065 | 1.00 | 0.00 | 1MOD |
| ATOM | 268 | CZ | PHE | 32 | −9.566 | −31.636 | −28.180 | 1.00 | 0.00 | 1MOD |
| ATOM | 269 | N | LEU | 33 | −6.625 | −32.917 | −30.473 | 1.00 | 0.00 | 1MOD |
| ATOM | 270 | CA | LEU | 33 | −6.420 | −33.977 | −29.535 | 1.00 | 0.00 | 1MOD |
| ATOM | 271 | C | LEU | 33 | −6.690 | −33.425 | −28.167 | 1.00 | 0.00 | 1MOD |
| ATOM | 272 | O | LEU | 33 | −6.540 | −32.228 | −27.933 | 1.00 | 0.00 | 1MOD |
| ATOM | 273 | CB | LEU | 33 | −4.980 | −34.472 | −29.551 | 1.00 | 0.00 | 1MOD |
| ATOM | 274 | CG | LEU | 33 | −4.543 | −35.064 | −30.899 | 1.00 | 0.00 | 1MOD |
| ATOM | 275 | CD1 | LEU | 33 | −3.096 | −35.572 | −30.831 | 1.00 | 0.00 | 1MOD |
| ATOM | 276 | CD2 | LEU | 33 | −5.530 | −36.134 | −31.389 | 1.00 | 0.00 | 1MOD |
| ATOM | 277 | N | LEU | 34 | −7.066 | −34.298 | −27.208 | 1.00 | 0.00 | 1MOD |
| ATOM | 278 | CA | LEU | 34 | −7.439 | −33.836 | −25.900 | 1.00 | 0.00 | 1MOD |
| ATOM | 279 | C | LEU | 34 | −6.253 | −33.220 | −25.234 | 1.00 | 0.00 | 1MOD |
| ATOM | 280 | O | LEU | 34 | −5.132 | −33.717 | −25.321 | 1.00 | 0.00 | 1MOD |
| ATOM | 281 | CB | LEU | 34 | −7.978 | −34.956 | −24.990 | 1.00 | 0.00 | 1MOD |
| ATOM | 282 | CG | LEU | 34 | −8.392 | −34.477 | −23.585 | 1.00 | 0.00 | 1MOD |
| ATOM | 283 | CD1 | LEU | 34 | −9.572 | −33.493 | −23.651 | 1.00 | 0.00 | 1MOD |
| ATOM | 284 | CD2 | LEU | 34 | −8.662 | −35.666 | −22.651 | 1.00 | 0.00 | 1MOD |
| ATOM | 285 | N | ASP | 35 | −6.496 | −32.099 | −24.534 | 1.00 | 0.00 | 1MOD |
| ATOM | 286 | CA | ASP | 35 | −5.476 | −31.350 | −23.864 | 1.00 | 0.00 | 1MOD |
| ATOM | 287 | C | ASP | 35 | −4.894 | −32.227 | −22.811 | 1.00 | 0.00 | 1MOD |
| ATOM | 288 | O | ASP | 35 | −3.727 | −32.089 | −22.446 | 1.00 | 0.00 | 1MOD |
| ATOM | 289 | CB | ASP | 35 | −6.038 | −30.123 | −23.133 | 1.00 | 0.00 | 1MOD |
| ATOM | 290 | CG | ASP | 35 | −6.596 | −29.166 | −24.172 | 1.00 | 0.00 | 1MOD |
| ATOM | 291 | OD1 | ASP | 35 | −5.945 | −28.994 | −25.239 | 1.00 | 0.00 | 1MOD |
| ATOM | 292 | OD2 | ASP | 35 | −7.685 | −28.593 | −23.912 | 1.00 | 0.00 | 1MOD |
| ATOM | 293 | N | ALA | 36 | −5.726 | −33.103 | −22.225 | 1.00 | 0.00 | 1MOD |
| ATOM | 294 | CA | ALA | 36 | −5.230 | −33.957 | −21.192 | 1.00 | 0.00 | 1MOD |
| ATOM | 295 | C | ALA | 36 | −4.214 | −34.938 | −21.715 | 1.00 | 0.00 | 1MOD |
| ATOM | 296 | O | ALA | 36 | −3.189 | −35.153 | −21.072 | 1.00 | 0.00 | 1MOD |
| ATOM | 297 | CB | ALA | 36 | −6.350 | −34.785 | −20.535 | 1.00 | 0.00 | 1MOD |
| ATOM | 298 | N | ALA | 37 | −4.502 | −35.662 | −22.821 | 1.00 | 0.00 | 1MOD |
| ATOM | 299 | CA | ALA | 37 | −3.511 | −36.620 | −23.247 | 1.00 | 0.00 | 1MOD |
| ATOM | 300 | C | ALA | 37 | −2.352 | −36.120 | −24.091 | 1.00 | 0.00 | 1MOD |
| ATOM | 301 | O | ALA | 37 | −1.203 | −36.331 | −23.705 | 1.00 | 0.00 | 1MOD |
| ATOM | 302 | CB | ALA | 37 | −4.124 | −37.900 | −23.863 | 1.00 | 0.00 | 1MOD |
| ATOM | 303 | N | PRO | 38 | −2.564 | −35.467 | −25.223 | 1.00 | 0.00 | 1MOD |
| ATOM | 304 | CA | PRO | 38 | −1.395 | −35.121 | −26.009 | 1.00 | 0.00 | 1MOD |
| ATOM | 305 | C | PRO | 38 | −1.030 | −33.672 | −26.011 | 1.00 | 0.00 | 1MOD |
| ATOM | 306 | O | PRO | 38 | −1.765 | −32.854 | −25.463 | 1.00 | 0.00 | 1MOD |
| ATOM | 307 | CB | PRO | 38 | −1.621 | −35.674 | −27.413 | 1.00 | 0.00 | 1MOD |
| ATOM | 308 | CG | PRO | 38 | −3.142 | −35.725 | −27.527 | 1.00 | 0.00 | 1MOD |
| ATOM | 309 | CD | PRO | 38 | −3.616 | −35.969 | −26.096 | 1.00 | 0.00 | 1MOD |
| ATOM | 310 | N | CYS | 39 | 0.095 | −33.343 | −26.678 | 1.00 | 0.00 | 1MOD |
| ATOM | 311 | CA | CYS | 39 | 0.563 | −31.997 | −26.793 | 1.00 | 0.00 | 1MOD |
| ATOM | 312 | C | CYS | 39 | −0.359 | −31.278 | −27.731 | 1.00 | 0.00 | 1MOD |
| ATOM | 313 | O | CYS | 39 | −0.988 | −31.882 | −28.597 | 1.00 | 0.00 | 1MOD |
| ATOM | 314 | CB | CYS | 39 | 1.978 | −31.913 | −27.393 | 1.00 | 0.00 | 1MOD |
| ATOM | 315 | SG | CYS | 39 | 3.186 | −32.913 | −26.472 | 1.00 | 0.00 | 1MOD |
| ATOM | 316 | N | GLU | 40 | −0.454 | −29.944 | −27.543 | 1.00 | 0.00 | 1MOD |
| ATOM | 317 | CA | GLU | 40 | −1.151 | −28.881 | −28.234 | 1.00 | 0.00 | 1MOD |
| ATOM | 318 | C | GLU | 40 | −0.404 | −28.301 | −29.418 | 1.00 | 0.00 | 1MOD |
| ATOM | 319 | O | GLU | 40 | −0.748 | −27.172 | −29.762 | 1.00 | 0.00 | 1MOD |
| ATOM | 320 | CB | GLU | 40 | −1.562 | −27.698 | −27.336 | 1.00 | 0.00 | 1MOD |
| ATOM | 321 | CG | GLU | 40 | −2.862 | −27.935 | −26.558 | 1.00 | 0.00 | 1MOD |
| ATOM | 322 | CD | GLU | 40 | −2.550 | −28.583 | −25.221 | 1.00 | 0.00 | 1MOD |
| ATOM | 323 | OE1 | GLU | 40 | −1.896 | −27.905 | −24.384 | 1.00 | 0.00 | 1MOD |
| ATOM | 324 | OE2 | GLU | 40 | −2.968 | −29.751 | −25.010 | 1.00 | 0.00 | 1MOD |
| ATOM | 325 | N | PRO | 41 | 0.623 | −28.904 | −29.994 | 1.00 | 0.00 | 1MOD |
| ATOM | 326 | CA | PRO | 41 | 1.511 | −28.258 | −30.934 | 1.00 | 0.00 | 1MOD |
| ATOM | 327 | C | PRO | 41 | 0.825 | −27.710 | −32.151 | 1.00 | 0.00 | 1MOD |
| ATOM | 328 | O | PRO | 41 | 1.528 | −27.248 | −33.046 | 1.00 | 0.00 | 1MOD |
| ATOM | 329 | CB | PRO | 41 | 2.463 | −29.350 | −31.394 | 1.00 | 0.00 | 1MOD |
| ATOM | 330 | CG | PRO | 41 | 1.528 | −30.561 | −31.492 | 1.00 | 0.00 | 1MOD |
| ATOM | 331 | CD | PRO | 41 | 0.477 | −30.293 | −30.406 | 1.00 | 0.00 | 1MOD |
| ATOM | 332 | N | GLU | 42 | −0.507 | −27.797 | −32.259 | 1.00 | 0.00 | 1MOD |
| ATOM | 333 | CA | GLU | 42 | −1.158 | −27.234 | −33.397 | 1.00 | 0.00 | 1MOD |
| ATOM | 334 | C | GLU | 42 | −0.750 | −25.780 | −33.471 | 1.00 | 0.00 | 1MOD |
| ATOM | 335 | O | GLU | 42 | −0.721 | −25.239 | −34.573 | 1.00 | 0.00 | 1MOD |
| ATOM | 336 | CB | GLU | 42 | −2.693 | −27.328 | −33.315 | 1.00 | 0.00 | 1MOD |
| ATOM | 337 | CG | GLU | 42 | −3.310 | −26.514 | −32.181 | 1.00 | 0.00 | 1MOD |
| ATOM | 338 | CD | GLU | 42 | −3.513 | −25.100 | −32.701 | 1.00 | 0.00 | 1MOD |
| ATOM | 339 | OE1 | GLU | 42 | −4.225 | −24.949 | −33.729 | 1.00 | 0.00 | 1MOD |
| ATOM | 340 | OE2 | GLU | 42 | −2.952 | −24.156 | −32.088 | 1.00 | 0.00 | 1MOD |
| ATOM | 341 | N | SER | 43 | −0.495 | −25.096 | −32.317 | 1.00 | 0.00 | 1MOD |
| ATOM | 342 | CA | SER | 43 | 0.058 | −23.755 | −32.312 | 1.00 | 0.00 | 1MOD |
| ATOM | 343 | C | SER | 43 | 1.553 | −23.903 | −32.351 | 1.00 | 0.00 | 1MOD |
| ATOM | 344 | O | SER | 43 | 2.083 | −24.983 | −32.116 | 1.00 | 0.00 | 1MOD |
| ATOM | 345 | CB | SER | 43 | −0.273 | −22.943 | −31.052 | 1.00 | 0.00 | 1MOD |
| ATOM | 346 | OG | SER | 43 | 0.301 | −21.649 | −31.156 | 1.00 | 0.00 | 1MOD |
| ATOM | 347 | N | LEU | 44 | 2.299 | −22.815 | −32.616 | 1.00 | 0.00 | 1MOD |
| ATOM | 348 | CA | LEU | 44 | 3.723 | −22.966 | −32.740 | 1.00 | 0.00 | 1MOD |
| ATOM | 349 | C | LEU | 44 | 4.290 | −23.412 | −31.428 | 1.00 | 0.00 | 1MOD |
| ATOM | 350 | O | LEU | 44 | 3.829 | −23.010 | −30.361 | 1.00 | 0.00 | 1MOD |
| ATOM | 351 | CB | LEU | 44 | 4.447 | −21.679 | −33.164 | 1.00 | 0.00 | 1MOD |
| ATOM | 352 | CG | LEU | 44 | 4.043 | −21.191 | −34.566 | 1.00 | 0.00 | 1MOD |
| ATOM | 353 | CD1 | LEU | 44 | 4.880 | −19.974 | −34.994 | 1.00 | 0.00 | 1MOD |
| ATOM | 354 | CD2 | LEU | 44 | 4.087 | −22.333 | −35.594 | 1.00 | 0.00 | 1MOD |
| ATOM | 355 | N | GLU | 45 | 5.311 | −24.290 | −31.499 | 1.00 | 0.00 | 1MOD |
| ATOM | 356 | CA | GLU | 45 | 5.958 | −24.855 | −30.348 | 1.00 | 0.00 | 1MOD |
| ATOM | 357 | C | GLU | 45 | 6.688 | −23.782 | −29.610 | 1.00 | 0.00 | 1MOD |
| ATOM | 358 | O | GLU | 45 | 6.741 | −23.784 | −28.381 | 1.00 | 0.00 | 1MOD |
| ATOM | 359 | CB | GLU | 45 | 6.986 | −25.945 | −30.705 | 1.00 | 0.00 | 1MOD |
| ATOM | 360 | CG | GLU | 45 | 7.651 | −26.570 | −29.476 | 1.00 | 0.00 | 1MOD |
| ATOM | 361 | CD | GLU | 45 | 8.394 | −27.828 | −29.909 | 1.00 | 0.00 | 1MOD |
| ATOM | 362 | OE1 | GLU | 45 | 9.308 | −27.717 | −30.767 | 1.00 | 0.00 | 1MOD |
| ATOM | 363 | OE2 | GLU | 45 | 8.053 | −28.921 | −29.379 | 1.00 | 0.00 | 1MOD |
| ATOM | 364 | N | ILE | 46 | 7.264 | −22.826 | −30.358 | 1.00 | 0.00 | 1MOD |
| ATOM | 365 | CA | ILE | 46 | 8.061 | −21.786 | −29.781 | 1.00 | 0.00 | 1MOD |
| ATOM | 366 | C | ILE | 46 | 7.193 | −21.022 | −28.840 | 1.00 | 0.00 | 1MOD |
| ATOM | 367 | O | ILE | 46 | 7.612 | −20.663 | −27.741 | 1.00 | 0.00 | 1MOD |
| ATOM | 368 | CB | ILE | 46 | 8.537 | −20.806 | −30.814 | 1.00 | 0.00 | 1MOD |
| ATOM | 369 | CG1 | ILE | 46 | 9.271 | −21.532 | −31.958 | 1.00 | 0.00 | 1MOD |
| ATOM | 370 | CG2 | ILE | 46 | 9.405 | −19.758 | −30.099 | 1.00 | 0.00 | 1MOD |
| ATOM | 371 | CD1 | ILE | 46 | 10.513 | −22.307 | −31.521 | 1.00 | 0.00 | 1MOD |
| ATOM | 372 | N | ASN | 47 | 5.936 | −20.775 | −29.249 | 1.00 | 0.00 | 1MOD |
| ATOM | 373 | CA | ASN | 47 | 5.021 | −19.996 | −28.471 | 1.00 | 0.00 | 1MOD |
| ATOM | 374 | C | ASN | 47 | 4.861 | −20.661 | −27.143 | 1.00 | 0.00 | 1MOD |
| ATOM | 375 | O | ASN | 47 | 4.798 | −19.997 | −26.110 | 1.00 | 0.00 | 1MOD |
| ATOM | 376 | CB | ASN | 47 | 3.633 | −19.913 | −29.127 | 1.00 | 0.00 | 1MOD |
| ATOM | 377 | CG | ASN | 47 | 2.834 | −18.825 | −28.425 | 1.00 | 0.00 | 1MOD |
| ATOM | 378 | OD1 | ASN | 47 | 3.073 | −18.519 | −27.258 | 1.00 | 0.00 | 1MOD |
| ATOM | 379 | ND2 | ASN | 47 | 1.857 | −18.223 | −29.152 | 1.00 | 0.00 | 1MOD |
| ATOM | 380 | N | LYS | 48 | 4.796 | −22.003 | −27.136 | 1.00 | 0.00 | 1MOD |
| ATOM | 381 | CA | LYS | 48 | 4.673 | −22.706 | −25.895 | 1.00 | 0.00 | 1MOD |
| ATOM | 382 | C | LYS | 48 | 5.898 | −22.377 | −25.103 | 1.00 | 0.00 | 1MOD |
| ATOM | 383 | O | LYS | 48 | 5.831 | −22.129 | −23.906 | 1.00 | 0.00 | 1MOD |
| ATOM | 384 | CB | LYS | 48 | 4.644 | −24.233 | −26.068 | 1.00 | 0.00 | 1MOD |
| ATOM | 385 | CG | LYS | 48 | 3.365 | −24.769 | −26.710 | 1.00 | 0.00 | 1MOD |
| ATOM | 386 | CD | LYS | 48 | 3.495 | −26.210 | −27.207 | 1.00 | 0.00 | 1MOD |
| ATOM | 387 | CE | LYS | 48 | 4.060 | −26.314 | −28.624 | 1.00 | 0.00 | 1MOD |
| ATOM | 388 | NZ | LYS | 48 | 3.119 | −25.692 | −29.583 | 1.00 | 0.00 | 1MOD |
| ATOM | 389 | N | TYR | 49 | 7.058 | −22.298 | −25.772 | 1.00 | 0.00 | 1MOD |
| ATOM | 390 | CA | TYR | 49 | 8.291 | −22.030 | −25.093 | 1.00 | 0.00 | 1MOD |
| ATOM | 391 | C | TYR | 49 | 8.151 | −20.737 | −24.364 | 1.00 | 0.00 | 1MOD |
| ATOM | 392 | O | TYR | 49 | 8.632 | −20.603 | −23.241 | 1.00 | 0.00 | 1MOD |
| ATOM | 393 | CB | TYR | 49 | 9.486 | −21.887 | −26.055 | 1.00 | 0.00 | 1MOD |
| ATOM | 394 | CG | TYR | 49 | 10.683 | −21.394 | −25.305 | 1.00 | 0.00 | 1MOD |
| ATOM | 395 | CD1 | TYR | 49 | 11.426 | −22.231 | −24.502 | 1.00 | 0.00 | 1MOD |
| ATOM | 396 | CD2 | TYR | 49 | 11.077 | −20.079 | −25.427 | 1.00 | 0.00 | 1MOD |
| ATOM | 397 | CE1 | TYR | 49 | 12.534 | −21.761 | −23.828 | 1.00 | 0.00 | 1MOD |
| ATOM | 398 | CE2 | TYR | 49 | 12.181 | −19.605 | −24.756 | 1.00 | 0.00 | 1MOD |
| ATOM | 399 | CZ | TYR | 49 | 12.913 | −20.445 | −23.954 | 1.00 | 0.00 | 1MOD |
| ATOM | 400 | OH | TYR | 49 | 14.046 | −19.951 | −23.270 | 1.00 | 0.00 | 1MOD |
| ATOM | 401 | N | PHE | 50 | 7.482 | −19.746 | −24.975 | 1.00 | 0.00 | 1MOD |
| ATOM | 402 | CA | PHE | 50 | 7.349 | −18.484 | −24.313 | 1.00 | 0.00 | 1MOD |
| ATOM | 403 | C | PHE | 50 | 6.626 | −18.674 | −23.021 | 1.00 | 0.00 | 1MOD |
| ATOM | 404 | O | PHE | 50 | 7.014 | −18.098 | −22.006 | 1.00 | 0.00 | 1MOD |
| ATOM | 405 | CB | PHE | 50 | 6.571 | −17.425 | −25.109 | 1.00 | 0.00 | 1MOD |
| ATOM | 406 | CG | PHE | 50 | 7.474 | −16.860 | −26.149 | 1.00 | 0.00 | 1MOD |
| ATOM | 407 | CD1 | PHE | 50 | 7.608 | −17.462 | −27.378 | 1.00 | 0.00 | 1MOD |
| ATOM | 408 | CD2 | PHE | 50 | 8.187 | −15.713 | −25.883 | 1.00 | 0.00 | 1MOD |
| ATOM | 409 | CE1 | PHE | 50 | 8.444 | −16.922 | −28.327 | 1.00 | 0.00 | 1MOD |
| ATOM | 410 | CE2 | PHE | 50 | 9.025 | −15.170 | −26.827 | 1.00 | 0.00 | 1MOD |
| ATOM | 411 | CZ | PHE | 50 | 9.152 | −15.777 | −28.053 | 1.00 | 0.00 | 1MOD |
| ATOM | 412 | N | VAL | 51 | 5.554 | −19.488 | −23.002 | 1.00 | 0.00 | 1MOD |
| ATOM | 413 | CA | VAL | 51 | 4.834 | −19.570 | −21.767 | 1.00 | 0.00 | 1MOD |
| ATOM | 414 | C | VAL | 51 | 5.706 | −20.134 | −20.685 | 1.00 | 0.00 | 1MOD |
| ATOM | 415 | O | VAL | 51 | 5.668 | −19.641 | −19.563 | 1.00 | 0.00 | 1MOD |
| ATOM | 416 | CB | VAL | 51 | 3.520 | −20.303 | −21.835 | 1.00 | 0.00 | 1MOD |
| ATOM | 417 | CG1 | VAL | 51 | 2.597 | −19.540 | −22.802 | 1.00 | 0.00 | 1MOD |
| ATOM | 418 | CG2 | VAL | 51 | 3.738 | −21.771 | −22.204 | 1.00 | 0.00 | 1MOD |
| ATOM | 419 | N | VAL | 52 | 6.556 | −21.131 | −20.991 | 1.00 | 0.00 | 1MOD |
| ATOM | 420 | CA | VAL | 52 | 7.395 | −21.778 | −20.011 | 1.00 | 0.00 | 1MOD |
| ATOM | 421 | C | VAL | 52 | 8.161 | −20.724 | −19.287 | 1.00 | 0.00 | 1MOD |
| ATOM | 422 | O | VAL | 52 | 8.089 | −20.587 | −18.066 | 1.00 | 0.00 | 1MOD |
| ATOM | 423 | CB | VAL | 52 | 8.555 | −22.473 | −20.668 | 1.00 | 0.00 | 1MOD |
| ATOM | 424 | CG1 | VAL | 52 | 9.287 | −23.314 | −19.615 | 1.00 | 0.00 | 1MOD |
| ATOM | 425 | CG2 | VAL | 52 | 8.167 | −23.102 | −22.015 | 1.00 | 0.00 | 1MOD |
| ATOM | 426 | N | ILE | 53 | 8.914 | −19.935 | −20.068 | 1.00 | 0.00 | 1MOD |
| ATOM | 427 | CA | ILE | 53 | 9.770 | −18.940 | −19.508 | 1.00 | 0.00 | 1MOD |
| ATOM | 428 | C | ILE | 53 | 8.932 | −17.915 | −18.837 | 1.00 | 0.00 | 1MOD |
| ATOM | 429 | O | ILE | 53 | 9.295 | −17.409 | −17.778 | 1.00 | 0.00 | 1MOD |
| ATOM | 430 | CB | ILE | 53 | 10.649 | −18.248 | −20.509 | 1.00 | 0.00 | 1MOD |
| ATOM | 431 | CG1 | ILE | 53 | 11.733 | −17.443 | −19.773 | 1.00 | 0.00 | 1MOD |
| ATOM | 432 | CG2 | ILE | 53 | 9.768 | −17.396 | −21.437 | 1.00 | 0.00 | 1MOD |
| ATOM | 433 | CD1 | ILE | 53 | 12.717 | −18.317 | −18.996 | 1.00 | 0.00 | 1MOD |
| ATOM | 434 | N | ILE | 54 | 7.780 | −17.570 | −19.437 | 1.00 | 0.00 | 1MOD |
| ATOM | 435 | CA | ILE | 54 | 7.005 | −16.534 | −18.826 | 1.00 | 0.00 | 1MOD |
| ATOM | 436 | C | ILE | 54 | 6.593 | −16.983 | −17.459 | 1.00 | 0.00 | 1MOD |
| ATOM | 437 | O | ILE | 54 | 6.766 | −16.249 | −16.487 | 1.00 | 0.00 | 1MOD |
| ATOM | 438 | CB | ILE | 54 | 5.744 | −16.207 | −19.577 | 1.00 | 0.00 | 1MOD |
| ATOM | 439 | CG1 | ILE | 54 | 6.068 | −15.648 | −20.973 | 1.00 | 0.00 | 1MOD |
| ATOM | 440 | CG2 | ILE | 54 | 4.911 | −15.255 | −18.700 | 1.00 | 0.00 | 1MOD |
| ATOM | 441 | CD1 | ILE | 54 | 4.848 | −15.569 | −21.890 | 1.00 | 0.00 | 1MOD |
| ATOM | 442 | N | TYR | 55 | 6.053 | −18.213 | −17.342 | 1.00 | 0.00 | 1MOD |
| ATOM | 443 | CA | TYR | 55 | 5.597 | −18.696 | −16.070 | 1.00 | 0.00 | 1MOD |
| ATOM | 444 | C | TYR | 55 | 6.767 | −18.874 | −15.168 | 1.00 | 0.00 | 1MOD |
| ATOM | 445 | O | TYR | 55 | 6.691 | −18.576 | −13.978 | 1.00 | 0.00 | 1MOD |
| ATOM | 446 | CB | TYR | 55 | 4.835 | −20.035 | −16.123 | 1.00 | 0.00 | 1MOD |
| ATOM | 447 | CG | TYR | 55 | 3.667 | −19.795 | −17.015 | 1.00 | 0.00 | 1MOD |
| ATOM | 448 | CD1 | TYR | 55 | 2.710 | −18.855 | −16.707 | 1.00 | 0.00 | 1MOD |
| ATOM | 449 | CD2 | TYR | 55 | 3.517 | −20.545 | −18.155 | 1.00 | 0.00 | 1MOD |
| ATOM | 450 | CE1 | TYR | 55 | 1.651 | −18.650 | −17.559 | 1.00 | 0.00 | 1MOD |
| ATOM | 451 | CE2 | TYR | 55 | 2.461 | −20.341 | −19.006 | 1.00 | 0.00 | 1MOD |
| ATOM | 452 | CZ | TYR | 55 | 1.521 | −19.388 | −18.711 | 1.00 | 0.00 | 1MOD |
| ATOM | 453 | OH | TYR | 55 | 0.436 | −19.175 | −19.587 | 1.00 | 0.00 | 1MOD |
| ATOM | 454 | N | ALA | 56 | 7.894 | −19.364 | −15.704 | 1.00 | 0.00 | 1MOD |
| ATOM | 455 | CA | ALA | 56 | 9.026 | −19.593 | −14.860 | 1.00 | 0.00 | 1MOD |
| ATOM | 456 | C | ALA | 56 | 9.403 | −18.282 | −14.242 | 1.00 | 0.00 | 1MOD |
| ATOM | 457 | O | ALA | 56 | 9.740 | −18.215 | −13.061 | 1.00 | 0.00 | 1MOD |
| ATOM | 458 | CB | ALA | 56 | 10.248 | −20.112 | −15.634 | 1.00 | 0.00 | 1MOD |
| ATOM | 459 | N | LEU | 57 | 9.348 | −17.195 | −15.032 | 1.00 | 0.00 | 1MOD |
| ATOM | 460 | CA | LEU | 57 | 9.707 | −15.901 | −14.529 | 1.00 | 0.00 | 1MOD |
| ATOM | 461 | C | LEU | 57 | 8.750 | −15.492 | −13.453 | 1.00 | 0.00 | 1MOD |
| ATOM | 462 | O | LEU | 57 | 9.164 | −15.032 | −12.390 | 1.00 | 0.00 | 1MOD |
| ATOM | 463 | CB | LEU | 57 | 9.635 | −14.797 | −15.597 | 1.00 | 0.00 | 1MOD |
| ATOM | 464 | CG | LEU | 57 | 10.628 | −14.970 | −16.758 | 1.00 | 0.00 | 1MOD |
| ATOM | 465 | CD1 | LEU | 57 | 10.524 | −13.792 | −17.738 | 1.00 | 0.00 | 1MOD |
| ATOM | 466 | CD2 | LEU | 57 | 12.060 | −15.202 | −16.250 | 1.00 | 0.00 | 1MOD |
| ATOM | 467 | N | VAL | 58 | 7.436 | −15.668 | −13.693 | 1.00 | 0.00 | 1MOD |
| ATOM | 468 | CA | VAL | 58 | 6.483 | −15.191 | −12.735 | 1.00 | 0.00 | 1MOD |
| ATOM | 469 | C | VAL | 58 | 6.666 | −15.923 | −11.453 | 1.00 | 0.00 | 1MOD |
| ATOM | 470 | O | VAL | 58 | 6.596 | −15.329 | −10.379 | 1.00 | 0.00 | 1MOD |
| ATOM | 471 | CB | VAL | 58 | 5.042 | −15.337 | −13.147 | 1.00 | 0.00 | 1MOD |
| ATOM | 472 | CG1 | VAL | 58 | 4.806 | −14.492 | −14.408 | 1.00 | 0.00 | 1MOD |
| ATOM | 473 | CG2 | VAL | 58 | 4.696 | −16.825 | −13.301 | 1.00 | 0.00 | 1MOD |
| ATOM | 474 | N | PHE | 59 | 6.911 | −17.240 | −11.530 | 1.00 | 0.00 | 1MOD |
| ATOM | 475 | CA | PHE | 59 | 7.049 | −18.006 | −10.333 | 1.00 | 0.00 | 1MOD |
| ATOM | 476 | C | PHE | 59 | 8.247 | −17.519 | −9.589 | 1.00 | 0.00 | 1MOD |
| ATOM | 477 | O | PHE | 59 | 8.214 | −17.353 | −8.372 | 1.00 | 0.00 | 1MOD |
| ATOM | 478 | CB | PHE | 59 | 7.226 | −19.508 | −10.596 | 1.00 | 0.00 | 1MOD |
| ATOM | 479 | CG | PHE | 59 | 7.288 | −20.152 | −9.259 | 1.00 | 0.00 | 1MOD |
| ATOM | 480 | CD1 | PHE | 59 | 6.133 | −20.459 | −8.581 | 1.00 | 0.00 | 1MOD |
| ATOM | 481 | CD2 | PHE | 59 | 8.496 | −20.426 | −8.668 | 1.00 | 0.00 | 1MOD |
| ATOM | 482 | CE1 | PHE | 59 | 6.185 | −21.050 | −7.340 | 1.00 | 0.00 | 1MOD |
| ATOM | 483 | CE2 | PHE | 59 | 8.555 | −21.018 | −7.429 | 1.00 | 0.00 | 1MOD |
| ATOM | 484 | CZ | PHE | 59 | 7.397 | −21.333 | −6.762 | 1.00 | 0.00 | 1MOD |
| ATOM | 485 | N | LEU | 60 | 9.344 | −17.253 | −10.314 | 1.00 | 0.00 | 1MOD |
| ATOM | 486 | CA | LEU | 60 | 10.557 | −16.872 | −9.659 | 1.00 | 0.00 | 1MOD |
| ATOM | 487 | C | LEU | 60 | 10.312 | −15.604 | −8.903 | 1.00 | 0.00 | 1MOD |
| ATOM | 488 | O | LEU | 60 | 10.650 | −15.512 | −7.725 | 1.00 | 0.00 | 1MOD |
| ATOM | 489 | CB | LEU | 60 | 11.685 | −16.605 | −10.680 | 1.00 | 0.00 | 1MOD |
| ATOM | 490 | CG | LEU | 60 | 13.092 | −16.370 | −10.093 | 1.00 | 0.00 | 1MOD |
| ATOM | 491 | CD1 | LEU | 60 | 13.185 | −15.086 | −9.250 | 1.00 | 0.00 | 1MOD |
| ATOM | 492 | CD2 | LEU | 60 | 13.582 | −17.619 | −9.346 | 1.00 | 0.00 | 1MOD |
| ATOM | 493 | N | LEU | 61 | 9.720 | −14.591 | −9.570 | 1.00 | 0.00 | 1MOD |
| ATOM | 494 | CA | LEU | 61 | 9.531 | −13.302 | −8.963 | 1.00 | 0.00 | 1MOD |
| ATOM | 495 | C | LEU | 61 | 8.488 | −13.309 | −7.887 | 1.00 | 0.00 | 1MOD |
| ATOM | 496 | O | LEU | 61 | 8.705 | −12.765 | −6.806 | 1.00 | 0.00 | 1MOD |
| ATOM | 497 | CB | LEU | 61 | 9.165 | −12.198 | −9.977 | 1.00 | 0.00 | 1MOD |
| ATOM | 498 | CG | LEU | 61 | 8.992 | −10.785 | −9.371 | 1.00 | 0.00 | 1MOD |
| ATOM | 499 | CD1 | LEU | 61 | 7.655 | −10.592 | −8.634 | 1.00 | 0.00 | 1MOD |
| ATOM | 500 | CD2 | LEU | 61 | 10.201 | −10.436 | −8.491 | 1.00 | 0.00 | 1MOD |
| ATOM | 501 | N | SER | 62 | 7.329 | −13.938 | −8.156 | 1.00 | 0.00 | 1MOD |
| ATOM | 502 | CA | SER | 62 | 6.193 | −13.888 | −7.278 | 1.00 | 0.00 | 1MOD |
| ATOM | 503 | C | SER | 62 | 6.510 | −14.510 | −5.957 | 1.00 | 0.00 | 1MOD |
| ATOM | 504 | O | SER | 62 | 6.066 | −14.021 | −4.920 | 1.00 | 0.00 | 1MOD |
| ATOM | 505 | CB | SER | 62 | 4.993 | −14.642 | −7.876 | 1.00 | 0.00 | 1MOD |
| ATOM | 506 | OG | SER | 62 | 3.878 | −14.591 | −7.000 | 1.00 | 0.00 | 1MOD |
| ATOM | 507 | N | LEU | 63 | 7.286 | −15.606 | −5.949 | 1.00 | 0.00 | 1MOD |
| ATOM | 508 | CA | LEU | 63 | 7.521 | −16.284 | −4.705 | 1.00 | 0.00 | 1MOD |
| ATOM | 509 | C | LEU | 63 | 8.266 | −15.376 | −3.772 | 1.00 | 0.00 | 1MOD |
| ATOM | 510 | O | LEU | 63 | 7.859 | −15.194 | −2.626 | 1.00 | 0.00 | 1MOD |
| ATOM | 511 | CB | LEU | 63 | 8.343 | −17.579 | −4.900 | 1.00 | 0.00 | 1MOD |
| ATOM | 512 | CG | LEU | 63 | 8.455 | −18.534 | −3.682 | 1.00 | 0.00 | 1MOD |
| ATOM | 513 | CD1 | LEU | 63 | 9.294 | −19.768 | −4.051 | 1.00 | 0.00 | 1MOD |
| ATOM | 514 | CD2 | LEU | 63 | 9.002 | −17.863 | −2.410 | 1.00 | 0.00 | 1MOD |
| ATOM | 515 | N | LEU | 64 | 9.360 | −14.754 | −4.246 | 1.00 | 0.00 | 1MOD |
| ATOM | 516 | CA | LEU | 64 | 10.184 | −13.965 | −3.374 | 1.00 | 0.00 | 1MOD |
| ATOM | 517 | C | LEU | 64 | 9.431 | −12.790 | −2.848 | 1.00 | 0.00 | 1MOD |
| ATOM | 518 | O | LEU | 64 | 9.409 | −12.543 | −1.641 | 1.00 | 0.00 | 1MOD |
| ATOM | 519 | CB | LEU | 64 | 11.418 | −13.389 | −4.086 | 1.00 | 0.00 | 1MOD |
| ATOM | 520 | CG | LEU | 64 | 12.248 | −12.453 | −3.189 | 1.00 | 0.00 | 1MOD |
| ATOM | 521 | CD1 | LEU | 64 | 12.901 | −13.212 | −2.024 | 1.00 | 0.00 | 1MOD |
| ATOM | 522 | CD2 | LEU | 64 | 13.245 | −11.634 | −4.020 | 1.00 | 0.00 | 1MOD |
| ATOM | 523 | N | GLY | 65 | 8.767 | −12.042 | −3.743 | 1.00 | 0.00 | 1MOD |
| ATOM | 524 | CA | GLY | 65 | 8.143 | −10.827 | −3.317 | 1.00 | 0.00 | 1MOD |
| ATOM | 525 | C | GLY | 65 | 7.084 | −11.138 | −2.316 | 1.00 | 0.00 | 1MOD |
| ATOM | 526 | O | GLY | 65 | 6.967 | −10.463 | −1.295 | 1.00 | 0.00 | 1MOD |
| ATOM | 527 | N | ASN | 66 | 6.267 | −12.167 | −2.594 | 1.00 | 0.00 | 1MOD |
| ATOM | 528 | CA | ASN | 66 | 5.197 | −12.518 | −1.707 | 1.00 | 0.00 | 1MOD |
| ATOM | 529 | C | ASN | 66 | 5.731 | −13.090 | −0.432 | 1.00 | 0.00 | 1MOD |
| ATOM | 530 | O | ASN | 66 | 5.234 | −12.792 | −0.651 | 1.00 | 0.00 | 1MOD |
| ATOM | 531 | CB | ASN | 66 | 4.234 | −13.545 | −2.321 | 1.00 | 0.00 | 1MOD |
| ATOM | 532 | CG | ASN | 66 | 3.442 | −12.812 | −3.394 | 1.00 | 0.00 | 1MOD |
| ATOM | 533 | OD1 | ASN | 66 | 2.833 | −11.778 | −3.127 | 1.00 | 0.00 | 1MOD |
| ATOM | 534 | ND2 | ASN | 66 | 3.462 | −13.347 | −4.642 | 1.00 | 0.00 | 1MOD |
| ATOM | 535 | N | SER | 67 | 6.755 | −13.951 | −0.522 | 1.00 | 0.00 | 1MOD |
| ATOM | 536 | CA | SER | 67 | 7.250 | −14.549 | 0.680 | 1.00 | 0.00 | 1MOD |
| ATOM | 537 | C | SER | 67 | 7.899 | −13.503 | 1.526 | 1.00 | 0.00 | 1MOD |
| ATOM | 538 | O | SER | 67 | 7.725 | −13.476 | 2.742 | 1.00 | 0.00 | 1MOD |
| ATOM | 539 | CB | SER | 67 | 8.246 | −15.691 | 0.416 | 1.00 | 0.00 | 1MOD |
| ATOM | 540 | OG | SER | 67 | 9.342 | −15.235 | −0.360 | 1.00 | 0.00 | 1MOD |
| ATOM | 541 | N | LEU | 68 | 8.653 | −12.587 | 0.899 | 1.00 | 0.00 | 1MOD |
| ATOM | 542 | CA | LEU | 68 | 9.351 | −11.588 | 1.646 | 1.00 | 0.00 | 1MOD |
| ATOM | 543 | C | LEU | 68 | 8.334 | −10.752 | 2.351 | 1.00 | 0.00 | 1MOD |
| ATOM | 544 | O | LEU | 68 | 8.482 | −10.454 | 3.534 | 1.00 | 0.00 | 1MOD |
| ATOM | 545 | CB | LEU | 68 | 10.181 | −10.679 | 0.725 | 1.00 | 0.00 | 1MOD |
| ATOM | 546 | CG | LEU | 68 | 11.013 | −9.600 | 1.439 | 1.00 | 0.00 | 1MOD |
| ATOM | 547 | CD1 | LEU | 68 | 12.037 | −10.220 | 2.405 | 1.00 | 0.00 | 1MOD |
| ATOM | 548 | CD2 | LEU | 68 | 11.682 | −8.674 | 0.408 | 1.00 | 0.00 | 1MOD |
| ATOM | 549 | N | VAL | 69 | 7.245 | −10.372 | 1.655 | 1.00 | 0.00 | 1MOD |
| ATOM | 550 | CA | VAL | 69 | 6.288 | −9.528 | 2.308 | 1.00 | 0.00 | 1MOD |
| ATOM | 551 | C | VAL | 69 | 5.723 | −10.251 | 3.488 | 1.00 | 0.00 | 1MOD |
| ATOM | 552 | O | VAL | 69 | 5.572 | −9.668 | 4.558 | 1.00 | 0.00 | 1MOD |
| ATOM | 553 | CB | VAL | 69 | 5.149 | −9.040 | 1.446 | 1.00 | 0.00 | 1MOD |
| ATOM | 554 | CG1 | VAL | 69 | 5.722 | −8.138 | 0.349 | 1.00 | 0.00 | 1MOD |
| ATOM | 555 | CG2 | VAL | 69 | 4.340 | −10.217 | 0.897 | 1.00 | 0.00 | 1MOD |
| ATOM | 556 | N | MET | 70 | 5.409 | −11.552 | 3.340 | 1.00 | 0.00 | 1MOD |
| ATOM | 557 | CA | MET | 70 | 4.845 | −12.283 | 4.439 | 1.00 | 0.00 | 1MOD |
| ATOM | 558 | C | MET | 70 | 5.820 | −12.303 | 5.572 | 1.00 | 0.00 | 1MOD |
| ATOM | 559 | O | MET | 70 | 5.451 | −12.056 | 6.718 | 1.00 | 0.00 | 1MOD |
| ATOM | 560 | CB | MET | 70 | 4.579 | −13.764 | 4.108 | 1.00 | 0.00 | 1MOD |
| ATOM | 561 | CG | MET | 70 | 4.290 | −14.603 | 5.358 | 1.00 | 0.00 | 1MOD |
| ATOM | 562 | SD | MET | 70 | 4.258 | −16.402 | 5.092 | 1.00 | 0.00 | 1MOD |
| ATOM | 563 | CE | MET | 70 | 2.465 | −16.499 | 4.837 | 1.00 | 0.00 | 1MOD |
| ATOM | 564 | N | LEU | 71 | 7.104 | −12.587 | 5.282 | 1.00 | 0.00 | 1MOD |
| ATOM | 565 | CA | LEU | 71 | 8.040 | −12.676 | 6.363 | 1.00 | 0.00 | 1MOD |
| ATOM | 566 | C | LEU | 71 | 8.177 | −11.370 | 7.056 | 1.00 | 0.00 | 1MOD |
| ATOM | 567 | O | LEU | 71 | 8.191 | −11.319 | 8.284 | 1.00 | 0.00 | 1MOD |
| ATOM | 568 | CB | LEU | 71 | 9.471 | −13.076 | 5.979 | 1.00 | 0.00 | 1MOD |
| ATOM | 569 | CG | LEU | 71 | 9.683 | −14.583 | 5.792 | 1.00 | 0.00 | 1MOD |
| ATOM | 570 | CD1 | LEU | 71 | 9.004 | −15.121 | 4.524 | 1.00 | 0.00 | 1MOD |
| ATOM | 571 | CD2 | LEU | 71 | 11.174 | −14.920 | 5.893 | 1.00 | 0.00 | 1MOD |
| ATOM | 572 | N | VAL | 72 | 8.270 | −10.272 | 6.289 | 1.00 | 0.00 | 1MOD |
| ATOM | 573 | CA | VAL | 72 | 8.518 | −9.009 | 6.920 | 1.00 | 0.00 | 1MOD |
| ATOM | 574 | C | VAL | 72 | 7.396 | −8.713 | 7.859 | 1.00 | 0.00 | 1MOD |
| ATOM | 575 | O | VAL | 72 | 7.626 | −8.259 | 8.979 | 1.00 | 0.00 | 1MOD |
| ATOM | 576 | CB | VAL | 72 | 8.611 | −7.843 | 5.963 | 1.00 | 0.00 | 1MOD |
| ATOM | 577 | CG1 | VAL | 72 | 9.780 | −8.084 | 5.011 | 1.00 | 0.00 | 1MOD |
| ATOM | 578 | CG2 | VAL | 72 | 7.276 | −7.640 | 5.232 | 1.00 | 0.00 | 1MOD |
| ATOM | 579 | N | ILE | 73 | 6.147 | −8.987 | 7.435 | 1.00 | 0.00 | 1MOD |
| ATOM | 580 | CA | ILE | 73 | 5.031 | −8.623 | 8.253 | 1.00 | 0.00 | 1MOD |
| ATOM | 581 | C | ILE | 73 | 5.103 | −9.371 | 9.549 | 1.00 | 0.00 | 1MOD |
| ATOM | 582 | O | ILE | 73 | 4.866 | −8.791 | 10.608 | 1.00 | 0.00 | 1MOD |
| ATOM | 583 | CB | ILE | 73 | 3.691 | −8.848 | 7.593 | 1.00 | 0.00 | 1MOD |
| ATOM | 584 | CG1 | ILE | 73 | 3.389 | −10.334 | 7.336 | 1.00 | 0.00 | 1MOD |
| ATOM | 585 | CG2 | ILE | 73 | 3.668 | −7.989 | 6.316 | 1.00 | 0.00 | 1MOD |
| ATOM | 586 | CD1 | ILE | 73 | 2.822 | −11.079 | 8.545 | 1.00 | 0.00 | 1MOD |
| ATOM | 587 | N | LEU | 74 | 5.439 | −10.676 | 9.499 | 1.00 | 0.00 | 1MOD |
| ATOM | 588 | CA | LEU | 74 | 5.498 | −11.484 | 10.685 | 1.00 | 0.00 | 1MOD |
| ATOM | 589 | C | LEU | 74 | 6.581 | −10.994 | 11.595 | 1.00 | 0.00 | 1MOD |
| ATOM | 590 | O | LEU | 74 | 6.357 | −10.800 | 12.789 | 1.00 | 0.00 | 1MOD |
| ATOM | 591 | CB | LEU | 74 | 5.826 | −12.957 | 10.377 | 1.00 | 0.00 | 1MOD |
| ATOM | 592 | CG | LEU | 74 | 6.054 | −13.803 | 11.646 | 1.00 | 0.00 | 1MOD |
| ATOM | 593 | CD1 | LEU | 74 | 4.771 | −13.916 | 12.482 | 1.00 | 0.00 | 1MOD |
| ATOM | 594 | CD2 | LEU | 74 | 6.691 | −15.164 | 11.318 | 1.00 | 0.00 | 1MOD |
| ATOM | 595 | N | TYR | 75 | 7.780 | −10.737 | 11.045 | 1.00 | 0.00 | 1MOD |
| ATOM | 596 | CA | TYR | 75 | 8.890 | −10.372 | 11.877 | 1.00 | 0.00 | 1MOD |
| ATOM | 597 | C | TYR | 75 | 8.599 | −9.121 | 12.630 | 1.00 | 0.00 | 1MOD |
| ATOM | 598 | O | TYR | 75 | 8.849 | −9.050 | 13.832 | 1.00 | 0.00 | 1MOD |
| ATOM | 599 | CB | TYR | 75 | 10.202 | −10.127 | 11.107 | 1.00 | 0.00 | 1MOD |
| ATOM | 600 | CG | TYR | 75 | 10.897 | −11.421 | 10.853 | 1.00 | 0.00 | 1MOD |
| ATOM | 601 | CD1 | TYR | 75 | 10.491 | −12.286 | 9.865 | 1.00 | 0.00 | 1MOD |
| ATOM | 602 | CD2 | TYR | 75 | 11.991 | −11.757 | 11.620 | 1.00 | 0.00 | 1MOD |
| ATOM | 603 | CE1 | TYR | 75 | 11.166 | −13.469 | 9.658 | 1.00 | 0.00 | 1MOD |
| ATOM | 604 | CE2 | TYR | 75 | 12.670 | −12.934 | 11.417 | 1.00 | 0.00 | 1MOD |
| ATOM | 605 | CZ | TYR | 75 | 12.257 | −13.795 | 10.430 | 1.00 | 0.00 | 1MOD |
| ATOM | 606 | OH | TYR | 75 | 12.947 | −15.008 | 10.211 | 1.00 | 0.00 | 1MOD |
| ATOM | 607 | N | SER | 76 | 8.049 | −8.093 | 11.967 | 1.00 | 0.00 | 1MOD |
| ATOM | 608 | CA | SER | 76 | 7.858 | −6.890 | 12.723 | 1.00 | 0.00 | 1MOD |
| ATOM | 609 | C | SER | 76 | 6.503 | −6.964 | 13.340 | 1.00 | 0.00 | 1MOD |
| ATOM | 610 | O | SER | 76 | 5.487 | −6.916 | 12.652 | 1.00 | 0.00 | 1MOD |
| ATOM | 611 | CB | SER | 76 | 7.899 | −5.616 | 11.865 | 1.00 | 0.00 | 1MOD |
| ATOM | 612 | OG | SER | 76 | 9.175 | −5.484 | 11.254 | 1.00 | 0.00 | 1MOD |
| ATOM | 613 | N | ARG | 77 | 6.467 | −7.040 | 14.681 | 1.00 | 0.00 | 1MOD |
| ATOM | 614 | CA | ARG | 77 | 5.261 | −7.204 | 15.440 | 1.00 | 0.00 | 1MOD |
| ATOM | 615 | C | ARG | 77 | 4.373 | −6.006 | 15.312 | 1.00 | 0.00 | 1MOD |
| ATOM | 616 | O | ARG | 77 | 3.150 | −6.129 | 15.352 | 1.00 | 0.00 | 1MOD |
| ATOM | 617 | CB | ARG | 77 | 5.528 | −7.458 | 16.932 | 1.00 | 0.00 | 1MOD |
| ATOM | 618 | CG | ARG | 77 | 6.138 | −8.837 | 17.188 | 1.00 | 0.00 | 1MOD |
| ATOM | 619 | CD | ARG | 77 | 7.538 | −9.011 | 16.595 | 1.00 | 0.00 | 1MOD |
| ATOM | 620 | NE | ARG | 77 | 8.500 | −8.446 | 17.582 | 1.00 | 0.00 | 1MOD |
| ATOM | 621 | CZ | ARG | 77 | 8.959 | −9.238 | 18.593 | 1.00 | 0.00 | 1MOD |
| ATOM | 622 | NH1 | ARG | 77 | 8.578 | −10.548 | 18.648 | 1.00 | 0.00 | 1MOD |
| ATOM | 623 | NH2 | ARG | 77 | 9.795 | −8.724 | 19.540 | 1.00 | 0.00 | 1MOD |
| ATOM | 624 | N | VAL | 78 | 4.963 | −4.811 | 15.168 | 1.00 | 0.00 | 1MOD |
| ATOM | 625 | CA | VAL | 78 | 4.210 | −3.593 | 15.130 | 1.00 | 0.00 | 1MOD |
| ATOM | 626 | C | VAL | 78 | 3.271 | −3.572 | 13.965 | 1.00 | 0.00 | 1MOD |
| ATOM | 627 | O | VAL | 78 | 2.160 | −3.064 | 14.104 | 1.00 | 0.00 | 1MOD |
| ATOM | 628 | CB | VAL | 78 | 5.080 | −2.375 | 15.015 | 1.00 | 0.00 | 1MOD |
| ATOM | 629 | CG1 | VAL | 78 | 4.177 | −1.133 | 14.912 | 1.00 | 0.00 | 1MOD |
| ATOM | 630 | CG2 | VAL | 78 | 6.051 | −2.342 | 16.207 | 1.00 | 0.00 | 1MOD |
| ATOM | 631 | N | GLY | 79 | 3.687 | −4.087 | 12.786 | 1.00 | 0.00 | 1MOD |
| ATOM | 632 | CA | GLY | 79 | 2.854 | −3.992 | 11.613 | 1.00 | 0.00 | 1MOD |
| ATOM | 633 | C | GLY | 79 | 1.605 | −4.801 | 11.784 | 1.00 | 0.00 | 1MOD |
| ATOM | 634 | O | GLY | 79 | 1.526 | −5.942 | 11.329 | 1.00 | 0.00 | 1MOD |
| ATOM | 635 | N | ARG | 80 | 0.562 | −4.201 | 12.395 | 1.00 | 0.00 | 1MOD |
| ATOM | 636 | CA | ARG | 80 | −0.662 | −4.919 | 12.566 | 1.00 | 0.00 | 1MOD |
| ATOM | 637 | C | ARG | 80 | −1.780 | −3.992 | 12.213 | 1.00 | 0.00 | 1MOD |
| ATOM | 638 | O | ARG | 80 | −2.799 | −3.947 | 12.903 | 1.00 | 0.00 | 1MOD |
| ATOM | 639 | CB | ARG | 80 | −0.912 | −5.343 | 14.024 | 1.00 | 0.00 | 1MOD |
| ATOM | 640 | CG | ARG | 80 | 0.174 | −6.255 | 14.596 | 1.00 | 0.00 | 1MOD |
| ATOM | 641 | CD | ARG | 80 | 0.567 | −7.398 | 13.661 | 1.00 | 0.00 | 1MOD |
| ATOM | 642 | NE | ARG | 80 | 1.571 | −8.235 | 14.375 | 1.00 | 0.00 | 1MOD |
| ATOM | 643 | CZ | ARG | 80 | 2.566 | −8.860 | 13.679 | 1.00 | 0.00 | 1MOD |
| ATOM | 644 | NH1 | ARG | 80 | 2.692 | −8.658 | 12.336 | 1.00 | 0.00 | 1MOD |
| ATOM | 645 | NH2 | ARG | 80 | 3.436 | −9.684 | 14.330 | 1.00 | 0.00 | 1MOD |
| ATOM | 646 | N | SER | 81 | −1.633 | −3.234 | 11.114 | 1.00 | 0.00 | 1MOD |
| ATOM | 647 | CA | SER | 81 | −2.706 | −2.375 | 10.721 | 1.00 | 0.00 | 1MOD |
| ATOM | 648 | C | SER | 81 | −3.642 | −3.242 | 9.965 | 1.00 | 0.00 | 1MOD |
| ATOM | 649 | O | SER | 81 | −3.316 | −4.378 | 9.629 | 1.00 | 0.00 | 1MOD |
| ATOM | 650 | CB | SER | 81 | −2.297 | −1.232 | 9.777 | 1.00 | 0.00 | 1MOD |
| ATOM | 651 | OG | SER | 81 | −3.443 | −0.481 | 9.402 | 1.00 | 0.00 | 1MOD |
| ATOM | 652 | N | VAL | 82 | −4.854 | −2.729 | 9.704 | 1.00 | 0.00 | 1MOD |
| ATOM | 653 | CA | VAL | 82 | −5.784 | −3.492 | 8.938 | 1.00 | 0.00 | 1MOD |
| ATOM | 654 | C | VAL | 82 | −5.168 | −3.664 | 7.587 | 1.00 | 0.00 | 1MOD |
| ATOM | 655 | O | VAL | 82 | −5.256 | −4.723 | 6.973 | 1.00 | 0.00 | 1MOD |
| ATOM | 656 | CB | VAL | 82 | −7.089 | −2.779 | 8.768 | 1.00 | 0.00 | 1MOD |
| ATOM | 657 | CG1 | VAL | 82 | −7.983 | −3.624 | 7.854 | 1.00 | 0.00 | 1MOD |
| ATOM | 658 | CG2 | VAL | 82 | −7.694 | −2.518 | 10.157 | 1.00 | 0.00 | 1MOD |
| ATOM | 659 | N | THR | 83 | −4.519 | −2.604 | 7.078 | 1.00 | 0.00 | 1MOD |
| ATOM | 660 | CA | THR | 83 | −3.917 | −2.683 | 5.779 | 1.00 | 0.00 | 1MOD |
| ATOM | 661 | C | THR | 83 | −2.797 | −3.670 | 5.816 | 1.00 | 0.00 | 1MOD |
| ATOM | 662 | O | THR | 83 | −2.612 | −4.454 | 4.888 | 1.00 | 0.00 | 1MOD |
| ATOM | 663 | CB | THR | 83 | −3.361 | −1.366 | 5.306 | 1.00 | 0.00 | 1MOD |
| ATOM | 664 | OG1 | THR | 83 | −2.885 | −1.483 | 3.973 | 1.00 | 0.00 | 1MOD |
| ATOM | 665 | CG2 | THR | 83 | −2.223 | −0.923 | 6.241 | 1.00 | 0.00 | 1MOD |
| ATOM | 666 | N | ASP | 84 | −2.022 | −3.661 | 6.913 | 1.00 | 0.00 | 1MOD |
| ATOM | 667 | CA | ASP | 84 | −0.897 | −4.540 | 7.022 | 1.00 | 0.00 | 1MOD |
| ATOM | 668 | C | ASP | 84 | −1.419 | −5.938 | 6.987 | 1.00 | 0.00 | 1MOD |
| ATOM | 669 | O | ASP | 84 | −0.811 | −6.824 | 6.387 | 1.00 | 0.00 | 1MOD |
| ATOM | 670 | CB | ASP | 84 | −0.144 | −4.362 | 8.353 | 1.00 | 0.00 | 1MOD |
| ATOM | 671 | CG | ASP | 84 | 0.436 | −2.954 | 8.390 | 1.00 | 0.00 | 1MOD |
| ATOM | 672 | OD1 | ASP | 84 | 0.257 | −2.211 | 7.389 | 1.00 | 0.00 | 1MOD |
| ATOM | 673 | OD2 | ASP | 84 | 1.070 | −2.603 | 9.421 | 1.00 | 0.00 | 1MOD |
| ATOM | 674 | N | VAL | 85 | −2.569 | −6.178 | 7.644 | 1.00 | 0.00 | 1MOD |
| ATOM | 675 | CA | VAL | 85 | −3.075 | −7.517 | 7.693 | 1.00 | 0.00 | 1MOD |
| ATOM | 676 | C | VAL | 85 | −3.506 | −7.955 | 6.333 | 1.00 | 0.00 | 1MOD |
| ATOM | 677 | O | VAL | 85 | −3.252 | −9.094 | 5.947 | 1.00 | 0.00 | 1MOD |
| ATOM | 678 | CB | VAL | 85 | −4.204 | −7.709 | 8.639 | 1.00 | 0.00 | 1MOD |
| ATOM | 679 | CG1 | VAL | 85 | −3.746 | −7.082 | 9.963 | 1.00 | 0.00 | 1MOD |
| ATOM | 680 | CG2 | VAL | 85 | −5.519 | −7.218 | 8.021 | 1.00 | 0.00 | 1MOD |
| ATOM | 681 | N | TYR | 86 | −4.161 | −7.067 | 5.558 | 1.00 | 0.00 | 1MOD |
| ATOM | 682 | CA | TYR | 86 | −4.599 | −7.496 | 4.260 | 1.00 | 0.00 | 1MOD |
| ATOM | 683 | C | TYR | 86 | −3.451 | −7.893 | 3.406 | 1.00 | 0.00 | 1MOD |
| ATOM | 684 | O | TYR | 86 | −3.527 | −8.913 | 2.723 | 1.00 | 0.00 | 1MOD |
| ATOM | 685 | CB | TYR | 86 | −5.433 | −6.496 | 3.445 | 1.00 | 0.00 | 1MOD |
| ATOM | 686 | CG | TYR | 86 | −6.836 | −6.893 | 3.698 | 1.00 | 0.00 | 1MOD |
| ATOM | 687 | CD1 | TYR | 86 | −7.231 | −8.117 | 3.217 | 1.00 | 0.00 | 1MOD |
| ATOM | 688 | CD2 | TYR | 86 | −7.740 | −6.091 | 4.349 | 1.00 | 0.00 | 1MOD |
| ATOM | 689 | CE1 | TYR | 86 | −8.509 | −8.569 | 3.402 | 1.00 | 0.00 | 1MOD |
| ATOM | 690 | CE2 | TYR | 86 | −9.024 | −6.545 | 4.532 | 1.00 | 0.00 | 1MOD |
| ATOM | 691 | CZ | TYR | 86 | −9.404 | −7.779 | 4.062 | 1.00 | 0.00 | 1MOD |
| ATOM | 692 | OH | TYR | 86 | −10.710 | −8.256 | 4.247 | 1.00 | 0.00 | 1MOD |
| ATOM | 693 | N | LEU | 87 | −2.358 | −7.117 | 3.430 | 1.00 | 0.00 | 1MOD |
| ATOM | 694 | CA | LEU | 87 | −1.242 | −7.393 | 2.569 | 1.00 | 0.00 | 1MOD |
| ATOM | 695 | C | LEU | 87 | −0.740 | −8.752 | 2.922 | 1.00 | 0.00 | 1MOD |
| ATOM | 696 | O | LEU | 87 | −0.325 | −9.516 | 2.053 | 1.00 | 0.00 | 1MOD |
| ATOM | 697 | CB | LEU | 87 | −0.054 | −6.420 | 2.783 | 1.00 | 0.00 | 1MOD |
| ATOM | 698 | CG | LEU | 87 | 1.173 | −6.536 | 1.832 | 1.00 | 0.00 | 1MOD |
| ATOM | 699 | CD1 | LEU | 87 | 1.881 | −7.896 | 1.887 | 1.00 | 0.00 | 1MOD |
| ATOM | 700 | CD2 | LEU | 87 | 0.843 | −6.111 | 0.400 | 1.00 | 0.00 | 1MOD |
| ATOM | 701 | N | LEU | 88 | −0.761 | −9.091 | 4.222 | 1.00 | 0.00 | 1MOD |
| ATOM | 702 | CA | LEU | 88 | −0.257 | −10.373 | 4.610 | 1.00 | 0.00 | 1MOD |
| ATOM | 703 | C | LEU | 88 | −1.084 | −11.399 | 3.916 | 1.00 | 0.00 | 1MOD |
| ATOM | 704 | O | LEU | 88 | −0.561 | −12.388 | 3.401 | 1.00 | 0.00 | 1MOD |
| ATOM | 705 | CB | LEU | 88 | −0.371 | −10.623 | 6.123 | 1.00 | 0.00 | 1MOD |
| ATOM | 706 | CG | LEU | 88 | 0.064 | −12.037 | 6.546 | 1.00 | 0.00 | 1MOD |
| ATOM | 707 | CD1 | LEU | 88 | 1.533 | −12.309 | 6.188 | 1.00 | 0.00 | 1MOD |
| ATOM | 708 | CD2 | LEU | 88 | −0.243 | −12.287 | 8.031 | 1.00 | 0.00 | 1MOD |
| ATOM | 709 | N | ASN | 89 | −2.408 | −11.163 | 3.873 | 1.00 | 0.00 | 1MOD |
| ATOM | 710 | CA | ASN | 89 | −3.337 | −12.075 | 3.277 | 1.00 | 0.00 | 1MOD |
| ATOM | 711 | C | ASN | 89 | −3.012 | −12.226 | 1.823 | 1.00 | 0.00 | 1MOD |
| ATOM | 712 | O | ASN | 89 | −2.914 | −13.343 | 1.319 | 1.00 | 0.00 | 1MOD |
| ATOM | 713 | CB | ASN | 89 | −4.785 | −11.569 | 3.376 | 1.00 | 0.00 | 1MOD |
| ATOM | 714 | CG | ASN | 89 | −5.721 | −12.699 | 2.981 | 1.00 | 0.00 | 1MOD |
| ATOM | 715 | OD1 | ASN | 89 | −5.313 | −13.682 | 2.367 | 1.00 | 0.00 | 1MOD |
| ATOM | 716 | ND2 | ASN | 89 | −7.024 | −12.550 | 3.339 | 1.00 | 0.00 | 1MOD |
| ATOM | 717 | N | LEU | 90 | −2.792 | −11.101 | 1.116 | 1.00 | 0.00 | 1MOD |
| ATOM | 718 | CA | LEU | 90 | −2.511 | −11.181 | −0.291 | 1.00 | 0.00 | 1MOD |
| ATOM | 719 | C | LEU | 90 | −1.267 | −11.986 | −0.476 | 1.00 | 0.00 | 1MOD |
| ATOM | 720 | O | LEU | 90 | −1.182 | −12.826 | −1.371 | 1.00 | 0.00 | 1MOD |
| ATOM | 721 | CB | LEU | 90 | −2.117 | −9.847 | −0.962 | 1.00 | 0.00 | 1MOD |
| ATOM | 722 | CG | LEU | 90 | −3.177 | −8.754 | −1.186 | 1.00 | 0.00 | 1MOD |
| ATOM | 723 | CD1 | LEU | 90 | −4.327 | −9.248 | −2.080 | 1.00 | 0.00 | 1MOD |
| ATOM | 724 | CD2 | LEU | 90 | −3.579 | −8.068 | 0.122 | 1.00 | 0.00 | 1MOD |
| ATOM | 725 | N | ALA | 91 | −0.261 | −11.745 | 0.383 | 1.00 | 0.00 | 1MOD |
| ATOM | 726 | CA | ALA | 91 | 1.014 | −12.369 | 0.198 | 1.00 | 0.00 | 1MOD |
| ATOM | 727 | C | ALA | 91 | 0.841 | −13.849 | 0.254 | 1.00 | 0.00 | 1MOD |
| ATOM | 728 | O | ALA | 91 | 1.433 | −14.573 | −0.544 | 1.00 | 0.00 | 1MOD |
| ATOM | 729 | CB | ALA | 91 | 2.025 | −11.997 | 1.294 | 1.00 | 0.00 | 1MOD |
| ATOM | 730 | N | LEU | 92 | 0.036 | −14.340 | 1.211 | 1.00 | 0.00 | 1MOD |
| ATOM | 731 | CA | LEU | 92 | −0.150 | −15.755 | 1.349 | 1.00 | 0.00 | 1MOD |
| ATOM | 732 | C | LEU | 92 | −0.885 | −16.282 | 0.151 | 1.00 | 0.00 | 1MOD |
| ATOM | 733 | O | LEU | 92 | −0.487 | −17.287 | −0.435 | 1.00 | 0.00 | 1MOD |
| ATOM | 734 | CB | LEU | 92 | −0.945 | −16.093 | 2.630 | 1.00 | 0.00 | 1MOD |
| ATOM | 735 | CG | LEU | 92 | −1.167 | −17.587 | 2.971 | 1.00 | 0.00 | 1MOD |
| ATOM | 736 | CD1 | LEU | 92 | −1.827 | −17.713 | 4.354 | 1.00 | 0.00 | 1MOD |
| ATOM | 737 | CD2 | LEU | 92 | −1.979 | −18.354 | 1.911 | 1.00 | 0.00 | 1MOD |
| ATOM | 738 | N | ALA | 93 | −1.963 | −15.592 | −0.268 | 1.00 | 0.00 | 1MOD |
| ATOM | 739 | CA | ALA | 93 | −2.782 | −16.068 | −1.346 | 1.00 | 0.00 | 1MOD |
| ATOM | 740 | C | ALA | 93 | −1.920 | −16.164 | −2.556 | 1.00 | 0.00 | 1MOD |
| ATOM | 741 | O | ALA | 93 | −2.011 | −17.114 | −3.331 | 1.00 | 0.00 | 1MOD |
| ATOM | 742 | CB | ALA | 93 | −3.939 | −15.108 | −1.680 | 1.00 | 0.00 | 1MOD |
| ATOM | 743 | N | ASP | 94 | −1.037 | −15.170 | −2.727 | 1.00 | 0.00 | 1MOD |
| ATOM | 744 | CA | ASP | 94 | −0.173 | −15.106 | −3.862 | 1.00 | 0.00 | 1MOD |
| ATOM | 745 | C | ASP | 94 | 0.703 | −16.313 | −3.851 | 1.00 | 0.00 | 1MOD |
| ATOM | 746 | O | ASP | 94 | 0.963 | −16.911 | −4.893 | 1.00 | 0.00 | 1MOD |
| ATOM | 747 | CB | ASP | 94 | 0.714 | −13.852 | −3.840 | 1.00 | 0.00 | 1MOD |
| ATOM | 748 | CG | ASP | 94 | −0.198 | −12.645 | −4.025 | 1.00 | 0.00 | 1MOD |
| ATOM | 749 | OD1 | ASP | 94 | −1.410 | −12.858 | −4.303 | 1.00 | 0.00 | 1MOD |
| ATOM | 750 | OD2 | ASP | 94 | 0.306 | −11.497 | −3.897 | 1.00 | 0.00 | 1MOD |
| ATOM | 751 | N | LEU | 95 | 1.165 | −16.721 | −2.658 | 1.00 | 0.00 | 1MOD |
| ATOM | 752 | CA | LEU | 95 | 2.027 | −17.861 | −2.577 | 1.00 | 0.00 | 1MOD |
| ATOM | 753 | C | LEU | 95 | 1.251 | −19.039 | −3.069 | 1.00 | 0.00 | 1MOD |
| ATOM | 754 | O | LEU | 95 | 1.807 | −19.945 | −3.688 | 1.00 | 0.00 | 1MOD |
| ATOM | 755 | CB | LEU | 95 | 2.519 | −18.145 | −1.150 | 1.00 | 0.00 | 1MOD |
| ATOM | 756 | CG | LEU | 95 | 3.390 | −17.004 | −0.588 | 1.00 | 0.00 | 1MOD |
| ATOM | 757 | CD1 | LEU | 95 | 3.876 | −17.313 | 0.835 | 1.00 | 0.00 | 1MOD |
| ATOM | 758 | CD2 | LEU | 95 | 4.545 | −16.652 | −1.540 | 1.00 | 0.00 | 1MOD |
| ATOM | 759 | N | LEU | 96 | −0.067 | −19.054 | −2.792 | 1.00 | 0.00 | 1MOD |
| ATOM | 760 | CA | LEU | 96 | −0.900 | −20.134 | −3.230 | 1.00 | 0.00 | 1MOD |
| ATOM | 761 | C | LEU | 96 | −0.888 | −20.142 | −4.732 | 1.00 | 0.00 | 1MOD |
| ATOM | 762 | O | LEU | 96 | −0.771 | −21.200 | −5.348 | 1.00 | 0.00 | 1MOD |
| ATOM | 763 | CB | LEU | 96 | −2.369 | −19.977 | −2.801 | 1.00 | 0.00 | 1MOD |
| ATOM | 764 | CG | LEU | 96 | −2.582 | −19.890 | −1.278 | 1.00 | 0.00 | 1MOD |
| ATOM | 765 | CD1 | LEU | 96 | −4.078 | −19.820 | −0.933 | 1.00 | 0.00 | 1MOD |
| ATOM | 766 | CD2 | LEU | 96 | −1.849 | −21.017 | −0.533 | 1.00 | 0.00 | 1MOD |
| ATOM | 767 | N | PHE | 97 | −0.976 | −18.950 | −5.364 | 1.00 | 0.00 | 1MOD |
| ATOM | 768 | CA | PHE | 97 | −1.006 | −18.876 | −6.807 | 1.00 | 0.00 | 1MOD |
| ATOM | 769 | C | PHE | 97 | 0.238 | −19.523 | −7.287 | 1.00 | 0.00 | 1MOD |
| ATOM | 770 | O | PHE | 97 | 0.227 | −20.345 | −8.198 | 1.00 | 0.00 | 1MOD |
| ATOM | 771 | CB | PHE | 97 | −0.700 | −17.503 | −7.455 | 1.00 | 0.00 | 1MOD |
| ATOM | 772 | CG | PHE | 97 | −1.632 | −16.331 | −7.434 | 1.00 | 0.00 | 1MOD |
| ATOM | 773 | CD1 | PHE | 97 | −1.657 | −15.455 | −6.377 | 1.00 | 0.00 | 1MOD |
| ATOM | 774 | CD2 | PHE | 97 | −2.432 | −16.061 | −8.523 | 1.00 | 0.00 | 1MOD |
| ATOM | 775 | CE1 | PHE | 97 | −2.476 | −14.349 | −6.382 | 1.00 | 0.00 | 1MOD |
| ATOM | 776 | CE2 | PHE | 97 | −3.255 | −14.958 | −8.536 | 1.00 | 0.00 | 1MOD |
| ATOM | 777 | CZ | PHE | 97 | −3.281 | −14.093 | −7.466 | 1.00 | 0.00 | 1MOD |
| ATOM | 778 | N | ALA | 98 | 1.357 | −19.161 | −6.647 | 1.00 | 0.00 | 1MOD |
| ATOM | 779 | CA | ALA | 98 | 2.647 | −19.573 | −7.096 | 1.00 | 0.00 | 1MOD |
| ATOM | 780 | C | ALA | 98 | 2.651 | −21.054 | −7.152 | 1.00 | 0.00 | 1MOD |
| ATOM | 781 | O | ALA | 98 | 3.418 | −21.612 | −7.921 | 1.00 | 0.00 | 1MOD |
| ATOM | 782 | CB | ALA | 98 | 3.790 | −19.130 | −6.165 | 1.00 | 0.00 | 1MOD |
| ATOM | 783 | N | LEU | 99 | 1.862 | −21.716 | −6.284 | 1.00 | 0.00 | 1MOD |
| ATOM | 784 | CA | LEU | 99 | 1.706 | −23.148 | −6.258 | 1.00 | 0.00 | 1MOD |
| ATOM | 785 | C | LEU | 99 | 1.032 | −23.634 | −7.517 | 1.00 | 0.00 | 1MOD |
| ATOM | 786 | O | LEU | 99 | 1.439 | −24.633 | −8.109 | 1.00 | 0.00 | 1MOD |
| ATOM | 787 | CB | LEU | 99 | 0.845 | −23.571 | −5.049 | 1.00 | 0.00 | 1MOD |
| ATOM | 788 | CG | LEU | 99 | 0.580 | −25.082 | −4.878 | 1.00 | 0.00 | 1MOD |
| ATOM | 789 | CD1 | LEU | 99 | −0.405 | −25.640 | −5.922 | 1.00 | 0.00 | 1MOD |
| ATOM | 790 | CD2 | LEU | 99 | 1.900 | −25.862 | −4.820 | 1.00 | 0.00 | 1MOD |
| ATOM | 791 | N | THR | 100 | −0.032 | −22.934 | −7.958 | 1.00 | 0.00 | 1MOD |
| ATOM | 792 | CA | THR | 100 | −0.811 | −23.308 | −9.109 | 1.00 | 0.00 | 1MOD |
| ATOM | 793 | C | THR | 100 | 0.083 | −23.196 | −10.296 | 1.00 | 0.00 | 1MOD |
| ATOM | 794 | O | THR | 100 | −0.101 | −23.860 | −11.312 | 1.00 | 0.00 | 1MOD |
| ATOM | 795 | CB | THR | 100 | −1.983 | −22.401 | −9.358 | 1.00 | 0.00 | 1MOD |
| ATOM | 796 | OG1 | THR | 100 | −2.923 | −23.062 | −10.179 | 1.00 | 0.00 | 1MOD |
| ATOM | 797 | CG2 | THR | 100 | −1.519 | −21.148 | −10.119 | 1.00 | 0.00 | 1MOD |
| ATOM | 798 | N | LEU | 101 | 1.078 | −22.308 | −10.170 | 1.00 | 0.00 | 1MOD |
| ATOM | 799 | CA | LEU | 101 | 1.991 | −21.970 | −11.217 | 1.00 | 0.00 | 1MOD |
| ATOM | 800 | C | LEU | 101 | 2.744 | −23.183 | −11.694 | 1.00 | 0.00 | 1MOD |
| ATOM | 801 | O | LEU | 101 | 2.968 | −23.269 | −12.898 | 1.00 | 0.00 | 1MOD |
| ATOM | 802 | CB | LEU | 101 | 3.011 | −20.905 | −10.754 | 1.00 | 0.00 | 1MOD |
| ATOM | 803 | CG | LEU | 101 | 3.967 | −20.357 | −11.838 | 1.00 | 0.00 | 1MOD |
| ATOM | 804 | CD1 | LEU | 101 | 5.001 | −21.396 | −12.294 | 1.00 | 0.00 | 1MOD |
| ATOM | 805 | CD2 | LEU | 101 | 3.191 | −19.740 | −13.011 | 1.00 | 0.00 | 1MOD |
| ATOM | 806 | N | PRO | 102 | 3.189 | −24.119 | −10.892 | 1.00 | 0.00 | 1MOD |
| ATOM | 807 | CA | PRO | 102 | 3.928 | −25.202 | −11.472 | 1.00 | 0.00 | 1MOD |
| ATOM | 808 | C | PRO | 102 | 3.150 | −26.081 | −12.377 | 1.00 | 0.00 | 1MOD |
| ATOM | 809 | O | PRO | 102 | 3.740 | −26.608 | −13.312 | 1.00 | 0.00 | 1MOD |
| ATOM | 810 | CB | PRO | 102 | 4.618 | −25.931 | −10.325 | 1.00 | 0.00 | 1MOD |
| ATOM | 811 | CG | PRO | 102 | 4.846 | −24.809 | −9.304 | 1.00 | 0.00 | 1MOD |
| ATOM | 812 | CD | PRO | 102 | 3.696 | −23.825 | −9.572 | 1.00 | 0.00 | 1MOD |
| ATOM | 813 | N | ILE | 103 | 1.851 | −26.297 | −12.124 | 1.00 | 0.00 | 1MOD |
| ATOM | 814 | CA | ILE | 103 | 1.141 | −27.090 | −13.076 | 1.00 | 0.00 | 1MOD |
| ATOM | 815 | C | ILE | 103 | 1.060 | −26.316 | −14.345 | 1.00 | 0.00 | 1MOD |
| ATOM | 816 | O | ILE | 103 | 1.177 | −26.875 | −15.435 | 1.00 | 0.00 | 1MOD |
| ATOM | 817 | CB | ILE | 103 | −0.217 | −27.551 | −12.626 | 1.00 | 0.00 | 1MOD |
| ATOM | 818 | CG1 | ILE | 103 | −0.997 | −28.105 | −13.832 | 1.00 | 0.00 | 1MOD |
| ATOM | 819 | CG2 | ILE | 103 | −0.894 | −26.430 | −11.830 | 1.00 | 0.00 | 1MOD |
| ATOM | 820 | CD1 | ILE | 103 | −0.304 | −29.277 | −14.523 | 1.00 | 0.00 | 1MOD |
| ATOM | 821 | N | TRP | 104 | 0.854 | −24.995 | −14.224 | 1.00 | 0.00 | 1MOD |
| ATOM | 822 | CA | TRP | 104 | 0.720 | −24.148 | −15.369 | 1.00 | 0.00 | 1MOD |
| ATOM | 823 | C | TRP | 104 | 2.019 | −24.162 | −16.116 | 1.00 | 0.00 | 1MOD |
| ATOM | 824 | O | TRP | 104 | 2.054 | −24.249 | −17.341 | 1.00 | 0.00 | 1MOD |
| ATOM | 825 | CB | TRP | 104 | 0.381 | −22.705 | −14.960 | 1.00 | 0.00 | 1MOD |
| ATOM | 826 | CG | TRP | 104 | −0.147 | −21.842 | −16.077 | 1.00 | 0.00 | 1MOD |
| ATOM | 827 | CD1 | TRP | 104 | 0.024 | −21.940 | −17.427 | 1.00 | 0.00 | 1MOD |
| ATOM | 828 | CD2 | TRP | 104 | −1.036 | −20.734 | −15.860 | 1.00 | 0.00 | 1MOD |
| ATOM | 829 | NE1 | TRP | 104 | −0.714 | −20.967 | −18.063 | 1.00 | 0.00 | 1MOD |
| ATOM | 830 | CE2 | TRP | 104 | −1.370 | −20.218 | −17.110 | 1.00 | 0.00 | 1MOD |
| ATOM | 831 | CE3 | TRP | 104 | −1.544 | −20.200 | −14.710 | 1.00 | 0.00 | 1MOD |
| ATOM | 832 | CZ2 | TRP | 104 | −2.220 | −19.154 | −17.231 | 1.00 | 0.00 | 1MOD |
| ATOM | 833 | CZ3 | TRP | 104 | −2.392 | −19.122 | −14.834 | 1.00 | 0.00 | 1MOD |
| ATOM | 834 | CH2 | TRP | 104 | −2.726 | −18.610 | −16.070 | 1.00 | 0.00 | 1MOD |
| ATOM | 835 | N | ALA | 105 | 3.137 | −24.049 | −15.379 | 1.00 | 0.00 | 1MOD |
| ATOM | 836 | CA | ALA | 105 | 4.438 | −24.064 | −15.979 | 1.00 | 0.00 | 1MOD |
| ATOM | 837 | C | ALA | 105 | 4.697 | −25.413 | −16.563 | 1.00 | 0.00 | 1MOD |
| ATOM | 838 | O | ALA | 105 | 5.162 | −25.527 | −17.689 | 1.00 | 0.00 | 1MOD |
| ATOM | 839 | CB | ALA | 105 | 5.559 | −23.784 | −14.966 | 1.00 | 0.00 | 1MOD |
| ATOM | 840 | N | ALA | 106 | 4.384 | −26.487 | −15.824 | 1.00 | 0.00 | 1MOD |
| ATOM | 841 | CA | ALA | 106 | 4.670 | −27.813 | −16.286 | 1.00 | 0.00 | 1MOD |
| ATOM | 842 | C | ALA | 106 | 3.887 | −28.053 | −17.527 | 1.00 | 0.00 | 1MOD |
| ATOM | 843 | O | ALA | 106 | 4.378 | −28.636 | −18.493 | 1.00 | 0.00 | 1MOD |
| ATOM | 844 | CB | ALA | 106 | 4.261 | −28.900 | −15.277 | 1.00 | 0.00 | 1MOD |
| ATOM | 845 | N | SER | 107 | 2.629 | −27.590 | −17.530 | 1.00 | 0.00 | 1MOD |
| ATOM | 846 | CA | SER | 107 | 1.781 | −27.829 | −18.655 | 1.00 | 0.00 | 1MOD |
| ATOM | 847 | C | SER | 107 | 2.322 | −27.127 | −19.855 | 1.00 | 0.00 | 1MOD |
| ATOM | 848 | O | SER | 107 | 2.253 | −27.648 | −20.966 | 1.00 | 0.00 | 1MOD |
| ATOM | 849 | CB | SER | 107 | 0.350 | −27.316 | −18.440 | 1.00 | 0.00 | 1MOD |
| ATOM | 850 | OG | SER | 107 | 0.344 | −25.901 | −18.341 | 1.00 | 0.00 | 1MOD |
| ATOM | 851 | N | LYS | 108 | 2.954 | −25.960 | −19.642 | 1.00 | 0.00 | 1MOD |
| ATOM | 852 | CA | LYS | 108 | 3.361 | −25.093 | −20.710 | 1.00 | 0.00 | 1MOD |
| ATOM | 853 | C | LYS | 108 | 4.217 | −25.867 | −21.647 | 1.00 | 0.00 | 1MOD |
| ATOM | 854 | O | LYS | 108 | 4.291 | −25.558 | −22.837 | 1.00 | 0.00 | 1MOD |
| ATOM | 855 | CB | LYS | 108 | 4.273 | −23.931 | −20.285 | 1.00 | 0.00 | 1MOD |
| ATOM | 856 | CG | LYS | 108 | 5.743 | −24.110 | −20.712 | 1.00 | 0.00 | 1MOD |
| ATOM | 857 | CD | LYS | 108 | 6.644 | −25.054 | −19.898 | 1.00 | 0.00 | 1MOD |
| ATOM | 858 | CE | LYS | 108 | 7.833 | −25.671 | −20.641 | 1.00 | 0.00 | 1MOD |
| ATOM | 859 | NZ | LYS | 108 | 8.835 | −26.182 | −19.674 | 1.00 | 0.00 | 1MOD |
| ATOM | 860 | N | VAL | 109 | 4.911 | −26.884 | −21.117 | 1.00 | 0.00 | 1MOD |
| ATOM | 861 | CA | VAL | 109 | 5.830 | −27.634 | −21.909 | 1.00 | 0.00 | 1MOD |
| ATOM | 862 | C | VAL | 109 | 5.089 | −28.173 | −23.083 | 1.00 | 0.00 | 1MOD |
| ATOM | 863 | O | VAL | 109 | 5.547 | −28.033 | −24.214 | 1.00 | 0.00 | 1MOD |
| ATOM | 864 | CB | VAL | 109 | 6.369 | −28.845 | −21.200 | 1.00 | 0.00 | 1MOD |
| ATOM | 865 | CG1 | VAL | 109 | 7.335 | −29.585 | −22.145 | 1.00 | 0.00 | 1MOD |
| ATOM | 866 | CG2 | VAL | 109 | 7.000 | −28.415 | −19.869 | 1.00 | 0.00 | 1MOD |
| ATOM | 867 | N | ASN | 110 | 3.921 | −28.806 | −22.854 | 1.00 | 0.00 | 1MOD |
| ATOM | 868 | CA | ASN | 110 | 3.243 | −29.378 | −23.979 | 1.00 | 0.00 | 1MOD |
| ATOM | 869 | C | ASN | 110 | 1.776 | −29.529 | −23.717 | 1.00 | 0.00 | 1MOD |
| ATOM | 870 | O | ASN | 110 | 0.950 | −28.883 | −24.362 | 1.00 | 0.00 | 1MOD |
| ATOM | 871 | CB | ASN | 110 | 3.780 | −30.764 | −24.367 | 1.00 | 0.00 | 1MOD |
| ATOM | 872 | CG | ASN | 110 | 5.043 | −30.559 | −25.192 | 1.00 | 0.00 | 1MOD |
| ATOM | 873 | OD1 | ASN | 110 | 5.030 | −29.846 | −26.194 | 1.00 | 0.00 | 1MOD |
| ATOM | 874 | ND2 | ASN | 110 | 6.168 | −31.190 | −24.764 | 1.00 | 0.00 | 1MOD |
| ATOM | 875 | N | GLY | 111 | 1.400 | −30.428 | −22.787 | 1.00 | 0.00 | 1MOD |
| ATOM | 876 | CA | GLY | 111 | −0.003 | −30.680 | −22.619 | 1.00 | 0.00 | 1MOD |
| ATOM | 877 | C | GLY | 111 | −0.363 | −30.681 | −21.178 | 1.00 | 0.00 | 1MOD |
| ATOM | 878 | O | GLY | 111 | 0.484 | −30.561 | −20.295 | 1.00 | 0.00 | 1MOD |
| ATOM | 879 | N | TRP | 112 | −1.674 | −30.826 | −20.920 | 1.00 | 0.00 | 1MOD |
| ATOM | 880 | CA | TRP | 112 | −2.163 | −30.806 | −19.585 | 1.00 | 0.00 | 1MOD |
| ATOM | 881 | C | TRP | 112 | −1.917 | −32.191 | −19.090 | 1.00 | 0.00 | 1MOD |
| ATOM | 882 | O | TRP | 112 | −2.828 | −33.010 | −18.969 | 1.00 | 0.00 | 1MOD |
| ATOM | 883 | CB | TRP | 112 | −3.661 | −30.537 | −19.576 | 1.00 | 0.00 | 1MOD |
| ATOM | 884 | CG | TRP | 112 | −4.014 | −29.895 | −18.294 | 1.00 | 0.00 | 1MOD |
| ATOM | 885 | CD1 | TRP | 112 | −4.245 | −30.451 | −17.086 | 1.00 | 0.00 | 1MOD |
| ATOM | 886 | CD2 | TRP | 112 | −4.126 | −28.477 | −18.128 | 1.00 | 0.00 | 1MOD |
| ATOM | 887 | NE1 | TRP | 112 | −4.468 | −29.469 | −16.171 | 1.00 | 0.00 | 1MOD |
| ATOM | 888 | CE2 | TRP | 112 | −4.409 | −28.248 | −16.788 | 1.00 | 0.00 | 1MOD |
| ATOM | 889 | CE3 | TRP | 112 | −3.977 | −27.455 | −19.013 | 1.00 | 0.00 | 1MOD |
| ATOM | 890 | CZ2 | TRP | 112 | −4.555 | −26.984 | −16.295 | 1.00 | 0.00 | 1MOD |
| ATOM | 891 | CZ3 | TRP | 112 | −4.161 | −26.185 | −18.522 | 1.00 | 0.00 | 1MOD |
| ATOM | 892 | CH2 | TRP | 112 | −4.441 | −25.949 | −17.192 | 1.00 | 0.00 | 1MOD |
| ATOM | 893 | N | ILE | 113 | −0.647 | −32.452 | −18.743 | 1.00 | 0.00 | 1MOD |
| ATOM | 894 | CA | ILE | 113 | −0.196 | −33.779 | −18.466 | 1.00 | 0.00 | 1MOD |
| ATOM | 895 | C | ILE | 113 | −0.905 | −34.408 | −17.319 | 1.00 | 0.00 | 1MOD |
| ATOM | 896 | O | ILE | 113 | −1.280 | −35.576 | −17.407 | 1.00 | 0.00 | 1MOD |
| ATOM | 897 | CB | ILE | 113 | 1.284 | −33.856 | −18.213 | 1.00 | 0.00 | 1MOD |
| ATOM | 898 | CG1 | ILE | 113 | 1.727 | −35.323 | −18.076 | 1.00 | 0.00 | 1MOD |
| ATOM | 899 | CG2 | ILE | 113 | 1.624 | −32.972 | −17.005 | 1.00 | 0.00 | 1MOD |
| ATOM | 900 | CD1 | ILE | 113 | 3.244 | −35.511 | −18.116 | 1.00 | 0.00 | 1MOD |
| ATOM | 901 | N | PHE | 114 | −1.123 | −33.702 | −16.199 | 1.00 | 0.00 | 1MOD |
| ATOM | 902 | CA | PHE | 114 | −1.715 | −34.508 | −15.179 | 1.00 | 0.00 | 1MOD |
| ATOM | 903 | C | PHE | 114 | −3.196 | −34.351 | −15.147 | 1.00 | 0.00 | 1MOD |
| ATOM | 904 | O | PHE | 114 | −3.788 | −34.123 | −14.093 | 1.00 | 0.00 | 1MOD |
| ATOM | 905 | CB | PHE | 114 | −1.153 | −34.230 | −13.784 | 1.00 | 0.00 | 1MOD |
| ATOM | 906 | CG | PHE | 114 | 0.237 | −34.760 | −13.768 | 1.00 | 0.00 | 1MOD |
| ATOM | 907 | CD1 | PHE | 114 | 1.293 | −34.037 | −14.275 | 1.00 | 0.00 | 1MOD |
| ATOM | 908 | CD2 | PHE | 114 | 0.482 | −36.000 | −13.236 | 1.00 | 0.00 | 1MOD |
| ATOM | 909 | CE1 | PHE | 114 | 2.570 | −34.550 | −14.253 | 1.00 | 0.00 | 1MOD |
| ATOM | 910 | CE2 | PHE | 114 | 1.755 | −36.517 | −13.211 | 1.00 | 0.00 | 1MOD |
| ATOM | 911 | CZ | PHE | 114 | 2.804 | −35.795 | −13.721 | 1.00 | 0.00 | 1MOD |
| ATOM | 912 | N | GLY | 115 | −3.837 | −34.507 | −16.316 | 1.00 | 0.00 | 1MOD |
| ATOM | 913 | CA | GLY | 115 | −5.264 | −34.563 | −16.398 | 1.00 | 0.00 | 1MOD |
| ATOM | 914 | C | GLY | 115 | −5.906 | −33.373 | −15.769 | 1.00 | 0.00 | 1MOD |
| ATOM | 915 | O | GLY | 115 | −5.274 | −32.369 | −15.455 | 1.00 | 0.00 | 1MOD |
| ATOM | 916 | N | THR | 116 | −7.217 | −33.529 | −15.519 | 1.00 | 0.00 | 1MOD |
| ATOM | 917 | CA | THR | 116 | −8.105 | −32.530 | −15.007 | 1.00 | 0.00 | 1MOD |
| ATOM | 918 | C | THR | 116 | −7.664 | −32.085 | −13.651 | 1.00 | 0.00 | 1MOD |
| ATOM | 919 | O | THR | 116 | −7.856 | −30.925 | −13.299 | 1.00 | 0.00 | 1MOD |
| ATOM | 920 | CB | THR | 116 | −9.509 | −33.044 | −14.880 | 1.00 | 0.00 | 1MOD |
| ATOM | 921 | OG1 | THR | 116 | −10.395 | −31.989 | −14.539 | 1.00 | 0.00 | 1MOD |
| ATOM | 922 | CG2 | THR | 116 | −9.528 | −34.138 | −13.799 | 1.00 | 0.00 | 1MOD |
| ATOM | 923 | N | PHE | 117 | −7.075 | −32.982 | −12.841 | 1.00 | 0.00 | 1MOD |
| ATOM | 924 | CA | PHE | 117 | −6.721 | −32.585 | −11.506 | 1.00 | 0.00 | 1MOD |
| ATOM | 925 | C | PHE | 117 | −5.787 | −31.419 | −11.573 | 1.00 | 0.00 | 1MOD |
| ATOM | 926 | O | PHE | 117 | −5.984 | −30.413 | −10.894 | 1.00 | 0.00 | 1MOD |
| ATOM | 927 | CB | PHE | 117 | −6.009 | −33.690 | −10.710 | 1.00 | 0.00 | 1MOD |
| ATOM | 928 | CG | PHE | 117 | −5.758 | −33.143 | −9.347 | 1.00 | 0.00 | 1MOD |
| ATOM | 929 | CD1 | PHE | 117 | −6.727 | −33.221 | −8.373 | 1.00 | 0.00 | 1MOD |
| ATOM | 930 | CD2 | PHE | 117 | −4.553 | −32.551 | −9.042 | 1.00 | 0.00 | 1MOD |
| ATOM | 931 | CE1 | PHE | 117 | −6.497 | −32.715 | −7.115 | 1.00 | 0.00 | 1MOD |
| ATOM | 932 | CE2 | PHE | 117 | −4.318 | −32.045 | −7.784 | 1.00 | 0.00 | 1MOD |
| ATOM | 933 | CZ | PHE | 117 | −5.292 | −32.127 | −6.818 | 1.00 | 0.00 | 1MOD |
| ATOM | 934 | N | LEU | 118 | −4.744 | −31.504 | −12.409 | 1.00 | 0.00 | 1MOD |
| ATOM | 935 | CA | LEU | 118 | −3.864 | −30.383 | −12.495 | 1.00 | 0.00 | 1MOD |
| ATOM | 936 | C | LEU | 118 | −4.608 | −29.241 | −13.090 | 1.00 | 0.00 | 1MOD |
| ATOM | 937 | O | LEU | 118 | −4.236 | −28.083 | −12.912 | 1.00 | 0.00 | 1MOD |
| ATOM | 938 | CB | LEU | 118 | −2.530 | −30.683 | −13.185 | 1.00 | 0.00 | 1MOD |
| ATOM | 939 | CG | LEU | 118 | −1.593 | −31.372 | −12.176 | 1.00 | 0.00 | 1MOD |
| ATOM | 940 | CD1 | LEU | 118 | −2.239 | −32.626 | −11.574 | 1.00 | 0.00 | 1MOD |
| ATOM | 941 | CD2 | LEU | 118 | −0.210 | −31.636 | −12.771 | 1.00 | 0.00 | 1MOD |
| ATOM | 942 | N | CYS | 119 | −5.665 | −29.542 | −13.855 | 1.00 | 0.00 | 1MOD |
| ATOM | 943 | CA | CYS | 119 | −6.449 | −28.506 | −14.460 | 1.00 | 0.00 | 1MOD |
| ATOM | 944 | C | CYS | 119 | −7.105 | −27.751 | −13.346 | 1.00 | 0.00 | 1MOD |
| ATOM | 945 | O | CYS | 119 | −7.105 | −26.521 | −13.325 | 1.00 | 0.00 | 1MOD |
| ATOM | 946 | CB | CYS | 119 | −7.494 | −29.122 | −15.407 | 1.00 | 0.00 | 1MOD |
| ATOM | 947 | SG | CYS | 119 | −8.602 | −28.013 | −16.337 | 1.00 | 0.00 | 1MOD |
| ATOM | 948 | N | LYS | 120 | −7.668 | −28.488 | −12.369 | 1.00 | 0.00 | 1MOD |
| ATOM | 949 | CA | LYS | 120 | −8.305 | −27.890 | −11.235 | 1.00 | 0.00 | 1MOD |
| ATOM | 950 | C | LYS | 120 | −7.281 | −27.180 | −10.412 | 1.00 | 0.00 | 1MOD |
| ATOM | 951 | O | LYS | 120 | −7.551 | −26.111 | −9.874 | 1.00 | 0.00 | 1MOD |
| ATOM | 952 | CB | LYS | 120 | −9.027 | −28.901 | −10.328 | 1.00 | 0.00 | 1MOD |
| ATOM | 953 | CG | LYS | 120 | −10.408 | −29.306 | −10.849 | 1.00 | 0.00 | 1MOD |
| ATOM | 954 | CD | LYS | 120 | −10.375 | −30.091 | −12.161 | 1.00 | 0.00 | 1MOD |
| ATOM | 955 | CE | LYS | 120 | −11.760 | −30.312 | −12.773 | 1.00 | 0.00 | 1MOD |
| ATOM | 956 | NZ | LYS | 120 | −12.284 | −29.034 | −13.305 | 1.00 | 0.00 | 1MOD |
| ATOM | 957 | N | VAL | 121 | −6.075 | −27.757 | −10.258 | 1.00 | 0.00 | 1MOD |
| ATOM | 958 | CA | VAL | 121 | −5.119 | −27.068 | −9.443 | 1.00 | 0.00 | 1MOD |
| ATOM | 959 | C | VAL | 121 | −4.780 | −25.774 | −10.107 | 1.00 | 0.00 | 1MOD |
| ATOM | 960 | O | VAL | 121 | −4.755 | −24.728 | −9.464 | 1.00 | 0.00 | 1MOD |
| ATOM | 961 | CB | VAL | 121 | −3.850 | −27.845 | −9.178 | 1.00 | 0.00 | 1MOD |
| ATOM | 962 | CG1 | VAL | 121 | −3.032 | −27.997 | −10.465 | 1.00 | 0.00 | 1MOD |
| ATOM | 963 | CG2 | VAL | 121 | −3.068 | −27.119 | −8.072 | 1.00 | 0.00 | 1MOD |
| ATOM | 964 | N | VAL | 122 | −4.533 | −25.804 | −11.430 | 1.00 | 0.00 | 1MOD |
| ATOM | 965 | CA | VAL | 122 | −4.163 | −24.601 | −12.118 | 1.00 | 0.00 | 1MOD |
| ATOM | 966 | C | VAL | 122 | −5.275 | −23.606 | −12.019 | 1.00 | 0.00 | 1MOD |
| ATOM | 967 | O | VAL | 122 | −5.067 | −22.456 | −11.640 | 1.00 | 0.00 | 1MOD |
| ATOM | 968 | CB | VAL | 122 | −3.996 | −24.797 | −13.597 | 1.00 | 0.00 | 1MOD |
| ATOM | 969 | CG1 | VAL | 122 | −3.692 | −23.429 | −14.234 | 1.00 | 0.00 | 1MOD |
| ATOM | 970 | CG2 | VAL | 122 | −2.937 | −25.869 | −13.870 | 1.00 | 0.00 | 1MOD |
| ATOM | 971 | N | SER | 123 | −6.499 | −24.033 | −12.380 | 1.00 | 0.00 | 1MOD |
| ATOM | 972 | CA | SER | 123 | −7.593 | −23.115 | −12.447 | 1.00 | 0.00 | 1MOD |
| ATOM | 973 | C | SER | 123 | −8.025 | −22.653 | −11.095 | 1.00 | 0.00 | 1MOD |
| ATOM | 974 | O | SER | 123 | −8.267 | −21.465 | −10.900 | 1.00 | 0.00 | 1MOD |
| ATOM | 975 | CB | SER | 123 | −8.832 | −23.688 | −13.133 | 1.00 | 0.00 | 1MOD |
| ATOM | 976 | OG | SER | 123 | −9.873 | −22.724 | −13.156 | 1.00 | 0.00 | 1MOD |
| ATOM | 977 | N | LEU | 124 | −8.139 | −23.581 | −10.128 | 1.00 | 0.00 | 1MOD |
| ATOM | 978 | CA | LEU | 124 | −8.658 | −23.265 | −8.826 | 1.00 | 0.00 | 1MOD |
| ATOM | 979 | C | LEU | 124 | −7.792 | −22.280 | −8.114 | 1.00 | 0.00 | 1MOD |
| ATOM | 980 | O | LEU | 124 | −8.288 | −21.302 | −7.559 | 1.00 | 0.00 | 1MOD |
| ATOM | 981 | CB | LEU | 124 | −8.764 | −24.497 | −7.909 | 1.00 | 0.00 | 1MOD |
| ATOM | 982 | CG | LEU | 124 | −9.299 | −24.177 | −6.499 | 1.00 | 0.00 | 1MOD |
| ATOM | 983 | CD1 | LEU | 124 | −10.743 | −23.656 | −6.552 | 1.00 | 0.00 | 1MOD |
| ATOM | 984 | CD2 | LEU | 124 | −9.139 | −25.376 | −5.550 | 1.00 | 0.00 | 1MOD |
| ATOM | 985 | N | LEU | 125 | −6.468 | −22.501 | −8.120 | 1.00 | 0.00 | 1MOD |
| ATOM | 986 | CA | LEU | 125 | −5.595 | −21.639 | −7.376 | 1.00 | 0.00 | 1MOD |
| ATOM | 987 | C | LEU | 125 | −5.648 | −20.249 | −7.917 | 1.00 | 0.00 | 1MOD |
| ATOM | 988 | O | LEU | 125 | −5.544 | −19.285 | −7.161 | 1.00 | 0.00 | 1MOD |
| ATOM | 989 | CB | LEU | 125 | −4.129 | −22.108 | −7.357 | 1.00 | 0.00 | 1MOD |
| ATOM | 990 | CG | LEU | 125 | −3.885 | −23.300 | −6.409 | 1.00 | 0.00 | 1MOD |
| ATOM | 991 | CD1 | LEU | 125 | −4.034 | −22.873 | −4.941 | 1.00 | 0.00 | 1MOD |
| ATOM | 992 | CD2 | LEU | 125 | −4.797 | −24.490 | −6.737 | 1.00 | 0.00 | 1MOD |
| ATOM | 993 | N | LYS | 126 | −5.767 | −20.098 | −9.242 | 1.00 | 0.00 | 1MOD |
| ATOM | 994 | CA | LYS | 126 | −5.787 | −18.779 | −9.802 | 1.00 | 0.00 | 1MOD |
| ATOM | 995 | C | LYS | 126 | −7.017 | −18.056 | −9.343 | 1.00 | 0.00 | 1MOD |
| ATOM | 996 | O | LYS | 126 | −6.952 | −16.899 | −8.932 | 1.00 | 0.00 | 1MOD |
| ATOM | 997 | CB | LYS | 126 | −5.839 | −18.814 | −11.339 | 1.00 | 0.00 | 1MOD |
| ATOM | 998 | CG | LYS | 126 | −5.853 | −17.442 | −12.018 | 1.00 | 0.00 | 1MOD |
| ATOM | 999 | CD | LYS | 126 | −5.784 | −17.540 | −13.543 | 1.00 | 0.00 | 1MOD |
| ATOM | 1000 | CE | LYS | 126 | −5.664 | −16.192 | −14.255 | 1.00 | 0.00 | 1MOD |
| ATOM | 1001 | NZ | LYS | 126 | −4.238 | −15.878 | −14.495 | 1.00 | 0.00 | 1MOD |
| ATOM | 1002 | N | GLU | 127 | −8.181 | −18.730 | −9.406 | 1.00 | 0.00 | 1MOD |
| ATOM | 1003 | CA | GLU | 127 | −9.405 | −18.066 | −9.076 | 1.00 | 0.00 | 1MOD |
| ATOM | 1004 | C | GLU | 127 | −9.402 | −17.666 | −7.645 | 1.00 | 0.00 | 1MOD |
| ATOM | 1005 | O | GLU | 127 | −9.709 | −16.522 | −7.318 | 1.00 | 0.00 | 1MOD |
| ATOM | 1006 | CB | GLU | 127 | −10.648 | −18.922 | −9.371 | 1.00 | 0.00 | 1MOD |
| ATOM | 1007 | CG | GLU | 127 | −10.851 | −19.118 | −10.877 | 1.00 | 0.00 | 1MOD |
| ATOM | 1008 | CD | GLU | 127 | −12.013 | −20.069 | −11.120 | 1.00 | 0.00 | 1MOD |
| ATOM | 1009 | OE1 | GLU | 127 | −12.554 | −20.633 | −10.132 | 1.00 | 0.00 | 1MOD |
| ATOM | 1010 | OE2 | GLU | 127 | −12.374 | −20.239 | −12.315 | 1.00 | 0.00 | 1MOD |
| ATOM | 1011 | N | VAL | 128 | −8.987 | −18.583 | −6.755 | 1.00 | 0.00 | 1MOD |
| ATOM | 1012 | CA | VAL | 128 | −9.015 | −18.285 | −5.356 | 1.00 | 0.00 | 1MOD |
| ATOM | 1013 | C | VAL | 128 | −8.169 | −17.088 | −5.127 | 1.00 | 0.00 | 1MOD |
| ATOM | 1014 | O | VAL | 128 | −8.501 | −16.210 | −4.335 | 1.00 | 0.00 | 1MOD |
| ATOM | 1015 | CB | VAL | 128 | −8.493 | −19.394 | −4.486 | 1.00 | 0.00 | 1MOD |
| ATOM | 1016 | CG1 | VAL | 128 | −9.472 | −20.570 | −4.548 | 1.00 | 0.00 | 1MOD |
| ATOM | 1017 | CG2 | VAL | 128 | −7.078 | −19.767 | −4.943 | 1.00 | 0.00 | 1MOD |
| ATOM | 1018 | N | ASN | 129 | −7.034 | −17.028 | −5.826 | 1.00 | 0.00 | 1MOD |
| ATOM | 1019 | CA | ASN | 129 | −6.131 | −15.949 | −5.628 | 1.00 | 0.00 | 1MOD |
| ATOM | 1020 | C | ASN | 129 | −6.715 | −14.649 | −6.117 | 1.00 | 0.00 | 1MOD |
| ATOM | 1021 | O | ASN | 129 | −6.539 | −13.615 | −5.472 | 1.00 | 0.00 | 1MOD |
| ATOM | 1022 | CB | ASN | 129 | −4.784 | −16.281 | −6.267 | 1.00 | 0.00 | 1MOD |
| ATOM | 1023 | CG | ASN | 129 | −4.218 | −17.424 | −5.441 | 1.00 | 0.00 | 1MOD |
| ATOM | 1024 | OD1 | ASN | 129 | −3.539 | −18.319 | −5.938 | 1.00 | 0.00 | 1MOD |
| ATOM | 1025 | ND2 | ASN | 129 | −4.535 | −17.402 | −4.119 | 1.00 | 0.00 | 1MOD |
| ATOM | 1026 | N | PHE | 130 | −7.410 | −14.631 | −7.275 | 1.00 | 0.00 | 1MOD |
| ATOM | 1027 | CA | PHE | 130 | −7.908 | −13.356 | −7.712 | 1.00 | 0.00 | 1MOD |
| ATOM | 1028 | C | PHE | 130 | −9.091 | −12.922 | −6.897 | 1.00 | 0.00 | 1MOD |
| ATOM | 1029 | O | PHE | 130 | −9.232 | −11.732 | −6.614 | 1.00 | 0.00 | 1MOD |
| ATOM | 1030 | CB | PHE | 130 | −8.218 | −13.224 | −9.217 | 1.00 | 0.00 | 1MOD |
| ATOM | 1031 | CG | PHE | 130 | −9.487 | −13.875 | −9.639 | 1.00 | 0.00 | 1MOD |
| ATOM | 1032 | CD1 | PHE | 130 | −9.582 | −15.226 | −9.856 | 1.00 | 0.00 | 1MOD |
| ATOM | 1033 | CD2 | PHE | 130 | −10.596 | −13.094 | −9.858 | 1.00 | 0.00 | 1MOD |
| ATOM | 1034 | CE1 | PHE | 130 | −10.774 | −15.772 | −10.272 | 1.00 | 0.00 | 1MOD |
| ATOM | 1035 | CE2 | PHE | 130 | −11.787 | −13.639 | −10.270 | 1.00 | 0.00 | 1MOD |
| ATOM | 1036 | CZ | PHE | 130 | −11.879 | −14.990 | −10.476 | 1.00 | 0.00 | 1MOD |
| ATOM | 1037 | N | TYR | 131 | −9.982 | −13.862 | −6.506 | 1.00 | 0.00 | 1MOD |
| ATOM | 1038 | CA | TYR | 131 | −11.154 | −13.488 | −5.756 | 1.00 | 0.00 | 1MOD |
| ATOM | 1039 | C | TYR | 131 | −10.719 | −12.922 | −4.446 | 1.00 | 0.00 | 1MOD |
| ATOM | 1040 | O | TYR | 131 | −11.273 | −11.936 | −3.964 | 1.00 | 0.00 | 1MOD |
| ATOM | 1041 | CB | TYR | 131 | −12.121 | −14.637 | −5.380 | 1.00 | 0.00 | 1MOD |
| ATOM | 1042 | CG | TYR | 131 | −12.704 | −15.302 | −6.582 | 1.00 | 0.00 | 1MOD |
| ATOM | 1043 | CD1 | TYR | 131 | −13.547 | −14.624 | −7.427 | 1.00 | 0.00 | 1MOD |
| ATOM | 1044 | CD2 | TYR | 131 | −12.397 | −16.607 | −6.876 | 1.00 | 0.00 | 1MOD |
| ATOM | 1045 | CE1 | TYR | 131 | −14.076 | −15.244 | −8.535 | 1.00 | 0.00 | 1MOD |
| ATOM | 1046 | CE2 | TYR | 131 | −12.920 | −17.236 | −7.981 | 1.00 | 0.00 | 1MOD |
| ATOM | 1047 | CZ | TYR | 131 | −13.770 | −16.553 | −8.815 | 1.00 | 0.00 | 1MOD |
| ATOM | 1048 | OH | TYR | 131 | −14.314 | −17.190 | −9.951 | 1.00 | 0.00 | 1MOD |
| ATOM | 1049 | N | SER | 132 | −9.708 | −13.543 | −3.820 | 1.00 | 0.00 | 1MOD |
| ATOM | 1050 | CA | SER | 132 | −9.286 | −13.073 | −2.537 | 1.00 | 0.00 | 1MOD |
| ATOM | 1051 | C | SER | 132 | −8.869 | −11.652 | −2.683 | 1.00 | 0.00 | 1MOD |
| ATOM | 1052 | O | SER | 132 | −9.137 | −10.830 | −1.811 | 1.00 | 0.00 | 1MOD |
| ATOM | 1053 | CB | SER | 132 | −8.122 | −13.899 | −1.966 | 1.00 | 0.00 | 1MOD |
| ATOM | 1054 | OG | SER | 132 | −7.042 | −13.945 | −2.887 | 1.00 | 0.00 | 1MOD |
| ATOM | 1055 | N | GLY | 133 | −8.217 | −11.325 | −3.810 | 1.00 | 0.00 | 1MOD |
| ATOM | 1056 | CA | GLY | 133 | −7.778 | −9.985 | −4.035 | 1.00 | 0.00 | 1MOD |
| ATOM | 1057 | C | GLY | 133 | −8.972 | −9.095 | −4.162 | 1.00 | 0.00 | 1MOD |
| ATOM | 1058 | O | GLY | 133 | −8.978 | −7.979 | −3.644 | 1.00 | 0.00 | 1MOD |
| ATOM | 1059 | N | ILE | 134 | −10.020 | −9.555 | −4.877 | 1.00 | 0.00 | 1MOD |
| ATOM | 1060 | CA | ILE | 134 | −11.133 | −8.674 | −5.072 | 1.00 | 0.00 | 1MOD |
| ATOM | 1061 | C | ILE | 134 | −11.764 | −8.412 | −3.743 | 1.00 | 0.00 | 1MOD |
| ATOM | 1062 | O | ILE | 134 | −12.099 | −7.279 | −3.406 | 1.00 | 0.00 | 1MOD |
| ATOM | 1063 | CB | ILE | 134 | −12.226 | −9.199 | −5.982 | 1.00 | 0.00 | 1MOD |
| ATOM | 1064 | CG1 | ILE | 134 | −13.011 | −10.371 | −5.378 | 1.00 | 0.00 | 1MOD |
| ATOM | 1065 | CG2 | ILE | 134 | −11.561 | −9.603 | −7.301 | 1.00 | 0.00 | 1MOD |
| ATOM | 1066 | CD1 | ILE | 134 | −14.351 | −10.620 | −6.059 | 1.00 | 0.00 | 1MOD |
| ATOM | 1067 | N | LEU | 135 | −11.933 | −9.480 | −2.953 | 1.00 | 0.00 | 1MOD |
| ATOM | 1068 | CA | LEU | 135 | −12.615 | −9.429 | −1.700 | 1.00 | 0.00 | 1MOD |
| ATOM | 1069 | C | LEU | 135 | −11.834 | −8.587 | −0.755 | 1.00 | 0.00 | 1MOD |
| ATOM | 1070 | O | LEU | 135 | −12.406 | −7.848 | 0.045 | 1.00 | 0.00 | 1MOD |
| ATOM | 1071 | CB | LEU | 135 | −12.749 | −10.826 | −1.094 | 1.00 | 0.00 | 1MOD |
| ATOM | 1072 | CG | LEU | 135 | −13.411 | −11.787 | −2.089 | 1.00 | 0.00 | 1MOD |
| ATOM | 1073 | CD1 | LEU | 135 | −13.681 | −13.157 | −1.461 | 1.00 | 0.00 | 1MOD |
| ATOM | 1074 | CD2 | LEU | 135 | −14.644 | −11.143 | −2.734 | 1.00 | 0.00 | 1MOD |
| ATOM | 1075 | N | LEU | 136 | −10.495 | −8.684 | −0.800 | 1.00 | 0.00 | 1MOD |
| ATOM | 1076 | CA | LEU | 136 | −9.728 | −7.918 | 0.140 | 1.00 | 0.00 | 1MOD |
| ATOM | 1077 | C | LEU | 136 | −9.991 | −6.481 | −0.161 | 1.00 | 0.00 | 1MOD |
| ATOM | 1078 | O | LEU | 136 | −10.121 | −5.653 | 0.738 | 1.00 | 0.00 | 1MOD |
| ATOM | 1079 | CB | LEU | 136 | −8.197 | −8.082 | 0.020 | 1.00 | 0.00 | 1MOD |
| ATOM | 1080 | CG | LEU | 136 | −7.647 | −9.522 | 0.058 | 1.00 | 0.00 | 1MOD |
| ATOM | 1081 | CD1 | LEU | 136 | −6.132 | −9.534 | 0.215 | 1.00 | 0.00 | 1MOD |
| ATOM | 1082 | CD2 | LEU | 136 | −8.303 | −10.405 | 1.115 | 1.00 | 0.00 | 1MOD |
| ATOM | 1083 | N | LEU | 137 | −10.095 | −6.157 | −1.462 | 1.00 | 0.00 | 1MOD |
| ATOM | 1084 | CA | LEU | 137 | −10.269 | −4.802 | −1.884 | 1.00 | 0.00 | 1MOD |
| ATOM | 1085 | C | LEU | 137 | −11.515 | −4.267 | −1.268 | 1.00 | 0.00 | 1MOD |
| ATOM | 1086 | O | LEU | 137 | −11.579 | −3.098 | −0.897 | 1.00 | 0.00 | 1MOD |
| ATOM | 1087 | CB | LEU | 137 | −10.415 | −4.671 | −3.408 | 1.00 | 0.00 | 1MOD |
| ATOM | 1088 | CG | LEU | 137 | −10.608 | −3.220 | −3.885 | 1.00 | 0.00 | 1MOD |
| ATOM | 1089 | CD1 | LEU | 137 | −9.381 | −2.358 | −3.551 | 1.00 | 0.00 | 1MOD |
| ATOM | 1090 | CD2 | LEU | 137 | −10.994 | −3.168 | −5.373 | 1.00 | 0.00 | 1MOD |
| ATOM | 1091 | N | ALA | 138 | −12.561 | −5.100 | −1.166 | 1.00 | 0.00 | 1MOD |
| ATOM | 1092 | CA | ALA | 138 | −13.777 | −4.608 | −0.593 | 1.00 | 0.00 | 1MOD |
| ATOM | 1093 | C | ALA | 138 | −13.548 | −4.240 | 0.844 | 1.00 | 0.00 | 1MOD |
| ATOM | 1094 | O | ALA | 138 | −13.982 | −3.186 | 1.304 | 1.00 | 0.00 | 1MOD |
| ATOM | 1095 | CB | ALA | 138 | −14.925 | −5.633 | −0.633 | 1.00 | 0.00 | 1MOD |
| ATOM | 1096 | N | CYS | 139 | −12.852 | −5.095 | 1.609 | 1.00 | 0.00 | 1MOD |
| ATOM | 1097 | CA | CYS | 139 | −12.691 | −4.773 | 2.996 | 1.00 | 0.00 | 1MOD |
| ATOM | 1098 | C | CYS | 139 | −11.817 | −3.572 | 3.160 | 1.00 | 0.00 | 1MOD |
| ATOM | 1099 | O | CYS | 139 | −12.044 | −2.743 | 4.037 | 1.00 | 0.00 | 1MOD |
| ATOM | 1100 | CB | CYS | 139 | −12.167 | −5.938 | 3.836 | 1.00 | 0.00 | 1MOD |
| ATOM | 1101 | SG | CYS | 139 | −13.412 | −7.264 | 3.918 | 1.00 | 0.00 | 1MOD |
| ATOM | 1102 | N | ILE | 140 | −10.784 | −3.429 | 2.316 | 1.00 | 0.00 | 1MOD |
| ATOM | 1103 | CA | ILE | 140 | −9.936 | −2.281 | 2.439 | 1.00 | 0.00 | 1MOD |
| ATOM | 1104 | C | ILE | 140 | −10.764 | −1.075 | 2.142 | 1.00 | 0.00 | 1MOD |
| ATOM | 1105 | O | ILE | 140 | −10.592 | −0.026 | 2.760 | 1.00 | 0.00 | 1MOD |
| ATOM | 1106 | CB | ILE | 140 | −8.748 | −2.307 | 1.518 | 1.00 | 0.00 | 1MOD |
| ATOM | 1107 | CG1 | ILE | 140 | −7.696 | −1.280 | 1.968 | 1.00 | 0.00 | 1MOD |
| ATOM | 1108 | CG2 | ILE | 140 | −9.235 | −2.081 | 0.081 | 1.00 | 0.00 | 1MOD |
| ATOM | 1109 | CD1 | ILE | 140 | −6.331 | −1.473 | 1.304 | 1.00 | 0.00 | 1MOD |
| ATOM | 1110 | N | SER | 141 | −11.693 | −1.192 | 1.174 | 1.00 | 0.00 | 1MOD |
| ATOM | 1111 | CA | SER | 141 | −12.469 | −0.041 | 0.831 | 1.00 | 0.00 | 1MOD |
| ATOM | 1112 | C | SER | 141 | −13.208 | 0.410 | 2.052 | 1.00 | 0.00 | 1MOD |
| ATOM | 1113 | O | SER | 141 | −13.350 | 1.612 | 2.265 | 1.00 | 0.00 | 1MOD |
| ATOM | 1114 | CB | SER | 141 | −13.497 | −0.269 | −0.292 | 1.00 | 0.00 | 1MOD |
| ATOM | 1115 | OG | SER | 141 | −14.636 | −0.960 | 0.195 | 1.00 | 0.00 | 1MOD |
| ATOM | 1116 | N | VAL | 142 | −13.731 | −0.528 | 2.871 | 1.00 | 0.00 | 1MOD |
| ATOM | 1117 | CA | VAL | 142 | −14.427 | −0.114 | 4.059 | 1.00 | 0.00 | 1MOD |
| ATOM | 1118 | C | VAL | 142 | −13.483 | 0.399 | 5.109 | 1.00 | 0.00 | 1MOD |
| ATOM | 1119 | O | VAL | 142 | −13.754 | 1.420 | 5.739 | 1.00 | 0.00 | 1MOD |
| ATOM | 1120 | CB | VAL | 142 | −15.321 | −1.163 | 4.674 | 1.00 | 0.00 | 1MOD |
| ATOM | 1121 | CG1 | VAL | 142 | −16.402 | −1.525 | 3.643 | 1.00 | 0.00 | 1MOD |
| ATOM | 1122 | CG2 | VAL | 142 | −14.497 | −2.357 | 5.176 | 1.00 | 0.00 | 1MOD |
| ATOM | 1123 | N | ASP | 143 | −12.335 | −0.278 | 5.324 | 1.00 | 0.00 | 1MOD |
| ATOM | 1124 | CA | ASP | 143 | −11.464 | 0.146 | 6.385 | 1.00 | 0.00 | 1MOD |
| ATOM | 1125 | C | ASP | 143 | −11.019 | 1.532 | 6.082 | 1.00 | 0.00 | 1MOD |
| ATOM | 1126 | O | ASP | 143 | −10.795 | 2.332 | 6.988 | 1.00 | 0.00 | 1MOD |
| ATOM | 1127 | CB | ASP | 143 | −10.223 | −0.750 | 6.620 | 1.00 | 0.00 | 1MOD |
| ATOM | 1128 | CG | ASP | 143 | −9.220 | −0.713 | 5.469 | 1.00 | 0.00 | 1MOD |
| ATOM | 1129 | OD1 | ASP | 143 | −8.718 | 0.388 | 5.119 | 1.00 | 0.00 | 1MOD |
| ATOM | 1130 | OD2 | ASP | 143 | −8.922 | −1.816 | 4.941 | 1.00 | 0.00 | 1MOD |
| ATOM | 1131 | N | ARG | 144 | −10.857 | 1.843 | 4.787 | 1.00 | 0.00 | 1MOD |
| ATOM | 1132 | CA | ARG | 144 | −10.448 | 3.149 | 4.376 | 1.00 | 0.00 | 1MOD |
| ATOM | 1133 | C | ARG | 144 | −11.546 | 4.113 | 4.689 | 1.00 | 0.00 | 1MOD |
| ATOM | 1134 | O | ARG | 144 | −11.291 | 5.253 | 5.070 | 1.00 | 0.00 | 1MOD |
| ATOM | 1135 | CB | ARG | 144 | −10.176 | 3.247 | 2.864 | 1.00 | 0.00 | 1MOD |
| ATOM | 1136 | CG | ARG | 144 | −8.813 | 2.694 | 2.435 | 1.00 | 0.00 | 1MOD |
| ATOM | 1137 | CD | ARG | 144 | −7.667 | 3.711 | 2.489 | 1.00 | 0.00 | 1MOD |
| ATOM | 1138 | NE | ARG | 144 | −6.880 | 3.434 | 3.725 | 1.00 | 0.00 | 1MOD |
| ATOM | 1139 | CZ | ARG | 144 | −6.878 | 4.313 | 4.771 | 1.00 | 0.00 | 1MOD |
| ATOM | 1140 | NH1 | ARG | 144 | −7.566 | 5.486 | 4.678 | 1.00 | 0.00 | 1MOD |
| ATOM | 1141 | NH2 | ARG | 144 | −6.170 | 4.018 | 5.901 | 1.00 | 0.00 | 1MOD |
| ATOM | 1142 | N | TYR | 145 | −12.807 | 3.673 | 4.521 | 1.00 | 0.00 | 1MOD |
| ATOM | 1143 | CA | TYR | 145 | −13.930 | 4.551 | 4.677 | 1.00 | 0.00 | 1MOD |
| ATOM | 1144 | C | TYR | 145 | −13.973 | 5.060 | 6.081 | 1.00 | 0.00 | 1MOD |
| ATOM | 1145 | O | TYR | 145 | −14.183 | 6.249 | 6.311 | 1.00 | 0.00 | 1MOD |
| ATOM | 1146 | CB | TYR | 145 | −15.269 | 3.850 | 4.387 | 1.00 | 0.00 | 1MOD |
| ATOM | 1147 | CG | TYR | 145 | −16.363 | 4.867 | 4.422 | 1.00 | 0.00 | 1MOD |
| ATOM | 1148 | CD1 | TYR | 145 | −16.592 | 5.685 | 3.340 | 1.00 | 0.00 | 1MOD |
| ATOM | 1149 | CD2 | TYR | 145 | −17.168 | 4.994 | 5.530 | 1.00 | 0.00 | 1MOD |
| ATOM | 1150 | CE1 | TYR | 145 | −17.604 | 6.616 | 3.368 | 1.00 | 0.00 | 1MOD |
| ATOM | 1151 | CE2 | TYR | 145 | −18.182 | 5.923 | 5.565 | 1.00 | 0.00 | 1MOD |
| ATOM | 1152 | CZ | TYR | 145 | −18.402 | 6.734 | 4.479 | 1.00 | 0.00 | 1MOD |
| ATOM | 1153 | OH | TYR | 145 | −19.440 | 7.690 | 4.498 | 1.00 | 0.00 | 1MOD |
| ATOM | 1154 | N | LEU | 146 | −13.760 | 4.168 | 7.061 | 1.00 | 0.00 | 1MOD |
| ATOM | 1155 | CA | LEU | 146 | −13.871 | 4.547 | 8.437 | 1.00 | 0.00 | 1MOD |
| ATOM | 1156 | C | LEU | 146 | −12.831 | 5.569 | 8.792 | 1.00 | 0.00 | 1MOD |
| ATOM | 1157 | O | LEU | 146 | −13.116 | 6.511 | 9.527 | 1.00 | 0.00 | 1MOD |
| ATOM | 1158 | CB | LEU | 146 | −13.703 | 3.348 | 9.383 | 1.00 | 0.00 | 1MOD |
| ATOM | 1159 | CG | LEU | 146 | −14.691 | 2.214 | 9.055 | 1.00 | 0.00 | 1MOD |
| ATOM | 1160 | CD1 | LEU | 146 | −14.737 | 1.159 | 10.170 | 1.00 | 0.00 | 1MOD |
| ATOM | 1161 | CD2 | LEU | 146 | −16.070 | 2.771 | 8.679 | 1.00 | 0.00 | 1MOD |
| ATOM | 1162 | N | ALA | 147 | −11.584 | 5.394 | 8.312 | 1.00 | 0.00 | 1MOD |
| ATOM | 1163 | CA | ALA | 147 | −10.534 | 6.304 | 8.691 | 1.00 | 0.00 | 1MOD |
| ATOM | 1164 | C | ALA | 147 | −10.720 | 7.697 | 8.162 | 1.00 | 0.00 | 1MOD |
| ATOM | 1165 | O | ALA | 147 | −10.619 | 8.668 | 8.909 | 1.00 | 0.00 | 1MOD |
| ATOM | 1166 | CB | ALA | 147 | −9.150 | 5.834 | 8.210 | 1.00 | 0.00 | 1MOD |
| ATOM | 1167 | N | ILE | 148 | −10.944 | 7.861 | 6.846 | 1.00 | 0.00 | 1MOD |
| ATOM | 1168 | CA | ILE | 148 | −11.069 | 9.200 | 6.350 | 1.00 | 0.00 | 1MOD |
| ATOM | 1169 | C | ILE | 148 | −12.399 | 9.801 | 6.657 | 1.00 | 0.00 | 1MOD |
| ATOM | 1170 | O | ILE | 148 | −12.482 | 10.923 | 7.152 | 1.00 | 0.00 | 1MOD |
| ATOM | 1171 | CB | ILE | 148 | −10.809 | 9.320 | 4.879 | 1.00 | 0.00 | 1MOD |
| ATOM | 1172 | CG1 | ILE | 148 | −10.764 | 10.813 | 4.532 | 1.00 | 0.00 | 1MOD |
| ATOM | 1173 | CG2 | ILE | 148 | −11.859 | 8.504 | 4.100 | 1.00 | 0.00 | 1MOD |
| ATOM | 1174 | CD1 | ILE | 148 | −9.730 | 11.568 | 5.369 | 1.00 | 0.00 | 1MOD |
| ATOM | 1175 | N | VAL | 149 | −13.477 | 9.055 | 6.344 | 1.00 | 0.00 | 1MOD |
| ATOM | 1176 | CA | VAL | 149 | −14.818 | 9.531 | 6.501 | 1.00 | 0.00 | 1MOD |
| ATOM | 1177 | C | VAL | 149 | −15.122 | 9.693 | 7.955 | 1.00 | 0.00 | 1MOD |
| ATOM | 1178 | O | VAL | 149 | −15.728 | 10.685 | 8.354 | 1.00 | 0.00 | 1MOD |
| ATOM | 1179 | CB | VAL | 149 | −15.831 | 8.608 | 5.896 | 1.00 | 0.00 | 1MOD |
| ATOM | 1180 | CG1 | VAL | 149 | −17.236 | 9.166 | 6.175 | 1.00 | 0.00 | 1MOD |
| ATOM | 1181 | CG2 | VAL | 149 | −15.508 | 8.474 | 4.399 | 1.00 | 0.00 | 1MOD |
| ATOM | 1182 | N | HIS | 150 | −14.694 | 8.726 | 8.791 | 1.00 | 0.00 | 1MOD |
| ATOM | 1183 | CA | HIS | 150 | −14.980 | 8.794 | 10.200 | 1.00 | 0.00 | 1MOD |
| ATOM | 1184 | C | HIS | 150 | −16.453 | 8.961 | 10.403 | 1.00 | 0.00 | 1MOD |
| ATOM | 1185 | O | HIS | 150 | −16.885 | 9.886 | 11.091 | 1.00 | 0.00 | 1MOD |
| ATOM | 1186 | CB | HIS | 150 | −14.288 | 9.956 | 10.933 | 1.00 | 0.00 | 1MOD |
| ATOM | 1187 | CG | HIS | 150 | −12.842 | 9.703 | 11.225 | 1.00 | 0.00 | 1MOD |
| ATOM | 1188 | ND1 | HIS | 150 | −11.937 | 10.678 | 11.579 | 1.00 | 0.00 | 1MOD |
| ATOM | 1189 | CD2 | HIS | 150 | −12.149 | 8.533 | 11.224 | 1.00 | 0.00 | 1MOD |
| ATOM | 1190 | CE1 | HIS | 150 | −10.746 | 10.055 | 11.768 | 1.00 | 0.00 | 1MOD |
| ATOM | 1191 | NE2 | HIS | 150 | −10.826 | 8.752 | 11.564 | 1.00 | 0.00 | 1MOD |
| ATOM | 1192 | N | ALA | 151 | −17.264 | 8.074 | 9.799 | 1.00 | 0.00 | 1MOD |
| ATOM | 1193 | CA | ALA | 151 | −18.694 | 8.171 | 9.896 | 1.00 | 0.00 | 1MOD |
| ATOM | 1194 | C | ALA | 151 | −19.181 | 7.976 | 11.302 | 1.00 | 0.00 | 1MOD |
| ATOM | 1195 | O | ALA | 151 | −20.038 | 8.725 | 11.770 | 1.00 | 0.00 | 1MOD |
| ATOM | 1196 | CB | ALA | 151 | −19.415 | 7.133 | 9.015 | 1.00 | 0.00 | 1MOD |
| ATOM | 1197 | N | THR | 152 | −18.654 | 6.971 | 12.027 | 1.00 | 0.00 | 1MOD |
| ATOM | 1198 | CA | THR | 152 | −19.195 | 6.689 | 13.326 | 1.00 | 0.00 | 1MOD |
| ATOM | 1199 | C | THR | 152 | −18.400 | 7.391 | 14.377 | 1.00 | 0.00 | 1MOD |
| ATOM | 1200 | O | THR | 152 | −17.507 | 8.184 | 14.086 | 1.00 | 0.00 | 1MOD |
| ATOM | 1201 | CB | THR | 152 | −19.219 | 5.221 | 13.649 | 1.00 | 0.00 | 1MOD |
| ATOM | 1202 | OG1 | THR | 152 | −20.056 | 4.973 | 14.769 | 1.00 | 0.00 | 1MOD |
| ATOM | 1203 | CG2 | THR | 152 | −17.783 | 4.757 | 13.942 | 1.00 | 0.00 | 1MOD |
| ATOM | 1204 | N | ARG | 153 | −18.734 | 7.103 | 15.649 | 1.00 | 0.00 | 1MOD |
| ATOM | 1205 | CA | ARG | 153 | −18.064 | 7.701 | 16.762 | 1.00 | 0.00 | 1MOD |
| ATOM | 1206 | C | ARG | 153 | −16.634 | 7.303 | 16.624 | 1.00 | 0.00 | 1MOD |
| ATOM | 1207 | O | ARG | 153 | −16.340 | 6.285 | 16.002 | 1.00 | 0.00 | 1MOD |
| ATOM | 1208 | CB | ARG | 153 | −18.606 | 7.201 | 18.113 | 1.00 | 0.00 | 1MOD |
| ATOM | 1209 | CG | ARG | 153 | −18.220 | 8.069 | 19.310 | 1.00 | 0.00 | 1MOD |
| ATOM | 1210 | CD | ARG | 153 | −18.998 | 7.714 | 20.579 | 1.00 | 0.00 | 1MOD |
| ATOM | 1211 | NE | ARG | 153 | −20.450 | 7.804 | 20.249 | 1.00 | 0.00 | 1MOD |
| ATOM | 1212 | CZ | ARG | 153 | −21.125 | 6.698 | 19.815 | 1.00 | 0.00 | 1MOD |
| ATOM | 1213 | NH1 | ARG | 153 | −20.491 | 5.490 | 19.748 | 1.00 | 0.00 | 1MOD |
| ATOM | 1214 | NH2 | ARG | 153 | −22.437 | 6.796 | 19.453 | 1.00 | 0.00 | 1MOD |
| ATOM | 1215 | N | THR | 154 | −15.711 | 8.117 | 17.176 | 1.00 | 0.00 | 1MOD |
| ATOM | 1216 | CA | THR | 154 | −14.311 | 7.863 | 16.994 | 1.00 | 0.00 | 1MOD |
| ATOM | 1217 | C | THR | 154 | −13.994 | 6.463 | 17.396 | 1.00 | 0.00 | 1MOD |
| ATOM | 1218 | O | THR | 154 | −14.078 | 6.083 | 18.563 | 1.00 | 0.00 | 1MOD |
| ATOM | 1219 | CB | THR | 154 | −13.417 | 8.804 | 17.754 | 1.00 | 0.00 | 1MOD |
| ATOM | 1220 | OG1 | THR | 154 | −12.055 | 8.497 | 17.493 | 1.00 | 0.00 | 1MOD |
| ATOM | 1221 | CG2 | THR | 154 | −13.713 | 8.705 | 19.261 | 1.00 | 0.00 | 1MOD |
| ATOM | 1222 | N | LEU | 155 | −13.652 | 5.639 | 16.388 | 1.00 | 0.00 | 1MOD |
| ATOM | 1223 | CA | LEU | 155 | −13.313 | 4.277 | 16.647 | 1.00 | 0.00 | 1MOD |
| ATOM | 1224 | C | LEU | 155 | −12.400 | 3.860 | 15.538 | 1.00 | 0.00 | 1MOD |
| ATOM | 1225 | O | LEU | 155 | −12.608 | 4.235 | 14.385 | 1.00 | 0.00 | 1MOD |
| ATOM | 1226 | CB | LEU | 155 | −14.548 | 3.357 | 16.666 | 1.00 | 0.00 | 1MOD |
| ATOM | 1227 | CG | LEU | 155 | −14.270 | 1.912 | 17.114 | 1.00 | 0.00 | 1MOD |
| ATOM | 1228 | CD1 | LEU | 155 | −13.435 | 1.150 | 16.076 | 1.00 | 0.00 | 1MOD |
| ATOM | 1229 | CD2 | LEU | 155 | −13.658 | 1.881 | 18.525 | 1.00 | 0.00 | 1MOD |
| ATOM | 1230 | N | THR | 156 | −11.348 | 3.084 | 15.858 | 1.00 | 0.00 | 1MOD |
| ATOM | 1231 | CA | THR | 156 | −10.419 | 2.670 | 14.846 | 1.00 | 0.00 | 1MOD |
| ATOM | 1232 | C | THR | 156 | −10.392 | 1.174 | 14.866 | 1.00 | 0.00 | 1MOD |
| ATOM | 1233 | O | THR | 156 | −10.770 | 0.551 | 15.857 | 1.00 | 0.00 | 1MOD |
| ATOM | 1234 | CB | THR | 156 | −9.025 | 3.165 | 15.096 | 1.00 | 0.00 | 1MOD |
| ATOM | 1235 | OG1 | THR | 156 | −8.521 | 2.607 | 16.300 | 1.00 | 0.00 | 1MOD |
| ATOM | 1236 | CG2 | THR | 156 | −9.062 | 4.698 | 15.212 | 1.00 | 0.00 | 1MOD |
| ATOM | 1237 | N | GLN | 157 | −9.947 | 0.546 | 13.760 | 1.00 | 0.00 | 1MOD |
| ATOM | 1238 | CA | GLN | 157 | −10.012 | −0.888 | 13.694 | 1.00 | 0.00 | 1MOD |
| ATOM | 1239 | C | GLN | 157 | −8.842 | −1.540 | 14.367 | 1.00 | 0.00 | 1MOD |
| ATOM | 1240 | O | GLN | 157 | −7.693 | −1.380 | 13.952 | 1.00 | 0.00 | 1MOD |
| ATOM | 1241 | CB | GLN | 157 | −10.062 | −1.427 | 12.256 | 1.00 | 0.00 | 1MOD |
| ATOM | 1242 | CG | GLN | 157 | −11.287 | −0.944 | 11.478 | 1.00 | 0.00 | 1MOD |
| ATOM | 1243 | CD | GLN | 157 | −11.235 | −1.563 | 10.090 | 1.00 | 0.00 | 1MOD |
| ATOM | 1244 | OE1 | GLN | 157 | −10.422 | −2.447 | 9.819 | 1.00 | 0.00 | 1MOD |
| ATOM | 1245 | NE2 | GLN | 157 | −12.134 | −1.094 | 9.185 | 1.00 | 0.00 | 1MOD |
| ATOM | 1246 | N | LYS | 158 | −9.172 | −2.340 | 15.408 | 1.00 | 0.00 | 1MOD |
| ATOM | 1247 | CA | LYS | 158 | −8.289 | −3.129 | 16.221 | 1.00 | 0.00 | 1MOD |
| ATOM | 1248 | C | LYS | 158 | −7.927 | −4.318 | 15.386 | 1.00 | 0.00 | 1MOD |
| ATOM | 1249 | O | LYS | 158 | −8.490 | −4.523 | 14.313 | 1.00 | 0.00 | 1MOD |
| ATOM | 1250 | CB | LYS | 158 | −8.968 | −3.609 | 17.522 | 1.00 | 0.00 | 1MOD |
| ATOM | 1251 | CG | LYS | 158 | −8.005 | −4.120 | 18.597 | 1.00 | 0.00 | 1MOD |
| ATOM | 1252 | CD | LYS | 158 | −8.626 | −4.199 | 19.997 | 1.00 | 0.00 | 1MOD |
| ATOM | 1253 | CE | LYS | 158 | −9.608 | −5.358 | 20.182 | 1.00 | 0.00 | 1MOD |
| ATOM | 1254 | NZ | LYS | 158 | −10.028 | −5.445 | 21.599 | 1.00 | 0.00 | 1MOD |
| ATOM | 1255 | N | ARG | 159 | −6.949 | −5.119 | 15.847 | 1.00 | 0.00 | 1MOD |
| ATOM | 1256 | CA | ARG | 159 | −6.492 | −6.273 | 15.121 | 1.00 | 0.00 | 1MOD |
| ATOM | 1257 | C | ARG | 159 | −7.627 | −7.239 | 14.995 | 1.00 | 0.00 | 1MOD |
| ATOM | 1258 | O | ARG | 159 | −7.765 | −7.936 | 13.991 | 1.00 | 0.00 | 1MOD |
| ATOM | 1259 | CB | ARG | 159 | −5.325 | −6.975 | 15.830 | 1.00 | 0.00 | 1MOD |
| ATOM | 1260 | CG | ARG | 159 | −5.670 | −7.410 | 17.253 | 1.00 | 0.00 | 1MOD |
| ATOM | 1261 | CD | ARG | 159 | −4.458 | −7.891 | 18.050 | 1.00 | 0.00 | 1MOD |
| ATOM | 1262 | NE | ARG | 159 | −4.929 | −8.181 | 19.432 | 1.00 | 0.00 | 1MOD |
| ATOM | 1263 | CZ | ARG | 159 | −4.212 | −9.012 | 20.242 | 1.00 | 0.00 | 1MOD |
| ATOM | 1264 | NH1 | ARG | 159 | −3.064 | −9.595 | 19.785 | 1.00 | 0.00 | 1MOD |
| ATOM | 1265 | NH2 | ARG | 159 | −4.653 | −9.264 | 21.508 | 1.00 | 0.00 | 1MOD |
| ATOM | 1266 | N | TYR | 160 | −8.488 | −7.301 | 16.018 | 1.00 | 0.00 | 1MOD |
| ATOM | 1267 | CA | TYR | 160 | −9.577 | −8.227 | 15.997 | 1.00 | 0.00 | 1MOD |
| ATOM | 1268 | C | TYR | 160 | −10.434 | −7.924 | 14.801 | 1.00 | 0.00 | 1MOD |
| ATOM | 1269 | O | TYR | 160 | −10.855 | −8.834 | 14.087 | 1.00 | 0.00 | 1MOD |
| ATOM | 1270 | CB | TYR | 160 | −10.444 | −8.079 | 17.251 | 1.00 | 0.00 | 1MOD |
| ATOM | 1271 | CG | TYR | 160 | −11.505 | −9.116 | 17.196 | 1.00 | 0.00 | 1MOD |
| ATOM | 1272 | CD1 | TYR | 160 | −11.233 | −10.395 | 17.624 | 1.00 | 0.00 | 1MOD |
| ATOM | 1273 | CD2 | TYR | 160 | −12.759 | −8.810 | 16.723 | 1.00 | 0.00 | 1MOD |
| ATOM | 1274 | CE1 | TYR | 160 | −12.207 | −11.361 | 17.582 | 1.00 | 0.00 | 1MOD |
| ATOM | 1275 | CE2 | TYR | 160 | −13.735 | −9.775 | 16.680 | 1.00 | 0.00 | 1MOD |
| ATOM | 1276 | CZ | TYR | 160 | −13.459 | −11.050 | 17.110 | 1.00 | 0.00 | 1MOD |
| ATOM | 1277 | OH | TYR | 160 | −14.460 | −12.042 | 17.068 | 1.00 | 0.00 | 1MOD |
| ATOM | 1278 | N | LEU | 161 | −10.708 | −6.631 | 14.540 | 1.00 | 0.00 | 1MOD |
| ATOM | 1279 | CA | LEU | 161 | −11.540 | −6.255 | 13.428 | 1.00 | 0.00 | 1MOD |
| ATOM | 1280 | C | LEU | 161 | −10.849 | −6.687 | 12.172 | 1.00 | 0.00 | 1MOD |
| ATOM | 1281 | O | LEU | 161 | −11.472 | −7.160 | 11.223 | 1.00 | 0.00 | 1MOD |
| ATOM | 1282 | CB | LEU | 161 | −11.774 | −4.736 | 13.336 | 1.00 | 0.00 | 1MOD |
| ATOM | 1283 | CG | LEU | 161 | −12.566 | −4.154 | 14.522 | 1.00 | 0.00 | 1MOD |
| ATOM | 1284 | CD1 | LEU | 161 | −11.799 | −4.320 | 15.842 | 1.00 | 0.00 | 1MOD |
| ATOM | 1285 | CD2 | LEU | 161 | −12.981 | −2.699 | 14.257 | 1.00 | 0.00 | 1MOD |
| ATOM | 1286 | N | VAL | 162 | −9.518 | −6.543 | 12.166 | 1.00 | 0.00 | 1MOD |
| ATOM | 1287 | CA | VAL | 162 | −8.654 | −6.896 | 11.079 | 1.00 | 0.00 | 1MOD |
| ATOM | 1288 | C | VAL | 162 | −8.816 | −8.328 | 10.690 | 1.00 | 0.00 | 1MOD |
| ATOM | 1289 | O | VAL | 162 | −8.908 | −8.662 | 9.508 | 1.00 | 0.00 | 1MOD |
| ATOM | 1290 | CB | VAL | 162 | −7.253 | −6.814 | 11.544 | 1.00 | 0.00 | 1MOD |
| ATOM | 1291 | CG1 | VAL | 162 | −6.474 | −7.825 | 10.714 | 1.00 | 0.00 | 1MOD |
| ATOM | 1292 | CG2 | VAL | 162 | −6.755 | −5.365 | 11.422 | 1.00 | 0.00 | 1MOD |
| ATOM | 1293 | N | LYS | 163 | −8.826 | −9.221 | 11.686 | 1.00 | 0.00 | 1MOD |
| ATOM | 1294 | CA | LYS | 163 | −8.857 | −10.628 | 11.428 | 1.00 | 0.00 | 1MOD |
| ATOM | 1295 | C | LYS | 163 | −10.119 | −10.999 | 10.718 | 1.00 | 0.00 | 1MOD |
| ATOM | 1296 | O | LYS | 163 | −10.121 | −11.876 | 9.856 | 1.00 | 0.00 | 1MOD |
| ATOM | 1297 | CB | LYS | 163 | −8.781 | −11.446 | 12.729 | 1.00 | 0.00 | 1MOD |
| ATOM | 1298 | CG | LYS | 163 | −8.718 | −12.964 | 12.541 | 1.00 | 0.00 | 1MOD |
| ATOM | 1299 | CD | LYS | 163 | −8.271 | −13.708 | 13.803 | 1.00 | 0.00 | 1MOD |
| ATOM | 1300 | CE | LYS | 163 | −8.405 | −15.228 | 13.700 | 1.00 | 0.00 | 1MOD |
| ATOM | 1301 | NZ | LYS | 163 | −7.577 | −15.736 | 12.585 | 1.00 | 0.00 | 1MOD |
| ATOM | 1302 | N | PHE | 164 | −11.240 | −10.347 | 11.071 | 1.00 | 0.00 | 1MOD |
| ATOM | 1303 | CA | PHE | 164 | −12.498 | −10.737 | 10.508 | 1.00 | 0.00 | 1MOD |
| ATOM | 1304 | C | PHE | 164 | −12.502 | −10.525 | 9.025 | 1.00 | 0.00 | 1MOD |
| ATOM | 1305 | O | PHE | 164 | −12.937 | −11.394 | 8.272 | 1.00 | 0.00 | 1MOD |
| ATOM | 1306 | CB | PHE | 164 | −13.681 | −9.962 | 11.109 | 1.00 | 0.00 | 1MOD |
| ATOM | 1307 | CG | PHE | 164 | −14.922 | −10.581 | 10.569 | 1.00 | 0.00 | 1MOD |
| ATOM | 1308 | CD1 | PHE | 164 | −15.456 | −11.708 | 11.149 | 1.00 | 0.00 | 1MOD |
| ATOM | 1309 | CD2 | PHE | 164 | −15.552 | −10.031 | 9.477 | 1.00 | 0.00 | 1MOD |
| ATOM | 1310 | CE1 | PHE | 164 | −16.604 | −12.275 | 10.646 | 1.00 | 0.00 | 1MOD |
| ATOM | 1311 | CE2 | PHE | 164 | −16.698 | −10.593 | 8.970 | 1.00 | 0.00 | 1MOD |
| ATOM | 1312 | CZ | PHE | 164 | −17.226 | −11.717 | 9.555 | 1.00 | 0.00 | 1MOD |
| ATOM | 1313 | N | ILE | 165 | −12.004 | −9.363 | 8.566 | 1.00 | 0.00 | 1MOD |
| ATOM | 1314 | CA | ILE | 165 | −12.025 | −9.027 | 7.172 | 1.00 | 0.00 | 1MOD |
| ATOM | 1315 | C | ILE | 165 | −11.206 | −10.000 | 6.376 | 1.00 | 0.00 | 1MOD |
| ATOM | 1316 | O | ILE | 165 | −11.620 | −10.417 | 5.295 | 1.00 | 0.00 | 1MOD |
| ATOM | 1317 | CB | ILE | 165 | −11.612 | −7.600 | 6.943 | 1.00 | 0.00 | 1MOD |
| ATOM | 1318 | CG1 | ILE | 165 | −10.209 | −7.341 | 7.496 | 1.00 | 0.00 | 1MOD |
| ATOM | 1319 | CG2 | ILE | 165 | −12.682 | −6.689 | 7.568 | 1.00 | 0.00 | 1MOD |
| ATOM | 1320 | CD1 | ILE | 165 | −9.753 | −5.902 | 7.293 | 1.00 | 0.00 | 1MOD |
| ATOM | 1321 | N | CYS | 166 | −10.032 | −10.409 | 6.887 | 1.00 | 0.00 | 1MOD |
| ATOM | 1322 | CA | CYS | 166 | −9.197 | −11.345 | 6.187 | 1.00 | 0.00 | 1MOD |
| ATOM | 1323 | C | CYS | 166 | −9.935 | −12.641 | 6.052 | 1.00 | 0.00 | 1MOD |
| ATOM | 1324 | O | CYS | 166 | −9.875 | −13.293 | 5.011 | 1.00 | 0.00 | 1MOD |
| ATOM | 1325 | CB | CYS | 166 | −7.892 | −11.655 | 6.942 | 1.00 | 0.00 | 1MOD |
| ATOM | 1326 | SG | CYS | 166 | −6.841 | −10.189 | 7.165 | 1.00 | 0.00 | 1MOD |
| ATOM | 1327 | N | LEU | 167 | −10.651 | −13.055 | 7.114 | 1.00 | 0.00 | 1MOD |
| ATOM | 1328 | CA | LEU | 167 | −11.351 | −14.306 | 7.065 | 1.00 | 0.00 | 1MOD |
| ATOM | 1329 | C | LEU | 167 | −12.418 | −14.256 | 6.021 | 1.00 | 0.00 | 1MOD |
| ATOM | 1330 | O | LEU | 167 | −12.626 | −15.229 | 5.299 | 1.00 | 0.00 | 1MOD |
| ATOM | 1331 | CB | LEU | 167 | −12.061 | −14.683 | 8.373 | 1.00 | 0.00 | 1MOD |
| ATOM | 1332 | CG | LEU | 167 | −12.875 | −15.987 | 8.242 | 1.00 | 0.00 | 1MOD |
| ATOM | 1333 | CD1 | LEU | 167 | −11.969 | −17.206 | 8.001 | 1.00 | 0.00 | 1MOD |
| ATOM | 1334 | CD2 | LEU | 167 | −13.836 | −16.166 | 9.427 | 1.00 | 0.00 | 1MOD |
| ATOM | 1335 | N | SER | 168 | −13.127 | −13.118 | 5.918 | 1.00 | 0.00 | 1MOD |
| ATOM | 1336 | CA | SER | 168 | −14.241 | −13.016 | 5.021 | 1.00 | 0.00 | 1MOD |
| ATOM | 1337 | C | SER | 168 | −13.791 | −13.211 | 3.611 | 1.00 | 0.00 | 1MOD |
| ATOM | 1338 | O | SER | 168 | −14.481 | −13.845 | 2.814 | 1.00 | 0.00 | 1MOD |
| ATOM | 1339 | CB | SER | 168 | −14.952 | −11.653 | 5.091 | 1.00 | 0.00 | 1MOD |
| ATOM | 1340 | OG | SER | 168 | −14.099 | −10.624 | 4.611 | 1.00 | 0.00 | 1MOD |
| ATOM | 1341 | N | ILE | 169 | −12.608 | −12.684 | 3.265 | 1.00 | 0.00 | 1MOD |
| ATOM | 1342 | CA | ILE | 169 | −12.150 | −12.758 | 1.915 | 1.00 | 0.00 | 1MOD |
| ATOM | 1343 | C | ILE | 169 | −11.994 | −14.193 | 1.548 | 1.00 | 0.00 | 1MOD |
| ATOM | 1344 | O | ILE | 169 | −12.295 | −14.589 | 0.423 | 1.00 | 0.00 | 1MOD |
| ATOM | 1345 | CB | ILE | 169 | −10.840 | −12.088 | 1.735 | 1.00 | 0.00 | 1MOD |
| ATOM | 1346 | CG1 | ILE | 169 | −10.954 | −10.678 | 2.343 | 1.00 | 0.00 | 1MOD |
| ATOM | 1347 | CG2 | ILE | 169 | −10.483 | −12.161 | 0.237 | 1.00 | 0.00 | 1MOD |
| ATOM | 1348 | CD1 | ILE | 169 | −12.176 | −9.899 | 1.860 | 1.00 | 0.00 | 1MOD |
| ATOM | 1349 | N | TRP | 170 | −11.490 | −15.010 | 2.490 | 1.00 | 0.00 | 1MOD |
| ATOM | 1350 | CA | TRP | 170 | −11.275 | −16.403 | 2.232 | 1.00 | 0.00 | 1MOD |
| ATOM | 1351 | C | TRP | 170 | −12.584 | −17.086 | 1.977 | 1.00 | 0.00 | 1MOD |
| ATOM | 1352 | O | TRP | 170 | −12.680 | −17.940 | 1.098 | 1.00 | 0.00 | 1MOD |
| ATOM | 1353 | CB | TRP | 170 | −10.572 | −17.145 | 3.387 | 1.00 | 0.00 | 1MOD |
| ATOM | 1354 | CG | TRP | 170 | −9.123 | −16.756 | 3.606 | 1.00 | 0.00 | 1MOD |
| ATOM | 1355 | CD1 | TRP | 170 | −8.570 | −15.956 | 4.564 | 1.00 | 0.00 | 1MOD |
| ATOM | 1356 | CD2 | TRP | 170 | −8.038 | −17.194 | 2.771 | 1.00 | 0.00 | 1MOD |
| ATOM | 1357 | NE1 | TRP | 170 | −7.209 | −15.874 | 4.383 | 1.00 | 0.00 | 1MOD |
| ATOM | 1358 | CE2 | TRP | 170 | −6.869 | −16.629 | 3.281 | 1.00 | 0.00 | 1MOD |
| ATOM | 1359 | CE3 | TRP | 170 | −8.017 | −18.002 | 1.671 | 1.00 | 0.00 | 1MOD |
| ATOM | 1360 | CZ2 | TRP | 170 | −5.656 | −16.866 | 2.698 | 1.00 | 0.00 | 1MOD |
| ATOM | 1361 | CZ3 | TRP | 170 | −6.794 | −18.234 | 1.080 | 1.00 | 0.00 | 1MOD |
| ATOM | 1362 | CH2 | TRP | 170 | −5.636 | −17.678 | 1.585 | 1.00 | 0.00 | 1MOD |
| ATOM | 1363 | N | GLY | 171 | −13.629 | −16.761 | 2.756 | 1.00 | 0.00 | 1MOD |
| ATOM | 1364 | CA | GLY | 171 | −14.873 | −17.452 | 2.565 | 1.00 | 0.00 | 1MOD |
| ATOM | 1365 | C | GLY | 171 | −15.464 | −17.146 | 1.222 | 1.00 | 0.00 | 1MOD |
| ATOM | 1366 | O | GLY | 171 | −15.885 | −18.050 | 0.502 | 1.00 | 0.00 | 1MOD |
| ATOM | 1367 | N | LEU | 172 | −15.516 | −15.852 | 0.852 | 1.00 | 0.00 | 1MOD |
| ATOM | 1368 | CA | LEU | 172 | −16.160 | −15.476 | −0.372 | 1.00 | 0.00 | 1MOD |
| ATOM | 1369 | C | LEU | 172 | −15.386 | −16.010 | −1.535 | 1.00 | 0.00 | 1MOD |
| ATOM | 1370 | O | LEU | 172 | −15.965 | −16.486 | −2.510 | 1.00 | 0.00 | 1MOD |
| ATOM | 1371 | CB | LEU | 172 | −16.342 | −13.957 | −0.503 | 1.00 | 0.00 | 1MOD |
| ATOM | 1372 | CG | LEU | 172 | −17.203 | −13.534 | −1.706 | 1.00 | 0.00 | 1MOD |
| ATOM | 1373 | CD1 | LEU | 172 | −18.601 | −14.176 | −1.649 | 1.00 | 0.00 | 1MOD |
| ATOM | 1374 | CD2 | LEU | 172 | −17.294 | −12.004 | −1.802 | 1.00 | 0.00 | 1MOD |
| ATOM | 1375 | N | SER | 173 | −14.044 | −15.957 | −1.460 | 1.00 | 0.00 | 1MOD |
| ATOM | 1376 | CA | SER | 173 | −13.225 | −16.410 | −2.547 | 1.00 | 0.00 | 1MOD |
| ATOM | 1377 | C | SER | 173 | −13.461 | −17.874 | −2.751 | 1.00 | 0.00 | 1MOD |
| ATOM | 1378 | O | SER | 173 | −13.533 | −18.349 | −3.882 | 1.00 | 0.00 | 1MOD |
| ATOM | 1379 | CB | SER | 173 | −11.720 | −16.220 | −2.292 | 1.00 | 0.00 | 1MOD |
| ATOM | 1380 | OG | SER | 173 | −11.295 | −17.066 | −1.235 | 1.00 | 0.00 | 1MOD |
| ATOM | 1381 | N | LEU | 174 | −13.590 | −18.627 | −1.646 | 1.00 | 0.00 | 1MOD |
| ATOM | 1382 | CA | LEU | 174 | −13.796 | −20.047 | −1.691 | 1.00 | 0.00 | 1MOD |
| ATOM | 1383 | C | LEU | 174 | −15.129 | −20.283 | −2.322 | 1.00 | 0.00 | 1MOD |
| ATOM | 1384 | O | LEU | 174 | −15.319 | −21.243 | −3.069 | 1.00 | 0.00 | 1MOD |
| ATOM | 1385 | CB | LEU | 174 | −13.767 | −20.648 | −0.263 | 1.00 | 0.00 | 1MOD |
| ATOM | 1386 | CG | LEU | 174 | −13.922 | −22.177 | −0.092 | 1.00 | 0.00 | 1MOD |
| ATOM | 1387 | CD1 | LEU | 174 | −13.841 | −22.569 | 1.391 | 1.00 | 0.00 | 1MOD |
| ATOM | 1388 | CD2 | LEU | 174 | −15.203 | −22.736 | −0.732 | 1.00 | 0.00 | 1MOD |
| ATOM | 1389 | N | LEU | 175 | −16.093 | −19.396 | −2.028 | 1.00 | 0.00 | 1MOD |
| ATOM | 1390 | CA | LEU | 175 | −17.437 | −19.552 | −2.492 | 1.00 | 0.00 | 1MOD |
| ATOM | 1391 | C | LEU | 175 | −17.381 | −19.626 | −3.979 | 1.00 | 0.00 | 1MOD |
| ATOM | 1392 | O | LEU | 175 | −17.997 | −20.500 | −4.586 | 1.00 | 0.00 | 1MOD |
| ATOM | 1393 | CB | LEU | 175 | −18.272 | −18.313 | −2.110 | 1.00 | 0.00 | 1MOD |
| ATOM | 1394 | CG | LEU | 175 | −19.785 | −18.373 | −2.400 | 1.00 | 0.00 | 1MOD |
| ATOM | 1395 | CD1 | LEU | 175 | −20.098 | −18.480 | −3.902 | 1.00 | 0.00 | 1MOD |
| ATOM | 1396 | CD2 | LEU | 175 | −20.463 | −19.459 | −1.553 | 1.00 | 0.00 | 1MOD |
| ATOM | 1397 | N | LEU | 176 | −16.661 | −18.693 | −4.621 | 1.00 | 0.00 | 1MOD |
| ATOM | 1398 | CA | LEU | 176 | −16.589 | −18.760 | −6.049 | 1.00 | 0.00 | 1MOD |
| ATOM | 1399 | C | LEU | 176 | −15.726 | −19.893 | −6.527 | 1.00 | 0.00 | 1MOD |
| ATOM | 1400 | O | LEU | 176 | −16.109 | −20.636 | −7.432 | 1.00 | 0.00 | 1MOD |
| ATOM | 1401 | CB | LEU | 176 | −16.106 | −17.450 | −6.699 | 1.00 | 0.00 | 1MOD |
| ATOM | 1402 | CG | LEU | 176 | −17.159 | −16.327 | −6.583 | 1.00 | 0.00 | 1MOD |
| ATOM | 1403 | CD1 | LEU | 176 | −17.404 | −15.935 | −5.119 | 1.00 | 0.00 | 1MOD |
| ATOM | 1404 | CD2 | LEU | 176 | −16.828 | −15.125 | −7.481 | 1.00 | 0.00 | 1MOD |
| ATOM | 1405 | N | ALA | 177 | −14.524 | −20.036 | −5.939 | 1.00 | 0.00 | 1MOD |
| ATOM | 1406 | CA | ALA | 177 | −13.540 | −20.987 | −6.391 | 1.00 | 0.00 | 1MOD |
| ATOM | 1407 | C | ALA | 177 | −13.887 | −22.431 | −6.164 | 1.00 | 0.00 | 1MOD |
| ATOM | 1408 | O | ALA | 177 | −13.751 | −23.250 | −7.074 | 1.00 | 0.00 | 1MOD |
| ATOM | 1409 | CB | ALA | 177 | −12.165 | −20.751 | −5.739 | 1.00 | 0.00 | 1MOD |
| ATOM | 1410 | N | LEU | 178 | −14.329 | −22.788 | −4.945 | 1.00 | 0.00 | 1MOD |
| ATOM | 1411 | CA | LEU | 178 | −14.509 | −24.172 | −4.596 | 1.00 | 0.00 | 1MOD |
| ATOM | 1412 | C | LEU | 178 | −15.625 | −24.872 | −5.314 | 1.00 | 0.00 | 1MOD |
| ATOM | 1413 | O | LEU | 178 | −15.486 | −26.049 | −5.638 | 1.00 | 0.00 | 1MOD |
| ATOM | 1414 | CB | LEU | 178 | −14.592 | −24.421 | −3.082 | 1.00 | 0.00 | 1MOD |
| ATOM | 1415 | CG | LEU | 178 | −13.193 | −24.346 | −2.432 | 1.00 | 0.00 | 1MOD |
| ATOM | 1416 | CD1 | LEU | 178 | −12.572 | −22.948 | −2.584 | 1.00 | 0.00 | 1MOD |
| ATOM | 1417 | CD2 | LEU | 178 | −13.194 | −24.885 | −0.991 | 1.00 | 0.00 | 1MOD |
| ATOM | 1418 | N | PRO | 179 | −16.715 | −24.233 | −5.594 | 1.00 | 0.00 | 1MOD |
| ATOM | 1419 | CA | PRO | 179 | −17.798 | −24.945 | −6.214 | 1.00 | 0.00 | 1MOD |
| ATOM | 1420 | C | PRO | 179 | −17.473 | −25.482 | −7.568 | 1.00 | 0.00 | 1MOD |
| ATOM | 1421 | O | PRO | 179 | −18.208 | −26.342 | −8.047 | 1.00 | 0.00 | 1MOD |
| ATOM | 1422 | CB | PRO | 179 | −18.993 | −24.003 | −6.147 | 1.00 | 0.00 | 1MOD |
| ATOM | 1423 | CG | PRO | 179 | −18.734 | −23.216 | −4.848 | 1.00 | 0.00 | 1MOD |
| ATOM | 1424 | CD | PRO | 179 | −17.201 | −23.199 | −4.703 | 1.00 | 0.00 | 1MOD |
| ATOM | 1425 | N | VAL | 180 | −16.398 | −25.010 | −8.214 | 1.00 | 0.00 | 1MOD |
| ATOM | 1426 | CA | VAL | 180 | −16.056 | −25.522 | −9.506 | 1.00 | 0.00 | 1MOD |
| ATOM | 1427 | C | VAL | 180 | −15.821 | −26.987 | −9.359 | 1.00 | 0.00 | 1MOD |
| ATOM | 1428 | O | VAL | 180 | −16.193 | −27.776 | −10.226 | 1.00 | 0.00 | 1MOD |
| ATOM | 1429 | CB | VAL | 180 | −14.801 | −24.898 | −10.046 | 1.00 | 0.00 | 1MOD |
| ATOM | 1430 | CG1 | VAL | 180 | −13.650 | −25.158 | −9.060 | 1.00 | 0.00 | 1MOD |
| ATOM | 1431 | CG2 | VAL | 180 | −14.539 | −25.463 | −11.451 | 1.00 | 0.00 | 1MOD |
| ATOM | 1432 | N | LEU | 181 | −15.172 | −27.398 | −8.258 | 1.00 | 0.00 | 1MOD |
| ATOM | 1433 | CA | LEU | 181 | −14.926 | −28.796 | −8.093 | 1.00 | 0.00 | 1MOD |
| ATOM | 1434 | C | LEU | 181 | −16.247 | −29.504 | −7.990 | 1.00 | 0.00 | 1MOD |
| ATOM | 1435 | O | LEU | 181 | −16.509 | −30.445 | −8.735 | 1.00 | 0.00 | 1MOD |
| ATOM | 1436 | CB | LEU | 181 | −14.209 | −29.120 | −6.770 | 1.00 | 0.00 | 1MOD |
| ATOM | 1437 | CG | LEU | 181 | −12.831 | −28.456 | −6.598 | 1.00 | 0.00 | 1MOD |
| ATOM | 1438 | CD1 | LEU | 181 | −12.198 | −28.839 | −5.248 | 1.00 | 0.00 | 1MOD |
| ATOM | 1439 | CD2 | LEU | 181 | −11.908 | −28.747 | −7.792 | 1.00 | 0.00 | 1MOD |
| ATOM | 1440 | N | LEU | 182 | −17.116 | −29.048 | −7.058 | 1.00 | 0.00 | 1MOD |
| ATOM | 1441 | CA | LEU | 182 | −18.360 | −29.715 | −6.765 | 1.00 | 0.00 | 1MOD |
| ATOM | 1442 | C | LEU | 182 | −19.327 | −29.678 | −7.902 | 1.00 | 0.00 | 1MOD |
| ATOM | 1443 | O | LEU | 182 | −19.782 | −30.716 | −8.381 | 1.00 | 0.00 | 1MOD |
| ATOM | 1444 | CB | LEU | 182 | −19.096 | −29.089 | −5.566 | 1.00 | 0.00 | 1MOD |
| ATOM | 1445 | CG | LEU | 182 | −18.316 | −29.149 | −4.239 | 1.00 | 0.00 | 1MOD |
| ATOM | 1446 | CD1 | LEU | 182 | −18.028 | −30.602 | −3.829 | 1.00 | 0.00 | 1MOD |
| ATOM | 1447 | CD2 | LEU | 182 | −17.061 | −28.263 | −4.276 | 1.00 | 0.00 | 1MOD |
| ATOM | 1448 | N | PHE | 183 | −19.665 | −28.472 | −8.386 | 1.00 | 0.00 | 1MOD |
| ATOM | 1449 | CA | PHE | 183 | −20.592 | −28.419 | −9.462 | 1.00 | 0.00 | 1MOD |
| ATOM | 1450 | C | PHE | 183 | −19.820 | −28.864 | −10.614 | 1.00 | 0.00 | 1MOD |
| ATOM | 1451 | O | PHE | 183 | −18.651 | −28.525 | −10.772 | 1.00 | 0.00 | 1MOD |
| ATOM | 1452 | CB | PHE | 183 | −21.151 | −27.020 | −9.772 | 1.00 | 0.00 | 1MOD |
| ATOM | 1453 | CG | PHE | 183 | −22.044 | −26.626 | −8.649 | 1.00 | 0.00 | 1MOD |
| ATOM | 1454 | CD1 | PHE | 183 | −23.361 | −27.027 | −8.631 | 1.00 | 0.00 | 1MOD |
| ATOM | 1455 | CD2 | PHE | 183 | −21.560 | −25.867 | −7.611 | 1.00 | 0.00 | 1MOD |
| ATOM | 1456 | CE1 | PHE | 183 | −24.186 | −26.666 | −7.592 | 1.00 | 0.00 | 1MOD |
| ATOM | 1457 | CE2 | PHE | 183 | −22.381 | −25.502 | −6.572 | 1.00 | 0.00 | 1MOD |
| ATOM | 1458 | CZ | PHE | 183 | −23.696 | −25.901 | −6.561 | 1.00 | 0.00 | 1MOD |
| ATOM | 1459 | N | ARG | 184 | −20.477 | −29.577 | −11.525 | 1.00 | 0.00 | 1MOD |
| ATOM | 1460 | CA | ARG | 184 | −19.728 | −30.006 | −12.651 | 1.00 | 0.00 | 1MOD |
| ATOM | 1461 | C | ARG | 184 | −19.302 | −28.774 | −13.402 | 1.00 | 0.00 | 1MOD |
| ATOM | 1462 | O | ARG | 184 | −18.476 | −28.867 | −14.308 | 1.00 | 0.00 | 1MOD |
| ATOM | 1463 | CB | ARG | 184 | −20.383 | −31.129 | −13.467 | 1.00 | 0.00 | 1MOD |
| ATOM | 1464 | CG | ARG | 184 | −20.514 | −32.410 | −12.641 | 1.00 | 0.00 | 1MOD |
| ATOM | 1465 | CD | ARG | 184 | −20.643 | −33.684 | −13.475 | 1.00 | 0.00 | 1MOD |
| ATOM | 1466 | NE | ARG | 184 | −19.364 | −33.837 | −14.219 | 1.00 | 0.00 | 1MOD |
| ATOM | 1467 | CZ | ARG | 184 | −19.279 | −33.434 | −15.520 | 1.00 | 0.00 | 1MOD |
| ATOM | 1468 | NH1 | ARG | 184 | −20.404 | −33.023 | −16.176 | 1.00 | 0.00 | 1MOD |
| ATOM | 1469 | NH2 | ARG | 184 | −18.078 | −33.446 | −16.166 | 1.00 | 0.00 | 1MOD |
| ATOM | 1470 | N | ARG | 185 | −19.896 | −27.594 | −13.053 | 1.00 | 0.00 | 1MOD |
| ATOM | 1471 | CA | ARG | 185 | −19.364 | −26.363 | −13.530 | 1.00 | 0.00 | 1MOD |
| ATOM | 1472 | C | ARG | 185 | −17.949 | −26.477 | −13.117 | 1.00 | 0.00 | 1MOD |
| ATOM | 1473 | O | ARG | 185 | −17.616 | −26.488 | −11.932 | 1.00 | 0.00 | 1MOD |
| ATOM | 1474 | CB | ARG | 185 | −19.974 | −25.119 | −12.869 | 1.00 | 0.00 | 1MOD |
| ATOM | 1475 | CG | ARG | 185 | −19.673 | −23.835 | −13.641 | 1.00 | 0.00 | 1MOD |
| ATOM | 1476 | CD | ARG | 185 | −18.262 | −23.297 | −13.411 | 1.00 | 0.00 | 1MOD |
| ATOM | 1477 | NE | ARG | 185 | −18.220 | −22.746 | −12.028 | 1.00 | 0.00 | 1MOD |
| ATOM | 1478 | CZ | ARG | 185 | −18.573 | −21.445 | −11.817 | 1.00 | 0.00 | 1MOD |
| ATOM | 1479 | NH1 | ARG | 185 | −18.973 | −20.668 | −12.866 | 1.00 | 0.00 | 1MOD |
| ATOM | 1480 | NH2 | ARG | 185 | −18.534 | −20.924 | −10.556 | 1.00 | 0.00 | 1MOD |
| ATOM | 1481 | N | THR | 186 | −17.071 | −26.626 | −14.114 | 1.00 | 0.00 | 1MOD |
| ATOM | 1482 | CA | THR | 186 | −15.743 | −26.920 | −13.712 | 1.00 | 0.00 | 1MOD |
| ATOM | 1483 | C | THR | 186 | −14.767 | −26.107 | −14.463 | 1.00 | 0.00 | 1MOD |
| ATOM | 1484 | O | THR | 186 | −15.073 | −25.145 | −15.164 | 1.00 | 0.00 | 1MOD |
| ATOM | 1485 | CB | THR | 186 | −15.311 | −28.347 | −13.917 | 1.00 | 0.00 | 1MOD |
| ATOM | 1486 | OG1 | THR | 186 | −15.445 | −28.707 | −15.284 | 1.00 | 0.00 | 1MOD |
| ATOM | 1487 | CG2 | THR | 186 | −16.114 | −29.284 | −12.998 | 1.00 | 0.00 | 1MOD |
| ATOM | 1488 | N | VAL | 187 | −13.521 | −26.532 | −14.253 | 1.00 | 0.00 | 1MOD |
| ATOM | 1489 | CA | VAL | 187 | −12.320 | −25.976 | −14.752 | 1.00 | 0.00 | 1MOD |
| ATOM | 1490 | C | VAL | 187 | −11.938 | −26.706 | −15.997 | 1.00 | 0.00 | 1MOD |
| ATOM | 1491 | O | VAL | 187 | −11.792 | −27.926 | −15.985 | 1.00 | 0.00 | 1MOD |
| ATOM | 1492 | CB | VAL | 187 | −11.256 | −26.248 | −13.750 | 1.00 | 0.00 | 1MOD |
| ATOM | 1493 | CG1 | VAL | 187 | −9.919 | −26.074 | −14.430 | 1.00 | 0.00 | 1MOD |
| ATOM | 1494 | CG2 | VAL | 187 | −11.470 | −25.336 | −12.531 | 1.00 | 0.00 | 1MOD |
| ATOM | 1495 | N | TYR | 188 | −11.752 | −25.963 | −17.108 | 1.00 | 0.00 | 1MOD |
| ATOM | 1496 | CA | TYR | 188 | −11.340 | −26.584 | −18.333 | 1.00 | 0.00 | 1MOD |
| ATOM | 1497 | C | TYR | 188 | −10.225 | −25.724 | −18.857 | 1.00 | 0.00 | 1MOD |
| ATOM | 1498 | O | TYR | 188 | −10.152 | −24.538 | −18.542 | 1.00 | 0.00 | 1MOD |
| ATOM | 1499 | CB | TYR | 188 | −12.476 | −26.629 | −19.372 | 1.00 | 0.00 | 1MOD |
| ATOM | 1500 | CG | TYR | 188 | −12.574 | −25.318 | −20.072 | 1.00 | 0.00 | 1MOD |
| ATOM | 1501 | CD1 | TYR | 188 | −12.735 | −24.137 | −19.389 | 1.00 | 0.00 | 1MOD |
| ATOM | 1502 | CD2 | TYR | 188 | −12.445 | −25.277 | −21.440 | 1.00 | 0.00 | 1MOD |
| ATOM | 1503 | CE1 | TYR | 188 | −12.806 | −22.942 | −20.070 | 1.00 | 0.00 | 1MOD |
| ATOM | 1504 | CE2 | TYR | 188 | −12.518 | −24.089 | −22.125 | 1.00 | 0.00 | 1MOD |
| ATOM | 1505 | CZ | TYR | 188 | −12.705 | −22.913 | −21.442 | 1.00 | 0.00 | 1MOD |
| ATOM | 1506 | OH | TYR | 188 | −12.779 | −21.692 | −22.147 | 1.00 | 0.00 | 1MOD |
| ATOM | 1507 | N | SER | 189 | −9.307 | −26.303 | −19.657 | 1.00 | 0.00 | 1MOD |
| ATOM | 1508 | CA | SER | 189 | −8.165 | −25.571 | −20.136 | 1.00 | 0.00 | 1MOD |
| ATOM | 1509 | C | SER | 189 | −8.596 | −24.559 | −21.145 | 1.00 | 0.00 | 1MOD |
| ATOM | 1510 | O | SER | 189 | −9.615 | −24.722 | −21.811 | 1.00 | 0.00 | 1MOD |
| ATOM | 1511 | CB | SER | 189 | −7.126 | −26.451 | −20.844 | 1.00 | 0.00 | 1MOD |
| ATOM | 1512 | OG | SER | 189 | −6.600 | −27.405 | −19.937 | 1.00 | 0.00 | 1MOD |
| ATOM | 1513 | N | SER | 190 | −7.804 | −23.475 | −21.288 | 1.00 | 0.00 | 1MOD |
| ATOM | 1514 | CA | SER | 190 | −8.156 | −22.460 | −22.238 | 1.00 | 0.00 | 1MOD |
| ATOM | 1515 | C | SER | 190 | −6.939 | −22.084 | −23.020 | 1.00 | 0.00 | 1MOD |
| ATOM | 1516 | O | SER | 190 | −5.814 | −22.376 | −22.612 | 1.00 | 0.00 | 1MOD |
| ATOM | 1517 | CB | SER | 190 | −8.682 | −21.163 | −21.596 | 1.00 | 0.00 | 1MOD |
| ATOM | 1518 | OG | SER | 190 | −9.908 | −21.407 | −20.921 | 1.00 | 0.00 | 1MOD |
| ATOM | 1519 | N | ASN | 191 | −7.153 | −21.451 | −24.200 | 1.00 | 0.00 | 1MOD |
| ATOM | 1520 | CA | ASN | 191 | −6.076 | −20.886 | −24.968 | 1.00 | 0.00 | 1MOD |
| ATOM | 1521 | C | ASN | 191 | −5.364 | −22.007 | −25.635 | 1.00 | 0.00 | 1MOD |
| ATOM | 1522 | O | ASN | 191 | −5.923 | −22.838 | −26.348 | 1.00 | 0.00 | 1MOD |
| ATOM | 1523 | CB | ASN | 191 | −4.968 | −20.242 | −24.103 | 1.00 | 0.00 | 1MOD |
| ATOM | 1524 | CG | ASN | 191 | −5.556 | −19.490 | −22.916 | 1.00 | 0.00 | 1MOD |
| ATOM | 1525 | OD1 | ASN | 191 | −6.760 | −19.275 | −22.806 | 1.00 | 0.00 | 1MOD |
| ATOM | 1526 | ND2 | ASN | 191 | −4.667 | −19.121 | −21.955 | 1.00 | 0.00 | 1MOD |
| ATOM | 1527 | N | VAL | 192 | −4.050 | −22.022 | −25.351 | 1.00 | 0.00 | 1MOD |
| ATOM | 1528 | CA | VAL | 192 | −3.065 | −22.991 | −25.724 | 1.00 | 0.00 | 1MOD |
| ATOM | 1529 | C | VAL | 192 | −3.419 | −24.230 | −24.959 | 1.00 | 0.00 | 1MOD |
| ATOM | 1530 | O | VAL | 192 | −2.847 | −25.301 | −25.160 | 1.00 | 0.00 | 1MOD |
| ATOM | 1531 | CB | VAL | 192 | −1.669 | −22.576 | −25.336 | 1.00 | 0.00 | 1MOD |
| ATOM | 1532 | CG1 | VAL | 192 | −0.680 | −23.706 | −25.673 | 1.00 | 0.00 | 1MOD |
| ATOM | 1533 | CG2 | VAL | 192 | −1.352 | −21.231 | −26.010 | 1.00 | 0.00 | 1MOD |
| ATOM | 1534 | N | SER | 193 | −4.384 | −24.079 | −24.031 | 1.00 | 0.00 | 1MOD |
| ATOM | 1535 | CA | SER | 193 | −4.731 | −25.018 | −23.019 | 1.00 | 0.00 | 1MOD |
| ATOM | 1536 | C | SER | 193 | −3.635 | −25.097 | −21.980 | 1.00 | 0.00 | 1MOD |
| ATOM | 1537 | O | SER | 193 | −3.537 | −26.186 | −21.407 | 1.00 | 0.00 | 1MOD |
| ATOM | 1538 | CB | SER | 193 | −4.960 | −26.439 | −23.568 | 1.00 | 0.00 | 1MOD |
| ATOM | 1539 | OG | SER | 193 | −6.077 | −26.449 | −24.444 | 1.00 | 0.00 | 1MOD |
| ATOM | 1540 | N | PRO | 194 | −2.754 | −24.113 | −21.688 | 1.00 | 0.00 | 1MOD |
| ATOM | 1541 | CA | PRO | 194 | −1.828 | −24.249 | −20.603 | 1.00 | 0.00 | 1MOD |
| ATOM | 1542 | C | PRO | 194 | −2.458 | −23.928 | −19.289 | 1.00 | 0.00 | 1MOD |
| ATOM | 1543 | O | PRO | 194 | −1.901 | −24.323 | −18.267 | 1.00 | 0.00 | 1MOD |
| ATOM | 1544 | CB | PRO | 194 | −0.683 | −23.274 | −20.857 | 1.00 | 0.00 | 1MOD |
| ATOM | 1545 | CG | PRO | 194 | −1.354 | −22.148 | −21.639 | 1.00 | 0.00 | 1MOD |
| ATOM | 1546 | CD | PRO | 194 | −2.434 | −22.889 | −22.430 | 1.00 | 0.00 | 1MOD |
| ATOM | 1547 | N | ALA | 195 | −3.589 | −23.192 | −19.276 | 1.00 | 0.00 | 1MOD |
| ATOM | 1548 | CA | ALA | 195 | −4.174 | −22.784 | −18.031 | 1.00 | 0.00 | 1MOD |
| ATOM | 1549 | C | ALA | 195 | −5.590 | −23.247 | −18.026 | 1.00 | 0.00 | 1MOD |
| ATOM | 1550 | O | ALA | 195 | −6.148 | −23.582 | −19.068 | 1.00 | 0.00 | 1MOD |
| ATOM | 1551 | CB | ALA | 195 | −4.205 | −21.258 | −17.845 | 1.00 | 0.00 | 1MOD |
| ATOM | 1552 | N | CYS | 196 | −6.202 | −23.309 | −16.827 | 1.00 | 0.00 | 1MOD |
| ATOM | 1553 | CA | CYS | 196 | −7.553 | −23.765 | −16.753 | 1.00 | 0.00 | 1MOD |
| ATOM | 1554 | C | CYS | 196 | −8.404 | −22.722 | −16.100 | 1.00 | 0.00 | 1MOD |
| ATOM | 1555 | O | CYS | 196 | −7.975 | −22.028 | −15.180 | 1.00 | 0.00 | 1MOD |
| ATOM | 1556 | CB | CYS | 196 | −7.680 | −25.111 | −16.022 | 1.00 | 0.00 | 1MOD |
| ATOM | 1557 | SG | CYS | 196 | −7.450 | −26.533 | −17.138 | 1.00 | 0.00 | 1MOD |
| ATOM | 1558 | N | TYR | 197 | −9.651 | −22.584 | −16.603 | 1.00 | 0.00 | 1MOD |
| ATOM | 1559 | CA | TYR | 197 | −10.574 | −21.594 | −16.132 | 1.00 | 0.00 | 1MOD |
| ATOM | 1560 | C | TYR | 197 | −11.908 | −22.250 | −15.971 | 1.00 | 0.00 | 1MOD |
| ATOM | 1561 | O | TYR | 197 | −12.029 | −23.461 | −16.141 | 1.00 | 0.00 | 1MOD |
| ATOM | 1562 | CB | TYR | 197 | −10.703 | −20.394 | −17.079 | 1.00 | 0.00 | 1MOD |
| ATOM | 1563 | CG | TYR | 197 | −9.349 | −19.780 | −17.068 | 1.00 | 0.00 | 1MOD |
| ATOM | 1564 | CD1 | TYR | 197 | −8.382 | −20.231 | −17.936 | 1.00 | 0.00 | 1MOD |
| ATOM | 1565 | CD2 | TYR | 197 | −9.047 | −18.778 | −16.175 | 1.00 | 0.00 | 1MOD |
| ATOM | 1566 | CE1 | TYR | 197 | −7.126 | −19.677 | −17.930 | 1.00 | 0.00 | 1MOD |
| ATOM | 1567 | CE2 | TYR | 197 | −7.790 | −18.221 | −16.166 | 1.00 | 0.00 | 1MOD |
| ATOM | 1568 | CZ | TYR | 197 | −6.830 | −18.674 | −17.042 | 1.00 | 0.00 | 1MOD |
| ATOM | 1569 | OH | TYR | 197 | −5.541 | −18.106 | −17.040 | 1.00 | 0.00 | 1MOD |
| ATOM | 1570 | N | GLU | 198 | −12.942 | −21.472 | −15.591 | 1.00 | 0.00 | 1MOD |
| ATOM | 1571 | CA | GLU | 198 | −14.254 | −22.031 | −15.405 | 1.00 | 0.00 | 1MOD |
| ATOM | 1572 | C | GLU | 198 | −14.792 | −22.344 | −16.759 | 1.00 | 0.00 | 1MOD |
| ATOM | 1573 | O | GLU | 198 | −14.236 | −21.905 | −17.764 | 1.00 | 0.00 | 1MOD |
| ATOM | 1574 | CB | GLU | 198 | −15.250 | −21.114 | −14.675 | 1.00 | 0.00 | 1MOD |
| ATOM | 1575 | CG | GLU | 198 | −14.883 | −20.882 | −13.209 | 1.00 | 0.00 | 1MOD |
| ATOM | 1576 | CD | GLU | 198 | −14.661 | −22.239 | −12.549 | 1.00 | 0.00 | 1MOD |
| ATOM | 1577 | OE1 | GLU | 198 | −15.624 | −23.049 | −12.521 | 1.00 | 0.00 | 1MOD |
| ATOM | 1578 | OE2 | GLU | 198 | −13.520 | −22.491 | −12.074 | 1.00 | 0.00 | 1MOD |
| ATOM | 1579 | N | ASP | 199 | −15.873 | −23.150 | −16.834 | 1.00 | 0.00 | 1MOD |
| ATOM | 1580 | CA | ASP | 199 | −16.283 | −23.517 | −18.150 | 1.00 | 0.00 | 1MOD |
| ATOM | 1581 | C | ASP | 199 | −17.421 | −22.688 | −18.657 | 1.00 | 0.00 | 1MOD |
| ATOM | 1582 | O | ASP | 199 | −18.446 | −22.477 | −18.012 | 1.00 | 0.00 | 1MOD |
| ATOM | 1583 | CB | ASP | 199 | −16.595 | −25.000 | −18.348 | 1.00 | 0.00 | 1MOD |
| ATOM | 1584 | CG | ASP | 199 | −16.543 | −25.255 | −19.845 | 1.00 | 0.00 | 1MOD |
| ATOM | 1585 | OD1 | ASP | 199 | −15.716 | −24.595 | −20.530 | 1.00 | 0.00 | 1MOD |
| ATOM | 1586 | OD2 | ASP | 199 | −17.320 | −26.128 | −20.315 | 1.00 | 0.00 | 1MOD |
| ATOM | 1587 | N | MET | 200 | −17.149 | −22.145 | −19.851 | 1.00 | 0.00 | 1MOD |
| ATOM | 1588 | CA | MET | 200 | −17.873 | −21.327 | −20.767 | 1.00 | 0.00 | 1MOD |
| ATOM | 1589 | C | MET | 200 | −18.834 | −22.111 | −21.616 | 1.00 | 0.00 | 1MOD |
| ATOM | 1590 | O | MET | 200 | −19.547 | −21.505 | −22.410 | 1.00 | 0.00 | 1MOD |
| ATOM | 1591 | CB | MET | 200 | −16.884 | −20.598 | −21.688 | 1.00 | 0.00 | 1MOD |
| ATOM | 1592 | CG | MET | 200 | −16.126 | −19.493 | −20.950 | 1.00 | 0.00 | 1MOD |
| ATOM | 1593 | SD | MET | 200 | −14.355 | −19.384 | −21.342 | 1.00 | 0.00 | 1MOD |
| ATOM | 1594 | CE | MET | 200 | −14.562 | −19.535 | −23.139 | 1.00 | 0.00 | 1MOD |
| ATOM | 1595 | N | GLY | 201 | −18.760 | −23.461 | −21.624 | 1.00 | 0.00 | 1MOD |
| ATOM | 1596 | CA | GLY | 201 | −19.613 | −24.269 | −22.464 | 1.00 | 0.00 | 1MOD |
| ATOM | 1597 | C | GLY | 201 | −20.961 | −24.426 | −21.825 | 1.00 | 0.00 | 1MOD |
| ATOM | 1598 | O | GLY | 201 | −21.116 | −24.199 | −20.627 | 1.00 | 0.00 | 1MOD |
| ATOM | 1599 | N | ASN | 202 | −21.984 | −24.839 | −22.615 | 1.00 | 0.00 | 1MOD |
| ATOM | 1600 | CA | ASN | 202 | −23.299 | −24.976 | −22.043 | 1.00 | 0.00 | 1MOD |
| ATOM | 1601 | C | ASN | 202 | −23.274 | −25.982 | −20.945 | 1.00 | 0.00 | 1MOD |
| ATOM | 1602 | O | ASN | 202 | −23.596 | −25.626 | −19.814 | 1.00 | 0.00 | 1MOD |
| ATOM | 1603 | CB | ASN | 202 | −24.395 | −25.410 | −23.034 | 1.00 | 0.00 | 1MOD |
| ATOM | 1604 | CG | ASN | 202 | −24.879 | −24.173 | −23.777 | 1.00 | 0.00 | 1MOD |
| ATOM | 1605 | OD1 | ASN | 202 | −24.300 | −23.096 | −23.650 | 1.00 | 0.00 | 1MOD |
| ATOM | 1606 | ND2 | ASN | 202 | −25.961 | −24.330 | −24.586 | 1.00 | 0.00 | 1MOD |
| ATOM | 1607 | N | ASN | 203 | −22.846 | −27.232 | −21.246 | 1.00 | 0.00 | 1MOD |
| ATOM | 1608 | CA | ASN | 203 | −22.715 | −28.284 | −20.277 | 1.00 | 0.00 | 1MOD |
| ATOM | 1609 | C | ASN | 203 | −23.889 | −28.275 | −19.328 | 1.00 | 0.00 | 1MOD |
| ATOM | 1610 | O | ASN | 203 | −23.857 | −27.709 | −18.243 | 1.00 | 0.00 | 1MOD |
| ATOM | 1611 | CB | ASN | 203 | −21.339 | −28.174 | −19.622 | 1.00 | 0.00 | 1MOD |
| ATOM | 1612 | CG | ASN | 203 | −20.348 | −28.499 | −20.726 | 1.00 | 0.00 | 1MOD |
| ATOM | 1613 | OD1 | ASN | 203 | −20.382 | −29.586 | −21.301 | 1.00 | 0.00 | 1MOD |
| ATOM | 1614 | ND2 | ASN | 203 | −19.461 | −27.526 | −21.058 | 1.00 | 0.00 | 1MOD |
| ATOM | 1615 | N | THR | 204 | −25.014 | −28.856 | −19.764 | 1.00 | 0.00 | 1MOD |
| ATOM | 1616 | CA | THR | 204 | −26.236 | −28.792 | −19.014 | 1.00 | 0.00 | 1MOD |
| ATOM | 1617 | C | THR | 204 | −26.163 | −29.464 | −17.669 | 1.00 | 0.00 | 1MOD |
| ATOM | 1618 | O | THR | 204 | −26.659 | −28.916 | −16.687 | 1.00 | 0.00 | 1MOD |
| ATOM | 1619 | CB | THR | 204 | −27.386 | −29.414 | −19.759 | 1.00 | 0.00 | 1MOD |
| ATOM | 1620 | OG1 | THR | 204 | −28.601 | −29.229 | −19.047 | 1.00 | 0.00 | 1MOD |
| ATOM | 1621 | CG2 | THR | 204 | −27.099 | −30.913 | −19.955 | 1.00 | 0.00 | 1MOD |
| ATOM | 1622 | N | ALA | 205 | −25.560 | −30.666 | −17.567 | 1.00 | 0.00 | 1MOD |
| ATOM | 1623 | CA | ALA | 205 | −25.622 | −31.414 | −16.333 | 1.00 | 0.00 | 1MOD |
| ATOM | 1624 | C | ALA | 205 | −24.981 | −30.673 | −15.201 | 1.00 | 0.00 | 1MOD |
| ATOM | 1625 | O | ALA | 205 | −25.542 | −30.562 | −14.111 | 1.00 | 0.00 | 1MOD |
| ATOM | 1626 | CB | ALA | 205 | −24.909 | −32.772 | −16.440 | 1.00 | 0.00 | 1MOD |
| ATOM | 1627 | N | ASN | 206 | −23.780 | −30.147 | −15.460 | 1.00 | 0.00 | 1MOD |
| ATOM | 1628 | CA | ASN | 206 | −22.934 | −29.403 | −14.577 | 1.00 | 0.00 | 1MOD |
| ATOM | 1629 | C | ASN | 206 | −23.555 | −28.089 | −14.244 | 1.00 | 0.00 | 1MOD |
| ATOM | 1630 | O | ASN | 206 | −23.338 | −27.554 | −13.157 | 1.00 | 0.00 | 1MOD |
| ATOM | 1631 | CB | ASN | 206 | −21.578 | −29.152 | −15.229 | 1.00 | 0.00 | 1MOD |
| ATOM | 1632 | CG | ASN | 206 | −21.849 | −28.710 | −16.632 | 1.00 | 0.00 | 1MOD |
| ATOM | 1633 | OD1 | ASN | 206 | −22.252 | −29.525 | −17.463 | 1.00 | 0.00 | 1MOD |
| ATOM | 1634 | ND2 | ASN | 206 | −21.629 | −27.398 | −16.890 | 1.00 | 0.00 | 1MOD |
| ATOM | 1635 | N | TRP | 207 | −24.362 | −27.544 | −15.169 | 1.00 | 0.00 | 1MOD |
| ATOM | 1636 | CA | TRP | 207 | −24.967 | −26.257 | −14.979 | 1.00 | 0.00 | 1MOD |
| ATOM | 1637 | C | TRP | 207 | −23.909 | −25.213 | −14.841 | 1.00 | 0.00 | 1MOD |
| ATOM | 1638 | O | TRP | 207 | −23.909 | −24.427 | −13.898 | 1.00 | 0.00 | 1MOD |
| ATOM | 1639 | CB | TRP | 207 | −25.818 | −26.133 | −13.708 | 1.00 | 0.00 | 1MOD |
| ATOM | 1640 | CG | TRP | 207 | −27.021 | −27.035 | −13.634 | 1.00 | 0.00 | 1MOD |
| ATOM | 1641 | CD1 | TRP | 207 | −28.252 | −26.905 | −14.207 | 1.00 | 0.00 | 1MOD |
| ATOM | 1642 | CD2 | TRP | 207 | −27.072 | −28.215 | −12.820 | 1.00 | 0.00 | 1MOD |
| ATOM | 1643 | NE1 | TRP | 207 | −29.066 | −27.938 | −13.809 | 1.00 | 0.00 | 1MOD |
| ATOM | 1644 | CE2 | TRP | 207 | −28.354 | −28.747 | −12.949 | 1.00 | 0.00 | 1MOD |
| ATOM | 1645 | CE3 | TRP | 207 | −26.129 | −28.797 | −12.018 | 1.00 | 0.00 | 1MOD |
| ATOM | 1646 | CZ2 | TRP | 207 | −28.714 | −29.878 | −12.276 | 1.00 | 0.00 | 1MOD |
| ATOM | 1647 | CZ3 | TRP | 207 | −26.492 | −29.942 | −11.347 | 1.00 | 0.00 | 1MOD |
| ATOM | 1648 | CH2 | TRP | 207 | −27.761 | −30.468 | −11.476 | 1.00 | 0.00 | 1MOD |
| ATOM | 1649 | N | ARG | 208 | −22.958 | −25.187 | −15.788 | 1.00 | 0.00 | 1MOD |
| ATOM | 1650 | CA | ARG | 208 | −21.954 | −24.167 | −15.748 | 1.00 | 0.00 | 1MOD |
| ATOM | 1651 | C | ARG | 208 | −22.611 | −22.846 | −15.963 | 1.00 | 0.00 | 1MOD |
| ATOM | 1652 | O | ARG | 208 | −22.158 | −21.835 | −15.433 | 1.00 | 0.00 | 1MOD |
| ATOM | 1653 | CB | ARG | 208 | −20.828 | −24.363 | −16.775 | 1.00 | 0.00 | 1MOD |
| ATOM | 1654 | CG | ARG | 208 | −19.772 | −25.339 | −16.255 | 1.00 | 0.00 | 1MOD |
| ATOM | 1655 | CD | ARG | 208 | −19.091 | −26.172 | −17.337 | 1.00 | 0.00 | 1MOD |
| ATOM | 1656 | NE | ARG | 208 | −18.428 | −27.330 | −16.667 | 1.00 | 0.00 | 1MOD |
| ATOM | 1657 | CZ | ARG | 208 | −18.101 | −28.450 | −17.378 | 1.00 | 0.00 | 1MOD |
| ATOM | 1658 | NH1 | ARG | 208 | −18.301 | −28.500 | −18.726 | 1.00 | 0.00 | 1MOD |
| ATOM | 1659 | NH2 | ARG | 208 | −17.579 | −29.538 | −16.744 | 1.00 | 0.00 | 1MOD |
| ATOM | 1660 | N | MET | 209 | −23.703 | −22.822 | −16.748 | 1.00 | 0.00 | 1MOD |
| ATOM | 1661 | CA | MET | 209 | −24.356 | −21.594 | −17.090 | 1.00 | 0.00 | 1MOD |
| ATOM | 1662 | C | MET | 209 | −24.826 | −20.900 | −15.847 | 1.00 | 0.00 | 1MOD |
| ATOM | 1663 | O | MET | 209 | −24.655 | −19.688 | −15.718 | 1.00 | 0.00 | 1MOD |
| ATOM | 1664 | CB | MET | 209 | −25.567 | −21.830 | −18.007 | 1.00 | 0.00 | 1MOD |
| ATOM | 1665 | CG | MET | 209 | −25.172 | −22.359 | −19.387 | 1.00 | 0.00 | 1MOD |
| ATOM | 1666 | SD | MET | 209 | −26.555 | −23.007 | −20.370 | 1.00 | 0.00 | 1MOD |
| ATOM | 1667 | CE | MET | 209 | −26.567 | −24.608 | −19.515 | 1.00 | 0.00 | 1MOD |
| ATOM | 1668 | N | LEU | 210 | −25.421 | −21.636 | −14.889 | 1.00 | 0.00 | 1MOD |
| ATOM | 1669 | CA | LEU | 210 | −25.909 | −20.994 | −13.700 | 1.00 | 0.00 | 1MOD |
| ATOM | 1670 | C | LEU | 210 | −24.759 | −20.395 | −12.960 | 1.00 | 0.00 | 1MOD |
| ATOM | 1671 | O | LEU | 210 | −24.807 | −19.240 | −12.540 | 1.00 | 0.00 | 1MOD |
| ATOM | 1672 | CB | LEU | 210 | −26.593 | −21.978 | −12.732 | 1.00 | 0.00 | 1MOD |
| ATOM | 1673 | CG | LEU | 210 | −27.905 | −22.578 | −13.271 | 1.00 | 0.00 | 1MOD |
| ATOM | 1674 | CD1 | LEU | 210 | −28.985 | −21.498 | −13.424 | 1.00 | 0.00 | 1MOD |
| ATOM | 1675 | CD2 | LEU | 210 | −27.671 | −23.385 | −14.558 | 1.00 | 0.00 | 1MOD |
| ATOM | 1676 | N | LEU | 211 | −23.677 | −21.172 | −12.801 | 1.00 | 0.00 | 1MOD |
| ATOM | 1677 | CA | LEU | 211 | −22.549 | −20.732 | −12.041 | 1.00 | 0.00 | 1MOD |
| ATOM | 1678 | C | LEU | 211 | −21.897 | −19.559 | −12.683 | 1.00 | 0.00 | 1MOD |
| ATOM | 1679 | O | LEU | 211 | −21.459 | −18.665 | −11.981 | 1.00 | 0.00 | 1MOD |
| ATOM | 1680 | CB | LEU | 211 | −21.496 | −21.829 | −11.816 | 1.00 | 0.00 | 1MOD |
| ATOM | 1681 | CG | LEU | 211 | −21.968 | −22.917 | −10.832 | 1.00 | 0.00 | 1MOD |
| ATOM | 1682 | CD1 | LEU | 211 | −22.185 | −22.329 | −9.428 | 1.00 | 0.00 | 1MOD |
| ATOM | 1683 | CD2 | LEU | 211 | −23.202 | −23.668 | −11.352 | 1.00 | 0.00 | 1MOD |
| ATOM | 1684 | N | ARG | 212 | −21.805 | −19.483 | −14.017 | 1.00 | 0.00 | 1MOD |
| ATOM | 1685 | CA | ARG | 212 | −21.073 | −18.393 | −14.604 | 1.00 | 0.00 | 1MOD |
| ATOM | 1686 | C | ARG | 212 | −21.685 | −17.101 | −14.187 | 1.00 | 0.00 | 1MOD |
| ATOM | 1687 | O | ARG | 212 | −20.972 | −16.155 | −13.854 | 1.00 | 0.00 | 1MOD |
| ATOM | 1688 | CB | ARG | 212 | −21.094 | −18.399 | −16.138 | 1.00 | 0.00 | 1MOD |
| ATOM | 1689 | CG | ARG | 212 | −22.501 | −18.275 | −16.721 | 1.00 | 0.00 | 1MOD |
| ATOM | 1690 | CD | ARG | 212 | −22.491 | −17.971 | −18.215 | 1.00 | 0.00 | 1MOD |
| ATOM | 1691 | NE | ARG | 212 | −23.901 | −17.913 | −18.704 | 1.00 | 0.00 | 1MOD |
| ATOM | 1692 | CZ | ARG | 212 | −24.382 | −16.782 | −19.304 | 1.00 | 0.00 | 1MOD |
| ATOM | 1693 | NH1 | ARG | 212 | −23.613 | −15.654 | −19.349 | 1.00 | 0.00 | 1MOD |
| ATOM | 1694 | NH2 | ARG | 212 | −25.619 | −16.791 | −19.883 | 1.00 | 0.00 | 1MOD |
| ATOM | 1695 | N | ILE | 213 | −23.025 | −17.028 | −14.191 | 1.00 | 0.00 | 1MOD |
| ATOM | 1696 | CA | ILE | 213 | −23.656 | −15.812 | −13.786 | 1.00 | 0.00 | 1MOD |
| ATOM | 1697 | C | ILE | 213 | −23.429 | −15.590 | −12.326 | 1.00 | 0.00 | 1MOD |
| ATOM | 1698 | O | ILE | 213 | −23.177 | −14.461 | −11.907 | 1.00 | 0.00 | 1MOD |
| ATOM | 1699 | CB | ILE | 213 | −25.133 | −15.753 | −14.046 | 1.00 | 0.00 | 1MOD |
| ATOM | 1700 | CG1 | ILE | 213 | −25.897 | −16.817 | −13.244 | 1.00 | 0.00 | 1MOD |
| ATOM | 1701 | CG2 | ILE | 213 | −25.350 | −15.838 | −15.564 | 1.00 | 0.00 | 1MOD |
| ATOM | 1702 | CD1 | ILE | 213 | −27.406 | −16.582 | −13.245 | 1.00 | 0.00 | 1MOD |
| ATOM | 1703 | N | LEU | 214 | −23.519 | −16.655 | −11.502 | 1.00 | 0.00 | 1MOD |
| ATOM | 1704 | CA | LEU | 214 | −23.418 | −16.431 | −10.088 | 1.00 | 0.00 | 1MOD |
| ATOM | 1705 | C | LEU | 214 | −22.076 | −15.812 | −9.787 | 1.00 | 0.00 | 1MOD |
| ATOM | 1706 | O | LEU | 214 | −22.038 | −14.768 | −9.140 | 1.00 | 0.00 | 1MOD |
| ATOM | 1707 | CB | LEU | 214 | −23.637 | −17.715 | −9.264 | 1.00 | 0.00 | 1MOD |
| ATOM | 1708 | CG | LEU | 214 | −25.005 | −18.381 | −9.503 | 1.00 | 0.00 | 1MOD |
| ATOM | 1709 | CD1 | LEU | 214 | −25.171 | −19.635 | −8.627 | 1.00 | 0.00 | 1MOD |
| ATOM | 1710 | CD2 | LEU | 214 | −26.158 | −17.376 | −9.354 | 1.00 | 0.00 | 1MOD |
| ATOM | 1711 | N | PRO | 215 | −20.970 | −16.384 | −10.193 | 1.00 | 0.00 | 1MOD |
| ATOM | 1712 | CA | PRO | 215 | −19.788 | −15.592 | −10.076 | 1.00 | 0.00 | 1MOD |
| ATOM | 1713 | C | PRO | 215 | −19.688 | −14.286 | −10.794 | 1.00 | 0.00 | 1MOD |
| ATOM | 1714 | O | PRO | 215 | −19.012 | −13.406 | −10.264 | 1.00 | 0.00 | 1MOD |
| ATOM | 1715 | CB | PRO | 215 | −18.616 | −16.547 | −10.243 | 1.00 | 0.00 | 1MOD |
| ATOM | 1716 | CG | PRO | 215 | −19.121 | −17.772 | −9.468 | 1.00 | 0.00 | 1MOD |
| ATOM | 1717 | CD | PRO | 215 | −20.653 | −17.664 | −9.555 | 1.00 | 0.00 | 1MOD |
| ATOM | 1718 | N | GLN | 216 | −20.314 | −14.092 | −11.968 | 1.00 | 0.00 | 1MOD |
| ATOM | 1719 | CA | GLN | 216 | −20.136 | −12.795 | −12.555 | 1.00 | 0.00 | 1MOD |
| ATOM | 1720 | C | GLN | 216 | −20.744 | −11.783 | −11.642 | 1.00 | 0.00 | 1MOD |
| ATOM | 1721 | O | GLN | 216 | −20.123 | −10.775 | −11.310 | 1.00 | 0.00 | 1MOD |
| ATOM | 1722 | CB | GLN | 216 | −20.872 | −12.597 | −13.889 | 1.00 | 0.00 | 1MOD |
| ATOM | 1723 | CG | GLN | 216 | −20.348 | −13.427 | −15.057 | 1.00 | 0.00 | 1MOD |
| ATOM | 1724 | CD | GLN | 216 | −21.189 | −13.050 | −16.266 | 1.00 | 0.00 | 1MOD |
| ATOM | 1725 | OE1 | GLN | 216 | −21.594 | −11.898 | −16.418 | 1.00 | 0.00 | 1MOD |
| ATOM | 1726 | NE2 | GLN | 216 | −21.464 | −14.047 | −17.149 | 1.00 | 0.00 | 1MOD |
| ATOM | 1727 | N | SER | 217 | −21.994 | −12.038 | −11.215 | 1.00 | 0.00 | 1MOD |
| ATOM | 1728 | CA | SER | 217 | −22.710 | −11.111 | −10.389 | 1.00 | 0.00 | 1MOD |
| ATOM | 1729 | C | SER | 217 | −22.070 | 11.007 | −9.048 | 1.00 | 0.00 | 1MOD |
| ATOM | 1730 | O | SER | 217 | −21.540 | −9.962 | −8.672 | 1.00 | 0.00 | 1MOD |
| ATOM | 1731 | CB | SER | 217 | −24.160 | −11.559 | −10.133 | 1.00 | 0.00 | 1MOD |
| ATOM | 1732 | OG | SER | 217 | −24.860 | −11.695 | −11.360 | 1.00 | 0.00 | 1MOD |
| ATOM | 1733 | N | PHE | 218 | −22.113 | −12.115 | −8.287 | 1.00 | 0.00 | 1MOD |
| ATOM | 1734 | CA | PHE | 218 | −21.631 | −12.077 | −6.944 | 1.00 | 0.00 | 1MOD |
| ATOM | 1735 | C | PHE | 218 | −20.161 | −11.829 | −6.929 | 1.00 | 0.00 | 1MOD |
| ATOM | 1736 | O | PHE | 218 | −19.677 | −10.984 | −6.177 | 1.00 | 0.00 | 1MOD |
| ATOM | 1737 | CB | PHE | 218 | −21.923 | −13.371 | −6.167 | 1.00 | 0.00 | 1MOD |
| ATOM | 1738 | CG | PHE | 218 | −23.407 | −13.479 | −6.080 | 1.00 | 0.00 | 1MOD |
| ATOM | 1739 | CD1 | PHE | 218 | −24.095 | −12.850 | −5.067 | 1.00 | 0.00 | 1MOD |
| ATOM | 1740 | CD2 | PHE | 218 | −24.112 | −14.191 | −7.023 | 1.00 | 0.00 | 1MOD |
| ATOM | 1741 | CE1 | PHE | 218 | −25.466 | −12.944 | −4.989 | 1.00 | 0.00 | 1MOD |
| ATOM | 1742 | CE2 | PHE | 218 | −25.482 | −14.289 | −6.949 | 1.00 | 0.00 | 1MOD |
| ATOM | 1743 | CZ | PHE | 218 | −26.162 | −13.666 | −5.930 | 1.00 | 0.00 | 1MOD |
| ATOM | 1744 | N | GLY | 219 | −19.397 | −12.580 | −7.738 | 1.00 | 0.00 | 1MOD |
| ATOM | 1745 | CA | GLY | 219 | −17.980 | −12.394 | −7.681 | 1.00 | 0.00 | 1MOD |
| ATOM | 1746 | C | GLY | 219 | −17.528 | −11.078 | −8.245 | 1.00 | 0.00 | 1MOD |
| ATOM | 1747 | O | GLY | 219 | −16.747 | −10.379 | −7.603 | 1.00 | 0.00 | 1MOD |
| ATOM | 1748 | N | PHE | 220 | −17.919 | −10.759 | −9.502 | 1.00 | 0.00 | 1MOD |
| ATOM | 1749 | CA | PHE | 220 | −17.451 | −9.553 | −10.139 | 1.00 | 0.00 | 1MOD |
| ATOM | 1750 | C | PHE | 220 | −18.171 | −8.277 | −9.799 | 1.00 | 0.00 | 1MOD |
| ATOM | 1751 | O | PHE | 220 | −17.581 | −7.330 | −9.279 | 1.00 | 0.00 | 1MOD |
| ATOM | 1752 | CB | PHE | 220 | −17.517 | −9.643 | −11.673 | 1.00 | 0.00 | 1MOD |
| ATOM | 1753 | CG | PHE | 220 | −16.654 | −10.776 | −12.108 | 1.00 | 0.00 | 1MOD |
| ATOM | 1754 | CD1 | PHE | 220 | −17.155 | −12.058 | −12.125 | 1.00 | 0.00 | 1MOD |
| ATOM | 1755 | CD2 | PHE | 220 | −15.354 | −10.563 | −12.503 | 1.00 | 0.00 | 1MOD |
| ATOM | 1756 | CE1 | PHE | 220 | −16.370 | −13.114 | −12.525 | 1.00 | 0.00 | 1MOD |
| ATOM | 1757 | CE2 | PHE | 220 | −14.565 | −11.614 | −12.903 | 1.00 | 0.00 | 1MOD |
| ATOM | 1758 | CB | PHE | 220 | −15.072 | −12.892 | −12.916 | 1.00 | 0.00 | 1MOD |
| ATOM | 1759 | N | ILE | 221 | −19.486 | −8.231 | −10.108 | 1.00 | 0.00 | 1MOD |
| ATOM | 1760 | CA | ILE | 221 | −20.190 | −6.975 | −10.108 | 1.00 | 0.00 | 1MOD |
| ATOM | 1761 | C | ILE | 221 | −20.440 | −6.384 | −8.758 | 1.00 | 0.00 | 1MOD |
| ATOM | 1762 | O | ILE | 221 | −20.170 | −5.202 | −8.545 | 1.00 | 0.00 | 1MOD |
| ATOM | 1763 | CB | ILE | 221 | −21.470 | −7.001 | −10.916 | 1.00 | 0.00 | 1MOD |
| ATOM | 1764 | CG1 | ILE | 221 | −22.572 | −7.888 | −10.312 | 1.00 | 0.00 | 1MOD |
| ATOM | 1765 | CG2 | ILE | 221 | −21.079 | −7.425 | −12.343 | 1.00 | 0.00 | 1MOD |
| ATOM | 1766 | CD1 | ILE | 221 | −23.388 | −7.208 | −9.213 | 1.00 | 0.00 | 1MOD |
| ATOM | 1767 | N | VAL | 222 | −20.953 | −7.171 | −7.798 | 1.00 | 0.00 | 1MOD |
| ATOM | 1768 | CA | VAL | 222 | −21.291 | −6.545 | −6.556 | 1.00 | 0.00 | 1MOD |
| ATOM | 1769 | C | VAL | 222 | −20.081 | −6.025 | −5.845 | 1.00 | 0.00 | 1MOD |
| ATOM | 1770 | O | VAL | 222 | −20.167 | −4.938 | −5.275 | 1.00 | 0.00 | 1MOD |
| ATOM | 1771 | CB | VAL | 222 | −22.210 | −7.345 | −5.656 | 1.00 | 0.00 | 1MOD |
| ATOM | 1772 | CG1 | VAL | 222 | −23.618 | −7.266 | −6.268 | 1.00 | 0.00 | 1MOD |
| ATOM | 1773 | CG2 | VAL | 222 | −21.754 | −8.809 | −5.542 | 1.00 | 0.00 | 1MOD |
| ATOM | 1774 | N | PRO | 223 | −18.951 | −6.680 | −5.831 | 1.00 | 0.00 | 1MOD |
| ATOM | 1775 | CA | PRO | 223 | −17.841 | −6.100 | −5.133 | 1.00 | 0.00 | 1MOD |
| ATOM | 1776 | C | PRO | 223 | −17.380 | −4.874 | −5.845 | 1.00 | 0.00 | 1MOD |
| ATOM | 1777 | O | PRO | 223 | −16.747 | −4.019 | −5.232 | 1.00 | 0.00 | 1MOD |
| ATOM | 1778 | CB | PRO | 223 | −16.782 | −7.191 | −5.034 | 1.00 | 0.00 | 1MOD |
| ATOM | 1779 | CG | PRO | 223 | −17.604 | −8.490 | −5.066 | 1.00 | 0.00 | 1MOD |
| ATOM | 1780 | CD | PRO | 223 | −18.850 | −8.129 | −5.892 | 1.00 | 0.00 | 1MOD |
| ATOM | 1781 | N | LEU | 224 | −17.643 | −4.798 | −7.156 | 1.00 | 0.00 | 1MOD |
| ATOM | 1782 | CA | LEU | 224 | −17.229 | −3.670 | −7.926 | 1.00 | 0.00 | 1MOD |
| ATOM | 1783 | C | LEU | 224 | −18.055 | −2.490 | −7.521 | 1.00 | 0.00 | 1MOD |
| ATOM | 1784 | O | LEU | 224 | −17.533 | −1.395 | −7.322 | 1.00 | 0.00 | 1MOD |
| ATOM | 1785 | CB | LEU | 224 | −17.423 | −3.910 | −9.433 | 1.00 | 0.00 | 1MOD |
| ATOM | 1786 | CG | LEU | 224 | −16.779 | −2.842 | −10.332 | 1.00 | 0.00 | 1MOD |
| ATOM | 1787 | CD1 | LEU | 224 | −17.485 | −1.482 | −10.226 | 1.00 | 0.00 | 1MOD |
| ATOM | 1788 | CD2 | LEU | 224 | −15.268 | −2.752 | −10.061 | 1.00 | 0.00 | 1MOD |
| ATOM | 1789 | N | LEU | 225 | −19.380 | −2.689 | −7.381 | 1.00 | 0.00 | 1MOD |
| ATOM | 1790 | CA | LEU | 225 | −20.225 | −1.577 | −7.062 | 1.00 | 0.00 | 1MOD |
| ATOM | 1791 | C | LEU | 225 | −19.854 | −1.053 | −5.721 | 1.00 | 0.00 | 1MOD |
| ATOM | 1792 | O | LEU | 225 | −19.728 | 0.156 | −5.529 | 1.00 | 0.00 | 1MOD |
| ATOM | 1793 | CB | LEU | 225 | −21.719 | −1.920 | −6.983 | 1.00 | 0.00 | 1MOD |
| ATOM | 1794 | CG | LEU | 225 | −22.570 | −0.722 | −6.517 | 1.00 | 0.00 | 1MOD |
| ATOM | 1795 | CD1 | LEU | 225 | −22.544 | 0.429 | −7.534 | 1.00 | 0.00 | 1MOD |
| ATOM | 1796 | CD2 | LEU | 225 | −23.994 | −1.161 | −6.146 | 1.00 | 0.00 | 1MOD |
| ATOM | 1797 | N | ILE | 226 | −19.651 | −1.963 | −4.757 | 1.00 | 0.00 | 1MOD |
| ATOM | 1798 | CA | ILE | 226 | −19.346 | −1.531 | −3.433 | 1.00 | 0.00 | 1MOD |
| ATOM | 1799 | C | ILE | 226 | −18.065 | −0.770 | −3.430 | 1.00 | 0.00 | 1MOD |
| ATOM | 1800 | O | ILE | 226 | −17.977 | 0.291 | −2.814 | 1.00 | 0.00 | 1MOD |
| ATOM | 1801 | CB | ILE | 226 | −19.238 | −2.663 | −2.453 | 1.00 | 0.00 | 1MOD |
| ATOM | 1802 | CG1 | ILE | 226 | −18.749 | −2.121 | −1.112 | 1.00 | 0.00 | 1MOD |
| ATOM | 1803 | CG2 | ILE | 226 | −18.337 | −3.762 | −3.026 | 1.00 | 0.00 | 1MOD |
| ATOM | 1804 | CD1 | ILE | 226 | −19.699 | −1.114 | −0.481 | 1.00 | 0.00 | 1MOD |
| ATOM | 1805 | N | MET | 227 | −17.038 | −1.278 | −4.128 | 1.00 | 0.00 | 1MOD |
| ATOM | 1806 | CA | MET | 227 | −15.767 | −0.621 | −4.089 | 1.00 | 0.00 | 1MOD |
| ATOM | 1807 | C | MET | 227 | −15.896 | 0.757 | −4.660 | 1.00 | 0.00 | 1MOD |
| ATOM | 1808 | O | MET | 227 | −15.387 | 1.717 | −4.085 | 1.00 | 0.00 | 1MOD |
| ATOM | 1809 | CB | MET | 227 | −14.687 | −1.364 | −4.899 | 1.00 | 0.00 | 1MOD |
| ATOM | 1810 | CG | MET | 227 | −13.255 | −0.888 | −4.626 | 1.00 | 0.00 | 1MOD |
| ATOM | 1811 | SD | MET | 227 | −12.870 | 0.794 | −5.204 | 1.00 | 0.00 | 1MOD |
| ATOM | 1812 | CE | MET | 227 | −11.149 | 0.792 | −4.619 | 1.00 | 0.00 | 1MOD |
| ATOM | 1813 | N | LEU | 228 | −16.570 | 0.898 | −5.819 | 1.00 | 0.00 | 1MOD |
| ATOM | 1814 | CA | LEU | 228 | −16.690 | 2.196 | −6.429 | 1.00 | 0.00 | 1MOD |
| ATOM | 1815 | C | LEU | 228 | −17.582 | 3.120 | −5.657 | 1.00 | 0.00 | 1MOD |
| ATOM | 1816 | O | LEU | 228 | −17.266 | 4.296 | −5.491 | 1.00 | 0.00 | 1MOD |
| ATOM | 1817 | CB | LEU | 228 | −17.180 | 2.167 | −7.887 | 1.00 | 0.00 | 1MOD |
| ATOM | 1818 | CG | LEU | 228 | −16.088 | 1.715 | −8.871 | 1.00 | 0.00 | 1MOD |
| ATOM | 1819 | CD1 | LEU | 228 | −14.967 | 2.765 | −8.971 | 1.00 | 0.00 | 1MOD |
| ATOM | 1820 | CD2 | LEU | 228 | −15.561 | 0.318 | −8.509 | 1.00 | 0.00 | 1MOD |
| ATOM | 1821 | N | PHE | 229 | −18.724 | 2.615 | −5.162 | 1.00 | 0.00 | 1MOD |
| ATOM | 1822 | CA | PHE | 229 | −19.691 | 3.453 | −4.511 | 1.00 | 0.00 | 1MOD |
| ATOM | 1823 | C | PHE | 229 | −19.102 | 4.046 | −3.268 | 1.00 | 0.00 | 1MOD |
| ATOM | 1824 | O | PHE | 229 | −19.274 | 5.233 | −2.991 | 1.00 | 0.00 | 1MOD |
| ATOM | 1825 | CB | PHE | 229 | −20.974 | 2.668 | −4.179 | 1.00 | 0.00 | 1MOD |
| ATOM | 1826 | CG | PHE | 229 | −21.958 | 3.561 | −3.506 | 1.00 | 0.00 | 1MOD |
| ATOM | 1827 | CD1 | PHE | 229 | −22.706 | 4.461 | −4.237 | 1.00 | 0.00 | 1MOD |
| ATOM | 1828 | CD2 | PHE | 229 | −22.122 | 3.509 | −2.145 | 1.00 | 0.00 | 1MOD |
| ATOM | 1829 | CE1 | PHE | 229 | −23.615 | 5.288 | −3.620 | 1.00 | 0.00 | 1MOD |
| ATOM | 1830 | CE2 | PHE | 229 | −23.032 | 4.330 | −1.526 | 1.00 | 0.00 | 1MOD |
| ATOM | 1831 | CZ | PHE | 229 | −23.778 | 5.220 | −2.259 | 1.00 | 0.00 | 1MOD |
| ATOM | 1832 | N | CYS | 230 | −18.363 | 3.230 | −2.496 | 1.00 | 0.00 | 1MOD |
| ATOM | 1833 | CA | CYS | 230 | −17.776 | 3.694 | −1.274 | 1.00 | 0.00 | 1MOD |
| ATOM | 1834 | C | CYS | 230 | −16.827 | 4.793 | −1.623 | 1.00 | 0.00 | 1MOD |
| ATOM | 1835 | O | CYS | 230 | −16.655 | 5.752 | −0.875 | 1.00 | 0.00 | 1MOD |
| ATOM | 1836 | CB | CYS | 230 | −16.981 | 2.592 | −0.553 | 1.00 | 0.00 | 1MOD |
| ATOM | 1837 | SG | CYS | 230 | −16.232 | 3.167 | 1.000 | 1.00 | 0.00 | 1MOD |
| ATOM | 1838 | N | TYR | 231 | −16.200 | 4.670 | −2.800 | 1.00 | 0.00 | 1MOD |
| ATOM | 1839 | CA | TYR | 231 | −15.226 | 5.590 | −3.304 | 1.00 | 0.00 | 1MOD |
| ATOM | 1840 | C | TYR | 231 | −15.844 | 6.945 | −3.427 | 1.00 | 0.00 | 1MOD |
| ATOM | 1841 | O | TYR | 231 | −15.216 | 7.945 | −3.091 | 1.00 | 0.00 | 1MOD |
| ATOM | 1842 | CB | TYR | 231 | −14.727 | 5.132 | −4.684 | 1.00 | 0.00 | 1MOD |
| ATOM | 1843 | CG | TYR | 231 | −13.830 | 6.152 | −5.293 | 1.00 | 0.00 | 1MOD |
| ATOM | 1844 | CD1 | TYR | 231 | −12.549 | 6.341 | −4.828 | 1.00 | 0.00 | 1MOD |
| ATOM | 1845 | CD2 | TYR | 231 | −14.265 | 6.895 | −6.365 | 1.00 | 0.00 | 1MOD |
| ATOM | 1846 | CE1 | TYR | 231 | −11.726 | 7.280 | −5.406 | 1.00 | 0.00 | 1MOD |
| ATOM | 1847 | CE2 | TYR | 231 | −13.444 | 7.832 | −6.948 | 1.00 | 0.00 | 1MOD |
| ATOM | 1848 | CZ | TYR | 231 | −12.173 | 8.028 | −6.467 | 1.00 | 0.00 | 1MOD |
| ATOM | 1849 | OH | TYR | 231 | −11.328 | 8.989 | −7.061 | 1.00 | 0.00 | 1MOD |
| ATOM | 1850 | N | GLY | 232 | −17.104 | 7.017 | −3.882 | 1.00 | 0.00 | 1MOD |
| ATOM | 1851 | CA | GLY | 232 | −17.724 | 8.290 | −4.110 | 1.00 | 0.00 | 1MOD |
| ATOM | 1852 | C | GLY | 232 | −17.715 | 9.096 | −2.847 | 1.00 | 0.00 | 1MOD |
| ATOM | 1853 | O | GLY | 232 | −17.502 | 10.306 | −2.887 | 1.00 | 0.00 | 1MOD |
| ATOM | 1854 | N | PHE | 233 | −17.964 | 8.457 | −1.688 | 1.00 | 0.00 | 1MOD |
| ATOM | 1855 | CA | PHE | 233 | −18.002 | 9.184 | −0.448 | 1.00 | 0.00 | 1MOD |
| ATOM | 1856 | C | PHE | 233 | −16.654 | 9.765 | −0.154 | 1.00 | 0.00 | 1MOD |
| ATOM | 1857 | O | PHE | 233 | −16.547 | 10.912 | 0.277 | 1.00 | 0.00 | 1MOD |
| ATOM | 1858 | CB | PHE | 233 | −18.361 | 8.313 | 0.767 | 1.00 | 0.00 | 1MOD |
| ATOM | 1859 | CG | PHE | 233 | −19.782 | 7.896 | 0.635 | 1.00 | 0.00 | 1MOD |
| ATOM | 1860 | CD1 | PHE | 233 | −20.087 | 6.736 | −0.029 | 1.00 | 0.00 | 1MOD |
| ATOM | 1861 | CD2 | PHE | 233 | −20.806 | 8.655 | 1.165 | 1.00 | 0.00 | 1MOD |
| ATOM | 1862 | CE1 | PHE | 233 | −21.393 | 6.342 | −0.156 | 1.00 | 0.00 | 1MOD |
| ATOM | 1863 | CE2 | PHE | 233 | −22.118 | 8.259 | 1.039 | 1.00 | 0.00 | 1MOD |
| ATOM | 1864 | CZ | PHE | 233 | −22.412 | 7.094 | 0.375 | 1.00 | 0.00 | 1MOD |
| ATOM | 1865 | N | THR | 234 | −15.590 | 8.976 | −0.380 | 1.00 | 0.00 | 1MOD |
| ATOM | 1866 | CA | THR | 234 | −14.244 | 9.349 | −0.058 | 1.00 | 0.00 | 1MOD |
| ATOM | 1867 | C | THR | 234 | −13.771 | 10.497 | −0.888 | 1.00 | 0.00 | 1MOD |
| ATOM | 1868 | O | THR | 234 | −12.833 | 11.177 | −0.482 | 1.00 | 0.00 | 1MOD |
| ATOM | 1869 | CB | THR | 234 | −13.231 | 8.245 | −0.211 | 1.00 | 0.00 | 1MOD |
| ATOM | 1870 | OG1 | THR | 234 | −13.141 | 7.826 | −1.564 | 1.00 | 0.00 | 1MOD |
| ATOM | 1871 | CG2 | THR | 234 | −13.630 | 7.066 | 0.689 | 1.00 | 0.00 | 1MOD |
| ATOM | 1872 | N | LEU | 235 | −14.360 | 10.742 | −2.077 | 1.00 | 0.00 | 1MOD |
| ATOM | 1873 | CA | LEU | 235 | −13.822 | 11.788 | −2.906 | 1.00 | 0.00 | 1MOD |
| ATOM | 1874 | C | LEU | 235 | −13.776 | 13.098 | −2.180 | 1.00 | 0.00 | 1MOD |
| ATOM | 1875 | O | LEU | 235 | −12.796 | 13.830 | −2.303 | 1.00 | 0.00 | 1MOD |
| ATOM | 1876 | CB | LEU | 235 | −14.585 | 12.075 | −4.214 | 1.00 | 0.00 | 1MOD |
| ATOM | 1877 | CG | LEU | 235 | −14.294 | 11.124 | −5.389 | 1.00 | 0.00 | 1MOD |
| ATOM | 1878 | CD1 | LEU | 235 | −12.811 | 11.171 | −5.795 | 1.00 | 0.00 | 1MOD |
| ATOM | 1879 | CD2 | LEU | 235 | −14.810 | 9.709 | −5.133 | 1.00 | 0.00 | 1MOD |
| ATOM | 1880 | N | ARG | 236 | −14.824 | 13.445 | −1.418 | 1.00 | 0.00 | 1MOD |
| ATOM | 1881 | CA | ARG | 236 | −14.838 | 14.731 | −0.779 | 1.00 | 0.00 | 1MOD |
| ATOM | 1882 | C | ARG | 236 | −13.745 | 14.888 | 0.242 | 1.00 | 0.00 | 1MOD |
| ATOM | 1883 | O | ARG | 236 | −12.991 | 15.860 | 0.190 | 1.00 | 0.00 | 1MOD |
| ATOM | 1884 | CB | ARG | 236 | −16.159 | 15.004 | −0.038 | 1.00 | 0.00 | 1MOD |
| ATOM | 1885 | CG | ARG | 236 | −17.394 | 15.007 | −0.945 | 1.00 | 0.00 | 1MOD |
| ATOM | 1886 | CD | ARG | 236 | −18.718 | 15.156 | −0.186 | 1.00 | 0.00 | 1MOD |
| ATOM | 1887 | NE | ARG | 236 | −19.161 | 16.574 | −0.294 | 1.00 | 0.00 | 1MOD |
| ATOM | 1888 | CZ | ARG | 236 | −19.991 | 17.104 | 0.652 | 1.00 | 0.00 | 1MOD |
| ATOM | 1889 | NH1 | ARG | 236 | −20.342 | 16.366 | 1.746 | 1.00 | 0.00 | 1MOD |
| ATOM | 1890 | NH2 | ARG | 236 | −20.469 | 18.373 | 0.500 | 1.00 | 0.00 | 1MOD |
| ATOM | 1891 | N | THR | 237 | −13.618 | 13.943 | 1.199 | 1.00 | 0.00 | 1MOD |
| ATOM | 1892 | CA | THR | 237 | −12.702 | 14.160 | 2.288 | 1.00 | 0.00 | 1MOD |
| ATOM | 1893 | C | THR | 237 | −11.293 | 14.246 | 1.807 | 1.00 | 0.00 | 1MOD |
| ATOM | 1894 | O | THR | 237 | −10.615 | 15.248 | 2.029 | 1.00 | 0.00 | 1MOD |
| ATOM | 1895 | CB | THR | 237 | −12.715 | 13.062 | 3.295 | 1.00 | 0.00 | 1MOD |
| ATOM | 1896 | OG1 | THR | 237 | −14.039 | 12.820 | 3.734 | 1.00 | 0.00 | 1MOD |
| ATOM | 1897 | CG2 | THR | 237 | −11.876 | 13.541 | 4.487 | 1.00 | 0.00 | 1MOD |
| ATOM | 1898 | N | LEU | 238 | −10.811 | 13.186 | 1.128 | 1.00 | 0.00 | 1MOD |
| ATOM | 1899 | CA | LEU | 238 | −9.469 | 13.207 | 0.630 | 1.00 | 0.00 | 1MOD |
| ATOM | 1900 | C | LEU | 238 | −9.607 | 13.007 | −0.841 | 1.00 | 0.00 | 1MOD |
| ATOM | 1901 | O | LEU | 238 | −9.576 | 11.879 | −1.329 | 1.00 | 0.00 | 1MOD |
| ATOM | 1902 | CB | LEU | 238 | −8.611 | 12.041 | 1.150 | 1.00 | 0.00 | 1MOD |
| ATOM | 1903 | CG | LEU | 238 | −8.588 | 11.937 | 2.687 | 1.00 | 0.00 | 1MOD |
| ATOM | 1904 | CD1 | LEU | 238 | −7.628 | 10.840 | 3.180 | 1.00 | 0.00 | 1MOD |
| ATOM | 1905 | CD2 | LEU | 238 | −8.361 | 13.304 | 3.356 | 1.00 | 0.00 | 1MOD |
| ATOM | 1906 | N | PHE | 239 | −9.823 | 14.098 | −1.592 | 1.00 | 0.00 | 1MOD |
| ATOM | 1907 | CA | PHE | 239 | −10.043 | 13.891 | −2.990 | 1.00 | 0.00 | 1MOD |
| ATOM | 1908 | C | PHE | 239 | −8.801 | 13.469 | −3.726 | 1.00 | 0.00 | 1MOD |
| ATOM | 1909 | O | PHE | 239 | −8.711 | 12.344 | −4.217 | 1.00 | 0.00 | 1MOD |
| ATOM | 1910 | CB | PHE | 239 | −10.559 | 15.167 | −3.676 | 1.00 | 0.00 | 1MOD |
| ATOM | 1911 | CG | PHE | 239 | −10.977 | 14.815 | −5.059 | 1.00 | 0.00 | 1MOD |
| ATOM | 1912 | CD1 | PHE | 239 | −12.245 | 14.340 | −5.292 | 1.00 | 0.00 | 1MOD |
| ATOM | 1913 | CD2 | PHE | 239 | −10.107 | 14.960 | −6.116 | 1.00 | 0.00 | 1MOD |
| ATOM | 1914 | CE1 | PHE | 239 | −12.645 | 14.012 | −6.566 | 1.00 | 0.00 | 1MOD |
| ATOM | 1915 | CE2 | PHE | 239 | −10.503 | 14.633 | −7.391 | 1.00 | 0.00 | 1MOD |
| ATOM | 1916 | CZ | PHE | 239 | −11.774 | 14.160 | −7.618 | 1.00 | 0.00 | 1MOD |
| ATOM | 1917 | N | LYS | 240 | −7.823 | 14.400 | −3.804 | 1.00 | 0.00 | 1MOD |
| ATOM | 1918 | CA | LYS | 240 | −6.601 | 14.294 | −4.565 | 1.00 | 0.00 | 1MOD |
| ATOM | 1919 | C | LYS | 240 | −5.571 | 13.404 | −3.948 | 1.00 | 0.00 | 1MOD |
| ATOM | 1920 | O | LYS | 240 | −5.045 | 12.485 | −4.577 | 1.00 | 0.00 | 1MOD |
| ATOM | 1921 | CB | LYS | 240 | −5.955 | 15.679 | −4.742 | 1.00 | 0.00 | 1MOD |
| ATOM | 1922 | CG | LYS | 240 | −6.853 | 16.659 | −5.501 | 1.00 | 0.00 | 1MOD |
| ATOM | 1923 | CD | LYS | 240 | −6.494 | 18.131 | −5.290 | 1.00 | 0.00 | 1MOD |
| ATOM | 1924 | CE | LYS | 240 | −7.340 | 19.088 | −6.133 | 1.00 | 0.00 | 1MOD |
| ATOM | 1925 | NZ | LYS | 240 | −8.772 | 18.739 | −6.012 | 1.00 | 0.00 | 1MOD |
| ATOM | 1926 | N | ALA | 241 | −5.310 | 13.629 | −2.653 | 1.00 | 0.00 | 1MOD |
| ATOM | 1927 | CA | ALA | 241 | −4.267 | 12.955 | −1.932 | 1.00 | 0.00 | 1MOD |
| ATOM | 1928 | C | ALA | 241 | −2.938 | 12.921 | −2.641 | 1.00 | 0.00 | 1MOD |
| ATOM | 1929 | O | ALA | 241 | −2.734 | 12.151 | −3.582 | 1.00 | 0.00 | 1MOD |
| ATOM | 1930 | CB | ALA | 241 | −4.623 | 11.507 | −1.542 | 1.00 | 0.00 | 1MOD |
| ATOM | 1931 | N | HIS | 242 | −2.019 | 13.825 | −2.203 | 1.00 | 0.00 | 1MOD |
| ATOM | 1932 | CA | HIS | 242 | −0.662 | 13.915 | −2.679 | 1.00 | 0.00 | 1MOD |
| ATOM | 1933 | C | HIS | 242 | 0.290 | 13.402 | −1.605 | 1.00 | 0.00 | 1MOD |
| ATOM | 1934 | O | HIS | 242 | 1.249 | 12.714 | −1.956 | 1.00 | 0.00 | 1MOD |
| ATOM | 1935 | CB | HIS | 242 | −0.223 | 15.349 | −3.027 | 1.00 | 0.00 | 1MOD |
| ATOM | 1936 | CG | HIS | 242 | −0.887 | 15.882 | −4.264 | 1.00 | 0.00 | 1MOD |
| ATOM | 1937 | ND1 | HIS | 242 | −0.599 | 15.448 | −5.540 | 1.00 | 0.00 | 1MOD |
| ATOM | 1938 | CD2 | HIS | 242 | −1.842 | 16.840 | −4.406 | 1.00 | 0.00 | 1MOD |
| ATOM | 1939 | CE1 | HIS | 242 | −1.388 | 16.158 | −6.385 | 1.00 | 0.00 | 1MOD |
| ATOM | 1940 | NE2 | HIS | 242 | −2.160 | 17.015 | −5.741 | 1.00 | 0.00 | 1MOD |
| ATOM | 1941 | N | MET | 243 | 0.082 | 13.743 | −0.295 | 1.00 | 0.00 | 1MOD |
| ATOM | 1942 | CA | MET | 243 | 0.872 | 13.275 | 0.848 | 1.00 | 0.00 | 1MOD |
| ATOM | 1943 | C | MET | 243 | 0.043 | 13.471 | 2.145 | 1.00 | 0.00 | 1MOD |
| ATOM | 1944 | O | MET | 243 | −0.712 | 14.431 | 2.214 | 1.00 | 0.00 | 1MOD |
| ATOM | 1945 | CB | MET | 243 | 2.186 | 14.042 | 1.091 | 1.00 | 0.00 | 1MOD |
| ATOM | 1946 | CG | MET | 243 | 3.248 | 13.879 | 0.005 | 1.00 | 0.00 | 1MOD |
| ATOM | 1947 | SD | MET | 243 | 4.872 | 14.537 | 0.488 | 1.00 | 0.00 | 1MOD |
| ATOM | 1948 | CE | MET | 243 | 4.314 | 16.253 | 0.689 | 1.00 | 0.00 | 1MOD |
| ATOM | 1949 | N | GLY | 244 | 0.213 | 12.650 | 3.241 | 1.00 | 0.00 | 1MOD |
| ATOM | 1950 | CA | GLY | 244 | −0.528 | 12.610 | 4.504 | 1.00 | 0.00 | 1MOD |
| ATOM | 1951 | C | GLY | 244 | −0.647 | 11.132 | 4.795 | 1.00 | 0.00 | 1MOD |
| ATOM | 1952 | O | GLY | 244 | −0.521 | 10.341 | 3.869 | 1.00 | 0.00 | 1MOD |
| ATOM | 1953 | N | GLN | 245 | −0.779 | 10.693 | 6.074 | 1.00 | 0.00 | 1MOD |
| ATOM | 1954 | CA | GLN | 245 | −0.765 | 9.267 | 6.349 | 1.00 | 0.00 | 1MOD |
| ATOM | 1955 | C | GLN | 245 | −2.002 | 8.525 | 5.932 | 1.00 | 0.00 | 1MOD |
| ATOM | 1956 | O | GLN | 245 | −1.937 | 7.574 | 5.154 | 1.00 | 0.00 | 1MOD |
| ATOM | 1957 | CB | GLN | 245 | −0.538 | 8.946 | 7.838 | 1.00 | 0.00 | 1MOD |
| ATOM | 1958 | CG | GLN | 245 | −1.663 | 9.404 | 8.771 | 1.00 | 0.00 | 1MOD |
| ATOM | 1959 | CD | GLN | 245 | −1.535 | 10.903 | 9.006 | 1.00 | 0.00 | 1MOD |
| ATOM | 1960 | OE1 | GLN | 245 | −1.937 | 11.728 | 8.186 | 1.00 | 0.00 | 1MOD |
| ATOM | 1961 | NE2 | GLN | 245 | −0.964 | 11.273 | 10.181 | 1.00 | 0.00 | 1MOD |
| ATOM | 1962 | N | LYS | 246 | −3.177 | 8.947 | 6.433 | 1.00 | 0.00 | 1MOD |
| ATOM | 1963 | CA | LYS | 246 | −4.394 | 8.252 | 6.126 | 1.00 | 0.00 | 1MOD |
| ATOM | 1964 | C | LYS | 246 | −4.625 | 8.482 | 4.685 | 1.00 | 0.00 | 1MOD |
| ATOM | 1965 | O | LYS | 246 | −5.103 | 7.619 | 3.951 | 1.00 | 0.00 | 1MOD |
| ATOM | 1966 | CB | LYS | 246 | −5.604 | 8.823 | 6.876 | 1.00 | 0.00 | 1MOD |
| ATOM | 1967 | CG | LYS | 246 | −5.488 | 8.666 | 8.391 | 1.00 | 0.00 | 1MOD |
| ATOM | 1968 | CD | LYS | 246 | −6.519 | 9.481 | 9.174 | 1.00 | 0.00 | 1MOD |
| ATOM | 1969 | CE | LYS | 246 | −6.428 | 9.300 | 10.690 | 1.00 | 0.00 | 1MOD |
| ATOM | 1970 | NZ | LYS | 246 | −7.488 | 10.088 | 11.357 | 1.00 | 0.00 | 1MOD |
| ATOM | 1971 | N | HIS | 247 | −4.268 | 9.700 | 4.259 | 1.00 | 0.00 | 1MOD |
| ATOM | 1972 | CA | HIS | 247 | −4.454 | 10.110 | 2.910 | 1.00 | 0.00 | 1MOD |
| ATOM | 1973 | C | HIS | 247 | −3.637 | 9.210 | 2.042 | 1.00 | 0.00 | 1MOD |
| ATOM | 1974 | O | HIS | 247 | −4.085 | 8.801 | 0.974 | 1.00 | 0.00 | 1MOD |
| ATOM | 1975 | CB | HIS | 247 | −3.931 | 11.536 | 2.658 | 1.00 | 0.00 | 1MOD |
| ATOM | 1976 | CG | HIS | 247 | −4.597 | 12.583 | 3.497 | 1.00 | 0.00 | 1MOD |
| ATOM | 1977 | ND1 | HIS | 247 | −4.469 | 12.665 | 4.865 | 1.00 | 0.00 | 1MOD |
| ATOM | 1978 | CD2 | HIS | 247 | −5.407 | 13.616 | 3.138 | 1.00 | 0.00 | 1MOD |
| ATOM | 1979 | CE1 | HIS | 247 | −5.207 | 13.732 | 5.264 | 1.00 | 0.00 | 1MOD |
| ATOM | 1980 | NE2 | HIS | 247 | −5.796 | 14.340 | 4.253 | 1.00 | 0.00 | 1MOD |
| ATOM | 1981 | N | ARG | 248 | −2.400 | 8.905 | 2.483 | 1.00 | 0.00 | 1MOD |
| ATOM | 1982 | CA | ARG | 248 | −1.460 | 8.150 | 1.709 | 1.00 | 0.00 | 1MOD |
| ATOM | 1983 | C | ARG | 248 | −1.930 | 6.745 | 1.525 | 1.00 | 0.00 | 1MOD |
| ATOM | 1984 | O | ARG | 248 | −1.879 | 6.202 | 0.423 | 1.00 | 0.00 | 1MOD |
| ATOM | 1985 | CB | ARG | 248 | −0.061 | 8.108 | 2.344 | 1.00 | 0.00 | 1MOD |
| ATOM | 1986 | CG | ARG | 248 | 1.044 | 7.751 | 1.349 | 1.00 | 0.00 | 1MOD |
| ATOM | 1987 | CD | ARG | 248 | 2.451 | 8.018 | 1.885 | 1.00 | 0.00 | 1MOD |
| ATOM | 1988 | NE | ARG | 248 | 3.412 | 7.713 | 0.788 | 1.00 | 0.00 | 1MOD |
| ATOM | 1989 | CZ | ARG | 248 | 4.325 | 8.654 | 0.409 | 1.00 | 0.00 | 1MOD |
| ATOM | 1990 | NH1 | ARG | 248 | 4.350 | 9.866 | 1.034 | 1.00 | 0.00 | 1MOD |
| ATOM | 1991 | NH2 | ARG | 248 | 5.200 | 8.393 | −0.606 | 1.00 | 0.00 | 1MOD |
| ATOM | 1992 | N | ALA | 249 | −2.430 | 6.119 | 2.603 | 1.00 | 0.00 | 1MOD |
| ATOM | 1993 | CA | ALA | 249 | −2.834 | 4.753 | 2.516 | 1.00 | 0.00 | 1MOD |
| ATOM | 1994 | C | ALA | 249 | −3.929 | 4.649 | 1.503 | 1.00 | 0.00 | 1MOD |
| ATOM | 1995 | O | ALA | 249 | −3.981 | 3.702 | 0.724 | 1.00 | 0.00 | 1MOD |
| ATOM | 1996 | CB | ALA | 249 | −3.368 | 4.207 | 3.852 | 1.00 | 0.00 | 1MOD |
| ATOM | 1997 | N | MET | 250 | −4.839 | 5.633 | 1.477 | 1.00 | 0.00 | 1MOD |
| ATOM | 1998 | CA | MET | 250 | −5.947 | 5.599 | 0.569 | 1.00 | 0.00 | 1MOD |
| ATOM | 1999 | C | MET | 250 | −5.430 | 5.665 | −0.831 | 1.00 | 0.00 | 1MOD |
| ATOM | 2000 | O | MET | 250 | −5.986 | 5.064 | −1.749 | 1.00 | 0.00 | 1MOD |
| ATOM | 2001 | CB | MET | 250 | −6.911 | 6.777 | 0.783 | 1.00 | 0.00 | 1MOD |
| ATOM | 2002 | CG | MET | 250 | −8.188 | 6.700 | −0.056 | 1.00 | 0.00 | 1MOD |
| ATOM | 2003 | SD | MET | 250 | −9.402 | 8.003 | 0.308 | 1.00 | 0.00 | 1MOD |
| ATOM | 2004 | CE | MET | 250 | −9.929 | 7.267 | 1.884 | 1.00 | 0.00 | 1MOD |
| ATOM | 2005 | N | ARG | 251 | −4.339 | 6.412 | −1.036 | 1.00 | 0.00 | 1MOD |
| ATOM | 2006 | CA | ARG | 251 | −3.793 | 6.556 | −2.348 | 1.00 | 0.00 | 1MOD |
| ATOM | 2007 | C | ARG | 251 | −3.467 | 5.182 | −2.838 | 1.00 | 0.00 | 1MOD |
| ATOM | 2008 | O | ARG | 251 | −3.765 | 4.845 | −3.983 | 1.00 | 0.00 | 1MOD |
| ATOM | 2009 | CB | ARG | 251 | −2.488 | 7.377 | −2.321 | 1.00 | 0.00 | 1MOD |
| ATOM | 2010 | CG | ARG | 251 | −1.829 | 7.585 | −3.684 | 1.00 | 0.00 | 1MOD |
| ATOM | 2011 | CD | ARG | 251 | −2.604 | 8.520 | −4.611 | 1.00 | 0.00 | 1MOD |
| ATOM | 2012 | NE | ARG | 251 | −1.821 | 8.626 | −5.873 | 1.00 | 0.00 | 1MOD |
| ATOM | 2013 | CZ | ARG | 251 | −2.469 | 8.860 | −7.051 | 1.00 | 0.00 | 1MOD |
| ATOM | 2014 | NH1 | ARG | 251 | −3.828 | 8.970 | −7.073 | 1.00 | 0.00 | 1MOD |
| ATOM | 2015 | NH2 | ARG | 251 | −1.756 | 8.978 | −8.207 | 1.00 | 0.00 | 1MOD |
| ATOM | 2016 | N | VAL | 252 | −2.849 | 4.347 | −1.979 | 1.00 | 0.00 | 1MOD |
| ATOM | 2017 | CA | VAL | 252 | −2.477 | 3.025 | −2.397 | 1.00 | 0.00 | 1MOD |
| ATOM | 2018 | C | VAL | 252 | −3.690 | 2.181 | −2.610 | 1.00 | 0.00 | 1MOD |
| ATOM | 2019 | O | VAL | 252 | −3.739 | 1.400 | −3.558 | 1.00 | 0.00 | 1MOD |
| ATOM | 2020 | CB | VAL | 252 | −1.574 | 2.270 | −1.452 | 1.00 | 0.00 | 1MOD |
| ATOM | 2021 | CG1 | VAL | 252 | −2.373 | 1.734 | −0.257 | 1.00 | 0.00 | 1MOD |
| ATOM | 2022 | CG2 | VAL | 252 | −0.919 | 1.116 | −2.229 | 1.00 | 0.00 | 1MOD |
| ATOM | 2023 | N | ILE | 253 | −4.712 | 2.312 | −1.741 | 1.00 | 0.00 | 1MOD |
| ATOM | 2024 | CA | ILE | 253 | −5.855 | 1.452 | −1.850 | 1.00 | 0.00 | 1MOD |
| ATOM | 2025 | C | ILE | 253 | −6.446 | 1.677 | −3.202 | 1.00 | 0.00 | 1MOD |
| ATOM | 2026 | O | ILE | 253 | −6.923 | 0.740 | −3.841 | 1.00 | 0.00 | 1MOD |
| ATOM | 2027 | CB | ILE | 253 | −6.900 | 1.690 | −0.785 | 1.00 | 0.00 | 1MOD |
| ATOM | 2028 | CG1 | ILE | 253 | −7.836 | 0.478 | −0.635 | 1.00 | 0.00 | 1MOD |
| ATOM | 2029 | CG2 | ILE | 253 | −7.652 | 2.988 | −1.113 | 1.00 | 0.00 | 1MOD |
| ATOM | 2030 | CD1 | ILE | 253 | −8.713 | 0.192 | −1.851 | 1.00 | 0.00 | 1MOD |
| ATOM | 2031 | N | PHE | 254 | −6.437 | 2.941 | −3.666 | 1.00 | 0.00 | 1MOD |
| ATOM | 2032 | CA | PHE | 254 | −6.946 | 3.280 | −4.963 | 1.00 | 0.00 | 1MOD |
| ATOM | 2033 | C | PHE | 254 | −6.110 | 2.647 | −6.031 | 1.00 | 0.00 | 1MOD |
| ATOM | 2034 | O | PHE | 254 | −6.634 | 2.093 | −6.994 | 1.00 | 0.00 | 1MOD |
| ATOM | 2035 | CB | PHE | 254 | −6.949 | 4.793 | −5.235 | 1.00 | 0.00 | 1MOD |
| ATOM | 2036 | CG | PHE | 254 | −7.491 | 4.978 | −6.608 | 1.00 | 0.00 | 1MOD |
| ATOM | 2037 | CD1 | PHE | 254 | −8.844 | 4.897 | −6.845 | 1.00 | 0.00 | 1MOD |
| ATOM | 2038 | CD2 | PHE | 254 | −6.645 | 5.236 | −7.662 | 1.00 | 0.00 | 1MOD |
| ATOM | 2039 | CE1 | PHE | 254 | −9.345 | 5.069 | −8.115 | 1.00 | 0.00 | 1MOD |
| ATOM | 2040 | CE2 | PHE | 254 | −7.138 | 5.411 | −8.934 | 1.00 | 0.00 | 1MOD |
| ATOM | 2041 | CZ | PHE | 254 | −8.491 | 5.329 | −9.160 | 1.00 | 0.00 | 1MOD |
| ATOM | 2042 | N | ALA | 255 | −4.777 | 2.703 | −5.888 | 1.00 | 0.00 | 1MOD |
| ATOM | 2043 | CA | ALA | 255 | −3.922 | 2.193 | −6.921 | 1.00 | 0.00 | 1MOD |
| ATOM | 2044 | C | ALA | 255 | −4.207 | 0.737 | −7.103 | 1.00 | 0.00 | 1MOD |
| ATOM | 2045 | O | ALA | 255 | −4.211 | 0.226 | −8.223 | 1.00 | 0.00 | 1MOD |
| ATOM | 2046 | CB | ALA | 255 | −2.431 | 2.333 | −6.579 | 1.00 | 0.00 | 1MOD |
| ATOM | 2047 | N | VAL | 256 | −4.463 | 0.034 | −5.991 | 1.00 | 0.00 | 1MOD |
| ATOM | 2048 | CA | VAL | 256 | −4.707 | −1.375 | −6.020 | 1.00 | 0.00 | 1MOD |
| ATOM | 2049 | C | VAL | 256 | −5.893 | −1.663 | −6.884 | 1.00 | 0.00 | 1MOD |
| ATOM | 2050 | O | VAL | 256 | −5.889 | −2.631 | −7.644 | 1.00 | 0.00 | 1MOD |
| ATOM | 2051 | CB | VAL | 256 | −5.034 | −1.912 | −4.659 | 1.00 | 0.00 | 1MOD |
| ATOM | 2052 | CG1 | VAL | 256 | −5.512 | −3.356 | −4.823 | 1.00 | 0.00 | 1MOD |
| ATOM | 2053 | CG2 | VAL | 256 | −3.810 | −1.765 | −3.740 | 1.00 | 0.00 | 1MOD |
| ATOM | 2054 | N | VAL | 257 | −6.958 | −0.849 | −6.777 | 1.00 | 0.00 | 1MOD |
| ATOM | 2055 | CA | VAL | 257 | −8.144 | −1.132 | −7.532 | 1.00 | 0.00 | 1MOD |
| ATOM | 2056 | C | VAL | 257 | −7.884 | −0.983 | −9.001 | 1.00 | 0.00 | 1MOD |
| ATOM | 2057 | O | VAL | 257 | −8.364 | −1.779 | −9.806 | 1.00 | 0.00 | 1MOD |
| ATOM | 2058 | CB | VAL | 257 | −9.320 | −0.265 | −7.172 | 1.00 | 0.00 | 1MOD |
| ATOM | 2059 | CG1 | VAL | 257 | −9.102 | 1.154 | −7.715 | 1.00 | 0.00 | 1MOD |
| ATOM | 2060 | CG2 | VAL | 257 | −10.595 | −0.934 | −7.710 | 1.00 | 0.00 | 1MOD |
| ATOM | 2061 | N | LEU | 258 | −7.107 | 0.043 | −9.390 | 1.00 | 0.00 | 1MOD |
| ATOM | 2062 | CA | LEU | 258 | −6.873 | 0.306 | −10.782 | 1.00 | 0.00 | 1MOD |
| ATOM | 2063 | C | LEU | 258 | −6.176 | −0.875 | −11.381 | 1.00 | 0.00 | 1MOD |
| ATOM | 2064 | O | LEU | 258 | −6.522 | −1.334 | −12.469 | 1.00 | 0.00 | 1MOD |
| ATOM | 2065 | CB | LEU | 258 | −5.971 | 1.536 | −10.979 | 1.00 | 0.00 | 1MOD |
| ATOM | 2066 | CG | LEU | 258 | −5.746 | 1.925 | −12.450 | 1.00 | 0.00 | 1MOD |
| ATOM | 2067 | CD1 | LEU | 258 | −7.058 | 2.384 | −13.106 | 1.00 | 0.00 | 1MOD |
| ATOM | 2068 | CD2 | LEU | 258 | −4.617 | 2.960 | −12.584 | 1.00 | 0.00 | 1MOD |
| ATOM | 2069 | N | ILE | 259 | −5.177 | −1.403 | −10.658 | 1.00 | 0.00 | 1MOD |
| ATOM | 2070 | CA | ILE | 259 | −4.379 | −2.509 | −11.106 | 1.00 | 0.00 | 1MOD |
| ATOM | 2071 | C | ILE | 259 | −5.262 | −3.703 | −11.266 | 1.00 | 0.00 | 1MOD |
| ATOM | 2072 | O | ILE | 259 | −5.141 | −4.467 | −12.224 | 1.00 | 0.00 | 1MOD |
| ATOM | 2073 | CB | ILE | 259 | −3.321 | −2.873 | −10.107 | 1.00 | 0.00 | 1MOD |
| ATOM | 2074 | CG1 | ILE | 259 | −2.383 | −1.685 | −9.829 | 1.00 | 0.00 | 1MOD |
| ATOM | 2075 | CG2 | ILE | 259 | −2.597 | −4.112 | −10.647 | 1.00 | 0.00 | 1MOD |
| ATOM | 2076 | CD1 | ILE | 259 | −1.514 | −1.893 | −8.587 | 1.00 | 0.00 | 1MOD |
| ATOM | 2077 | N | PHE | 260 | −6.186 | −3.887 | −10.312 | 1.00 | 0.00 | 1MOD |
| ATOM | 2078 | CA | PHE | 260 | −7.064 | −5.016 | −10.299 | 1.00 | 0.00 | 1MOD |
| ATOM | 2079 | C | PHE | 260 | −7.827 | −5.055 | −11.582 | 1.00 | 0.00 | 1MOD |
| ATOM | 2080 | O | PHE | 260 | −7.888 | −6.090 | −12.244 | 1.00 | 0.00 | 1MOD |
| ATOM | 2081 | CB | PHE | 260 | −8.091 | −4.885 | −9.164 | 1.00 | 0.00 | 1MOD |
| ATOM | 2082 | CG | PHE | 260 | −9.128 | −5.930 | −9.370 | 1.00 | 0.00 | 1MOD |
| ATOM | 2083 | CD1 | PHE | 260 | −8.872 | −7.232 | −9.021 | 1.00 | 0.00 | 1MOD |
| ATOM | 2084 | CD2 | PHE | 260 | −10.354 | −5.607 | −9.906 | 1.00 | 0.00 | 1MOD |
| ATOM | 2085 | CE1 | PHE | 260 | −9.828 | −8.197 | −9.212 | 1.00 | 0.00 | 1MOD |
| ATOM | 2086 | CE2 | PHE | 260 | −11.316 | −6.570 | −10.098 | 1.00 | 0.00 | 1MOD |
| ATOM | 2087 | CZ | PHE | 260 | −11.051 | −7.871 | −9.747 | 1.00 | 0.00 | 1MOD |
| ATOM | 2088 | N | LEU | 261 | −8.415 | −3.913 | −11.971 | 1.00 | 0.00 | 1MOD |
| ATOM | 2089 | CA | LEU | 261 | −9.230 | −3.849 | −13.146 | 1.00 | 0.00 | 1MOD |
| ATOM | 2090 | C | LEU | 261 | −8.405 | −4.137 | −14.357 | 1.00 | 0.00 | 1MOD |
| ATOM | 2091 | O | LEU | 261 | −8.829 | −4.861 | −15.252 | 1.00 | 0.00 | 1MOD |
| ATOM | 2092 | CB | LEU | 261 | −9.847 | −2.457 | −13.369 | 1.00 | 0.00 | 1MOD |
| ATOM | 2093 | CG | LEU | 261 | −10.834 | −2.024 | −12.270 | 1.00 | 0.00 | 1MOD |
| ATOM | 2094 | CD1 | LEU | 261 | −11.406 | −0.628 | −12.559 | 1.00 | 0.00 | 1MOD |
| ATOM | 2095 | CD2 | LEU | 261 | −11.935 | −3.072 | −12.063 | 1.00 | 0.00 | 1MOD |
| ATOM | 2096 | N | LEU | 262 | −7.185 | −3.578 | −14.406 | 1.00 | 0.00 | 1MOD |
| ATOM | 2097 | CA | LEU | 262 | −6.399 | −3.711 | −15.591 | 1.00 | 0.00 | 1MOD |
| ATOM | 2098 | C | LEU | 262 | −6.161 | −5.163 | −15.833 | 1.00 | 0.00 | 1MOD |
| ATOM | 2099 | O | LEU | 262 | −6.295 | −5.644 | −16.957 | 1.00 | 0.00 | 1MOD |
| ATOM | 2100 | CB | LEU | 262 | −5.037 | −3.008 | −15.444 | 1.00 | 0.00 | 1MOD |
| ATOM | 2101 | CG | LEU | 262 | −4.223 | −2.879 | −16.748 | 1.00 | 0.00 | 1MOD |
| ATOM | 2102 | CD1 | LEU | 262 | −3.778 | −4.243 | −17.296 | 1.00 | 0.00 | 1MOD |
| ATOM | 2103 | CD2 | LEU | 262 | −4.971 | −2.026 | −17.785 | 1.00 | 0.00 | 1MOD |
| ATOM | 2104 | N | CYS | 263 | −5.786 | −5.900 | −14.778 | 1.00 | 0.00 | 1MOD |
| ATOM | 2105 | CA | CYS | 263 | −5.483 | −7.290 | −14.922 | 1.00 | 0.00 | 1MOD |
| ATOM | 2106 | C | CYS | 263 | −6.683 | −8.095 | −15.325 | 1.00 | 0.00 | 1MOD |
| ATOM | 2107 | O | CYS | 263 | −6.653 | −8.816 | −16.321 | 1.00 | 0.00 | 1MOD |
| ATOM | 2108 | CB | CYS | 263 | −4.981 | −7.866 | −13.591 | 1.00 | 0.00 | 1MOD |
| ATOM | 2109 | SG | CYS | 263 | −4.590 | −9.629 | −13.688 | 1.00 | 0.00 | 1MOD |
| ATOM | 2110 | N | TRP | 264 | −7.759 | −8.014 | −14.524 | 1.00 | 0.00 | 1MOD |
| ATOM | 2111 | CA | TRP | 264 | −8.930 | −8.823 | −14.721 | 1.00 | 0.00 | 1MOD |
| ATOM | 2112 | C | TRP | 264 | −9.844 | −8.386 | −15.814 | 1.00 | 0.00 | 1MOD |
| ATOM | 2113 | O | TRP | 264 | −10.511 | −9.228 | −16.417 | 1.00 | 0.00 | 1MOD |
| ATOM | 2114 | CB | TRP | 264 | −9.731 | −9.062 | −13.435 | 1.00 | 0.00 | 1MOD |
| ATOM | 2115 | CG | TRP | 264 | −8.945 | −9.967 | −12.522 | 1.00 | 0.00 | 1MOD |
| ATOM | 2116 | CD1 | TRP | 264 | −8.426 | −9.750 | −11.281 | 1.00 | 0.00 | 1MOD |
| ATOM | 2117 | CD2 | TRP | 264 | −8.519 | −11.278 | −12.921 | 1.00 | 0.00 | 1MOD |
| ATOM | 2118 | NE1 | TRP | 264 | −7.709 | −10.851 | −10.874 | 1.00 | 0.00 | 1MOD |
| ATOM | 2119 | CE2 | TRP | 264 | −7.753 | −11.798 | −11.879 | 1.00 | 0.00 | 1MOD |
| ATOM | 2120 | CE3 | TRP | 264 | −8.736 | −11.982 | −14.070 | 1.00 | 0.00 | 1MOD |
| ATOM | 2121 | CZ2 | TRP | 264 | −7.188 | −13.039 | −11.973 | 1.00 | 0.00 | 1MOD |
| ATOM | 2122 | CZ3 | TRP | 264 | −8.180 | −13.237 | −14.157 | 1.00 | 0.00 | 1MOD |
| ATOM | 2123 | CH2 | TRP | 264 | −7.420 | −13.755 | −13.127 | 1.00 | 0.00 | 1MOD |
| ATOM | 2124 | N | LEU | 265 | −9.943 | −7.079 | −16.093 | 1.00 | 0.00 | 1MOD |
| ATOM | 2125 | CA | LEU | 265 | −10.965 | −6.646 | −17.001 | 1.00 | 0.00 | 1MOD |
| ATOM | 2126 | C | LEU | 265 | −10.799 | −7.299 | −18.339 | 1.00 | 0.00 | 1MOD |
| ATOM | 2127 | O | LEU | 265 | −11.786 | −7.748 | −18.912 | 1.00 | 0.00 | 1MOD |
| ATOM | 2128 | CB | LEU | 265 | −11.007 | −5.124 | −17.197 | 1.00 | 0.00 | 1MOD |
| ATOM | 2129 | CG | LEU | 265 | −12.228 | −4.664 | −18.012 | 1.00 | 0.00 | 1MOD |
| ATOM | 2130 | CD1 | LEU | 265 | −13.535 | −5.036 | −17.292 | 1.00 | 0.00 | 1MOD |
| ATOM | 2131 | CD2 | LEU | 265 | −12.148 | −3.165 | −18.341 | 1.00 | 0.00 | 1MOD |
| ATOM | 2132 | N | PRO | 266 | −9.615 | −7.389 | −18.874 | 1.00 | 0.00 | 1MOD |
| ATOM | 2133 | CA | PRO | 266 | −9.458 | −8.032 | −20.146 | 1.00 | 0.00 | 1MOD |
| ATOM | 2134 | C | PRO | 266 | −9.814 | −9.483 | −20.087 | 1.00 | 0.00 | 1MOD |
| ATOM | 2135 | O | PRO | 266 | −10.326 | −10.008 | −21.073 | 1.00 | 0.00 | 1MOD |
| ATOM | 2136 | CB | PRO | 266 | −8.024 | −7.736 | −20.596 | 1.00 | 0.00 | 1MOD |
| ATOM | 2137 | CG | PRO | 266 | −7.357 | −7.064 | −19.377 | 1.00 | 0.00 | 1MOD |
| ATOM | 2138 | CD | PRO | 266 | −8.537 | −6.460 | −18.598 | 1.00 | 0.00 | 1MOD |
| ATOM | 2139 | N | TYR | 267 | −9.516 | −10.156 | −18.960 | 1.00 | 0.00 | 1MOD |
| ATOM | 2140 | CA | TYR | 267 | −9.839 | −11.543 | −18.786 | 1.00 | 0.00 | 1MOD |
| ATOM | 2141 | C | TYR | 267 | −11.314 | −11.727 | −18.647 | 1.00 | 0.00 | 1MOD |
| ATOM | 2142 | O | TYR | 267 | −11.911 | −12.601 | −19.270 | 1.00 | 0.00 | 1MOD |
| ATOM | 2143 | CB | TYR | 267 | −9.181 | −12.129 | −17.534 | 1.00 | 0.00 | 1MOD |
| ATOM | 2144 | CG | TYR | 267 | −9.894 | −13.387 | −17.203 | 1.00 | 0.00 | 1MOD |
| ATOM | 2145 | CD1 | TYR | 267 | −9.737 | −14.508 | −17.978 | 1.00 | 0.00 | 1MOD |
| ATOM | 2146 | CD2 | TYR | 267 | −10.712 | −13.436 | −16.099 | 1.00 | 0.00 | 1MOD |
| ATOM | 2147 | CE1 | TYR | 267 | −10.394 | −15.670 | −17.656 | 1.00 | 0.00 | 1MOD |
| ATOM | 2148 | CE2 | TYR | 267 | −11.373 | −14.593 | −15.770 | 1.00 | 0.00 | 1MOD |
| ATOM | 2149 | CZ | TYR | 267 | −11.212 | −15.709 | −16.553 | 1.00 | 0.00 | 1MOD |
| ATOM | 2150 | OH | TYR | 267 | −11.886 | −16.902 | −16.225 | 1.00 | 0.00 | 1MOD |
| ATOM | 2151 | N | ASN | 268 | −11.956 | −10.900 | −17.807 | 1.00 | 0.00 | 1MOD |
| ATOM | 2152 | CA | ASN | 268 | −13.359 | −11.071 | −17.607 | 1.00 | 0.00 | 1MOD |
| ATOM | 2153 | C | ASN | 268 | −14.069 | −10.731 | −18.872 | 1.00 | 0.00 | 1MOD |
| ATOM | 2154 | O | ASN | 268 | −15.066 | −11.363 | −19.212 | 1.00 | 0.00 | 1MOD |
| ATOM | 2155 | CB | ASN | 268 | −13.948 | −10.255 | −16.435 | 1.00 | 0.00 | 1MOD |
| ATOM | 2156 | CG | ASN | 268 | −13.816 | −8.757 | −16.662 | 1.00 | 0.00 | 1MOD |
| ATOM | 2157 | OD1 | ASN | 268 | −14.340 | −8.197 | −17.623 | 1.00 | 0.00 | 1MOD |
| ATOM | 2158 | ND2 | ASN | 268 | −13.106 | −8.077 | −15.722 | 1.00 | 0.00 | 1MOD |
| ATOM | 2159 | N | LEU | 269 | −13.577 | −9.724 | −19.615 | 1.00 | 0.00 | 1MOD |
| ATOM | 2160 | CA | LEU | 269 | −14.244 | −9.326 | −20.820 | 1.00 | 0.00 | 1MOD |
| ATOM | 2161 | C | LEU | 269 | −14.232 | −10.463 | −21.795 | 1.00 | 0.00 | 1MOD |
| ATOM | 2162 | O | LEU | 269 | −15.258 | −10.821 | −22.370 | 1.00 | 0.00 | 1MOD |
| ATOM | 2163 | CB | LEU | 269 | −13.547 | −8.134 | −21.497 | 1.00 | 0.00 | 1MOD |
| ATOM | 2164 | CG | LEU | 269 | −14.223 | −7.666 | −22.797 | 1.00 | 0.00 | 1MOD |
| ATOM | 2165 | CD1 | LEU | 269 | −15.611 | −7.074 | −22.515 | 1.00 | 0.00 | 1MOD |
| ATOM | 2166 | CD2 | LEU | 269 | −13.309 | −6.709 | −23.576 | 1.00 | 0.00 | 1MOD |
| ATOM | 2167 | N | VAL | 270 | −13.056 | −11.083 | −21.991 | 1.00 | 0.00 | 1MOD |
| ATOM | 2168 | CA | VAL | 270 | −12.968 | −12.161 | −22.927 | 1.00 | 0.00 | 1MOD |
| ATOM | 2169 | C | VAL | 270 | −13.829 | −13.280 | −22.444 | 1.00 | 0.00 | 1MOD |
| ATOM | 2170 | O | VAL | 270 | −14.498 | −13.946 | −23.234 | 1.00 | 0.00 | 1MOD |
| ATOM | 2171 | CB | VAL | 270 | −11.563 | −12.657 | −23.122 | 1.00 | 0.00 | 1MOD |
| ATOM | 2172 | CG1 | VAL | 270 | −10.862 | −12.877 | −21.774 | 1.00 | 0.00 | 1MOD |
| ATOM | 2173 | CG2 | VAL | 270 | −11.681 | −13.962 | −23.902 | 1.00 | 0.00 | 1MOD |
| ATOM | 2174 | N | LEU | 271 | −13.832 | −13.498 | −21.119 | 1.00 | 0.00 | 1MOD |
| ATOM | 2175 | CA | LEU | 271 | −14.572 | −14.555 | −20.502 | 1.00 | 0.00 | 1MOD |
| ATOM | 2176 | C | LEU | 271 | −16.020 | −14.319 | −20.786 | 1.00 | 0.00 | 1MOD |
| ATOM | 2177 | O | LEU | 271 | −16.744 | −15.232 | −21.182 | 1.00 | 0.00 | 1MOD |
| ATOM | 2178 | CB | LEU | 271 | −14.353 | −14.531 | −18.971 | 1.00 | 0.00 | 1MOD |
| ATOM | 2179 | CG | LEU | 271 | −14.955 | −15.685 | −18.133 | 1.00 | 0.00 | 1MOD |
| ATOM | 2180 | CD1 | LEU | 271 | −14.625 | −15.478 | −16.645 | 1.00 | 0.00 | 1MOD |
| ATOM | 2181 | CD2 | LEU | 271 | −16.464 | −15.895 | −18.352 | 1.00 | 0.00 | 1MOD |
| ATOM | 2182 | N | LEU | 272 | −16.473 | −13.068 | −20.605 | 1.00 | 0.00 | 1MOD |
| ATOM | 2183 | CA | LEU | 272 | −17.861 | −12.744 | −20.735 | 1.00 | 0.00 | 1MOD |
| ATOM | 2184 | C | LEU | 272 | −18.288 | −12.970 | −22.142 | 1.00 | 0.00 | 1MOD |
| ATOM | 2185 | O | LEU | 272 | −19.351 | −13.533 | −22.395 | 1.00 | 0.00 | 1MOD |
| ATOM | 2186 | CB | LEU | 272 | −18.147 | −11.274 | −20.380 | 1.00 | 0.00 | 1MOD |
| ATOM | 2187 | CG | LEU | 272 | −19.641 | −10.909 | −20.237 | 1.00 | 0.00 | 1MOD |
| ATOM | 2188 | CD1 | LEU | 272 | −20.416 | −11.014 | −21.558 | 1.00 | 0.00 | 1MOD |
| ATOM | 2189 | CD2 | LEU | 272 | −20.294 | −11.704 | −19.097 | 1.00 | 0.00 | 1MOD |
| ATOM | 2190 | N | ALA | 273 | −17.457 | −12.538 | −23.104 | 1.00 | 0.00 | 1MOD |
| ATOM | 2191 | CA | ALA | 273 | −17.832 | −12.655 | −24.481 | 1.00 | 0.00 | 1MOD |
| ATOM | 2192 | C | ALA | 273 | −17.987 | −14.096 | −24.846 | 1.00 | 0.00 | 1MOD |
| ATOM | 2193 | O | ALA | 273 | −18.912 | −14.453 | −25.576 | 1.00 | 0.00 | 1MOD |
| ATOM | 2194 | CB | ALA | 273 | −16.810 | −12.017 | −25.440 | 1.00 | 0.00 | 1MOD |
| ATOM | 2195 | N | ASP | 274 | −17.095 | −14.970 | −24.347 | 1.00 | 0.00 | 1MOD |
| ATOM | 2196 | CA | ASP | 274 | −17.170 | −16.342 | −24.756 | 1.00 | 0.00 | 1MOD |
| ATOM | 2197 | C | ASP | 274 | −18.499 | −16.879 | −24.370 | 1.00 | 0.00 | 1MOD |
| ATOM | 2198 | O | ASP | 274 | −19.185 | −17.503 | −25.179 | 1.00 | 0.00 | 1MOD |
| ATOM | 2199 | CB | ASP | 274 | −16.095 | −17.220 | −24.113 | 1.00 | 0.00 | 1MOD |
| ATOM | 2200 | CG | ASP | 274 | −14.794 | −16.898 | −24.826 | 1.00 | 0.00 | 1MOD |
| ATOM | 2201 | OD1 | ASP | 274 | −14.868 | −16.363 | −25.963 | 1.00 | 0.00 | 1MOD |
| ATOM | 2202 | OD2 | ASP | 274 | −13.708 | −17.168 | −24.249 | 1.00 | 0.00 | 1MOD |
| ATOM | 2203 | N | THR | 275 | −18.912 | −16.628 | −23.121 | 1.00 | 0.00 | 1MOD |
| ATOM | 2204 | CA | THR | 275 | −20.192 | −17.097 | −22.699 | 1.00 | 0.00 | 1MOD |
| ATOM | 2205 | C | THR | 275 | −21.258 | −16.375 | −23.457 | 1.00 | 0.00 | 1MOD |
| ATOM | 2206 | O | THR | 275 | −22.262 | −16.962 | −23.859 | 1.00 | 0.00 | 1MOD |
| ATOM | 2207 | CB | THR | 275 | −20.453 | −16.836 | −21.259 | 1.00 | 0.00 | 1MOD |
| ATOM | 2208 | OG1 | THR | 275 | −19.537 | −17.547 | −20.439 | 1.00 | 0.00 | 1MOD |
| ATOM | 2209 | CG2 | THR | 275 | −21.897 | −17.249 | −20.994 | 1.00 | 0.00 | 1MOD |
| ATOM | 2210 | N | LEU | 276 | −21.052 | −15.067 | −23.678 | 1.00 | 0.00 | 1MOD |
| ATOM | 2211 | CA | LEU | 276 | −22.055 | −14.238 | −24.269 | 1.00 | 0.00 | 1MOD |
| ATOM | 2212 | C | LEU | 276 | −22.380 | −14.735 | −25.644 | 1.00 | 0.00 | 1MOD |
| ATOM | 2213 | O | LEU | 276 | −23.550 | −14.723 | −26.026 | 1.00 | 0.00 | 1MOD |
| ATOM | 2214 | CB | LEU | 276 | −21.613 | −12.760 | −24.331 | 1.00 | 0.00 | 1MOD |
| ATOM | 2215 | CG | LEU | 276 | −22.699 | −11.725 | −24.711 | 1.00 | 0.00 | 1MOD |
| ATOM | 2216 | CD1 | LEU | 276 | −22.124 | −10.304 | −24.639 | 1.00 | 0.00 | 1MOD |
| ATOM | 2217 | CD2 | LEU | 276 | −23.345 | −11.991 | −26.080 | 1.00 | 0.00 | 1MOD |
| ATOM | 2218 | N | MET | 277 | −21.373 | −15.191 | −26.421 | 1.00 | 0.00 | 1MOD |
| ATOM | 2219 | CA | MET | 277 | −21.647 | −15.610 | −27.771 | 1.00 | 0.00 | 1MOD |
| ATOM | 2220 | C | MET | 277 | −22.716 | −16.649 | −27.764 | 1.00 | 0.00 | 1MOD |
| ATOM | 2221 | O | MET | 277 | −22.504 | −17.806 | −27.398 | 1.00 | 0.00 | 1MOD |
| ATOM | 2222 | CB | MET | 277 | −20.427 | −16.197 | −28.505 | 1.00 | 0.00 | 1MOD |
| ATOM | 2223 | CG | MET | 277 | −20.735 | −16.640 | −29.939 | 1.00 | 0.00 | 1MOD |
| ATOM | 2224 | SD | MET | 277 | −21.059 | −15.291 | −31.115 | 1.00 | 0.00 | 1MOD |
| ATOM | 2225 | CE | MET | 277 | −21.423 | −16.389 | −32.516 | 1.00 | 0.00 | 1MOD |
| ATOM | 2226 | N | ARG | 278 | −23.917 | −16.216 | −28.190 | 1.00 | 0.00 | 1MOD |
| ATOM | 2227 | CA | ARG | 278 | −25.057 | −17.071 | −28.242 | 1.00 | 0.00 | 1MOD |
| ATOM | 2228 | C | ARG | 278 | −25.742 | −16.776 | −29.532 | 1.00 | 0.00 | 1MOD |
| ATOM | 2229 | O | ARG | 278 | −25.869 | −15.618 | −29.925 | 1.00 | 0.00 | 1MOD |
| ATOM | 2230 | CB | ARG | 278 | −26.085 | −16.747 | −27.148 | 1.00 | 0.00 | 1MOD |
| ATOM | 2231 | CG | ARG | 278 | −26.554 | −15.296 | −27.255 | 1.00 | 0.00 | 1MOD |
| ATOM | 2232 | CD | ARG | 278 | −27.615 | −14.869 | −26.241 | 1.00 | 0.00 | 1MOD |
| ATOM | 2233 | NE | ARG | 278 | −27.781 | −13.396 | −26.401 | 1.00 | 0.00 | 1MOD |
| ATOM | 2234 | CZ | ARG | 278 | −29.013 | −12.818 | −26.323 | 1.00 | 0.00 | 1MOD |
| ATOM | 2235 | NH1 | ARG | 278 | −30.119 | −13.587 | −26.099 | 1.00 | 0.00 | 1MOD |
| ATOM | 2236 | NH2 | ARG | 278 | −29.139 | −11.467 | −26.477 | 1.00 | 0.00 | 1MOD |
| ATOM | 2237 | N | THR | 279 | −26.189 | −17.832 | −30.233 | 1.00 | 0.00 | 1MOD |
| ATOM | 2238 | CA | THR | 279 | −26.947 | −17.651 | −31.432 | 1.00 | 0.00 | 1MOD |
| ATOM | 2239 | C | THR | 279 | −28.329 | −18.071 | −31.070 | 1.00 | 0.00 | 1MOD |
| ATOM | 2240 | O | THR | 279 | −28.747 | −17.913 | −29.924 | 1.00 | 0.00 | 1MOD |
| ATOM | 2241 | CB | THR | 279 | −26.485 | −18.488 | −32.587 | 1.00 | 0.00 | 1MOD |
| ATOM | 2242 | OG1 | THR | 279 | −26.560 | −19.867 | −32.255 | 1.00 | 0.00 | 1MOD |
| ATOM | 2243 | CG2 | THR | 279 | −25.040 | −18.093 | −32.941 | 1.00 | 0.00 | 1MOD |
| ATOM | 2244 | N | GLN | 280 | −29.091 | −18.609 | −32.037 | 1.00 | 0.00 | 1MOD |
| ATOM | 2245 | CA | GLN | 280 | −30.393 | −19.041 | −31.645 | 1.00 | 0.00 | 1MOD |
| ATOM | 2246 | C | GLN | 280 | −30.150 | −20.145 | −30.678 | 1.00 | 0.00 | 1MOD |
| ATOM | 2247 | O | GLN | 280 | −29.091 | −20.770 | −30.702 | 1.00 | 0.00 | 1MOD |
| ATOM | 2248 | CB | GLN | 280 | −31.266 | −19.580 | −32.786 | 1.00 | 0.00 | 1MOD |
| ATOM | 2249 | CG | GLN | 280 | −32.734 | −19.665 | −32.374 | 1.00 | 0.00 | 1MOD |
| ATOM | 2250 | CD | GLN | 280 | −33.173 | −18.232 | −32.104 | 1.00 | 0.00 | 1MOD |
| ATOM | 2251 | OE1 | GLN | 280 | −34.142 | −17.970 | −31.393 | 1.00 | 0.00 | 1MOD |
| ATOM | 2252 | NE2 | GLN | 280 | −32.416 | −17.262 | −32.686 | 1.00 | 0.00 | 1MOD |
| ATOM | 2253 | N | VAL | 281 | −31.115 | −20.391 | −29.776 | 1.00 | 0.00 | 1MOD |
| ATOM | 2254 | CA | VAL | 281 | −30.884 | −21.374 | −28.765 | 1.00 | 0.00 | 1MOD |
| ATOM | 2255 | C | VAL | 281 | −30.593 | −22.675 | −29.425 | 1.00 | 0.00 | 1MOD |
| ATOM | 2256 | O | VAL | 281 | −31.296 | −23.102 | −30.339 | 1.00 | 0.00 | 1MOD |
| ATOM | 2257 | CB | VAL | 281 | −32.058 | −21.584 | −27.854 | 1.00 | 0.00 | 1MOD |
| ATOM | 2258 | CG1 | VAL | 281 | −32.324 | −20.278 | −27.084 | 1.00 | 0.00 | 1MOD |
| ATOM | 2259 | CG2 | VAL | 281 | −33.248 | −22.070 | −28.700 | 1.00 | 0.00 | 1MOD |
| ATOM | 2260 | N | ILE | 282 | −29.503 | −23.322 | −28.977 | 1.00 | 0.00 | 1MOD |
| ATOM | 2261 | CA | ILE | 282 | −29.163 | −24.610 | −29.488 | 1.00 | 0.00 | 1MOD |
| ATOM | 2262 | C | ILE | 282 | −28.896 | −25.463 | −28.295 | 1.00 | 0.00 | 1MOD |
| ATOM | 2263 | O | ILE | 282 | −28.361 | −24.998 | −27.288 | 1.00 | 0.00 | 1MOD |
| ATOM | 2264 | CB | ILE | 282 | −27.953 | −24.616 | −30.385 | 1.00 | 0.00 | 1MOD |
| ATOM | 2265 | CG1 | ILE | 282 | −27.790 | −25.973 | −31.093 | 1.00 | 0.00 | 1MOD |
| ATOM | 2266 | CG2 | ILE | 282 | −26.734 | −24.200 | −29.552 | 1.00 | 0.00 | 1MOD |
| ATOM | 2267 | CD1 | ILE | 282 | −28.884 | −26.246 | −32.119 | 1.00 | 0.00 | 1MOD |
| ATOM | 2268 | N | GLN | 283 | −29.288 | −26.744 | −28.376 | 1.00 | 0.00 | 1MOD |
| ATOM | 2269 | CA | GLN | 283 | −29.068 | −27.612 | −27.264 | 1.00 | 0.00 | 1MOD |
| ATOM | 2270 | C | GLN | 283 | −27.598 | −27.637 | −27.078 | 1.00 | 0.00 | 1MOD |
| ATOM | 2271 | O | GLN | 283 | −27.096 | −27.667 | −25.956 | 1.00 | 0.00 | 1MOD |
| ATOM | 2272 | CB | GLN | 283 | −29.531 | −29.052 | −27.515 | 1.00 | 0.00 | 1MOD |
| ATOM | 2273 | CG | GLN | 283 | −29.276 | −29.972 | −26.319 | 1.00 | 0.00 | 1MOD |
| ATOM | 2274 | CD | GLN | 283 | −30.059 | −29.447 | −25.123 | 1.00 | 0.00 | 1MOD |
| ATOM | 2275 | OE1 | GLN | 283 | −29.615 | −29.544 | −23.981 | 1.00 | 0.00 | 1MOD |
| ATOM | 2276 | NE2 | GLN | 283 | −31.265 | −28.877 | −25.389 | 1.00 | 0.00 | 1MOD |
| ATOM | 2277 | N | GLU | 284 | −26.868 | −27.601 | −28.205 | 1.00 | 0.00 | 1MOD |
| ATOM | 2278 | CA | GLU | 284 | −25.447 | −27.617 | −28.115 | 1.00 | 0.00 | 1MOD |
| ATOM | 2279 | C | GLU | 284 | −25.048 | −26.378 | −27.384 | 1.00 | 0.00 | 1MOD |
| ATOM | 2280 | O | GLU | 284 | −25.817 | −25.425 | −27.274 | 1.00 | 0.00 | 1MOD |
| ATOM | 2281 | CB | GLU | 284 | −24.734 | −27.685 | −29.477 | 1.00 | 0.00 | 1MOD |
| ATOM | 2282 | CG | GLU | 284 | −23.264 | −28.094 | −29.362 | 1.00 | 0.00 | 1MOD |
| ATOM | 2283 | CD | GLU | 284 | −22.790 | −28.572 | −30.730 | 1.00 | 0.00 | 1MOD |
| ATOM | 2284 | OE1 | GLU | 284 | −23.463 | −29.470 | −31.301 | 1.00 | 0.00 | 1MOD |
| ATOM | 2285 | OE2 | GLU | 284 | −21.740 | −28.065 | −31.210 | 1.00 | 0.00 | 1MOD |
| ATOM | 2286 | N | THR | 285 | −23.826 | −26.383 | −26.830 | 1.00 | 0.00 | 1MOD |
| ATOM | 2287 | CA | THR | 285 | −23.366 | −25.306 | −26.008 | 1.00 | 0.00 | 1MOD |
| ATOM | 2288 | C | THR | 285 | −23.363 | −24.019 | −26.770 | 1.00 | 0.00 | 1MOD |
| ATOM | 2289 | O | THR | 285 | −23.539 | −23.998 | −27.987 | 1.00 | 0.00 | 1MOD |
| ATOM | 2290 | CB | THR | 285 | −21.970 | −25.497 | −25.503 | 1.00 | 0.00 | 1MOD |
| ATOM | 2291 | OG1 | THR | 285 | −21.063 | −25.479 | −26.595 | 1.00 | 0.00 | 1MOD |
| ATOM | 2292 | CG2 | THR | 285 | −21.884 | −26.847 | −24.772 | 1.00 | 0.00 | 1MOD |
| ATOM | 2293 | N | CYS | 286 | −23.203 | −22.896 | −26.030 | 1.00 | 0.00 | 1MOD |
| ATOM | 2294 | CA | CYS | 286 | −23.151 | −21.579 | −26.612 | 1.00 | 0.00 | 1MOD |
| ATOM | 2295 | C | CYS | 286 | −21.762 | −21.046 | −26.412 | 1.00 | 0.00 | 1MOD |
| ATOM | 2296 | O | CYS | 286 | −21.568 | −19.968 | −25.856 | 1.00 | 0.00 | 1MOD |
| ATOM | 2297 | CB | CYS | 286 | −24.082 | −20.581 | −25.905 | 1.00 | 0.00 | 1MOD |
| ATOM | 2298 | SG | CYS | 286 | −25.826 | −21.080 | −25.960 | 1.00 | 0.00 | 1MOD |
| ATOM | 2299 | N | GLU | 287 | −20.757 | −21.872 | −26.730 | 1.00 | 0.00 | 1MOD |
| ATOM | 2300 | CA | GLU | 287 | −19.351 | −21.574 | −26.680 | 1.00 | 0.00 | 1MOD |
| ATOM | 2301 | C | GLU | 287 | −18.730 | −21.261 | −28.012 | 1.00 | 0.00 | 1MOD |
| ATOM | 2302 | O | GLU | 287 | −17.548 | −20.930 | −28.059 | 1.00 | 0.00 | 1MOD |
| ATOM | 2303 | CB | GLU | 287 | −18.540 | −22.718 | −26.029 | 1.00 | 0.00 | 1MOD |
| ATOM | 2304 | CG | GLU | 287 | −17.120 | −22.349 | −25.578 | 1.00 | 0.00 | 1MOD |
| ATOM | 2305 | CD | GLU | 287 | −16.117 | −22.774 | −26.639 | 1.00 | 0.00 | 1MOD |
| ATOM | 2306 | OE1 | GLU | 287 | −16.518 | −23.514 | −27.577 | 1.00 | 0.00 | 1MOD |
| ATOM | 2307 | OE2 | GLU | 287 | −14.934 | −22.358 | −26.528 | 1.00 | 0.00 | 1MOD |
| ATOM | 2308 | N | ARG | 288 | −19.433 | −21.486 | −29.138 | 1.00 | 0.00 | 1MOD |
| ATOM | 2309 | CA | ARG | 288 | −18.738 | −21.458 | −30.405 | 1.00 | 0.00 | 1MOD |
| ATOM | 2310 | C | ARG | 288 | −18.770 | −20.138 | −31.109 | 1.00 | 0.00 | 1MOD |
| ATOM | 2311 | O | ARG | 288 | −19.827 | −19.552 | −31.327 | 1.00 | 0.00 | 1MOD |
| ATOM | 2312 | CB | ARG | 288 | −19.340 | −22.413 | −31.442 | 1.00 | 0.00 | 1MOD |
| ATOM | 2313 | CG | ARG | 288 | −19.282 | −23.897 | −31.107 | 1.00 | 0.00 | 1MOD |
| ATOM | 2314 | CD | ARG | 288 | −20.035 | −24.717 | −32.154 | 1.00 | 0.00 | 1MOD |
| ATOM | 2315 | NE | ARG | 288 | −21.483 | −24.416 | −31.982 | 1.00 | 0.00 | 1MOD |
| ATOM | 2316 | CZ | ARG | 288 | −22.366 | −24.723 | −32.975 | 1.00 | 0.00 | 1MOD |
| ATOM | 2317 | NH1 | ARG | 288 | −21.913 | −25.309 | −34.120 | 1.00 | 0.00 | 1MOD |
| ATOM | 2318 | NH2 | ARG | 288 | −23.693 | −24.439 | −32.822 | 1.00 | 0.00 | 1MOD |
| ATOM | 2319 | N | ARG | 289 | −17.577 | −19.681 | −31.544 | 1.00 | 0.00 | 1MOD |
| ATOM | 2320 | CA | ARG | 289 | −17.417 | −18.493 | −32.337 | 1.00 | 0.00 | 1MOD |
| ATOM | 2321 | C | ARG | 289 | −16.052 | −18.606 | −32.924 | 1.00 | 0.00 | 1MOD |
| ATOM | 2322 | O | ARG | 289 | −15.293 | −19.497 | −32.548 | 1.00 | 0.00 | 1MOD |
| ATOM | 2323 | CB | ARG | 289 | −17.407 | −17.181 | −31.533 | 1.00 | 0.00 | 1MOD |
| ATOM | 2324 | CG | ARG | 289 | −17.311 | −15.934 | −32.417 | 1.00 | 0.00 | 1MOD |
| ATOM | 2325 | CD | ARG | 289 | −16.927 | −14.657 | −31.665 | 1.00 | 0.00 | 1MOD |
| ATOM | 2326 | NE | ARG | 289 | −16.879 | −13.552 | −32.664 | 1.00 | 0.00 | 1MOD |
| ATOM | 2327 | CZ | ARG | 289 | −16.843 | −12.252 | −32.249 | 1.00 | 0.00 | 1MOD |
| ATOM | 2328 | NH1 | ARG | 289 | −16.806 | −11.957 | −30.916 | 1.00 | 0.00 | 1MOD |
| ATOM | 2329 | NH2 | ARG | 289 | −16.855 | −11.241 | −33.163 | 1.00 | 0.00 | 1MOD |
| ATOM | 2330 | N | ASN | 290 | −15.689 | −17.717 | −33.872 | 1.00 | 0.00 | 1MOD |
| ATOM | 2331 | CA | ASN | 290 | −14.336 | −17.803 | −34.318 | 1.00 | 0.00 | 1MOD |
| ATOM | 2332 | C | ASN | 290 | −13.494 | −17.511 | −33.130 | 1.00 | 0.00 | 1MOD |
| ATOM | 2333 | O | ASN | 290 | −13.593 | −16.456 | −32.503 | 1.00 | 0.00 | 1MOD |
| ATOM | 2334 | CB | ASN | 290 | −13.924 | −16.814 | −35.421 | 1.00 | 0.00 | 1MOD |
| ATOM | 2335 | CG | ASN | 290 | −14.166 | −17.438 | −36.787 | 1.00 | 0.00 | 1MOD |
| ATOM | 2336 | OD1 | ASN | 290 | −13.217 | −17.623 | −37.549 | 1.00 | 0.00 | 1MOD |
| ATOM | 2337 | ND2 | ASN | 290 | −15.443 | −17.770 | −37.111 | 1.00 | 0.00 | 1MOD |
| ATOM | 2338 | N | HIS | 291 | −12.645 | −18.483 | −32.776 | 1.00 | 0.00 | 1MOD |
| ATOM | 2339 | CA | HIS | 291 | −11.766 | −18.293 | −31.672 | 1.00 | 0.00 | 1MOD |
| ATOM | 2340 | C | HIS | 291 | −10.469 | −18.932 | −32.002 | 1.00 | 0.00 | 1MOD |
| ATOM | 2341 | O | HIS | 291 | −10.387 | −19.850 | −32.816 | 1.00 | 0.00 | 1MOD |
| ATOM | 2342 | CB | HIS | 291 | −12.244 | −18.841 | −30.316 | 1.00 | 0.00 | 1MOD |
| ATOM | 2343 | CG | HIS | 291 | −13.296 | −17.976 | −29.683 | 1.00 | 0.00 | 1MOD |
| ATOM | 2344 | ND1 | HIS | 291 | −13.126 | −16.630 | −29.444 | 1.00 | 0.00 | 1MOD |
| ATOM | 2345 | CD2 | HIS | 291 | −14.539 | −18.280 | −29.221 | 1.00 | 0.00 | 1MOD |
| ATOM | 2346 | CE1 | HIS | 291 | −14.267 | −16.189 | −28.855 | 1.00 | 0.00 | 1MOD |
| ATOM | 2347 | NE2 | HIS | 291 | −15.153 | −17.155 | −28.699 | 1.00 | 0.00 | 1MOD |
| ATOM | 2348 | N | ILE | 292 | −9.403 | −18.422 | −31.373 | 1.00 | 0.00 | 1MOD |
| ATOM | 2349 | CA | ILE | 292 | −8.097 | −18.924 | −31.623 | 1.00 | 0.00 | 1MOD |
| ATOM | 2350 | C | ILE | 292 | −7.561 | −19.227 | −30.268 | 1.00 | 0.00 | 1MOD |
| ATOM | 2351 | O | ILE | 292 | −8.036 | −18.684 | −29.273 | 1.00 | 0.00 | 1MOD |
| ATOM | 2352 | CB | ILE | 292 | −7.239 | −17.888 | −32.307 | 1.00 | 0.00 | 1MOD |
| ATOM | 2353 | CG1 | ILE | 292 | −5.990 | −18.479 | −32.987 | 1.00 | 0.00 | 1MOD |
| ATOM | 2354 | CG2 | ILE | 292 | −6.917 | −16.805 | −31.261 | 1.00 | 0.00 | 1MOD |
| ATOM | 2355 | CD1 | ILE | 292 | −4.876 | −18.888 | −32.031 | 1.00 | 0.00 | 1MOD |
| ATOM | 2356 | N | ASP | 293 | −6.588 | −20.140 | −30.178 | 1.00 | 0.00 | 1MOD |
| ATOM | 2357 | CA | ASP | 293 | −6.035 | −20.441 | −28.897 | 1.00 | 0.00 | 1MOD |
| ATOM | 2358 | C | ASP | 293 | −5.405 | −19.198 | −28.363 | 1.00 | 0.00 | 1MOD |
| ATOM | 2359 | O | ASP | 293 | −5.352 | −18.991 | −27.153 | 1.00 | 0.00 | 1MOD |
| ATOM | 2360 | CB | ASP | 293 | −5.021 | −21.601 | −28.940 | 1.00 | 0.00 | 1MOD |
| ATOM | 2361 | CG | ASP | 293 | −4.028 | −21.365 | −30.071 | 1.00 | 0.00 | 1MOD |
| ATOM | 2362 | OD1 | ASP | 293 | −4.469 | −21.495 | −31.246 | 1.00 | 0.00 | 1MOD |
| ATOM | 2363 | OD2 | ASP | 293 | −2.839 | −21.050 | −29.795 | 1.00 | 0.00 | 1MOD |
| ATOM | 2364 | N | ARG | 294 | −4.920 | −18.330 | −29.264 | 1.00 | 0.00 | 1MOD |
| ATOM | 2365 | CA | ARG | 294 | −4.250 | −17.117 | −28.899 | 1.00 | 0.00 | 1MOD |
| ATOM | 2366 | C | ARG | 294 | −5.152 | −16.174 | −28.161 | 1.00 | 0.00 | 1MOD |
| ATOM | 2367 | O | ARG | 294 | −4.701 | −15.503 | −27.236 | 1.00 | 0.00 | 1MOD |
| ATOM | 2368 | CB | ARG | 294 | −3.741 | −16.314 | −30.108 | 1.00 | 0.00 | 1MOD |
| ATOM | 2369 | CG | ARG | 294 | −2.615 | −16.986 | −30.891 | 1.00 | 0.00 | 1MOD |
| ATOM | 2370 | CD | ARG | 294 | −1.892 | −16.029 | −31.842 | 1.00 | 0.00 | 1MOD |
| ATOM | 2371 | NE | ARG | 294 | −1.200 | −15.027 | −30.983 | 1.00 | 0.00 | 1MOD |
| ATOM | 2372 | CZ | ARG | 294 | −0.104 | −14.353 | −31.440 | 1.00 | 0.00 | 1MOD |
| ATOM | 2373 | NH1 | ARG | 294 | 0.350 | −14.566 | −32.708 | 1.00 | 0.00 | 1MOD |
| ATOM | 2374 | NH2 | ARG | 294 | 0.543 | −13.468 | −30.626 | 1.00 | 0.00 | 1MOD |
| ATOM | 2375 | N | ALA | 295 | −6.442 | −16.075 | −28.536 | 1.00 | 0.00 | 1MOD |
| ATOM | 2376 | CA | ALA | 295 | −7.233 | −15.008 | −27.980 | 1.00 | 0.00 | 1MOD |
| ATOM | 2377 | C | ALA | 295 | −7.305 | −15.051 | −26.483 | 1.00 | 0.00 | 1MOD |
| ATOM | 2378 | O | ALA | 295 | −7.052 | −14.043 | −25.825 | 1.00 | 0.00 | 1MOD |
| ATOM | 2379 | CB | ALA | 295 | −8.680 | −15.008 | −28.500 | 1.00 | 0.00 | 1MOD |
| ATOM | 2380 | N | LEU | 296 | −7.653 | −16.209 | −25.903 | 1.00 | 0.00 | 1MOD |
| ATOM | 2381 | CA | LEU | 296 | −7.804 | −16.368 | −24.485 | 1.00 | 0.00 | 1MOD |
| ATOM | 2382 | C | LEU | 296 | −6.462 | −16.367 | −23.810 | 1.00 | 0.00 | 1MOD |
| ATOM | 2383 | O | LEU | 296 | −6.322 | −15.915 | −22.675 | 1.00 | 0.00 | 1MOD |
| ATOM | 2384 | CB | LEU | 296 | −8.517 | −17.690 | −24.162 | 1.00 | 0.00 | 1MOD |
| ATOM | 2385 | CG | LEU | 296 | −9.862 | −17.859 | −24.896 | 1.00 | 0.00 | 1MOD |
| ATOM | 2386 | CD1 | LEU | 296 | −10.598 | −19.125 | −24.434 | 1.00 | 0.00 | 1MOD |
| ATOM | 2387 | CD2 | LEU | 296 | −10.725 | −16.598 | −24.790 | 1.00 | 0.00 | 1MOD |
| ATOM | 2388 | N | ASP | 297 | −5.433 | −16.889 | −24.498 | 1.00 | 0.00 | 1MOD |
| ATOM | 2389 | CA | ASP | 297 | −4.142 | −17.101 | −23.906 | 1.00 | 0.00 | 1MOD |
| ATOM | 2390 | C | ASP | 297 | −3.558 | −15.840 | −23.348 | 1.00 | 0.00 | 1MOD |
| ATOM | 2391 | O | ASP | 297 | −3.055 | −15.840 | −22.225 | 1.00 | 0.00 | 1MOD |
| ATOM | 2392 | CB | ASP | 297 | −3.128 | −17.665 | −24.915 | 1.00 | 0.00 | 1MOD |
| ATOM | 2393 | CG | ASP | 297 | −1.832 | −17.961 | −24.181 | 1.00 | 0.00 | 1MOD |
| ATOM | 2394 | OD1 | ASP | 297 | −1.850 | −17.959 | −22.922 | 1.00 | 0.00 | 1MOD |
| ATOM | 2395 | OD2 | ASP | 297 | −0.801 | −18.181 | −24.870 | 1.00 | 0.00 | 1MOD |
| ATOM | 2396 | N | ALA | 298 | −3.593 | −14.731 | −24.105 | 1.00 | 0.00 | 1MOD |
| ATOM | 2397 | CA | ALA | 298 | −2.938 | −13.547 | −23.630 | 1.00 | 0.00 | 1MOD |
| ATOM | 2398 | C | ALA | 298 | −3.575 | −13.021 | −22.379 | 1.00 | 0.00 | 1MOD |
| ATOM | 2399 | O | ALA | 298 | −2.895 | −12.774 | −21.388 | 1.00 | 0.00 | 1MOD |
| ATOM | 2400 | CB | ALA | 298 | −2.953 | −12.413 | −24.669 | 1.00 | 0.00 | 1MOD |
| ATOM | 2401 | N | THR | 299 | −4.907 | −12.871 | −22.358 | 1.00 | 0.00 | 1MOD |
| ATOM | 2402 | CA | THR | 299 | −5.557 | −12.268 | −21.230 | 1.00 | 0.00 | 1MOD |
| ATOM | 2403 | C | THR | 299 | −5.386 | −13.132 | −20.023 | 1.00 | 0.00 | 1MOD |
| ATOM | 2404 | O | THR | 299 | −5.197 | −12.630 | −18.916 | 1.00 | 0.00 | 1MOD |
| ATOM | 2405 | CB | THR | 299 | −7.023 | −12.058 | −21.470 | 1.00 | 0.00 | 1MOD |
| ATOM | 2406 | OG1 | THR | 299 | −7.590 | −11.302 | −20.411 | 1.00 | 0.00 | 1MOD |
| ATOM | 2407 | CG2 | THR | 299 | −7.705 | −13.433 | −21.593 | 1.00 | 0.00 | 1MOD |
| ATOM | 2408 | N | GLU | 300 | −5.470 | −14.464 | −20.205 | 1.00 | 0.00 | 1MOD |
| ATOM | 2409 | CA | GLU | 300 | −5.388 | −15.389 | −19.111 | 1.00 | 0.00 | 1MOD |
| ATOM | 2410 | C | GLU | 300 | −4.026 | −15.329 | −18.489 | 1.00 | 0.00 | 1MOD |
| ATOM | 2411 | O | GLU | 300 | −3.897 | −15.257 | −17.269 | 1.00 | 0.00 | 1MOD |
| ATOM | 2412 | CB | GLU | 300 | −5.561 | −16.857 | −19.552 | 1.00 | 0.00 | 1MOD |
| ATOM | 2413 | CG | GLU | 300 | −6.867 | −17.150 | −20.288 | 1.00 | 0.00 | 1MOD |
| ATOM | 2414 | CD | GLU | 300 | −8.024 | −16.668 | −19.431 | 1.00 | 0.00 | 1MOD |
| ATOM | 2415 | OE1 | GLU | 300 | −7.788 | −16.405 | −18.224 | 1.00 | 0.00 | 1MOD |
| ATOM | 2416 | OE2 | GLU | 300 | −9.154 | −16.538 | −19.973 | 1.00 | 0.00 | 1MOD |
| ATOM | 2417 | N | ILE | 301 | −2.976 | −15.366 | −19.331 | 1.00 | 0.00 | 1MOD |
| ATOM | 2418 | CA | ILE | 301 | −1.614 | −15.400 | −18.881 | 1.00 | 0.00 | 1MOD |
| ATOM | 2419 | C | ILE | 301 | −1.278 | −14.112 | −18.186 | 1.00 | 0.00 | 1MOD |
| ATOM | 2420 | O | ILE | 301 | −0.620 | −14.120 | −17.148 | 1.00 | 0.00 | 1MOD |
| ATOM | 2421 | CB | ILE | 301 | −0.653 | −15.637 | −20.017 | 1.00 | 0.00 | 1MOD |
| ATOM | 2422 | CG1 | ILE | 301 | 0.740 | −16.055 | −19.511 | 1.00 | 0.00 | 1MOD |
| ATOM | 2423 | CG2 | ILE | 301 | −0.670 | −14.404 | −20.930 | 1.00 | 0.00 | 1MOD |
| ATOM | 2424 | CD1 | ILE | 301 | 1.481 | −14.992 | −18.704 | 1.00 | 0.00 | 1MOD |
| ATOM | 2425 | N | LEU | 302 | −1.725 | −12.967 | −18.738 | 1.00 | 0.00 | 1MOD |
| ATOM | 2426 | CA | LEU | 302 | −1.423 | −11.681 | −18.171 | 1.00 | 0.00 | 1MOD |
| ATOM | 2427 | C | LEU | 302 | −2.002 | −11.572 | −16.798 | 1.00 | 0.00 | 1MOD |
| ATOM | 2428 | O | LEU | 302 | −1.418 | −10.939 | −15.920 | 1.00 | 0.00 | 1MOD |
| ATOM | 2429 | CB | LEU | 302 | −1.940 | −10.489 | −18.992 | 1.00 | 0.00 | 1MOD |
| ATOM | 2430 | CG | LEU | 302 | −1.008 | −10.061 | −20.140 | 1.00 | 0.00 | 1MOD |
| ATOM | 2431 | CD1 | LEU | 302 | 0.261 | −9.390 | −19.593 | 1.00 | 0.00 | 1MOD |
| ATOM | 2432 | CD2 | LEU | 302 | −0.674 | −11.227 | −21.076 | 1.00 | 0.00 | 1MOD |
| ATOM | 2433 | N | GLY | 303 | −3.168 | −12.194 | −16.579 | 1.00 | 0.00 | 1MOD |
| ATOM | 2434 | CA | GLY | 303 | −3.861 | −12.120 | −15.326 | 1.00 | 0.00 | 1MOD |
| ATOM | 2435 | C | GLY | 303 | −2.981 | −12.664 | −14.241 | 1.00 | 0.00 | 1MOD |
| ATOM | 2436 | O | GLY | 303 | −3.071 | −12.253 | −13.086 | 1.00 | 0.00 | 1MOD |
| ATOM | 2437 | N | ILE | 304 | −2.096 | −13.604 | −14.611 | 1.00 | 0.00 | 1MOD |
| ATOM | 2438 | CA | ILE | 304 | −1.203 | −14.349 | −13.763 | 1.00 | 0.00 | 1MOD |
| ATOM | 2439 | C | ILE | 304 | −0.365 | −13.393 | −12.966 | 1.00 | 0.00 | 1MOD |
| ATOM | 2440 | O | ILE | 304 | 0.127 | −13.734 | −11.892 | 1.00 | 0.00 | 1MOD |
| ATOM | 2441 | CB | ILE | 304 | −0.301 | −15.231 | −14.573 | 1.00 | 0.00 | 1MOD |
| ATOM | 2442 | CG1 | ILE | 304 | −1.168 | −16.204 | −15.382 | 1.00 | 0.00 | 1MOD |
| ATOM | 2443 | CG2 | ILE | 304 | 0.701 | −15.925 | −13.634 | 1.00 | 0.00 | 1MOD |
| ATOM | 2444 | CD1 | ILE | 304 | −0.427 | −16.832 | −16.552 | 1.00 | 0.00 | 1MOD |
| ATOM | 2445 | N | LEU | 305 | −0.168 | −12.179 | −13.502 | 1.00 | 0.00 | 1MOD |
| ATOM | 2446 | CA | LEU | 305 | 0.617 | −11.108 | −12.951 | 1.00 | 0.00 | 1MOD |
| ATOM | 2447 | C | LEU | 305 | 0.070 | −10.737 | −11.598 | 1.00 | 0.00 | 1MOD |
| ATOM | 2448 | O | LEU | 305 | 0.783 | −10.183 | −10.761 | 1.00 | 0.00 | 1MOD |
| ATOM | 2449 | CB | LEU | 305 | 0.529 | −9.861 | −13.850 | 1.00 | 0.00 | 1MOD |
| ATOM | 2450 | CG | LEU | 305 | 1.327 | −8.642 | −13.360 | 1.00 | 0.00 | 1MOD |
| ATOM | 2451 | CD1 | LEU | 305 | 2.841 | −8.902 | −13.410 | 1.00 | 0.00 | 1MOD |
| ATOM | 2452 | CD2 | LEU | 305 | 0.918 | −7.372 | −14.125 | 1.00 | 0.00 | 1MOD |
| ATOM | 2453 | N | HIS | 306 | −1.219 | −11.037 | −11.353 | 1.00 | 0.00 | 1MOD |
| ATOM | 2454 | CA | HIS | 306 | −1.940 | −10.685 | −10.162 | 1.00 | 0.00 | 1MOD |
| ATOM | 2455 | C | HIS | 306 | −1.233 | −11.230 | −8.958 | 1.00 | 0.00 | 1MOD |
| ATOM | 2456 | O | HIS | 306 | −1.431 | −10.753 | −7.842 | 1.00 | 0.00 | 1MOD |
| ATOM | 2457 | CB | HIS | 306 | −3.363 | −11.260 | −10.170 | 1.00 | 0.00 | 1MOD |
| ATOM | 2458 | CG | HIS | 306 | −4.225 | −10.713 | −9.078 | 1.00 | 0.00 | 1MOD |
| ATOM | 2459 | ND1 | HIS | 306 | −4.972 | −9.565 | −9.203 | 1.00 | 0.00 | 1MOD |
| ATOM | 2460 | CD2 | HIS | 306 | −4.461 | −11.179 | −7.821 | 1.00 | 0.00 | 1MOD |
| ATOM | 2461 | CE1 | HIS | 306 | −5.622 | −9.392 | −8.025 | 1.00 | 0.00 | 1MOD |
| ATOM | 2462 | NE2 | HIS | 306 | −5.342 | −10.347 | −7.155 | 1.00 | 0.00 | 1MOD |
| ATOM | 2463 | N | SER | 307 | −0.441 | −12.296 | −9.146 | 1.00 | 0.00 | 1MOD |
| ATOM | 2464 | CA | SER | 307 | 0.281 | −12.917 | −8.077 | 1.00 | 0.00 | 1MOD |
| ATOM | 2465 | C | SER | 307 | 1.319 | −12.001 | −7.475 | 1.00 | 0.00 | 1MOD |
| ATOM | 2466 | O | SER | 307 | 1.582 | −12.087 | −6.279 | 1.00 | 0.00 | 1MOD |
| ATOM | 2467 | CB | SER | 307 | 1.029 | −14.178 | −8.540 | 1.00 | 0.00 | 1MOD |
| ATOM | 2468 | OG | SER | 307 | 2.036 | −13.835 | −9.481 | 1.00 | 0.00 | 1MOD |
| ATOM | 2469 | N | CYS | 308 | 2.064 | −11.239 | −8.303 | 1.00 | 0.00 | 1MOD |
| ATOM | 2470 | CA | CYS | 308 | 3.124 | −10.367 | −7.841 | 1.00 | 0.00 | 1MOD |
| ATOM | 2471 | C | CYS | 308 | 2.830 | −8.906 | −7.596 | 1.00 | 0.00 | 1MOD |
| ATOM | 2472 | O | CYS | 308 | 3.475 | −8.278 | −6.758 | 1.00 | 0.00 | 1MOD |
| ATOM | 2473 | CB | CYS | 308 | 4.345 | −10.418 | −8.775 | 1.00 | 0.00 | 1MOD |
| ATOM | 2474 | SG | CYS | 308 | 3.960 | −9.822 | −10.450 | 1.00 | 0.00 | 1MOD |
| ATOM | 2475 | N | LEU | 309 | 1.898 | −8.300 | −8.352 | 1.00 | 0.00 | 1MOD |
| ATOM | 2476 | CA | LEU | 309 | 1.805 | −6.860 | −8.400 | 1.00 | 0.00 | 1MOD |
| ATOM | 2477 | C | LEU | 309 | 1.501 | −6.213 | −7.087 | 1.00 | 0.00 | 1MOD |
| ATOM | 2478 | O | LEU | 309 | 2.038 | −5.146 | −6.792 | 1.00 | 0.00 | 1MOD |
| ATOM | 2479 | CB | LEU | 309 | 0.803 | −6.369 | −9.464 | 1.00 | 0.00 | 1MOD |
| ATOM | 2480 | CG | LEU | 309 | −0.642 | −6.876 | −9.301 | 1.00 | 0.00 | 1MOD |
| ATOM | 2481 | CD1 | LEU | 309 | −1.363 | −6.175 | −8.136 | 1.00 | 0.00 | 1MOD |
| ATOM | 2482 | CD2 | LEU | 309 | −1.405 | −6.815 | −10.636 | 1.00 | 0.00 | 1MOD |
| ATOM | 2483 | N | ASN | 310 | 0.639 | −6.809 | −6.253 | 1.00 | 0.00 | 1MOD |
| ATOM | 2484 | CA | ASN | 310 | 0.275 | −6.107 | −5.061 | 1.00 | 0.00 | 1MOD |
| ATOM | 2485 | C | ASN | 310 | 1.441 | −5.868 | −4.143 | 1.00 | 0.00 | 1MOD |
| ATOM | 2486 | O | ASN | 310 | 1.481 | −4.800 | −3.537 | 1.00 | 0.00 | 1MOD |
| ATOM | 2487 | CB | ASN | 310 | −0.936 | −6.706 | −4.303 | 1.00 | 0.00 | 1MOD |
| ATOM | 2488 | CG | ASN | 310 | −0.743 | −8.182 | −4.006 | 1.00 | 0.00 | 1MOD |
| ATOM | 2489 | OD1 | ASN | 310 | −0.904 | −9.039 | −4.873 | 1.00 | 0.00 | 1MOD |
| ATOM | 2490 | ND2 | ASN | 310 | −0.343 | −8.486 | −2.742 | 1.00 | 0.00 | 1MOD |
| ATOM | 2491 | N | PRO | 311 | 2.400 | −6.734 | −3.960 | 1.00 | 0.00 | 1MOD |
| ATOM | 2492 | CA | PRO | 311 | 3.455 | −6.396 | −3.050 | 1.00 | 0.00 | 1MOD |
| ATOM | 2493 | C | PRO | 311 | 4.288 | −5.287 | −3.599 | 1.00 | 0.00 | 1MOD |
| ATOM | 2494 | O | PRO | 311 | 4.905 | −4.562 | −2.822 | 1.00 | 0.00 | 1MOD |
| ATOM | 2495 | CB | PRO | 311 | 4.229 | −7.688 | −2.806 | 1.00 | 0.00 | 1MOD |
| ATOM | 2496 | CG | PRO | 311 | 3.192 | −8.792 | −3.063 | 1.00 | 0.00 | 1MOD |
| ATOM | 2497 | CD | PRO | 311 | 2.252 | −8.171 | −4.107 | 1.00 | 0.00 | 1MOD |
| ATOM | 2498 | N | LEU | 312 | 4.337 | −5.157 | −4.937 | 1.00 | 0.00 | 1MOD |
| ATOM | 2499 | CA | LEU | 312 | 5.154 | −4.156 | −5.552 | 1.00 | 0.00 | 1MOD |
| ATOM | 2500 | C | LEU | 312 | 4.614 | −2.815 | −5.184 | 1.00 | 0.00 | 1MOD |
| ATOM | 2501 | O | LEU | 312 | 5.351 | −1.917 | −4.777 | 1.00 | 0.00 | 1MOD |
| ATOM | 2502 | CB | LEU | 312 | 5.120 | −4.233 | −7.087 | 1.00 | 0.00 | 1MOD |
| ATOM | 2503 | CG | LEU | 312 | 6.005 | −3.175 | −7.773 | 1.00 | 0.00 | 1MOD |
| ATOM | 2504 | CD1 | LEU | 312 | 7.494 | −3.494 | −7.582 | 1.00 | 0.00 | 1MOD |
| ATOM | 2505 | CD2 | LEU | 312 | 5.613 | −2.965 | −9.243 | 1.00 | 0.00 | 1MOD |
| ATOM | 2506 | N | ILE | 313 | 3.286 | −2.662 | −5.308 | 1.00 | 0.00 | 1MOD |
| ATOM | 2507 | CA | ILE | 313 | 2.672 | −1.400 | −5.058 | 1.00 | 0.00 | 1MOD |
| ATOM | 2508 | C | ILE | 313 | 2.858 | −1.064 | −3.616 | 1.00 | 0.00 | 1MOD |
| ATOM | 2509 | O | ILE | 313 | 3.117 | 0.085 | −3.262 | 1.00 | 0.00 | 1MOD |
| ATOM | 2510 | CB | ILE | 313 | 1.207 | −1.373 | −5.400 | 1.00 | 0.00 | 1MOD |
| ATOM | 2511 | CG1 | ILE | 313 | 0.736 | 0.078 | −5.540 | 1.00 | 0.00 | 1MOD |
| ATOM | 2512 | CG2 | ILE | 313 | 0.423 | −2.179 | −4.352 | 1.00 | 0.00 | 1MOD |
| ATOM | 2513 | CD1 | ILE | 313 | 1.338 | 0.791 | −6.751 | 1.00 | 0.00 | 1MOD |
| ATOM | 2514 | N | TYR | 314 | 2.745 | −2.085 | −2.751 | 1.00 | 0.00 | 1MOD |
| ATOM | 2515 | CA | TYR | 314 | 2.804 | −1.935 | −1.330 | 1.00 | 0.00 | 1MOD |
| ATOM | 2516 | C | TYR | 314 | 4.148 | −1.382 | −0.962 | 1.00 | 0.00 | 1MOD |
| ATOM | 2517 | O | TYR | 314 | 4.250 | −0.432 | −0.187 | 1.00 | 0.00 | 1MOD |
| ATOM | 2518 | CB | TYR | 314 | 2.605 | −3.315 | −0.668 | 1.00 | 0.00 | 1MOD |
| ATOM | 2519 | CG | TYR | 314 | 2.304 | −3.179 | 0.781 | 1.00 | 0.00 | 1MOD |
| ATOM | 2520 | CD1 | TYR | 314 | 1.043 | −2.814 | 1.191 | 1.00 | 0.00 | 1MOD |
| ATOM | 2521 | CD2 | TYR | 314 | 3.261 | −3.449 | 1.727 | 1.00 | 0.00 | 1MOD |
| ATOM | 2522 | CE1 | TYR | 314 | 0.746 | −2.694 | 2.527 | 1.00 | 0.00 | 1MOD |
| ATOM | 2523 | CE2 | TYR | 314 | 2.970 | −3.332 | 3.064 | 1.00 | 0.00 | 1MOD |
| ATOM | 2524 | CZ | TYR | 314 | 1.713 | −2.953 | 3.465 | 1.00 | 0.00 | 1MOD |
| ATOM | 2525 | OH | TYR | 314 | 1.416 | −2.835 | 4.838 | 1.00 | 0.00 | 1MOD |
| ATOM | 2526 | N | ALA | 315 | 5.225 | −1.949 | −1.532 | 1.00 | 0.00 | 1MOD |
| ATOM | 2527 | CA | ALA | 315 | 6.527 | −1.491 | −1.158 | 1.00 | 0.00 | 1MOD |
| ATOM | 2528 | C | ALA | 315 | 6.687 | −0.050 | −1.540 | 1.00 | 0.00 | 1MOD |
| ATOM | 2529 | O | ALA | 315 | 7.162 | 0.754 | −0.741 | 1.00 | 0.00 | 1MOD |
| ATOM | 2530 | CB | ALA | 315 | 7.656 | −2.281 | −1.841 | 1.00 | 0.00 | 1MOD |
| ATOM | 2531 | N | PHE | 316 | 6.301 | 0.330 | −2.776 | 1.00 | 0.00 | 1MOD |
| ATOM | 2532 | CA | PHE | 316 | 6.508 | 1.693 | −3.195 | 1.00 | 0.00 | 1MOD |
| ATOM | 2533 | C | PHE | 316 | 5.629 | 2.648 | −2.439 | 1.00 | 0.00 | 1MOD |
| ATOM | 2534 | O | PHE | 316 | 6.092 | 3.676 | −1.945 | 1.00 | 0.00 | 1MOD |
| ATOM | 2535 | CB | PHE | 316 | 6.247 | 1.931 | −4.696 | 1.00 | 0.00 | 1MOD |
| ATOM | 2536 | CG | PHE | 316 | 7.285 | 1.200 | −5.468 | 1.00 | 0.00 | 1MOD |
| ATOM | 2537 | CD1 | PHE | 316 | 8.500 | 1.791 | −5.724 | 1.00 | 0.00 | 1MOD |
| ATOM | 2538 | CD2 | PHE | 316 | 7.044 | −0.073 | −5.923 | 1.00 | 0.00 | 1MOD |
| ATOM | 2539 | CE1 | PHE | 316 | 9.460 | 1.112 | −6.437 | 1.00 | 0.00 | 1MOD |
| ATOM | 2540 | CE2 | PHE | 316 | 8.002 | −0.749 | −6.636 | 1.00 | 0.00 | 1MOD |
| ATOM | 2541 | CZ | PHE | 316 | 9.216 | −0.161 | −6.895 | 1.00 | 0.00 | 1MOD |
| ATOM | 2542 | N | ILE | 317 | 4.324 | 2.341 | −2.344 | 1.00 | 0.00 | 1MOD |
| ATOM | 2543 | CA | ILE | 317 | 3.393 | 3.245 | −1.742 | 1.00 | 0.00 | 1MOD |
| ATOM | 2544 | C | ILE | 317 | 3.564 | 3.394 | −0.265 | 1.00 | 0.00 | 1MOD |
| ATOM | 2545 | O | ILE | 317 | 3.415 | 4.504 | 0.245 | 1.00 | 0.00 | 1MOD |
| ATOM | 2546 | CB | ILE | 317 | 1.956 | 2.966 | −2.080 | 1.00 | 0.00 | 1MOD |
| ATOM | 2547 | CG1 | ILE | 317 | 1.756 | 3.241 | −3.584 | 1.00 | 0.00 | 1MOD |
| ATOM | 2548 | CG2 | ILE | 317 | 1.060 | 3.844 | −1.187 | 1.00 | 0.00 | 1MOD |
| ATOM | 2549 | CD1 | ILE | 317 | 0.345 | 3.000 | −4.109 | 1.00 | 0.00 | 1MOD |
| ATOM | 2550 | N | GLY | 318 | 3.857 | 2.308 | 0.477 | 1.00 | 0.00 | 1MOD |
| ATOM | 2551 | CA | GLY | 318 | 3.947 | 2.451 | 1.903 | 1.00 | 0.00 | 1MOD |
| ATOM | 2552 | C | GLY | 318 | 5.365 | 2.669 | 2.320 | 1.00 | 0.00 | 1MOD |
| ATOM | 2553 | O | GLY | 318 | 6.230 | 1.808 | 2.169 | 1.00 | 0.00 | 1MOD |
| ATOM | 2554 | N | GLN | 319 | 5.613 | 3.854 | 2.899 | 1.00 | 0.00 | 1MOD |
| ATOM | 2555 | CA | GLN | 319 | 6.899 | 4.238 | 3.387 | 1.00 | 0.00 | 1MOD |
| ATOM | 2556 | C | GLN | 319 | 7.262 | 3.371 | 4.546 | 1.00 | 0.00 | 1MOD |
| ATOM | 2557 | O | GLN | 319 | 8.390 | 2.892 | 4.645 | 1.00 | 0.00 | 1MOD |
| ATOM | 2558 | CB | GLN | 319 | 6.844 | 5.683 | 3.901 | 1.00 | 0.00 | 1MOD |
| ATOM | 2559 | CG | GLN | 319 | 8.070 | 6.144 | 4.679 | 1.00 | 0.00 | 1MOD |
| ATOM | 2560 | CD | GLN | 319 | 7.739 | 7.542 | 5.181 | 1.00 | 0.00 | 1MOD |
| ATOM | 2561 | OE1 | GLN | 319 | 7.681 | 8.493 | 4.404 | 1.00 | 0.00 | 1MOD |
| ATOM | 2562 | NE2 | GLN | 319 | 7.487 | 7.667 | 6.511 | 1.00 | 0.00 | 1MOD |
| ATOM | 2563 | N | LYS | 320 | 6.302 | 3.141 | 5.459 | 1.00 | 0.00 | 1MOD |
| ATOM | 2564 | CA | LYS | 320 | 6.591 | 2.405 | 6.656 | 1.00 | 0.00 | 1MOD |
| ATOM | 2565 | C | LYS | 320 | 7.003 | 1.007 | 6.336 | 1.00 | 0.00 | 1MOD |
| ATOM | 2566 | O | LYS | 320 | 8.011 | 0.525 | 6.848 | 1.00 | 0.00 | 1MOD |
| ATOM | 2567 | CB | LYS | 320 | 5.409 | 2.347 | 7.639 | 1.00 | 0.00 | 1MOD |
| ATOM | 2568 | CG | LYS | 320 | 4.862 | 0.941 | 7.900 | 1.00 | 0.00 | 1MOD |
| ATOM | 2569 | CD | LYS | 320 | 3.397 | 0.777 | 7.481 | 1.00 | 0.00 | 1MOD |
| ATOM | 2570 | CE | LYS | 320 | 2.769 | −0.561 | 7.874 | 1.00 | 0.00 | 1MOD |
| ATOM | 2571 | NZ | LYS | 320 | 1.292 | −0.476 | 7.764 | 1.00 | 0.00 | 1MOD |
| ATOM | 2572 | N | PHE | 321 | 6.246 | 0.333 | 5.455 | 1.00 | 0.00 | 1MOD |
| ATOM | 2573 | CA | PHE | 321 | 6.522 | −1.022 | 5.071 | 1.00 | 0.00 | 1MOD |
| ATOM | 2574 | C | PHE | 321 | 7.847 | −1.077 | 4.380 | 1.00 | 0.00 | 1MOD |
| ATOM | 2575 | O | PHE | 321 | 8.627 | −2.003 | 4.598 | 1.00 | 0.00 | 1MOD |
| ATOM | 2576 | CB | PHE | 321 | 5.456 | −1.579 | 4.102 | 1.00 | 0.00 | 1MOD |
| ATOM | 2577 | CG | PHE | 321 | 5.840 | −2.938 | 3.607 | 1.00 | 0.00 | 1MOD |
| ATOM | 2578 | CD1 | PHE | 321 | 5.659 | −4.063 | 4.380 | 1.00 | 0.00 | 1MOD |
| ATOM | 2579 | CD2 | PHE | 321 | 6.354 | −3.090 | 2.338 | 1.00 | 0.00 | 1MOD |
| ATOM | 2580 | CE1 | PHE | 321 | 6.007 | −5.307 | 3.902 | 1.00 | 0.00 | 1MOD |
| ATOM | 2581 | CE2 | PHE | 321 | 6.704 | −4.332 | 1.854 | 1.00 | 0.00 | 1MOD |
| ATOM | 2582 | CZ | PHE | 321 | 6.532 | −5.446 | 2.638 | 1.00 | 0.00 | 1MOD |
| ATOM | 2583 | N | ARG | 322 | 8.138 | −0.073 | 3.532 | 1.00 | 0.00 | 1MOD |
| ATOM | 2584 | CA | ARG | 322 | 9.341 | −0.049 | 2.749 | 1.00 | 0.00 | 1MOD |
| ATOM | 2585 | C | ARG | 322 | 10.510 | −0.131 | 3.680 | 1.00 | 0.00 | 1MOD |
| ATOM | 2586 | O | ARG | 322 | 11.429 | −0.922 | 3.470 | 1.00 | 0.00 | 1MOD |
| ATOM | 2587 | CB | ARG | 322 | 9.492 | 1.281 | 1.989 | 1.00 | 0.00 | 1MOD |
| ATOM | 2588 | CG | ARG | 322 | 10.752 | 1.381 | 1.128 | 1.00 | 0.00 | 1MOD |
| ATOM | 2589 | CD | ARG | 322 | 11.130 | 2.820 | 0.766 | 1.00 | 0.00 | 1MOD |
| ATOM | 2590 | NE | ARG | 322 | 11.869 | 3.378 | 1.935 | 1.00 | 0.00 | 1MOD |
| ATOM | 2591 | CZ | ARG | 322 | 11.214 | 4.046 | 2.931 | 1.00 | 0.00 | 1MOD |
| ATOM | 2592 | NH1 | ARG | 322 | 9.872 | 4.278 | 2.840 | 1.00 | 0.00 | 1MOD |
| ATOM | 2593 | NH2 | ARG | 322 | 11.907 | 4.478 | 4.024 | 1.00 | 0.00 | 1MOD |
| ATOM | 2594 | N | HIS | 323 | 10.483 | 0.690 | 4.746 | 1.00 | 0.00 | 1MOD |
| ATOM | 2595 | CA | HIS | 323 | 11.514 | 0.752 | 5.745 | 1.00 | 0.00 | 1MOD |
| ATOM | 2596 | C | HIS | 323 | 11.589 | −0.581 | 6.405 | 1.00 | 0.00 | 1MOD |
| ATOM | 2597 | O | HIS | 323 | 12.676 | −1.123 | 6.613 | 1.00 | 0.00 | 1MOD |
| ATOM | 2598 | CB | HIS | 323 | 11.139 | 1.766 | 6.849 | 1.00 | 0.00 | 1MOD |
| ATOM | 2599 | CG | HIS | 323 | 12.049 | 1.847 | 8.046 | 1.00 | 0.00 | 1MOD |
| ATOM | 2600 | ND1 | HIS | 323 | 12.438 | 3.033 | 8.630 | 1.00 | 0.00 | 1MOD |
| ATOM | 2601 | CD2 | HIS | 323 | 12.589 | 0.863 | 8.817 | 1.00 | 0.00 | 1MOD |
| ATOM | 2602 | CE1 | HIS | 323 | 13.183 | 2.711 | 9.717 | 1.00 | 0.00 | 1MOD |
| ATOM | 2603 | NE2 | HIS | 323 | 13.303 | 1.405 | 9.870 | 1.00 | 0.00 | 1MOD |
| ATOM | 2604 | N | GLY | 324 | 10.410 | −1.137 | 6.739 | 1.00 | 0.00 | 1MOD |
| ATOM | 2605 | CA | GLY | 324 | 10.316 | −2.377 | 7.441 | 1.00 | 0.00 | 1MOD |
| ATOM | 2606 | C | GLY | 324 | 10.973 | −3.439 | 6.627 | 1.00 | 0.00 | 1MOD |
| ATOM | 2607 | O | GLY | 324 | 11.647 | −4.315 | 7.165 | 1.00 | 0.00 | 1MOD |
| ATOM | 2608 | N | LEU | 325 | 10.759 | −3.397 | 5.303 | 1.00 | 0.00 | 1MOD |
| ATOM | 2609 | CA | LEU | 325 | 11.325 | −4.357 | 4.404 | 1.00 | 0.00 | 1MOD |
| ATOM | 2610 | C | LEU | 325 | 12.814 | −4.213 | 4.431 | 1.00 | 0.00 | 1MOD |
| ATOM | 2611 | O | LEU | 325 | 13.550 | −5.183 | 4.599 | 1.00 | 0.00 | 1MOD |
| ATOM | 2612 | CB | LEU | 325 | 10.880 | −4.055 | 2.967 | 1.00 | 0.00 | 1MOD |
| ATOM | 2613 | CG | LEU | 325 | 11.429 | −5.031 | 1.918 | 1.00 | 0.00 | 1MOD |
| ATOM | 2614 | CD1 | LEU | 325 | 10.821 | −6.433 | 2.108 | 1.00 | 0.00 | 1MOD |
| ATOM | 2615 | CD2 | LEU | 325 | 11.251 | −4.476 | 0.496 | 1.00 | 0.00 | 1MOD |
| ATOM | 2616 | N | LEU | 326 | 13.293 | −2.963 | 4.299 | 1.00 | 0.00 | 1MOD |
| ATOM | 2617 | CA | LEU | 326 | 14.698 | −2.688 | 4.233 | 1.00 | 0.00 | 1MOD |
| ATOM | 2618 | C | LEU | 326 | 15.329 | −3.108 | 5.515 | 1.00 | 0.00 | 1MOD |
| ATOM | 2619 | O | LEU | 326 | 16.426 | −3.665 | 5.523 | 1.00 | 0.00 | 1MOD |
| ATOM | 2620 | CB | LEU | 326 | 15.001 | −1.190 | 4.018 | 1.00 | 0.00 | 1MOD |
| ATOM | 2621 | CG | LEU | 326 | 16.503 | −0.836 | 3.944 | 1.00 | 0.00 | 1MOD |
| ATOM | 2622 | CD1 | LEU | 326 | 17.177 | −0.824 | 5.326 | 1.00 | 0.00 | 1MOD |
| ATOM | 2623 | CD2 | LEU | 326 | 17.229 | −1.766 | 2.962 | 1.00 | 0.00 | 1MOD |
| ATOM | 2624 | N | LYS | 327 | 14.648 | −2.856 | 6.641 | 1.00 | 0.00 | 1MOD |
| ATOM | 2625 | CA | LYS | 327 | 15.258 | −3.158 | 7.893 | 1.00 | 0.00 | 1MOD |
| ATOM | 2626 | C | LYS | 327 | 15.507 | −4.631 | 7.923 | 1.00 | 0.00 | 1MOD |
| ATOM | 2627 | O | LYS | 327 | 16.544 | −5.072 | 8.406 | 1.00 | 0.00 | 1MOD |
| ATOM | 2628 | CB | LYS | 327 | 14.385 | −2.775 | 9.105 | 1.00 | 0.00 | 1MOD |
| ATOM | 2629 | CG | LYS | 327 | 13.621 | −3.925 | 9.767 | 1.00 | 0.00 | 1MOD |
| ATOM | 2630 | CD | LYS | 327 | 14.519 | −4.747 | 10.696 | 1.00 | 0.00 | 1MOD |
| ATOM | 2631 | CE | LYS | 327 | 13.776 | −5.740 | 11.586 | 1.00 | 0.00 | 1MOD |
| ATOM | 2632 | NZ | LYS | 327 | 14.709 | −6.292 | 12.591 | 1.00 | 0.00 | 1MOD |
| ATOM | 2633 | N | ILE | 328 | 14.558 | −5.441 | 7.413 | 1.00 | 0.00 | 1MOD |
| ATOM | 2634 | CA | ILE | 328 | 14.742 | −6.865 | 7.419 | 1.00 | 0.00 | 1MOD |
| ATOM | 2635 | C | ILE | 328 | 15.875 | −7.244 | 6.513 | 1.00 | 0.00 | 1MOD |
| ATOM | 2636 | O | ILE | 328 | 16.736 | −8.036 | 6.894 | 1.00 | 0.00 | 1MOD |
| ATOM | 2637 | CB | ILE | 328 | 13.522 | −7.620 | 6.975 | 1.00 | 0.00 | 1MOD |
| ATOM | 2638 | CG1 | ILE | 328 | 12.392 | −7.424 | 7.997 | 1.00 | 0.00 | 1MOD |
| ATOM | 2639 | CG2 | ILE | 328 | 13.923 | −9.091 | 6.769 | 1.00 | 0.00 | 1MOD |
| ATOM | 2640 | CD1 | ILE | 328 | 12.753 | −7.936 | 9.390 | 1.00 | 0.00 | 1MOD |
| ATOM | 2641 | N | LEU | 329 | 15.916 | −6.687 | 5.286 | 1.00 | 0.00 | 1MOD |
| ATOM | 2642 | CA | LEU | 329 | 16.966 | −7.068 | 4.382 | 1.00 | 0.00 | 1MOD |
| ATOM | 2643 | C | LEU | 329 | 18.282 | −6.691 | 4.971 | 1.00 | 0.00 | 1MOD |
| ATOM | 2644 | O | LEU | 329 | 19.209 | −7.495 | 5.018 | 1.00 | 0.00 | 1MOD |
| ATOM | 2645 | CB | LEU | 329 | 16.890 | −6.368 | 3.010 | 1.00 | 0.00 | 1MOD |
| ATOM | 2646 | CG | LEU | 329 | 15.829 | −6.918 | 2.037 | 1.00 | 0.00 | 1MOD |
| ATOM | 2647 | CD1 | LEU | 329 | 14.412 | −6.865 | 2.622 | 1.00 | 0.00 | 1MOD |
| ATOM | 2648 | CD2 | LEU | 329 | 15.930 | −6.207 | 0.676 | 1.00 | 0.00 | 1MOD |
| ATOM | 2649 | N | ALA | 330 | 18.385 | −5.446 | 5.455 | 1.00 | 0.00 | 1MOD |
| ATOM | 2650 | CA | ALA | 330 | 19.608 | −4.955 | 6.010 | 1.00 | 0.00 | 1MOD |
| ATOM | 2651 | C | ALA | 330 | 19.924 | −5.737 | 7.241 | 1.00 | 0.00 | 1MOD |
| ATOM | 2652 | O | ALA | 330 | 21.088 | −5.954 | 7.572 | 1.00 | 0.00 | 1MOD |
| ATOM | 2653 | CB | ALA | 330 | 19.523 | −3.475 | 6.412 | 1.00 | 0.00 | 1MOD |
| ATOM | 2654 | N | ILE | 331 | 18.859 | −6.166 | 7.941 | 1.00 | 0.00 | 1MOD |
| ATOM | 2655 | CA | ILE | 331 | 18.886 | −6.725 | 9.258 | 1.00 | 0.00 | 1MOD |
| ATOM | 2656 | C | ILE | 331 | 19.829 | −5.844 | 9.998 | 1.00 | 0.00 | 1MOD |
| ATOM | 2657 | O | ILE | 331 | 20.961 | −6.181 | 10.332 | 1.00 | 0.00 | 1MOD |
| ATOM | 2658 | CB | ILE | 331 | 19.259 | −8.193 | 9.300 | 1.00 | 0.00 | 1MOD |
| ATOM | 2659 | CG1 | ILE | 331 | 18.991 | −8.788 | 10.695 | 1.00 | 0.00 | 1MOD |
| ATOM | 2660 | CG2 | ILE | 331 | 20.683 | −8.397 | 8.758 | 1.00 | 0.00 | 1MOD |
| ATOM | 2661 | CD1 | ILE | 331 | 19.799 | −8.162 | 11.829 | 1.00 | 0.00 | 1MOD |
| ATOM | 2662 | N | HIS | 332 | 19.305 | −4.655 | 10.321 | 1.00 | 0.00 | 1MOD |
| ATOM | 2663 | CA | HIS | 332 | 20.055 | −3.615 | 10.939 | 1.00 | 0.00 | 1MOD |
| ATOM | 2664 | C | HIS | 332 | 19.199 | −2.413 | 10.739 | 1.00 | 0.00 | 1MOD |
| ATOM | 2665 | O | HIS | 332 | 18.099 | −2.331 | 11.284 | 1.00 | 0.00 | 1MOD |
| ATOM | 2666 | CB | HIS | 332 | 21.428 | −3.365 | 10.281 | 1.00 | 0.00 | 1MOD |
| ATOM | 2667 | CG | HIS | 332 | 22.227 | −2.272 | 10.938 | 1.00 | 0.00 | 1MOD |
| ATOM | 2668 | ND1 | HIS | 332 | 22.919 | −2.412 | 12.120 | 1.00 | 0.00 | 1MOD |
| ATOM | 2669 | CD2 | HIS | 332 | 22.432 | −0.986 | 10.540 | 1.00 | 0.00 | 1MOD |
| ATOM | 2670 | CE1 | HIS | 332 | 23.503 | −1.212 | 12.377 | 1.00 | 0.00 | 1MOD |
| ATOM | 2671 | NE2 | HIS | 332 | 23.235 | −0.317 | 11.446 | 1.00 | 0.00 | 1MOD |
| ATOM | 2672 | N | GLY | 333 | 19.675 | −1.453 | 9.926 | 1.00 | 0.00 | 1MOD |
| ATOM | 2673 | CA | GLY | 333 | 18.905 | −0.267 | 9.710 | 1.00 | 0.00 | 1MOD |
| ATOM | 2674 | C | GLY | 333 | 19.466 | 0.445 | 8.526 | 1.00 | 0.00 | 1MOD |
| ATOM | 2675 | O | GLY | 333 | 19.841 | −0.168 | 7.527 | 1.00 | 0.00 | 1MOD |
| ATOM | 2676 | N | LEU | 334 | 19.566 | 1.779 | 8.650 | 1.00 | 0.00 | 1MOD |
| ATOM | 2677 | CA | LEU | 334 | 20.074 | 2.619 | 7.613 | 1.00 | 0.00 | 1MOD |
| ATOM | 2678 | C | LEU | 334 | 19.052 | 2.645 | 6.510 | 1.00 | 0.00 | 1MOD |
| ATOM | 2679 | O | LEU | 334 | 19.374 | 2.745 | 5.329 | 1.00 | 0.00 | 1MOD |
| ATOM | 2680 | CB | LEU | 334 | 21.420 | 2.080 | 7.079 | 1.00 | 0.00 | 1MOD |
| ATOM | 2681 | CG | LEU | 334 | 22.396 | 3.118 | 6.478 | 1.00 | 0.00 | 1MOD |
| ATOM | 2682 | CD1 | LEU | 334 | 21.857 | 3.863 | 5.250 | 1.00 | 0.00 | 1MOD |
| ATOM | 2683 | CD2 | LEU | 334 | 22.901 | 4.063 | 7.580 | 1.00 | 0.00 | 1MOD |
| ATOM | 2684 | N | ILE | 335 | 17.758 | 2.543 | 6.867 | 1.00 | 0.00 | 1MOD |
| ATOM | 2685 | CA | ILE | 335 | 16.779 | 2.706 | 5.834 | 1.00 | 0.00 | 1MOD |
| ATOM | 2686 | C | ILE | 335 | 16.390 | 4.144 | 5.886 | 1.00 | 0.00 | 1MOD |
| ATOM | 2687 | O | ILE | 335 | 15.282 | 4.503 | 6.276 | 1.00 | 0.00 | 1MOD |
| ATOM | 2688 | CB | ILE | 335 | 15.566 | 1.845 | 6.021 | 1.00 | 0.00 | 1MOD |
| ATOM | 2689 | CG1 | ILE | 335 | 14.522 | 2.140 | 4.931 | 1.00 | 0.00 | 1MOD |
| ATOM | 2690 | CG2 | ILE | 335 | 15.076 | 2.008 | 7.465 | 1.00 | 0.00 | 1MOD |
| ATOM | 2691 | CD1 | ILE | 335 | 14.965 | 1.775 | 3.514 | 1.00 | 0.00 | 1MOD |
| ATOM | 2692 | N | SER | 336 | 17.319 | 5.020 | 5.471 | 1.00 | 0.00 | 1MOD |
| ATOM | 2693 | CA | SER | 336 | 17.038 | 6.418 | 5.525 | 1.00 | 0.00 | 1MOD |
| ATOM | 2694 | C | SER | 336 | 16.195 | 6.744 | 4.348 | 1.00 | 0.00 | 1MOD |
| ATOM | 2695 | O | SER | 336 | 16.448 | 6.282 | 3.236 | 1.00 | 0.00 | 1MOD |
| ATOM | 2696 | CB | SER | 336 | 18.298 | 7.297 | 5.468 | 1.00 | 0.00 | 1MOD |
| ATOM | 2697 | OG | SER | 336 | 19.104 | 7.074 | 6.616 | 1.00 | 0.00 | 1MOD |
| ATOM | 2698 | N | LYS | 337 | 15.163 | 7.573 | 4.570 | 1.00 | 0.00 | 1MOD |
| ATOM | 2699 | CA | LYS | 337 | 14.284 | 7.912 | 3.496 | 1.00 | 0.00 | 1MOD |
| ATOM | 2700 | C | LYS | 337 | 14.038 | 9.400 | 3.574 | 1.00 | 0.00 | 1MOD |
| ATOM | 2701 | O | LYS | 337 | 14.949 | 10.133 | 3.948 | 1.00 | 0.00 | 1MOD |
| ATOM | 2702 | CB | LYS | 337 | 12.924 | 7.198 | 3.582 | 1.00 | 0.00 | 1MOD |
| ATOM | 2703 | CG | LYS | 337 | 12.203 | 7.091 | 2.236 | 1.00 | 0.00 | 1MOD |
| ATOM | 2704 | CD | LYS | 337 | 12.876 | 6.099 | 1.283 | 1.00 | 0.00 | 1MOD |
| ATOM | 2705 | CE | LYS | 337 | 12.187 | 5.963 | −0.076 | 1.00 | 0.00 | 1MOD |
| ATOM | 2706 | NZ | LYS | 337 | 12.895 | 4.964 | −0.912 | 1.00 | 0.00 | 1MOD |
| ATOM | 2707 | N | ASP | 338 | 12.805 | 9.858 | 3.209 | 1.00 | 0.00 | 1MOD |
| ATOM | 2708 | CA | ASP | 338 | 12.364 | 11.239 | 3.062 | 1.00 | 0.00 | 1MOD |
| ATOM | 2709 | C | ASP | 338 | 11.896 | 11.899 | 4.355 | 1.00 | 0.00 | 1MOD |
| ATOM | 2710 | O | ASP | 338 | 12.302 | 11.508 | 5.449 | 1.00 | 0.00 | 1MOD |
| ATOM | 2711 | CB | ASP | 338 | 11.185 | 11.389 | 2.085 | 1.00 | 0.00 | 1MOD |
| ATOM | 2712 | CG | ASP | 338 | 11.661 | 11.063 | 0.680 | 1.00 | 0.00 | 1MOD |
| ATOM | 2713 | OD1 | ASP | 338 | 12.887 | 10.833 | 0.498 | 1.00 | 0.00 | 1MOD |
| ATOM | 2714 | OD2 | ASP | 338 | 10.795 | 11.040 | −0.234 | 1.00 | 0.00 | 1MOD |
| ATOM | 2715 | N | SER | 339 | 11.072 | 12.988 | 4.202 | 1.00 | 0.00 | 1MOD |
| ATOM | 2716 | CA | SER | 339 | 10.542 | 13.940 | 5.177 | 1.00 | 0.00 | 1MOD |
| ATOM | 2717 | C | SER | 339 | 9.673 | 13.397 | 6.298 | 1.00 | 0.00 | 1MOD |
| ATOM | 2718 | O | SER | 339 | 10.177 | 13.161 | 7.394 | 1.00 | 0.00 | 1MOD |
| ATOM | 2719 | CB | SER | 339 | 9.706 | 15.050 | 4.521 | 1.00 | 0.00 | 1MOD |
| ATOM | 2720 | OG | SER | 339 | 9.227 | 15.937 | 5.521 | 1.00 | 0.00 | 1MOD |
| ATOM | 2721 | N | LEU | 340 | 8.342 | 13.212 | 6.068 | 1.00 | 0.00 | 1MOD |
| ATOM | 2722 | CA | LEU | 340 | 7.438 | 12.852 | 7.145 | 1.00 | 0.00 | 1MOD |
| ATOM | 2723 | C | LEU | 340 | 7.596 | 11.402 | 7.465 | 1.00 | 0.00 | 1MOD |
| ATOM | 2724 | O | LEU | 340 | 7.632 | 10.551 | 6.580 | 1.00 | 0.00 | 1MOD |
| ATOM | 2725 | CB | LEU | 340 | 5.955 | 13.110 | 6.834 | 1.00 | 0.00 | 1MOD |
| ATOM | 2726 | CG | LEU | 340 | 5.021 | 12.718 | 7.995 | 1.00 | 0.00 | 1MOD |
| ATOM | 2727 | CD1 | LEU | 340 | 5.316 | 13.536 | 9.265 | 1.00 | 0.00 | 1MOD |
| ATOM | 2728 | CD2 | LEU | 340 | 3.550 | 12.793 | 7.565 | 1.00 | 0.00 | 1MOD |
| ATOM | 2729 | N | PRO | 341 | 7.728 | 11.138 | 8.739 | 1.00 | 0.00 | 1MOD |
| ATOM | 2730 | CA | PRO | 341 | 7.921 | 9.786 | 9.200 | 1.00 | 0.00 | 1MOD |
| ATOM | 2731 | C | PRO | 341 | 6.673 | 9.040 | 9.535 | 1.00 | 0.00 | 1MOD |
| ATOM | 2732 | O | PRO | 341 | 5.614 | 9.649 | 9.679 | 1.00 | 0.00 | 1MOD |
| ATOM | 2733 | CB | PRO | 341 | 8.884 | 9.865 | 10.387 | 1.00 | 0.00 | 1MOD |
| ATOM | 2734 | CG | PRO | 341 | 8.798 | 11.325 | 10.850 | 1.00 | 0.00 | 1MOD |
| ATOM | 2735 | CD | PRO | 341 | 8.466 | 12.086 | 9.559 | 1.00 | 0.00 | 1MOD |
| ATOM | 2736 | N | LYS | 342 | 6.810 | 7.707 | 9.683 | 1.00 | 0.00 | 1MOD |
| ATOM | 2737 | CA | LYS | 342 | 5.751 | 6.833 | 10.075 | 1.00 | 0.00 | 1MOD |
| ATOM | 2738 | C | LYS | 342 | 6.203 | 6.275 | 11.388 | 1.00 | 0.00 | 1MOD |
| ATOM | 2739 | O | LYS | 342 | 7.189 | 6.742 | 11.957 | 1.00 | 0.00 | 1MOD |
| ATOM | 2740 | CB | LYS | 342 | 5.565 | 5.627 | 9.137 | 1.00 | 0.00 | 1MOD |
| ATOM | 2741 | CG | LYS | 342 | 4.210 | 4.929 | 9.281 | 1.00 | 0.00 | 1MOD |
| ATOM | 2742 | CD | LYS | 342 | 3.078 | 5.669 | 8.566 | 1.00 | 0.00 | 1MOD |
| ATOM | 2743 | CE | LYS | 342 | 3.132 | 7.187 | 8.739 | 1.00 | 0.00 | 1MOD |
| ATOM | 2744 | NZ | LYS | 342 | 2.230 | 7.838 | 7.761 | 1.00 | 0.00 | 1MOD |
| ATOM | 2745 | N | ASP | 343 | 5.485 | 5.264 | 11.906 | 1.00 | 0.00 | 1MOD |
| ATOM | 2746 | CA | ASP | 343 | 5.829 | 4.652 | 13.155 | 1.00 | 0.00 | 1MOD |
| ATOM | 2747 | C | ASP | 343 | 6.953 | 3.702 | 12.863 | 1.00 | 0.00 | 1MOD |
| ATOM | 2748 | O | ASP | 343 | 7.456 | 3.671 | 11.741 | 1.00 | 0.00 | 1MOD |
| ATOM | 2749 | CB | ASP | 343 | 4.665 | 3.821 | 13.729 | 1.00 | 0.00 | 1MOD |
| ATOM | 2750 | CG | ASP | 343 | 4.941 | 3.489 | 15.187 | 1.00 | 0.00 | 1MOD |
| ATOM | 2751 | OD1 | ASP | 343 | 5.900 | 4.067 | 15.762 | 1.00 | 0.00 | 1MOD |
| ATOM | 2752 | OD2 | ASP | 343 | 4.186 | 2.648 | 15.746 | 1.00 | 0.00 | 1MOD |
| ATOM | 2753 | N | SER | 344 | 7.392 | 2.922 | 13.877 | 1.00 | 0.00 | 1MOD |
| ATOM | 2754 | CA | SER | 344 | 8.441 | 1.960 | 13.691 | 1.00 | 0.00 | 1MOD |
| ATOM | 2755 | C | SER | 344 | 7.819 | 0.609 | 13.858 | 1.00 | 0.00 | 1MOD |
| ATOM | 2756 | O | SER | 344 | 6.707 | 0.494 | 14.373 | 1.00 | 0.00 | 1MOD |
| ATOM | 2757 | CB | SER | 344 | 9.573 | 2.052 | 14.731 | 1.00 | 0.00 | 1MOD |
| ATOM | 2758 | OG | SER | 344 | 10.536 | 1.034 | 14.499 | 1.00 | 0.00 | 1MOD |
| ATOM | 2759 | N | ARG | 345 | 8.514 | −0.443 | 13.373 | 1.00 | 0.00 | 1MOD |
| ATOM | 2760 | CA | ARG | 345 | 8.017 | −1.789 | 13.458 | 1.00 | 0.00 | 1MOD |
| ATOM | 2761 | C | ARG | 345 | 9.213 | −2.696 | 13.396 | 1.00 | 0.00 | 1MOD |
| ATOM | 2762 | O | ARG | 345 | 9.793 | −2.865 | 12.326 | 1.00 | 0.00 | 1MOD |
| ATOM | 2763 | CB | ARG | 345 | 7.172 | −2.149 | 12.226 | 1.00 | 0.00 | 1MOD |
| ATOM | 2764 | CG | ARG | 345 | 5.895 | −1.317 | 12.091 | 1.00 | 0.00 | 1MOD |
| ATOM | 2765 | CD | ARG | 345 | 5.460 | −1.081 | 10.641 | 1.00 | 0.00 | 1MOD |
| ATOM | 2766 | NE | ARG | 345 | 5.746 | −2.314 | 9.855 | 1.00 | 0.00 | 1MOD |
| ATOM | 2767 | CZ | ARG | 345 | 5.601 | −2.289 | 8.498 | 1.00 | 0.00 | 1MOD |
| ATOM | 2768 | NH1 | ARG | 345 | 5.152 | −1.155 | 7.888 | 1.00 | 0.00 | 1MOD |
| ATOM | 2769 | NH2 | ARG | 345 | 5.910 | −3.391 | 7.752 | 1.00 | 0.00 | 1MOD |
| ATOM | 2770 | N | PRO | 346 | 9.707 | −3.158 | 14.505 | 1.00 | 0.00 | 1MOD |
| ATOM | 2771 | CA | PRO | 346 | 10.774 | −4.128 | 14.368 | 1.00 | 0.00 | 1MOD |
| ATOM | 2772 | C | PRO | 346 | 10.587 | −5.291 | 15.297 | 1.00 | 0.00 | 1MOD |
| ATOM | 2773 | O | PRO | 346 | 9.646 | −5.279 | 16.089 | 1.00 | 0.00 | 1MOD |
| ATOM | 2774 | CB | PRO | 346 | 12.054 | −3.384 | 14.726 | 1.00 | 0.00 | 1MOD |
| ATOM | 2775 | CG | PRO | 346 | 11.576 | −2.400 | 15.802 | 1.00 | 0.00 | 1MOD |
| ATOM | 2776 | CD | PRO | 346 | 10.137 | −2.071 | 15.375 | 1.00 | 0.00 | 1MOD |
| ATOM | 2777 | N | SER | 347 | 11.469 | −6.315 | 15.213 | 1.00 | 0.00 | 1MOD |
| ATOM | 2778 | CA | SER | 347 | 11.502 | −7.328 | 16.233 | 1.00 | 0.00 | 1MOD |
| ATOM | 2779 | C | SER | 347 | 12.270 | −6.830 | 17.429 | 1.00 | 0.00 | 1MOD |
| ATOM | 2780 | O | SER | 347 | 11.707 | −6.336 | 18.406 | 1.00 | 0.00 | 1MOD |
| ATOM | 2781 | CB | SER | 347 | 12.200 | −8.620 | 15.775 | 1.00 | 0.00 | 1MOD |
| ATOM | 2782 | OG | SER | 347 | 12.205 | −9.568 | 16.834 | 1.00 | 0.00 | 1MOD |
| ATOM | 2783 | N | PHE | 348 | 13.617 | −6.965 | 17.346 | 1.00 | 0.00 | 1MOD |
| ATOM | 2784 | CA | PHE | 348 | 14.521 | −6.644 | 18.422 | 1.00 | 0.00 | 1MOD |
| ATOM | 2785 | C | PHE | 348 | 14.654 | −5.170 | 18.638 | 1.00 | 0.00 | 1MOD |
| ATOM | 2786 | O | PHE | 348 | 14.676 | −4.706 | 19.778 | 1.00 | 0.00 | 1MOD |
| ATOM | 2787 | CB | PHE | 348 | 15.915 | −7.282 | 18.267 | 1.00 | 0.00 | 1MOD |
| ATOM | 2788 | CG | PHE | 348 | 16.632 | −6.727 | 17.087 | 1.00 | 0.00 | 1MOD |
| ATOM | 2789 | CD1 | PHE | 348 | 16.416 | −7.237 | 15.825 | 1.00 | 0.00 | 1MOD |
| ATOM | 2790 | CD2 | PHE | 348 | 17.527 | −5.693 | 17.247 | 1.00 | 0.00 | 1MOD |
| ATOM | 2791 | CE1 | PHE | 348 | 17.085 | −6.726 | 14.739 | 1.00 | 0.00 | 1MOD |
| ATOM | 2792 | CE2 | PHE | 348 | 18.199 | −5.179 | 16.165 | 1.00 | 0.00 | 1MOD |
| ATOM | 2793 | CZ | PHE | 348 | 17.978 | −5.695 | 14.910 | 1.00 | 0.00 | 1MOD |
| ATOM | 2794 | N | VAL | 349 | 14.728 | −4.387 | 17.543 | 1.00 | 0.00 | 1MOD |
| ATOM | 2795 | CA | VAL | 349 | 14.856 | −2.954 | 17.611 | 1.00 | 0.00 | 1MOD |
| ATOM | 2796 | C | VAL | 349 | 13.541 | −2.484 | 18.146 | 1.00 | 0.00 | 1MOD |
| ATOM | 2797 | O | VAL | 349 | 12.673 | −3.323 | 18.362 | 1.00 | 0.00 | 1MOD |
| ATOM | 2798 | CB | VAL | 349 | 15.054 | −2.353 | 16.238 | 1.00 | 0.00 | 1MOD |
| ATOM | 2799 | CG1 | VAL | 349 | 15.191 | −0.819 | 16.303 | 1.00 | 0.00 | 1MOD |
| ATOM | 2800 | CG2 | VAL | 349 | 16.252 | −3.046 | 15.583 | 1.00 | 0.00 | 1MOD |
| ATOM | 2801 | N | GLY | 350 | 13.417 | −1.165 | 18.446 | 1.00 | 0.00 | 1MOD |
| ATOM | 2802 | CA | GLY | 350 | 12.257 | −0.422 | 18.891 | 1.00 | 0.00 | 1MOD |
| ATOM | 2803 | C | GLY | 350 | 11.027 | −1.279 | 18.949 | 1.00 | 0.00 | 1MOD |
| ATOM | 2804 | O | GLY | 350 | 10.907 | −2.185 | 19.752 | 1.00 | 0.00 | 1MOD |
| ATOM | 2805 | N | SER | 351 | 10.043 | −1.027 | 18.093 | 1.00 | 0.00 | 1MOD |
| ATOM | 2806 | CA | SER | 351 | 8.819 | −1.785 | 18.094 | 1.00 | 0.00 | 1MOD |
| ATOM | 2807 | C | SER | 351 | 8.109 | −1.595 | 19.383 | 1.00 | 0.00 | 1MOD |
| ATOM | 2808 | O | SER | 351 | 7.214 | −2.371 | 19.715 | 1.00 | 0.00 | 1MOD |
| ATOM | 2809 | CB | SER | 351 | 8.927 | −3.307 | 17.850 | 1.00 | 0.00 | 1MOD |
| ATOM | 2810 | OG | SER | 351 | 9.272 | −4.021 | 19.025 | 1.00 | 0.00 | 1MOD |
| ATOM | 2811 | N | SER | 352 | 8.476 | −0.548 | 20.140 | 1.00 | 0.00 | 1MOD |
| ATOM | 2812 | CA | SER | 352 | 7.757 | −0.317 | 21.349 | 1.00 | 0.00 | 1MOD |
| ATOM | 2813 | C | SER | 352 | 6.522 | 0.384 | 20.911 | 1.00 | 0.00 | 1MOD |
| ATOM | 2814 | O | SER | 352 | 6.553 | 1.153 | 19.954 | 1.00 | 0.00 | 1MOD |
| ATOM | 2815 | CB | SER | 352 | 8.503 | 0.585 | 22.348 | 1.00 | 0.00 | 1MOD |
| ATOM | 2816 | OG | SER | 352 | 7.720 | 0.763 | 23.517 | 1.00 | 0.00 | 1MOD |
| ATOM | 2817 | N | SER | 353 | 5.388 | 0.124 | 21.581 | 1.00 | 0.00 | 1MOD |
| ATOM | 2818 | CA | SER | 353 | 4.195 | 0.801 | 21.173 | 1.00 | 0.00 | 1MOD |
| ATOM | 2819 | C | SER | 353 | 4.238 | 2.166 | 21.777 | 1.00 | 0.00 | 1MOD |
| ATOM | 2820 | O | SER | 353 | 4.979 | 2.408 | 22.729 | 1.00 | 0.00 | 1MOD |
| ATOM | 2821 | CB | SER | 353 | 2.899 | 0.115 | 21.639 | 1.00 | 0.00 | 1MOD |
| ATOM | 2822 | OG | SER | 353 | 1.771 | 0.851 | 21.189 | 1.00 | 0.00 | 1MOD |
| ATOM | 2823 | N | GLY | 354 | 3.450 | 3.102 | 21.218 | 1.00 | 0.00 | 1MOD |
| ATOM | 2824 | CA | GLY | 354 | 3.425 | 4.431 | 21.750 | 1.00 | 0.00 | 1MOD |
| ATOM | 2825 | C | GLY | 354 | 2.895 | 4.329 | 23.136 | 1.00 | 0.00 | 1MOD |
| ATOM | 2826 | O | GLY | 354 | 3.399 | 4.972 | 24.055 | 1.00 | 0.00 | 1MOD |
| ATOM | 2827 | N | HIS | 355 | 1.850 | 3.501 | 23.323 | 1.00 | 0.00 | 1MOD |
| ATOM | 2828 | CA | HIS | 355 | 1.321 | 3.355 | 24.640 | 1.00 | 0.00 | 1MOD |
| ATOM | 2829 | C | HIS | 355 | 1.845 | 2.070 | 25.187 | 1.00 | 0.00 | 1MOD |
| ATOM | 2830 | O | HIS | 355 | 1.725 | 1.017 | 24.562 | 1.00 | 0.00 | 1MOD |
| ATOM | 2831 | CB | HIS | 355 | −0.210 | 3.272 | 24.705 | 1.00 | 0.00 | 1MOD |
| ATOM | 2832 | CG | HIS | 355 | −0.714 | 3.381 | 26.111 | 1.00 | 0.00 | 1MOD |
| ATOM | 2833 | ND1 | HIS | 355 | −0.653 | 2.369 | 27.041 | 1.00 | 0.00 | 1MOD |
| ATOM | 2834 | CD2 | HIS | 355 | −1.291 | 4.438 | 26.748 | 1.00 | 0.00 | 1MOD |
| ATOM | 2835 | CE1 | HIS | 355 | −1.191 | 2.859 | 28.187 | 1.00 | 0.00 | 1MOD |
| ATOM | 2836 | NE2 | HIS | 355 | −1.591 | 4.111 | 28.058 | 1.00 | 0.00 | 1MOD |
| ATOM | 2837 | N | THR | 356 | 2.472 | 2.140 | 26.373 | 1.00 | 0.00 | 1MOD |
| ATOM | 2838 | CA | THR | 356 | 2.976 | 0.966 | 27.015 | 1.00 | 0.00 | 1MOD |
| ATOM | 2839 | C | THR | 356 | 2.861 | 1.229 | 28.473 | 1.00 | 0.00 | 1MOD |
| ATOM | 2840 | O | THR | 356 | 2.546 | 2.345 | 28.883 | 1.00 | 0.00 | 1MOD |
| ATOM | 2841 | CB | THR | 356 | 4.425 | 0.699 | 26.735 | 1.00 | 0.00 | 1MOD |
| ATOM | 2842 | OG1 | THR | 356 | 5.226 | 1.761 | 27.233 | 1.00 | 0.00 | 1MOD |
| ATOM | 2843 | CG2 | THR | 356 | 4.620 | 0.554 | 25.214 | 1.00 | 0.00 | 1MOD |
| TER | 2844 | | THR | 356 | | | | | | 1MOD |

| | | |
|---|---|---|
| MASTER | 0 0 0 0 0 0 0 0 2843 1 0 | 1MOD |
| END | | 1MOD |

1. An assay for a candidate compound capable of allosterically modulating a GPCR, said assay comprising the steps of:
a) contacting said candidate compound with a GPCR or a mutant, variant, homologue, derivative or fragment thereof; and
b) detecting whether said candidate compound forms associations with one or more amino acid residues corresponding to any one of amino acid residues S81, V82 T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 or to 1, any one of amino acid residues 301 to 360 of CXCR2.

2. An assay as claimed in claim 1 wherein step b) comprises detecting whether said candidate compound forms associations with one or more amino acid residues corresponding to any one of amino acid residues S81, V82, T83, D84, Y86, L87, L90, G133, L136, I140, D143, R144, A147, Q157, Q245, K246, A249, V252, I253, V256, I259, L309, N310, P311, I313, Y314, I317, G318, Q319, K320, F321 or to any one of amino acid residues 301 to 360 of CXCR2.

3. An assay as claimed in claim 1 wherein step b) comprises detecting whether said candidate compound forms associations with one or more amino acid residues corresponding to any one of amino acid residues S81, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 or to any one of amino acid residues 304 to 326 of CXCR2.

4. An assay as claimed in claim 1 wherein step b) comprises detecting whether said candidate compound forms associations with one or more amino acid residues corresponding to any one of amino acid residues S81, V82. T83, D84, Y86, L87, L90, G133, L136, I140, D143, R144, A147, Q 157, Q245, K246, A249, V252, V 253, V256, I259, L309, N310, P311, I313, Y314, I317, G318, Q319, K320, F321 or to any one of amino acid residues 304 to 326 of CXCR2.

5. An assay as claimed in claim 1 wherein the GPCR in step a) is a polypeptide comprising amino acid residues corresponding to all or part of residues 301 to 360 of CXCR2.

6. An assay as claimed in claim 5 wherein the GPCR in step a) is a polypeptide comprising amino acid residues corresponding to residues 301 to 360 of CXCR2.

7. An assay as claimed in claim 3 wherein the GPCR in step a) is a polypeptide comprising amino acid residues corresponding to all or part residues 304 to 326 of CXCR2.

8. An assay as claimed in claim 7 wherein the GPCR in step a) is a polypeptide comprising amino acid residues corresponding to residues 304 to 326 of CXCR2.

9. An assay as claimed in claim 1 wherein step 1) comprises detecting whether said candidate compound forms associations with one or more amino acid residues corresponding to any one of amino acid residues S81, V82, T83, D84, Y86, L87, L90, G133, L136, L137, I140, D143, R144, A147, Q157, Q245, K246, A249, V252, I253, V256, I259, L309, N310, P311, I113, Y314, I317, G318, Q319, K320, F321 of CXCR2.

10. An assay as claimed in claim 1 wherein step b) comprises detecting whether said candidate compound forms associations with one or more amino acid residues corresponding to any one of amino acid residues S81, V82, T83, D84, Y86, L87, L₉₀, G133, L136, I140, D143, R144, A 147, Q157, Q245, K246, A249, V252, I253, V256, I259, L309, N310, P311, I133, Y314, I317, G318, Q319, K320, F321 of CXCR2.

11. An assay as claimed in claim 1 wherein step b) comprises detecting whether said candidate compound forms associations with one or more amino acid residues corresponding to any one of amino acid residues 301 to 360 of CXCR2.

12. An assay as claimed in claim 1 wherein step b) comprises detecting whether said candidate compound forms associations with one or more amino acid residues corresponding to any one of amino acid residues 304 to 326 of CXCR2.

13. An assay as claimed in claim 1 wherein step b) comprises detecting whether said candidate compound forms associations with an amino acid residue corresponding to amino acid residue K320 of CXCR2.

14. A competitive binding assay for a Candidate Compound X capable of allosterically modulating a GPCR which comprises the steps of:
i) providing a GPCR polypeptide comprising amino acid residues corresponding to all or part of residues 301 to 360 of CXCR2;
ii) contacting said polypeptide with a binding agent;
iii) contacting said polypeptide with a Candidate Compound X; and
iv) detecting displacement of the binding agent as an indication of the Candidate Compound X being capable of modulating said GPCR.

15. A competitive binding assay as claimed in claim 14 wherein the polypeptide comprises residues corresponding to residues 301 to 360 of CXCR2.

16. A competitive binding assay as claimed in claim 14 wherein the polypeptide comprises residues corresponding to residues 304 to 326 of CXCR2.

17. A competitive binding assay as claimed in claim 14 wherein the polypeptide comprises residues corresponding to residues 318 to 360 of CXCR2.

18. A competitive binding assay as claimed in claim 14 wherein the binding agent is a Candidate Compound Y identified by the assay as claimed in claim 1, or a pharmaceutically acceptable salt thereof.

19. A competitive binding assay as claimed in claim 14 wherein the binding agent is selected from the group consisting of Compound A or a pharmaceutically acceptable salt thereof, Compound B or a pharmaceutically acceptable salt thereof, Compound C or a pharmaceutically acceptable salt thereof and Compound F or a pharmaceutically acceptable salt thereof, wherein:
Compound A is 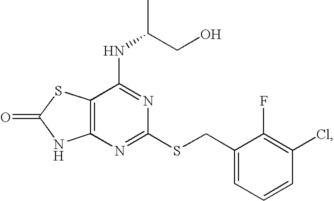
Compound B is 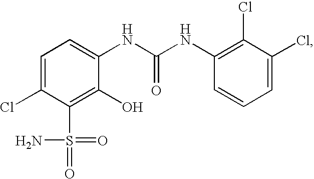
compound C is 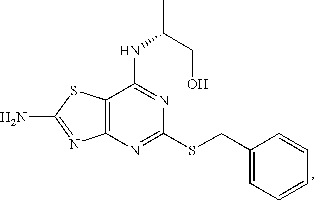
and Compound F is

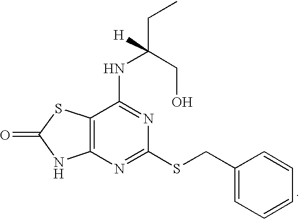

20. A competitive binding assay as claimed n claim 15 wherein the binding agent is Compound C or a pharmaceutically acceptable salt thereof.

21. An assay as claimed in claim 1 wherein the assay is a membrane assay.

22. An assay as claimed in claim 1 wherein the assay is a whole cell assay.

23. An assay as claimed in claim 1 wherein the GPCR is a Class A receptor.

24. An assay as claimed claim 1 wherein the GPCR is selected from the group consisting of CCR1, CCR2, CCR2A, CCR2B, CCR3, CCR4, CCR5, CCR6, CCR7, CCR8, CCR9, CCR10, CCR11, CXCR3, CXCR2, CXCR3, CXCR4, CXCR5 and CX₃CR1.

25. An assay as claimed in claim 1 wherein the GPCR is a Chemokine receptor and is selected from the group consisting of CCR1, CCR2, CX₃CR1, CCR4, CCR5 and CCR7 and a C—X—C family receptor.

26. An assay as claimed in claim 25 wherein the GPCR is selected from the group consisting of CXCR1 and CXCR2.

27. An assay as claimed in claim 26 wherein the GPCR is CXCR2.

28. A method of designing or screening for an intracellular allosteric modulator of a GPCR comprising the steps of:
a) providing at least a portion of the structure co-ordinates of the GPCR corresponding to those set out in Table 3;
b) employing at least a portion of the structure co-ordinates corresponding to those set out in Table 3 to design or select a putative allosteric modulator of the GPCR, wherein the portion employed corresponds to the amino acids which define the intracellular region of the GPCR;
c) obtaining or synthesising the putative allosteric modulator of the GPCR;
d) contacting the putative allosteric modulator of the GPCR with the GPCR or a mutant, variant, homologue, derivative or fragment thereof; and
e) determining whether said putative allosteric modulator of the GPCR modulates said GPCR.

29. A method as claimed in claim 28 wherein the putative allosteric modulator interacts with any one or more amino acid residues corresponding to any one of amino acid residues S81, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, F321 or to any one of amino acid residues 301 to 360 of CXCR2.

30. A method as claimed in claim 28 wherein the putative allosteric modulator interacts with any one or more amino acid residues corresponding to any one of amino acid residues S81, V82, T83, D84, Y86, L87, L90, G133, L136, I140, D143, R144, A147, Q157, Q245, K2461, A249, V252, I253, V256, I259, L309, N310, P311, I313, Y314, I317, G318, Q319, K320, F321 or to any one of amino acid residues 301 to 360 of CXCR2.

31. A method as claimed in claim 28 wherein the putative allosteric modulator interacts with any one or more amino acid residues corresponding to any one of amino acid residues S81, V82, T83, D84, Y86, L87, L90, G133, L136, L137, I140, D143, R144, A147, Q157, Q245, K246, A249, V252, I253, V256, I259, L309, N310, P311, I313, Y314, I317, G318, Q319, K320, or F321 of CXCR2.

32. A method as claimed in claim 28 wherein the putative allosteric modulator interacts with any one or more amino acid residues corresponding to any one of amino acid residues S81, V82, T83, D84, Y86, L87, L90, G133, L136, I140, D143, R144, A147, Q157, Q245, K246, A249, V252, I253, V256, I259, L309, N310, P313, Y314, I317, G318, Q319, K320, or F321 of CXCR2.

33. A method as claimed in claim 28 wherein the putative allosteric modulator interacts with a amino acid residue corresponding to K320 of CXCR2.

34. A method as claimed in claim 28 wherein the putative allosteric modulator interacts with any one or more amino acid residues corresponding to any one of amino acid residues 301 to 360 of CXCR2.

35. A method as claimed in claim 28 wherein the putative allosteric modulator interacts with any one or more amino acid residues corresponding to any one of amino acid residues 304 to 326 of CXCR2.

36. A method as claimed in claim 28 wherein the GPCR is a Class A receptor.

37. A method as claimed in claim 28 wherein the GPCR is selected from the group consisting of CCR1, CCR2, CCR2A, CCR2B, CCR3, CCR4, CCR5, CCR6, CCR7, CCR8, CCR9, CCR10, CCR11, CXCR1, CXCR2, CXCR3, CXCR4, CXCR5 and CX₃CR1.

38. A method as claimed in claim 28 wherein the GPCR is a Chemokine receptor and is selected from the group consisting of CCR1, CCR2, CX₃CR1, CCR4, CCR5 and CCR7 and a C—X—C family receptor.

39. A method as claimed in claim 38 wherein the GPCR is selected from the group consisting of CXCR1 and CXCR2.

40. A method as claimed in claim 39 wherein the GPCR is CXCR2.

41. A process comprising the steps of
(a) performing the assay of claim 1;
(b) identifying one or more modulators of a GPCR; and
(c) preparing a quantity of said one or more GPCR modulators.

42. A process comprising the steps of:
(a) performing the assay of claim 1;
(b) identifying one or more GPCR modulators, and
(c) preparing a pharmaceutical composition comprising said one or more identified GPCR modulators.

43. A process comprising the steps of:
(a) performing the assay of claim 1;
(b) identifying one or more GPCR modulators;
(c) modifying said one or more GPCR modulators; and
(d) optionally preparing a pharmaceutical composition comprising said one or more GPCR modulators.


SEQUENCE LISTING (62 sequences)

SEQ ID NO: 1 - protein, 350 aa, Homo sapiens [sequence omitted]

SEQ ID NO: 2 - protein, 360 aa, Homo sapiens
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 3 - protein, 119 aa, Homo sapiens
AHMGQKHRAMRVIFAVVLIFLLCWLPYNLVLLADTLMRTQVIQETCERRNNIGRALDATEILGFLHSCLNPIIYAFIGQNFRHGFLKILAMHGLVSKEFLARHRVTSYTSSSVNVSSNL

SEQ ID NO: 4 - protein, 120 aa, Homo sapiens
AHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 5 - protein, 31 aa, Homo sapiens
NMTSYLLEFLDRARKKALIIIVNPIYVGERF

SEQ ID NO: 6 - protein, 31 aa, Homo sapiens
CLTDYLLGFLDRARKRAVIIVINPIYVGEKF

SEQ ID NO: 7 - protein, 31 aa, Homo sapiens
IMTNYLLEFLDRARKKALIIVMNPIYVGERF

SEQ ID NO: 8 - protein, 31 aa, Homo sapiens
SMTDYLLGFMDRARKKAMIVLLNPIYLGEKF

SEQ ID NO: 9 - protein, 31 aa, Homo sapiens
SMTDYLLGFLDRARKRALIIVINPIYVGEKF

SEQ ID NO: 10 - protein, 31 aa, Homo sapiens
SMTDYLMGLIDRARKRAVIVVLNPLYIGQKF

SEQ ID NO: 11 - protein, 31 aa, Homo sapiens
TMTDYLLGLIDRARERAVIVVVNPLYIGVKF

SEQ ID NO: 12 - protein, 31 aa, Homo sapiens
SITDYLLSFMDRARNKALVVAVNPIYVGEKF

SEQ ID NO: 13 - protein, 31 aa, Homo sapiens
TMTDFLLCLIDRAKSKAVTVVLNPLYVGERF

SEQ ID NO: 14 - protein, 31 aa, Homo sapiens
SPTSHLLGFIDRASERAVVLALNPLYLGLRF

SEQ ID NO: 15 - protein, 31 aa, Homo sapiens
TKTDYILGFIDRAGKIPVLVVLNPLYMGASF

SEQ ID NO: 16 - protein, 31 aa, Homo sapiens
SVTDYLLGLIDRAQQKAVIVILNPIYIGQNF

SEQ ID NO: 17 - protein, 31 aa, Homo sapiens
SVTDYLLGLIDRAQQKAVIVILNPIYIGQKF

SEQ ID NO: 18 - protein, 31 aa, Homo sapiens
SSTDFLLGLIDRNGRRALVVALNPLYVGVKF

SEQ ID NO: 19 - protein, 31 aa, Homo sapiens
SMTDYRLSIIDRARQKATTLALNPLYLGAKF

SEQ ID NO: 20 - protein, 31 aa, Homo sapiens
SSTEFLLSLIDRARQRAVAVILNPLYAGVKF

SEQ ID NO: 21 - protein, 31 aa, Homo sapiens
SLTDFLLSIIDRVKQKSIIVVLNPLYVSLKF

SEQ ID NO: 22 - protein, 31 aa, Homo sapiens
SVTDYLLSFIDRARKKALIVVLNPIYAGEKF

SEQ ID NO: 23 - protein, 68 aa, Homo sapiens
IGRALDATEILGFLHSCLNPIIYAFIGQNFRHGFLKILAMHGLVSKEFLARHRVTSYTSSSVNVSSNL

SEQ ID NO: 24 - protein, 69 aa, Homo sapiens
IDRALDATEILGILHSCLNPLIYAFIGQKFRHGLLKILAIHGLISKDSLPKDSRPSFVGSSSGHTSTTL

SEQ ID NO: 25 - protein, 78 aa, Homo sapiens
LDQATQVTETLGMTHCCINPIIYAFVGEKFRRYLSVFFRKHITKRFCKQCPVFYRETVDGVTSTNTPSTGEQEVSAGL

SEQ ID NO: 26 - protein, 85 aa, Homo sapiens
LRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 27 - protein, 79 aa, Homo sapiens
LDYAIQATETLAFVHCCLNPIIYFFLGEKFRKYILQLFKTCRGLFVLCQYCGLLQIYSADTPSSSYTQSTMDHDLHDAL

SEQ ID NO: 28 - protein, 78 aa, Homo sapiens
LDQAMQVTETLGMTHCCINPIIYAFVGEKFRNYLLVFFQKHIAKRFCKCCSIFQQEAPERASSVYTRSTGEQEISVGL

SEQ ID NO: 29 - protein, 75 aa, Homo sapiens
LNIAYDVTYSLACVRCCVNPFLYAFIGVKFRNDLFKLFKDLGCLSQEQLRQWSSCRHIRRSSMSVEAETTTTFSP

SEQ ID NO: 30 - protein, 348 aa, Homo sapiens
MNGTEGPNFYVPFSNKTGVVRSPFEAPQYYLAEPWQFSMLAAYMFLLIMLGFPINFLTLYVTVQHKKLRTPLNYILLNLAVADLFMVFGGFTTTLYTSLHGYFVFGPTGCNLEGFFATLGGEIALWSLVVLAIERYVVVCKPMSNFRFGENHAIMGVAFTWVMALACAAPPLVGWSRYIPEGMQCSCGIDYYTPHEETNNESFVIYMFVVHFIIPLIVIFFCYGQLVFTVKEAAAQQQESATTQKAEKEVTRMVIIMVIAFLICWLPYAGVAFYIFTHQGSDFGPIFMTIPAFFAKTSAVYNPVIYIMMNKQFRNCMVTTLCCGKNPLGDDEASTTVSKTETSQVAPA

SEQ ID NO: 31 - protein, 355 aa, Homo sapiens
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 32 - protein, 374 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRSLFHIALGCRIAPLQKPVCGGPGVRPGKNVKVTTQGLLDGRGKGKSIGRAPEASLQDKEGA

SEQ ID NO: 33 - protein, 355 aa, Homo sapiens
MTTSLDTVETFGTTSYYDDVGLLCEKADTRALMAQFVPPLYSLVFTVGLLGNVVVVMILIKYRRLRIMTNIYLLNLAISDLLFLVTLPFWIHYVRGHNWVFGHGMCKLLSGFYHTGLYSEIFFIILLTIDRYLAIVHAVFALRARTVTFGVITSIVTWGLAVLAALPEFIFYETEELFEETLCSALYPEDTVYSWRHFHTLRMTIFCLVLPLLVMAICYTGIIKTLLRCPSKKKYKAIRLIFVIMAVFFIFWTPYNVAILLSSYQSILFGNDCERSKHLDLVMLVTEVIAYSHCCMNPVIYAFVGERFRKYLRHFFHRHLLMHLGRYIPFLPSEKLERTSSVSPSTAEPELSIVF

SEQ ID NO: 34 - protein, 360 aa, Homo sapiens
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 35 - protein, 352 aa, Homo sapiens [sequence omitted]

SEQ ID NO: 36 - protein, 374 aa, Homo sapiens
MSGESMNFSDVFDSSEDYFVSVNTSYYSVDSEMLLCSLQEVRQFSRLFVPIAYSLICVFGLLGNILVVITFAFYKKARSMTDVYLLNMAIADILFVLTLPFWAVSHATGAWVFSNATCKLLKGIYAINFNCGMLLLTCISMDRYIAIVQATKSFRLRSRTLPRSKIICLVVWGLSVIISSSTFVFNQKYNTQGSDVCEPKYQTVSEPIRWKLLMLGLELLFGFFIPLMFMIFCYTFIVKTLVQAQNSKRHKAIRVIIAVVLVFLACQIPHNMVLLVTAANLGKMNRSCQSEKLIGYTKTVTEVLAFLHCCLNPVLYAFIGQKFRNYFLKILKDLWCVRRKYKSSGFSCAGRYSENISRQTSETADNDNASSFTM

SEQ ID NO: 37 - protein, 378 aa, Homo sapiens
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 38 - protein, 355 aa, Homo sapiens
MDYTLDLSVTTVTDYYYPDIFSSPCDAELIQTNGKLLLAVFYCLLFVFSLLGNSLVILVLVVCKKLRSITDVYLLNLALSDLLFVFSFPFQTYYLLDQWVFGTVMCKVVSGFYYIGFYSSMFFITLMSVDRYLAVVHAVYALKVRTIRMGTTLCLAVWLTAIMATIPLLVFYQVASEDGVLQCYSFYNQQTLKWKIFTNFKMNILGLLIPFTIFMFCYIKILHQLKRCQNHNKTKAIRLVLIVVIASLLFWVPFNVVLFLTSLHSMHILDGCSISQQLTYATHVTEIISFTHCCVNPVIYAFVGEKFKKHLSEIFQKSCSQIFNYLGRQMPRESCEKSSSCQQHSSRSSSVDYIL

SEQ ID NO: 39 - protein, 357 aa, Homo sapiens
MADDYGSESTSSMEDYVNFNFTDFYCEKNNVRQFASHFLPPLYWLVFIVGALGNSLVILVYWYCTRVKTMTDMFLLNLAIADLLFLVTLPFWAIAAADQWKFQTFMCKVVNSMYKMNFYSCVLLIMCISVDRYIAIAQAMRAHTWREKRLLYSKMVCFTIWVLAAALCIPEILYSQIKEESGIAICTMVYPSDESTKLKSAVLTLKVILGFFLPFVVMACCYTIIIHTLIQAKKSSKHKALKVTITVLTVFVLSQFPYNCILLVQTIDAYAMFISNCAVSTNIDICFQVTQTIAFFHSCLNPVLYVFVGERFRRDLVKTLKNLGCISQAQWVSFTRREGSLKLSSMLLETTSGALSL

SEQ ID NO: 40 - protein, 362 aa, Homo sapiens
MGTEATEQVSWGHYSGDEEDAYSAEPLPELCYKADVQAFSRAFQPSVSLTVAALGLAGNGLVLATHLAARRAARSPTSAHLLQLALADLLLALTLPFAAAGALQGWSLGSATCRTISGLYSASFHAGFLFLACISADRYVAIARALPAGPRPSTPGRAHLVSVIVWLLSLLLALPALLFSQDGQREGQRRCRLIFPEGLTQTVKGASAVAQVALGFALPLGVMVACYALLGRTLLAARGPERRRALRVVVALVAAFVVLQLPYSLALLLDTADLLAARERSCPASKRKDVALLVTSGLALARCGLNPVLYAFLGLRFRQDLRRLLRGGSSPSGPQPRRGCPRRPRLSSCSAPTETHSLSWDN

SEQ ID NO: 41 - protein, 368 aa, Homo sapiens
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 42 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 43 - protein, 372 aa, Homo sapiens
MNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 44 - protein, 342 aa, Homo sapiens
MAEHDYHEDYGFSSFNDSSQEEHQDFLQFSKVFLPCMYLVVFVCGLVGNSLVLVISIFYHKLQSLTDVFLVNLPLADLVFVCTLPFWAYAGIHEWVFGQVMCKSLLGIYTINFYTSMLILTCITVDRFIVVVKATKAYNQQAKRMTWGKVTSLLIWVISLLVSLPQIIYGNVFNLDKLICGYHDEAISTVVLATQMTLGFFLPLLTMIVCYSVIIKTLLHAGGFQKHRSLKIIFLVMAVFLLTQMPFNLMKFIRSTHWEYYAMTSFHYTIMVTEAIAYLRACLNPVLYAFVSLKFRKNFWKLVKDIGCLPYLGVSHQWKSSEDNSKTFSASHNVEATSMFQL

SEQ ID NO: 45 - protein, 355 aa, Homo sapiens
MDQFPESVTENFEYDDLAEACYIGDIVVFGTVFLSIFYSVIFAIGLVGNLLVVFALTNSKKPKSVTDIYLLNLALSDLLFVATLPFWTHYLINEKGLHNAMCKFTTAFFFIGFFGSIFFITVISIDRYLAIVLAANSMNNRTVQHGVTISLGVWAAAILVAAPQFMFTKQKENECLGDYPEVLQEIWPVLRNVETNFLGFLLPLLIMSYCYFRIIQTLFSCKNHKKAKAIKLILLVVIVFFLFWTPYNVMIFLETLKLYDFFPSCDMRKDLRLALSVTETVAFSHCCLNPLIYAFAGEKFRRYLYHLYGKCLAVLCGRSVHVDFSSSESQRSRHGSVLSSNFTYHTSDGDALLLL

SEQ ID NO: 46 - protein, 333 aa, Homo sapiens
MESSGNPESTTFFYYDLQSQPCENQAWVFATLATTVLYCLVFLLSLVGNSLVLWVLVKYESLESLTNIFILNLCLSDLVFACLLPVWISPYHWGWVLGDFLCKLLNMIFSISLYSSIFFLTIMTIHRYLSVVSPLSTLRVPTLRCRVLVTMAVWVASILSSILDTIFHKVLSSGCDYSELTWYLTSVYQHNLFFLLSLGIILFCYVEILRTLFRSRSKRRHRTVKLIFAIVVAYFLSWGPYNFTLFLQTLFRTQIIRSCEAKQQLEYALLICRNLAFSHCCFNPVLYVFVGVKFRTHLKHVLRQFWFCRLQAPSPASIPHSPGAFAYEGASFY

SEQ ID NO: 47 - protein, 360 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 48 - DNA, 28 bp, Homo sapiens
ccatggattccttaagatcctggctatg

SEQ ID NO: 49 - DNA, 28 bp, Homo sapiens
catagccaggatcttaaggaatccatgg

SEQ ID NO: 50 - DNA, 30 bp, Homo sapiens
gccatggactccttaagattctagctatac

SEQ ID NO: 51 - DNA, 30 bp, Homo sapiens
gtatagctagaatcttaaggagtccatggc

SEQ ID NO: 52 - DNA, 31 bp, Homo sapiens
cttcatcggccaaaagtttcgccatggattc

SEQ ID NO: 53 - DNA, 31 bp, Homo sapiens
gaatccatggcgaaacttttggccgatgaag

SEQ ID NO: 54 - DNA, 34 bp, Homo sapiens
caaaattttcgccatggactcctcaagatcctgg

SEQ ID NO: 55 - DNA, 34 bp, Homo sapiens
ccaggatcttgaggagtccatggcgaaaattttg

SEQ ID NO: 56 - DNA, 32 bp, Homo sapiens
ccttcattggccagaattttcgccatggactc

SEQ ID NO: 57 - DNA, 32 bp, Homo sapiens
gagtccatggcgaaaattctggccaatgaagg

SEQ ID NO: 58 - DNA, 37 bp, Homo sapiens
catcggccaaaagtttcgccatggactcctcaagatc

SEQ ID NO: 59 - DNA, 37 bp, Homo sapiens
gatcttgaggagtccatggcgaaacttttggccgatg

SEQ ID NO: 60 - DNA, 41 bp, Homo sapiens
cttcattggccagaattttcgccatggattcctcaagattc

SEQ ID NO: 61 - DNA, 41 bp, Homo sapiens
gaatcttgaggaatccatggcgaaaattctggccaatgaag

SEQ ID NO: 62 - protein, 269 aa, Homo sapiens
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKGSLVPRGSGQKFRHGLLKILAIHGLISKDSLPKDSRPSFVGSSSGHTSTTL